(19) United States
(12) Patent Application Publication
(10) Pub. No.: US 2026/0022094 A1
(43) Pub. Date: Jan. 22, 2026

(54) Compounds for the Treatment of Neurodegenerative Diseases

(71) Applicant: Celgene Corporation, Princeton, NJ (US)

(72) Inventors: Jean-Francois Brazeau, San Diego, CA (US); Rulin Ma, Winchester, MA (US); Jeffrey M. Schkeryantz, Summit, NJ (US); Karin Worm, Summit, NJ (US); Patrick W. Papa, Carlsbad, CA (US)

(73) Assignee: Celgene Corporation, Princeton, NJ (US)

(21) Appl. No.: 18/992,821

(22) PCT Filed: Jul. 17, 2023
(86) PCT No.: PCT/US2023/027873

§ 371 Date: Jan. 9, 2025

Related U.S. Application Data
(60) Provisional application No. 63/390,069, filed on Jul. 18, 2022.

(51) Int. Cl. C07D 205/04; A61K 31/135; A61K 31/397; A61K 31/40; A61K 31/4025; A61K 31/4427; A61K 31/451; A61K 31/4523; C07C 217/74; C07D 207/12; C07D 211/48; C07D 401/10; C07D 401/14; C07D 403/10

(52) U.S. Cl. CPC C07D 205/04; A61K 31/135; A61K 31/397; A61K 31/40; A61K 31/4025; A61K 31/4427; A61K 31/451; A61K 31/4523; C07C 217/74; C07D 207/12; C07D 211/48; C07D 401/10; C07D 401/14; C07D 403/10; C07C 2602/08

(57) ABSTRACT

Provided herein are compounds and compositions thereof for modulating S1P5. In some embodiments, the compounds and compositions are provided for treatment of neurological diseases.

### CROSS-REFERENCE TO RELATED APPLICATIONS

This application claims priority to U.S. Provisional Application No. 63/390,069, filed on Jul. 18, 2022, the disclosure of which is incorporated herein by reference in its entirety for any purpose.

### FIELD

The present disclosure relates generally to compounds, compositions, and methods for their preparation and use of the compounds and compositions for treating neurological diseases.

### BACKGROUND

Sphingosine-1-phosphate (S1P; (2S,3R,4E)-2-amino-3-hydroxyoctadec-4-enyl-1-phosphate) is a bioactive sphingolipid that is synthesized by metabolic turnover of sphingolipids in cells and by the extracellular action of a secreted sphingosine kinase. S1P binds to and stimulates members of the endothelial cell differentiation gene family (EDG receptors), which are plasma membrane-localized G protein-coupled receptors. The five members of this family of receptors are S1P1 (EDG-1), S1P2 (EDG-5), S1P3 (EDG-3), S1P4 (EDG-6), and S1P5 (EDG-8). S1P mediates a wide variety of cellular responses including proliferation, cytoskeletal organization and migration, adherence- and tight junction assembly, and morphogenesis.

S1P5 is primarily expressed in the central nervous system. Specifically, S1P5 is highly expressed in oligodendrocytes (oligodendroglia) and oligodendrocyte progenitor cells (Jaillard, C. et al., J. Neuroscience, 2005, 25 (6), 1459-1469; Novgorodov, A. S. et al., FASEB J., 2007, 21, 1503-1514). Oligodendrocytes are glial cells that form myelin sheaths (myelin) by binding to the axons of nerve cells. Compounds that bind to S1P5 can modulate the function of S1P5 and may be useful for treating neurodegenerative diseases.

Accordingly, in one aspect, provided herein are compounds that modulate S1P5 for use in treating neurodegenerative diseases.

### SUMMARY

Described herein, in certain embodiments, are compounds and compositions thereof for modulating S1P5. In various embodiments, the compounds and compositions thereof may be used for treatment of neurodegenerative diseases.

The present embodiments can be understood more fully by reference to the detailed description and examples, which are intended to exemplify non-limiting embodiments.

Embodiment A1. A compound of Formula (I):

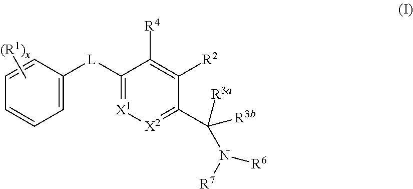

or a pharmaceutically acceptable salt thereof, wherein:
L is —C≡C—, —HC═CH—, —CH₂CH₂—, —CH₂O—,

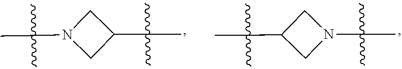 [SMILES: *C1CN(*)C1 ; *C1CN(*)C1]

or a bond;
each R¹ is independently halo, —CN, C₁-C₆ alkyl, C₁-C₆ haloalkyl, C₁-C₆ alkoxy, or C₃-C₆ cycloalkyl;
x is 0-5;
R² is H, halo, C₁-C₆ alkyl, C₃-C₆ cycloalkyl, or C₁-C₆ haloalkyl;
R^{3a} and R^{3b} are each H;
or R² and R^{3a} are taken together with the carbon atoms to which they are attached to form a fused cyclopentyl;
or R² and R⁴ are taken together with the carbon atoms to which they are attached to form a fused phenyl;
R⁴ is H, halo, —CN, C₁-C₆ alkyl, C₁-C₆ haloalkyl, C₁-C₆ alkoxy, or C₃-C₆ cycloalkyl;
X¹ and X² are independently N or CR⁵;
each R⁵ is independently H, halo, —CN, C₁-C₆ alkyl, C₁-C₆ haloalkyl, C₁-C₆ alkoxy, or C₃-C₆ cycloalkyl;
R⁶ is H;
R⁷ is C₁-C₆ alkyl-OH;
or R⁶ and R⁷ are taken together with the nitrogen atom to which they are attached to form a 4- to 6-membered heterocyclyl substituted with n R⁸ groups;
n is 1-5; and
each R⁸ is independently halo, —CN, C₁-C₆ alkyl, C₁-C₆ haloalkyl, C₁-C₆ alkoxy, or —OH, provided that at least one R⁸ is —OH.

Embodiment A2. The compound of embodiment A1, or a pharmaceutically acceptable salt thereof, wherein:
L is —C≡C—, —CH₂CH₂—, —CH₂O—, or a bond.

Embodiment A3. The compound of embodiment A1, or a pharmaceutically acceptable salt thereof, wherein:
L is

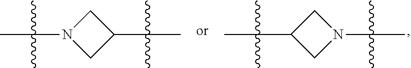 [SMILES: *C1CN(*)C1 ; *C1CN(*)C1]

Embodiment A4. The compound of any one of embodiments A1-A3, or a pharmaceutically acceptable salt thereof, wherein:
each R¹ is independently halo, —CN, C₁-C₃ alkyl, C₁-C₃ haloalkyl, C₁-C₃ alkoxy, or C₃-C₆ cycloalkyl.

Embodiment A5. The compound of any one of embodiments A1-A4, or a pharmaceutically acceptable salt thereof, wherein:

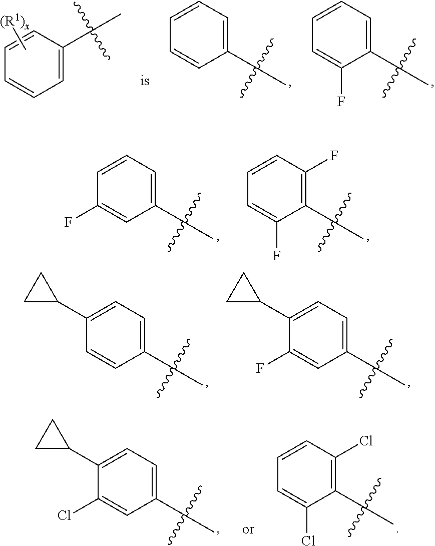

Embodiment A6. The compound of any one of embodiments A1-A5, or a pharmaceutically acceptable salt thereof, wherein:
R² is H, halo, C₁-C₃ alkyl, C₃-C₆ cycloalkyl, or C₁-C₃ haloalkyl; and
R^{3a} and R^{3b} are each H.

Embodiment A7. The compound of any one of embodiments A1-A5, or a pharmaceutically acceptable salt thereof, wherein:
R² and R^{3a} are taken together with the carbon atoms to which they are attached to form a fused cyclopentyl; and
R^{3b} is H.

Embodiment A8. The compound of any one of embodiments A1-A5, or a pharmaceutically acceptable salt thereof, wherein:
R² and R⁴ are taken together with the carbon atoms to which they are attached to form a fused phenyl.

Embodiment A9. The compound of any one of embodiments A1-A7, or a pharmaceutically acceptable salt thereof, wherein:
R⁴ is H, halo, —CN, C₁-C₃ alkyl, C₁-C₃ haloalkyl, C₁-C₃ alkoxy, or C₃-C₆ cycloalkyl.

Embodiment A10. The compound of any one of embodiments A1-A9, or a pharmaceutically acceptable salt thereof, wherein:
each R⁵ is independently H, halo, —CN, C₁-C₃ alkyl, C₁-C₃ haloalkyl, C₁-C₃ alkoxy, or C₃-C₆ cycloalkyl.

Embodiment A11. The compound of any one of embodiments A1-A10, or a pharmaceutically acceptable salt thereof, wherein:

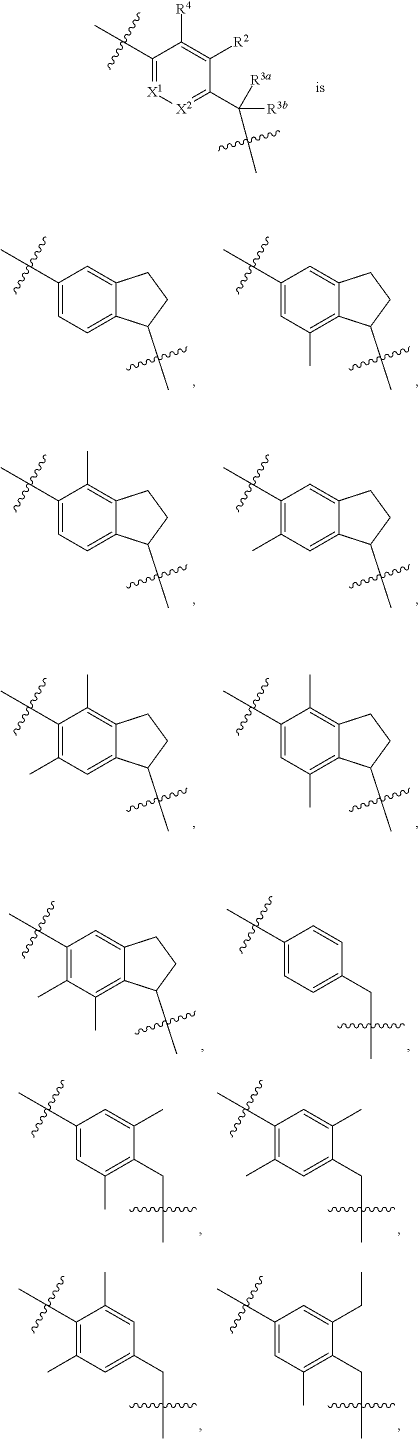

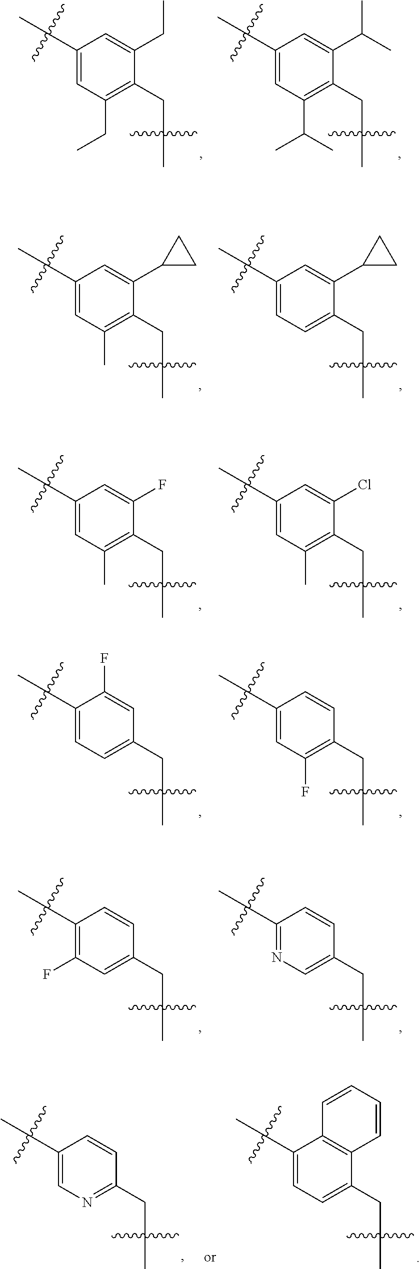

Embodiment A12. The compound of any one of embodiments A1-A11, or a pharmaceutically acceptable salt thereof, wherein:
R⁶ is H; and
R⁷ is C₁-C₆ alkyl-OH.

Embodiment A13. The compound of any one of embodiments A1-A11, or a pharmaceutically acceptable salt thereof, wherein:
R⁶ and R⁷ are taken together with the nitrogen atom to which they are attached to form

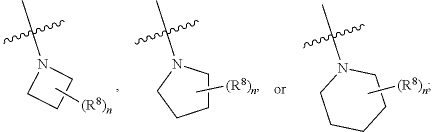

and
each R⁸ is independently halo, —CN, C₁-C₃ alkyl, C₁-C₃ haloalkyl, C₁-C₃ alkoxy, or —OH.

Embodiment A14. The compound of any one of embodiments A1-A11 and A13, or a pharmaceutically acceptable salt thereof, wherein:

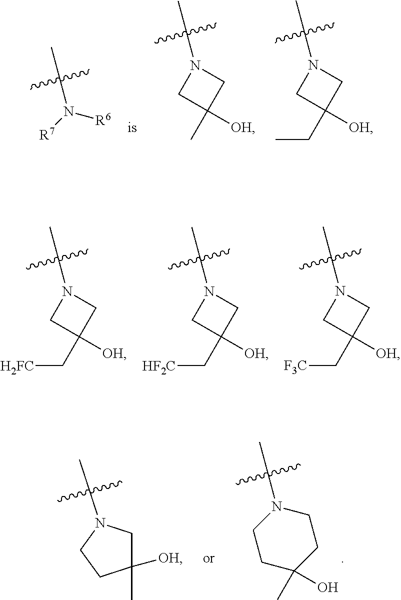

Embodiment A15. The compound of any one of embodiments A1-A5, A7, and A9-A14, or a pharmaceutically acceptable salt thereof, wherein the compound is of Formula (II):

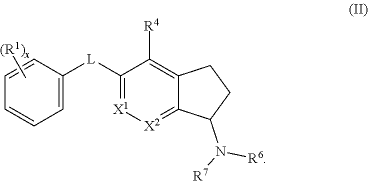

Embodiment A16. The compound of any one of embodiments A1-A6 and A8-A14, or a pharmaceutically acceptable salt thereof, wherein the compound is of Formula (III):

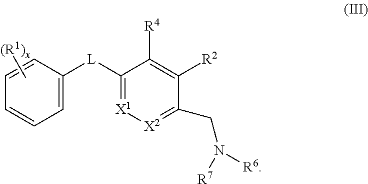

Embodiment A17. A compound selected from the compounds of Table 1 and pharmaceutically acceptable salts thereof.

Embodiment A18. A pharmaceutical composition comprising the compound of any one of embodiments A1-A17, or a pharmaceutically acceptable salt thereof, and a pharmaceutically acceptable excipient.

Embodiment A19. A method of modulating sphingosine 1-phosphate receptor 5 (S1P5) comprising contacting S1P5 with an effective amount of the compound of any one of embodiments A1-A17, or a pharmaceutically acceptable salt thereof, or the pharmaceutical composition of embodiment A18.

Embodiment A20. A method of treating a neurological disease in a subject in need thereof, comprising administering to the subject an effective amount of the compound of any one of embodiments A1-A17, or a pharmaceutically acceptable salt thereof, or the pharmaceutical composition of embodiment A18, optionally wherein the neurological disease is Alzheimer's disease, multiple sclerosis, migraine, and amyotrophic lateral sclerosis.

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, ±10%, ±5%, or ±1% of the indicated range, value, or structure, unless otherwise indicated.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈alkyl) or, in some embodiments, from 1 to 6 (C₁-C₆ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include-methyl, -ethyl, -n-propyl, -n-butyl, -n-pentyl and n-hexyl; while saturated branched alkyls include-isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH₃), —CH—C(CH₃)₂, —C(CH₃)—CH₂, —C(CH₃)—CH(CH₃), —C(CH₂CH₃)—CH₂, —C≡CH, —C≡C(CH₃), —C≡C(CH₂CH₃), —CH₂C≡CH, —CH₂C═C(CH₃) and —CH₂C═C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyloxy, cycloalkylalkyloxy, aralkyloxy, heterocyclylalkyloxy, heteroarylalkyloxy, heterocycloalkylalkyloxy; oxo (═O); amino, alkylamino, cycloalkylamino, arylamino, heterocyclylamino, heteroarylamino, heterocycloalkylamino, cycloalkylalkylamino, aralkylamino, heterocyclylalkylamino, heteroaralkylamino, heterocycloalkylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkyl-OH” group refers to an unbranched or branched alkyl group as defined above, wherein one or more hydrogen atoms are replaced by —OH. For example, “C₁-C₆ alkyl-OH” refers to a C₁-C₆ alkyl which is substituted by one or more-OH groups. An alkyl-OH may contain multiple hydroxy groups that are attached to the same carbon atom or to multiple carbon atoms.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings that can be optionally substituted. In some embodiments, the cycloalkyl group has 3 to 8 ring carbon atoms (C₃-C₈ cycloalkyl), whereas in other embodiments the number of ring carbon atoms ranges from 3 to 5 (C₃-C₈ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like In other embodiments, the cycloalkyl groups are unsaturated cycloalkyl groups. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

An “aryl” group is an aromatic carbocyclic group of from 6 to 14 carbon atoms (C₆-C₁₄ aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₄ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “halogen” or “halo” is fluorine, chlorine, bromine or iodine.

“Haloakyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl group has one to six carbon atoms and is substituted by one or more halo radicals (C₁-C₆ haloalkyl), or the haloalkyl group has one to three carbon atoms and is substituted by one or more halo radicals (C₁-C₃ haloalkyl). The halo radicals may be all the same or the halo radicals may be different. Unless specifically stated otherwise, a haloalkyl group is optionally substituted.

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1 (2H)-onyl), tetrahydroquinolinyl, quinoxalinyl, and quinazolinyl groups. A heteroaryl group can be substituted or unsubstituted.

A “heterocyclyl” is a non-aromatic cycloalkyl in which one to four of the ring carbon atoms are independently replaced with a heteroatom selected from O, S and N. In some embodiments, heterocyclyl groups include 3 to10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2 (1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

An “alkoxy” group is —O-(alkyl), wherein alkyl is defined above.

A “carboxy” group is a radical of the formula:—C(O) OH.

When the groups described herein, with the exception of alkyl group, are said to be “substituted,” they may be substituted with any appropriate substituent or substituents. IIIustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate, oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Embodiments of the disclosure are meant to encompass pharmaceutically acceptable salts, tautomers, isotopologues, and stereoisomers of the compounds provided herein, such as the compounds of Formula (I).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of formula (I) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag Gmbh & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuja, S., Chiral Separation Methods for Pharmaceutical and Biotechnological Products (John Wiley & Sons, 2011).

It should also be noted the compounds disclosed herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

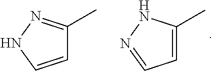

As readily understood by one skilled in the art, a wide variety of functional groups and other structures may exhibit tautomerism and all tautomers of compounds of Formula (I) are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (3H), iodine-125 (1251), sulfur-35 (35S), or carbon-14 (1+C), or may be isotopically enriched, such as with deuterium (2H), carbon-13 (13C), or nitrogen-15 (15N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically encriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or 2H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is a neurodegenerative disease, as described herein, or a symptom thereof.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder is a neurodegenerative disease, as described herein, or symptoms thereof.

The term “effective amount” in connection with a compound disclosed herein means an amount capable of treating or preventing a disorder, disease or condition, or symptoms thereof, disclosed herein.

The term “subject” or “patient” as used herein include an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having an S1P5 mediated disease, or a symptom thereof.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In one aspect, provided herein is a compound of Formula (I):

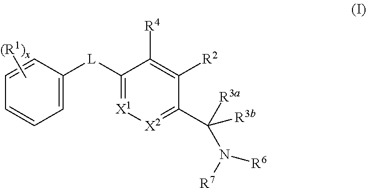

or a pharmaceutically acceptable salt thereof, wherein:
L is —C≡C—, —HC≡CH—, —CH₂CH₂—, —CH₂O—,

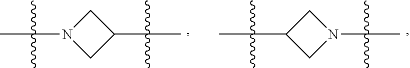 [SMILES: *C1CN(*)C1 ; *C1CN(*)C1]

or a bond;
each R¹ is independently halo, —CN, C₁-C₆ alkyl, C₁-C₆ haloalkyl, C₁-C₆ alkoxy, or C₃-C₆ cycloalkyl;
x is 0-5;
R² is H, halo, C₁-C₆ alkyl, C₃-C₆ cycloalkyl, or C₁-C₆ haloalkyl; R^{3a} and R^{3b} are each H;
or R² and R^{3a} are taken together with the carbon atoms to which they are attached to form a fused cyclopentyl;
or R² and R⁴ are taken together with the carbon atoms to which they are attached to form a fused phenyl;
R⁴ is H, halo, —CN, C₁-C₆ alkyl, C₁-C₆ haloalkyl, C₁-C₆ alkoxy, or C₃-C₆ cycloalkyl; X¹ and X² are independently N or CR⁵;
each R⁵ is independently H, halo, —CN, C₁-C₆ alkyl, C₁-C₆ haloalkyl, C₁-C₆ alkoxy, or C₃-C₆ cycloalkyl;
R⁶ is H;
R⁷ is C₁-C₆ alkyl-OH;
or R⁶ and R⁷ are taken together with the nitrogen atom to which they are attached to form a 4- to 6-membered heterocyclyl substituted with n R⁸ groups;
n is 1-5; and
each R⁸ is independently halo, —CN, C₁-C₆ alkyl, C₁-C₆ haloalkyl, C₁-C₆ alkoxy, or —OH, provided that at least one R⁸ is —OH.

In some embodiments, L is —C≡C—, —HC═CH—, —CH₂CH₂—, —CH₂O—,

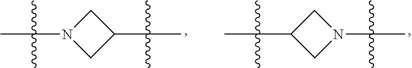 [SMILES: *C1CN(*)C1 ; *C1CN(*)C1]

or a bond. In some embodiments, L is —C≡C—, —CH₂CH₂—, or —CH₂O—. In some embodiments, L is

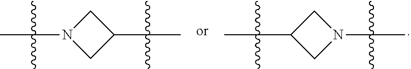 [SMILES: *C1CN(*)C1 ; *C1CN(*)C1]

In some embodiments, L is —C≡C—. In some embodiments, L is —HC═CH—. In some embodiments, L is —CH₂CH₂—. In some embodiments, L is —CH₂O—. In some embodiments, L is

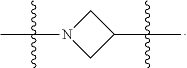 [SMILES: *C1CN(*)C1]

In some embodiments, L is

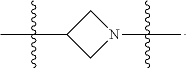 [SMILES: *C1CN(*)C1]

In some embodiments, L is a bond.

In some embodiments, each R¹ is independently halo, —CN, C₁-C₆ alkyl, C₁-C₆ haloalkyl, C₁-C₆ alkoxy, or C₃-C₆ cycloalkyl. In some embodiments, each R¹ is independently halo, —CN, C₁-C₃ alkyl, C₁-C₃ haloalkyl, C₁-C₃ alkoxy, or C₃-C₆ cycloalkyl. In some embodiments, each R¹ is independently F, C₁, or cyclopropyl.

In some embodiments, R¹ is halo. In some embodiments, R¹ is C₁, F, or Br. In some embodiments, R¹ is C₁. In some embodiments, R¹ is F. In some embodiments, R¹ is Br.

In some embodiments, R¹ is —CN.

In some embodiments, R¹ is C₁-C₆ alkyl. In some embodiments, R¹ is C₁-C₃ alkyl. In some embodiments, R¹ is methyl, ethyl, n-propyl, or isopropyl. In some embodiments, R¹ is methyl. In some embodiments, R¹ is ethyl. In some embodiments, R¹ is n-propyl. In some embodiments, R¹ is isopropyl.

In some embodiments, R¹ is C₁-C₆ haloalkyl. In some embodiments, R¹ is C₁-C₆ haloalkyl containing 1-13 halogen atoms. In some embodiments, R¹ is C₁-C₃ haloalkyl. In some embodiments, R¹ is C₁-C₃ haloalkyl containing 1-7 halogen atoms. In some embodiments, R¹ is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R¹ is —CF₃. In some embodiments, R¹ is —CHF₂.

In some embodiments, R¹ is C₁-C₆ alkoxy. In some embodiments, R¹ is C₁-C₃ alkoxy. In some embodiments, R¹ is —OCH3, —OCH₂CH₃, —OCH₂CH₂CH₃, or —OCH(CH₃)₂. In some embodiments, R¹ is —OCH₃. In some embodiments, R¹ is —OCH₂CH₃.

In some embodiments, R¹ is C₃-C₆ cycloalkyl. In some embodiments, R¹ is C₃-C₅ cycloalkyl. In some embodiments, R¹ is cyclopropyl, cyclobutyl, cyclopentyl, or cyclohexyl. In some embodiments, R¹ is cyclopropyl. In some embodiments, R¹ is cyclobutyl. In some embodiments, R¹ is cyclopentyl. In some embodiments, R¹ is cyclohexyl.

In some embodiments, x is 0-5. In some embodiments, x is 0, 1, or 2. In some embodiments, x is 0. In some embodiments, x is 1. In some embodiments, x is 2. In some embodiments, x is 3. In some embodiments, x is 4. In some embodiments, x is 5.

In some embodiments,

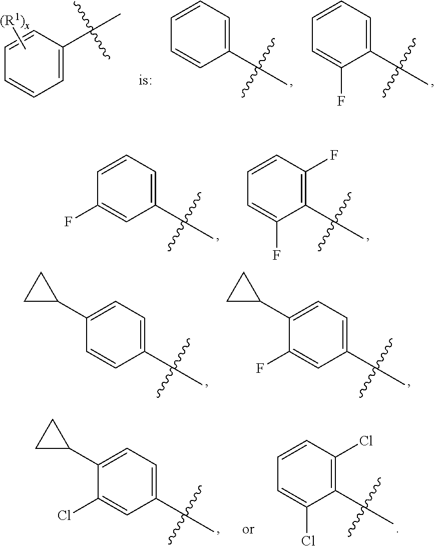

In some embodiments, R² is H, halo, C₁-C₆ alkyl, C₃-C₆ cycloalkyl, or C₁-C₆ haloalkyl. In some embodiments, R² is H, halo, C₁-C₃ alkyl, C₃-C₆ cycloalkyl, or C₁-C₃ haloalkyl. In some embodiments, R² is H, F, Cl, —CH₃, —CH₂CH₃, —CH(CH₃)₂, or cyclopropyl.

In some embodiments, R² is H.

In some embodiments, R² is halo. In some embodiments, R² is Cl, F, or Br. In some embodiments, R² is Cl. In some embodiments, R² is F. In some embodiments, R² is Br.

In some embodiments, R² is C₁-C₆ alkyl. In some embodiments, R² is C₁-C₃ alkyl. In some embodiments, R² is methyl, ethyl, n-propyl, or isopropyl. In some embodiments, R² is methyl. In some embodiments, R² is ethyl. In some embodiments, R² is n-propyl. In some embodiments, R² is isopropyl.

In some embodiments, R² is C₃-C₆ cycloalkyl. In some embodiments, R² is C₃-C₅ cycloalkyl. In some embodiments, R² is cyclopropyl, cyclobutyl, cyclopentyl, or cyclohexyl. In some embodiments, R² is cyclopropyl. In some embodiments, R² is cyclobutyl. In some embodiments, R² is cyclopentyl. In some embodiments, R² is cyclohexyl.

In some embodiments, R² is C₁-C₆ haloalkyl. In some embodiments, R² is C₁-C₆ haloalkyl containing 1-13 halogen atoms. In some embodiments, R² is C₁-C₃ haloalkyl. In some embodiments, R² is C₁-C₃ haloalkyl containing 1-7 halogen atoms. In some embodiments, R² is —CF₃, —CHF₂, —CH₂F, —CCl₃, —CHCl₂, —CH₂CI, —CF₂CI, —CFCl₂, —CH₂CF₃, —CH₂CHF₂, or —CH₂CCl₃. In some embodiments, R² is —CF₃. In some embodiments, R² is —CHF₂.

In some embodiments, R² and R^{3a} are taken together with the carbon atoms to which they are attached to form a fused cyclopentyl. In some variations, R^{3b} is H.

In some embodiments, R² and R⁴ are taken together with the carbon atoms to which they are attached to form a fused phenyl.

In some embodiments, R^{3a} and R^{3b} are each H.

In some embodiments, R⁴ is H, halo, —CN, C₁-C₆ alkyl, C₁-C₆ haloalkyl, C₁-C₆ alkoxy, or C₃-C₆ cycloalkyl. In some embodiments, Ris H, halo, —CN, C₁-C₃ alkyl, C₁-C₃ haloalkyl, C₁-C₃ alkoxy, or C₃-C₆ cycloalkyl. In some embodiments, R⁴ is H, F, or —CH₃.

In some embodiments, R⁴ is H.

In some embodiments, R⁴ is halo. In some embodiments, R⁴ is C₁, F, or Br. In some embodiments, R⁴ is C₁. In some embodiments, R⁴ is F. In some embodiments, R⁴ is Br.

In some embodiments, R⁴ is —CN.

In some embodiments, R⁴ is C₁-C₆ alkyl. In some embodiments, R⁴ is C₁-C₃ alkyl. In some embodiments, R⁴ is methyl, ethyl, n-propyl, or isopropyl. In some embodiments, R⁴ is methyl. In some embodiments, R⁴ is ethyl. In some embodiments, R⁴ is n-propyl. In some embodiments, R⁴ is isopropyl.

In some embodiments, R⁴ is C₁-C₆ haloalkyl. In some embodiments, R⁴ is C₁-C₆ haloalkyl containing 1-13 halogen atoms. In some embodiments, R⁴ is C₁-C₃ haloalkyl. In some embodiments, R⁴ is C₁-C₃ haloalkyl containing 1-7 halogen atoms. In some embodiments, R⁴ is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R⁴ is —CF₃. In some embodiments, R⁴ is —CHF₂.

In some embodiments, R⁴ is C₁-C₆ alkoxy. In some embodiments, R⁴ is C₁-C₃ alkoxy. In some embodiments, R⁴ is —OCH₃, —OCH₂CH₃, —OCH₂CH₂CH₃, or —OCH(CH₃)₂. In some embodiments, R⁴ is —OCH₃. In some embodiments, R⁴ is —OCH₂CH₃.

In some embodiments, R⁴ is C₃-C₆ cycloalkyl. In some embodiments, R⁴ is C₃-C₅ cycloalkyl. In some embodiments, R⁴ is cyclopropyl, cyclobutyl, cyclopentyl, or cyclohexyl. In some embodiments, R⁴ is cyclopropyl. In some embodiments, R⁴ is cyclobutyl. In some embodiments, R⁴ is cyclopentyl. In some embodiments, R⁴ is cyclohexyl.

In some embodiments, X¹ and X² are independently N or CR⁵. In some embodiments, X¹ and X² are independently CR⁵. In some embodiments, X¹ is N and X² is CR⁵. In some embodiments, X¹ is CR⁵, and X² is N. In some embodiments, X¹ and X² are each N.

In some embodiments, each R⁵ is independently H, halo, —CN, C₁-C₆ alkyl, C₁-C₆ haloalkyl, C₁-C₆ alkoxy, or C₃-C₆ cycloalkyl. In some embodiments, each R⁵ is independently H, halo, —CN, C₁-C₃ alkyl, C₁-C₃ haloalkyl, C₁-C₃ alkoxy, or C₃-C₆ cycloalkyl. In some embodiments, each R⁵ is independently H, F, —CH₃, —CH₂CH₃, or —CH(CH₃)₂.

In some embodiments, R⁵ is H.

In some embodiments, R⁵ is halo. In some embodiments, R⁵ is C₁, F, or Br. In some embodiments, R⁵ is C₁. In some embodiments, R⁵ is F. In some embodiments, R⁵ is Br.

In some embodiments, R⁵ is —CN.

In some embodiments, R⁵ is C₁-C₆ alkyl. In some embodiments, R⁵ is C₁-C₃ alkyl. In some embodiments, R⁵ is methyl, ethyl, n-propyl, or isopropyl. In some embodiments, R⁵ is methyl. In some embodiments, R⁵ is ethyl. In some embodiments, R⁵ is n-propyl. In some embodiments, R⁵ is isopropyl.

In some embodiments, R⁵ is C₁-C₆ haloalkyl. In some embodiments, R⁵ is C₁-C₆ haloalkyl containing 1-13 halogen atoms. In some embodiments, R⁵ is C₁-C₃ haloalkyl. In some embodiments, R⁵ is C₁-C₃ haloalkyl containing 1-7 halogen atoms. In some embodiments, R⁵ is —CF₃, —CHF₂, —CH₂F, —CCl₃, —CHCl₂, —CH₂CI, —CF₂CI, —CFCl₂, —CH₂CF₃, —CH₂CHF₂, or —CH₂CCl₃. In some embodiments, R⁵ is —CF₃. In some embodiments, R⁵ is —CHF₂.

In some embodiments, R⁵ is C₁-C₆ alkoxy. In some embodiments, R⁵ is C₁-C₃ alkoxy. In some embodiments, R⁵ is —OCH₃, —OCH₂CH₃, —OCH₂CH₂CH₃, or —OCH(CH₃)₂. In some embodiments, R⁵ is —OCH₃. In some embodiments, R⁵ is —OCH₂CH₃.

In some embodiments, R⁵ is C₃-C₆ cycloalkyl. In some embodiments, R⁵ is C₃-C₅ cycloalkyl. In some embodiments, R⁵ is cyclopropyl, cyclobutyl, cyclopentyl, or cyclohexyl. In some embodiments, R⁵ is cyclopropyl. In some embodiments, R⁴ is cyclobutyl. In some embodiments, R⁵ is cyclopentyl. In some embodiments, R⁵ is cyclohexyl.

In some embodiments,

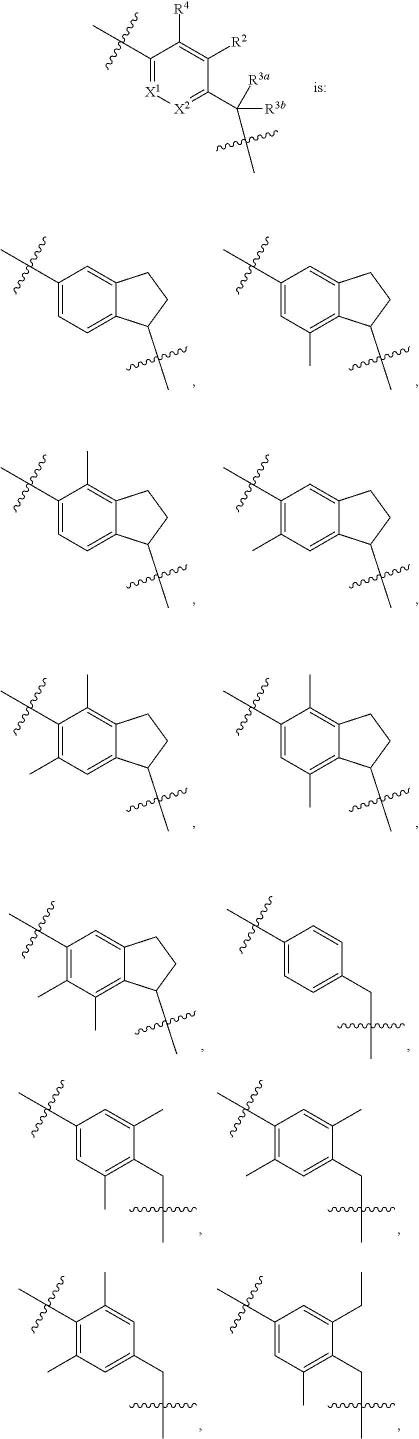

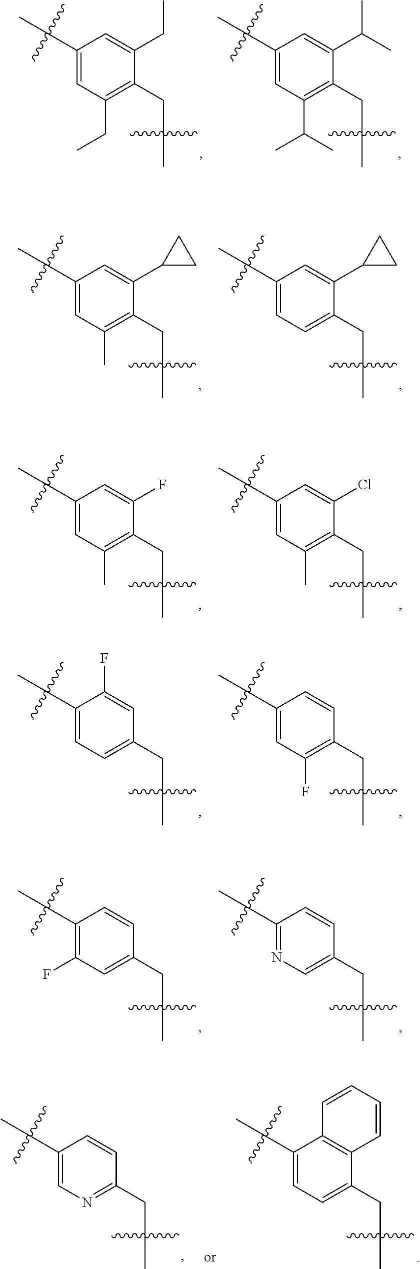

In some embodiments, R⁶ is H.

In some embodiments, R⁷ is C₁-C₆ alkyl-OH. In some embodiments, R⁷ is C₁—Cs alkyl-OH. In some embodiments, R⁷ is C₁-C₄ alkyl-OH. In some embodiments, R⁷ is C₃-C₆ alkyl-OH. In some embodiments, R⁷ is —CH₂OH, —CH₂CH₂OH, —CH₂CH₂CH₂OH, —CH(OH)CH₂CH₃, —C(CH₃)(OH)CH₂CH₃, —CH(OH)CH(CH₃)CH₃, —CH₂CH(OH)CH₃, —CH(CH₃)CH(OH)CH₃, —CH₂C(CH₃)(OH)CH₃, or —CH₂C(OH)(CH₃)₂. In some embodiments, R⁷ is —CH₂C(OH)(CH₃)₂.

In some embodiments, R⁶ and R⁷ are taken together with the nitrogen atom to which they are attached to form a 4- to 6-membered heterocyclyl substituted with n R⁸ groups. In some embodiments, R⁶ and R⁷ are taken together with the nitrogen atom to which they are attached to form a 4-membered heterocyclyl substituted with n R⁸ groups. In some embodiments, R⁶ and R⁷ are taken together with the nitrogen atom to which they are attached to form a 5-membered heterocyclyl substituted with n R⁸ groups. In some embodiments, R⁶ and R⁷ are taken together with the nitrogen atom to which they are attached to form a 6-membered heterocyclyl substituted with n R⁸ groups. In some embodiments, the heterocyclyl is azetidinyl, pyrrolidinyl, or piperidinyl, each of which is substituted by n R⁸ groups.

In some embodiments, R⁶ and R⁷ are taken together with the nitrogen atom to which they are attached to form

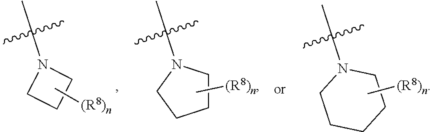

In some embodiments, n is 1-5. In some embodiments, n is 1-4. In some embodiments, n is 1-3. In some embodiments, n is 1-2. In some embodiments, n is 1. In some embodiments, n is 2. In some embodiments, n is 3. In some embodiments, n is 4. In some embodiments, n is 5.

In some embodiments, each R⁸ is independently halo, —CN, C₁-C₆ alkyl, C₁-C₆ haloalkyl, C₁-C₆ alkoxy, or —OH, provided that at least one R⁸ is —OH. In some embodiments, each R⁸ is independently halo, —CN, C₁-C₃ alkyl, C₁-C₃ haloalkyl, C₁-C₃ alkoxy, or —OH. In some embodiments, each R⁸ is independently-CH₃, —CH₂CH₃, —CFH2, —CF₂H, —CF₃, or —OH. In some embodiments, one R⁸ is —OH.

In some embodiments, R⁸ is halo. In some embodiments, R⁸ is C₁, F, or Br. In some embodiments, R⁸ is C₁. In some embodiments, R⁸ is F. In some embodiments, R⁸ is Br. In some embodiments, R⁸ is —CN.

In some embodiments, R⁸ is C₁-C₆ alkyl. In some embodiments, R⁸ is C₁-C₃ alkyl. In some embodiments, R⁸ is methyl, ethyl, n-propyl, or isopropyl. In some embodiments, R⁸ is methyl. In some embodiments, R⁸ is ethyl. In some embodiments, R⁸ is n-propyl. In some embodiments, R⁸ is isopropyl.

In some embodiments, R⁸ is C₁-C₆ haloalkyl. In some embodiments, R⁸ is C₁-C₆ haloalkyl containing 1-13 halogen atoms. In some embodiments, R⁸ is C₁-C₃ haloalkyl. In some embodiments, R⁸ is C₁-C₃ haloalkyl containing 1-7 halogen atoms. In some embodiments, R⁸ is —CF₃, —CHF₂, —CH₂F, —CFH₂, —CF₂H, —CCl₃, —CHCl₂, —CH₂Cl, —CF₂Cl, —CFCl₂, —CH₂CF₃, —CH₂CHF₂, or —CH₂CCl₃. In some embodiments, R⁸ is —CF₃. In some embodiments, R⁸ is —CFH₂. In some embodiments, R⁸ is —CF₂H.

In some embodiments, R⁸ is C₁-C₆ alkoxy. In some embodiments, R⁸ is C₁-C₃ alkoxy. In some embodiments, R⁸ is —OCH₃, —OCH₂CH₃, —OCH₂CH₂CH₃, or —OCH(CH₃)₂. In some embodiments, R⁸ is —OCH₃. In some embodiments, R⁸ is —OCH₂CH₃.

In some embodiments, R⁸ is —OH.

In some embodiments, two or more R⁸ groups are present and one R⁸ group is —OH. In some embodiments, two R⁸ groups are present and one R⁸ group is —OH. In some embodiments, three R⁸ groups are present and one R⁸ group is —OH.

In some embodiments,

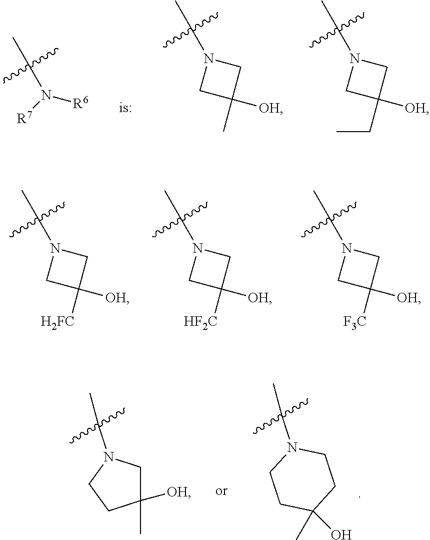

In some embodiments, the compound of Formula (I) is a compound of Formula (II):

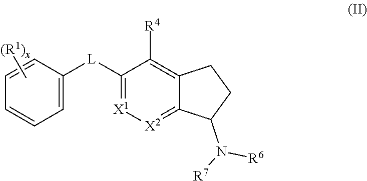

wherein R¹, R⁴, R⁶, R⁷, L, X¹, X², and x are as described for Formula (I).

In some embodiments, the compound of Formula (I) is a compound of Formula (IIa), (IIb), (IIc), (IId), (IIe), (IIf), or (IIg):

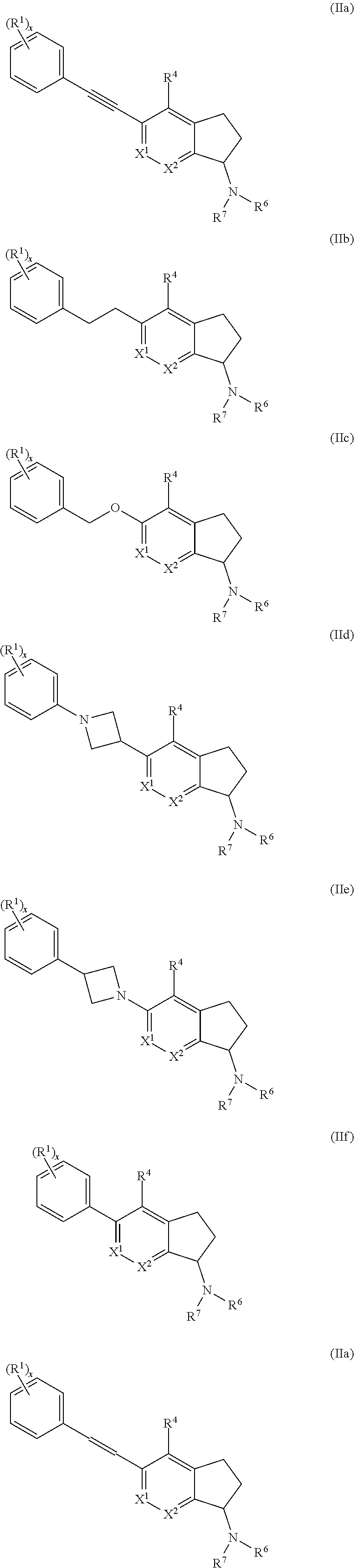

wherein R¹, R⁴, R⁶, R⁷, X¹, X², and x are as described for Formula (I).

In some embodiments, the compound of Formula (I) is a compound of Formula (II-A) or (II-B):

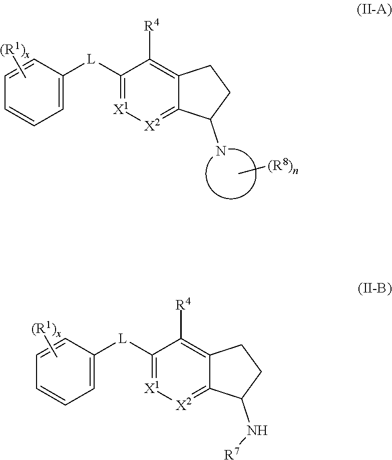

wherein R¹, R⁴, R⁸, L, X¹, X², n, and x are as described for Formula (I); and

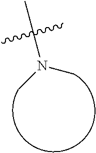

is a 4- to 6-membered heterocyclyl.

In some embodiments, the compound of Formula (I) is a compound of Formula (IIA), (IIB), (IIC), (IID), (TE), (IF), or (IIG):

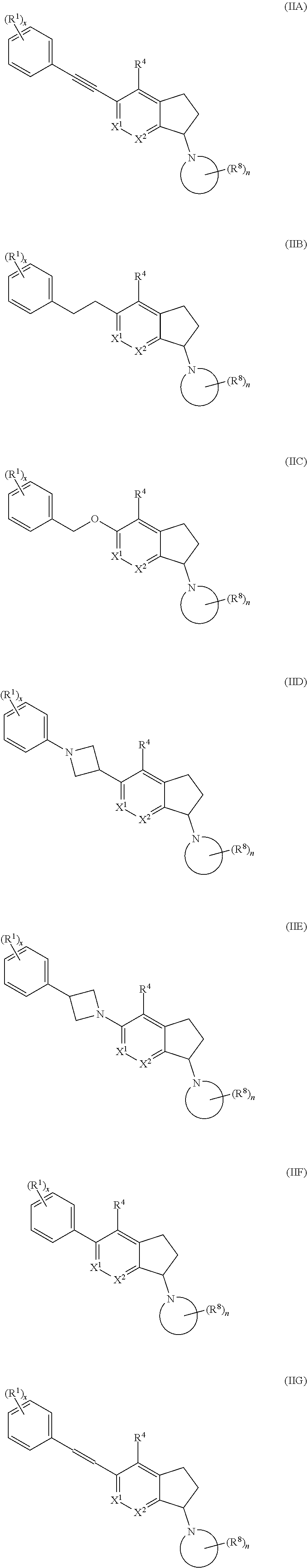

wherein R¹, R⁴, R⁸, X¹, X², x, and n are as described for Formula (I).

In some embodiments, the compound of Formula (I) is a compound of Formula (III):

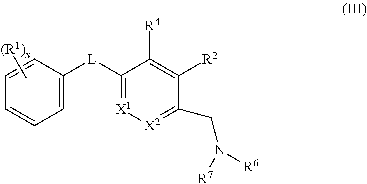

wherein R¹, R², R⁴, R⁸, R⁷, L, X¹, X², and x are as described for Formula (I).

In some embodiments, the compound of Formula (I) is a compound of Formula (IIIa), (IIIb), (IIIc), (IIId), (IIIe), (IIIf), or (IIIg):

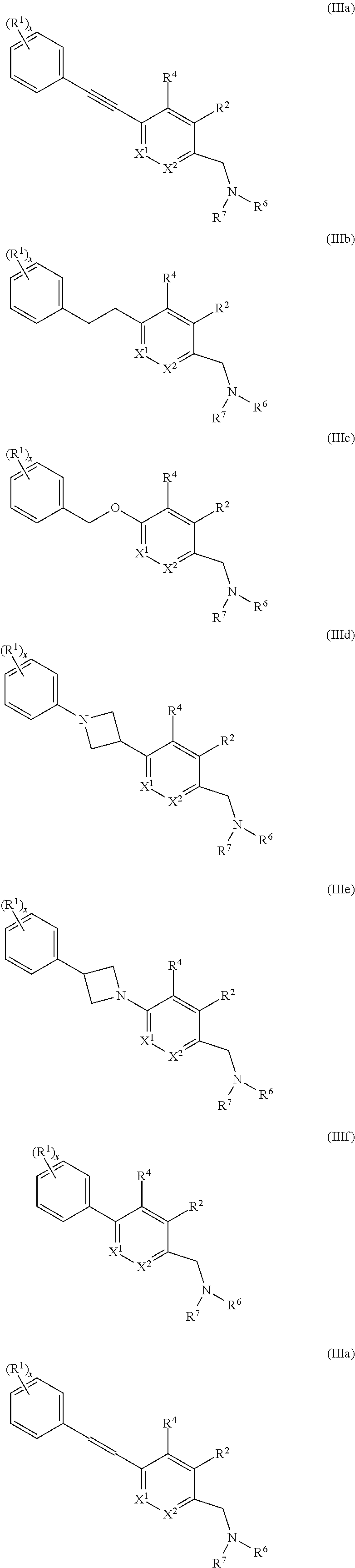

wherein R¹, R², R⁴, R⁸, R⁷, X¹, X², and x are as described for Formula (I).

In some embodiments, the compound of Formula (I) is a compound of Formula (IIIA), (IIIB), (IIIC), (IIID), (IIIE), (IIIF), or (IIIG):

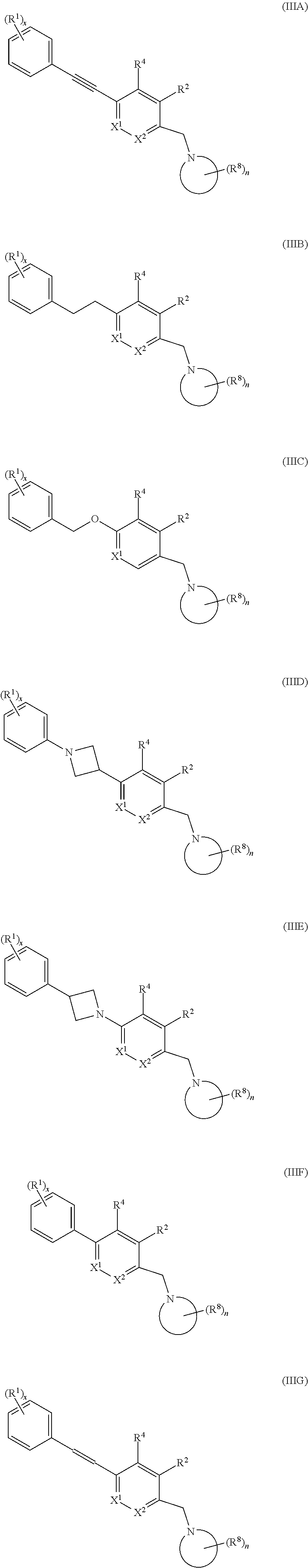

wherein R¹, R², R⁴, R⁸, X¹, X², x and n are as described for Formula (I).

In some embodiments, the compound of Formula (I) is a compound of Formula (III-A) or (III-B):

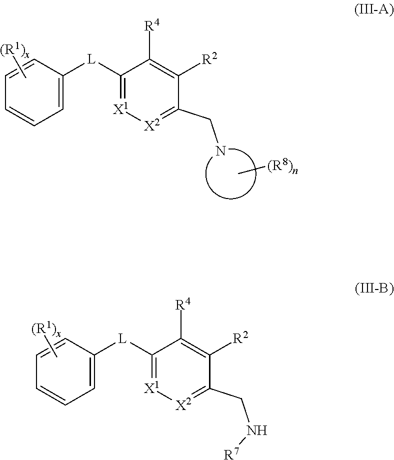

wherein R¹, R², R⁴, R⁷, R⁸, L, X¹, X², n and x are as described for Formula (I); and

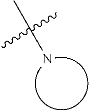 [SMILES: *N]

is a 4-to 6-membered heterocyclyl.

In some embodiments, the compound of Formula (I) is a compound of Formula (IVa), (IVb), (IVc), (IVd), (IVe), (IVf), or (IVg):

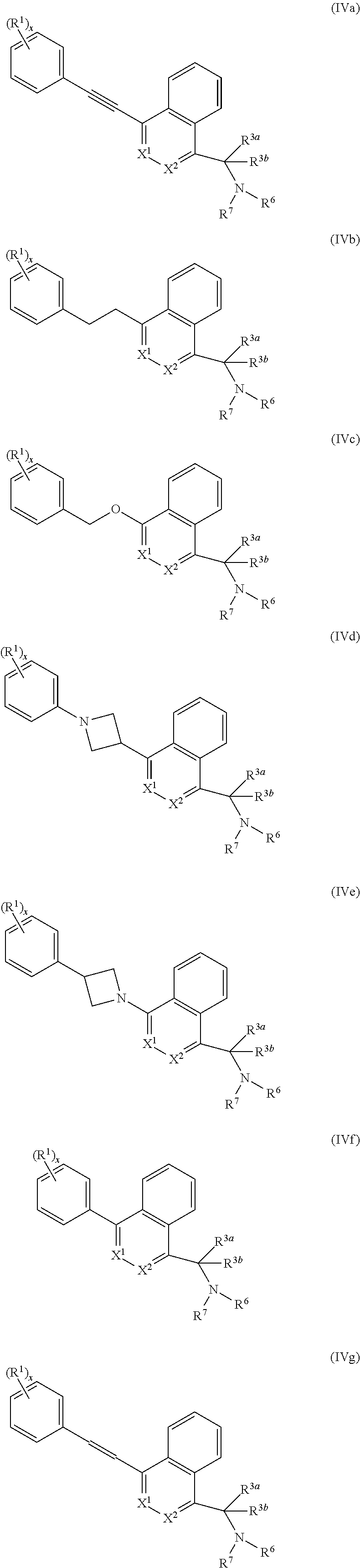

wherein R¹, R^{3a}, R^{3b}, R⁶, R⁷, X¹, X², and x are as described for Formula (I).

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to R¹ of Formula (I) may be combined with every description, variation, embodiment, or aspect of R², R^{3a}, R^{3b}, R⁴, R⁶, R⁷, R⁸, X¹, X², x and n, the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (II), (IIa), (IIb), (IIc), (IId), (IIe), (IIf), (IIg), (II-A), (II-B), (IIA), (IIB), (IIC), (IID), (IIE), (IIF), (IIG), (III), (IIIa), (IIIb), (IIIc), (IIId), (IIIe), (IIIf), (IIIg), (IIIA), (IIIB), (IIIC), (IIID), (IIIE), (IIIF), (IIIG), (III-A), (III-B), (IVa), (IVb), (IVc), (IVd), (IVe), (IVf), or (IVg) are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

TABLE 1

| Compound | | |
|---|---|---|
| No. | Structure | Name |
| 1a | 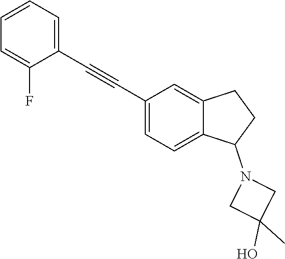 [SMILES: CC1(O)CN(C2CCc3cc(C#Cc4ccccc4F)ccc32)C1] | 1-(5-((2-fluoro-phenyl)ethynyl)-2,3- dihydro-1H-inden-1-yl)-3-methylazetidin- 3-ol Chiral separation 1 |
| 1b | 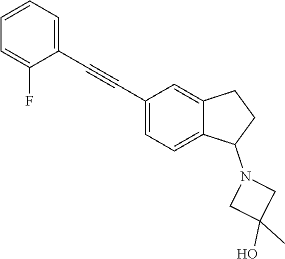 [SMILES: CC1(O)CN(C2CCc3cc(C#Cc4ccccc4F)ccc32)C1] | 1-(5-((2-fluoro-phenyl)ethynyl)-2,3- dihydro-1H-inden-1-yl)-3-methylazetidin- 3-ol Chiral separation 2 |
| 2a | 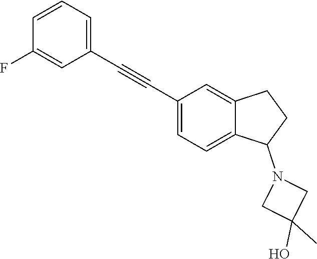 [SMILES: CC1(O)CN(C2CCc3cc(C#Cc4cccc(F)c4)ccc32)C1] | 1-[5-[2-(3-fluorophenyl)ethynyl]indan-1- yl]-3-methyl-azetidin-3-ol Chiral separation 1 |
| 2b | 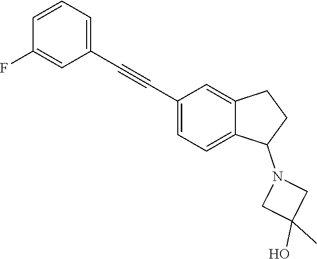 [SMILES: CC1(O)CN(C2CCc3cc(C#Cc4cccc(F)c4)ccc32)C1] | 1-[5-[2-(3-fluorophenyl)ethynyl]indan-1- yl]-3-methyl-azetidin-3-ol Chiral separation 2 |
| 3a | 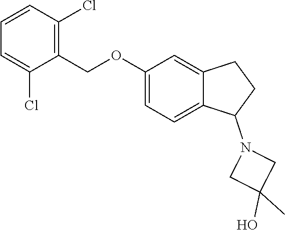 [SMILES: CC1(O)CN(C2CCc3cc(OCc4c(Cl)cccc4Cl)ccc32)C1] | 1-(5-((2,6-dichlorobenzyl)oxy)-2,3- dihydro-1H-inden-1-yl)-3-methylazetidin- 3-ol Chiral separation 1 |
| 3b | 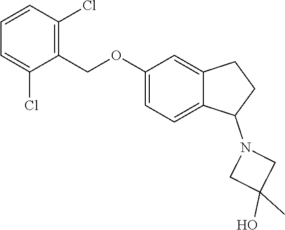 [SMILES: CC1(O)CN(C2CCc3cc(OCc4c(Cl)cccc4Cl)ccc32)C1] | 1-(5-((2,6-dichlorobenzyl)oxy)-2,3- dihydro-1H-inden-1-yl)-3-methylazetidin- 3-ol Chiral separation 2 |
| 4a | 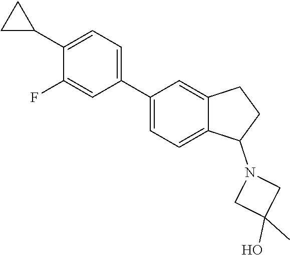 [SMILES: CC1(O)CN(C2CCc3cc(-c4ccc(C5CC5)c(F)c4)ccc32)C1] | 1-(5-(4-cyclopropyl-3-fluorophenyl)-2,3- dihydro-1H-inden-1-yl)-3-methyl azetidin-3-ol Chiral separation 1 |
| 4b | 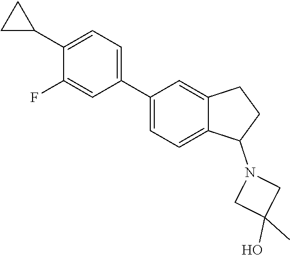 [SMILES: CC1(O)CN(C2CCc3cc(-c4ccc(C5CC5)c(F)c4)ccc32)C1] | 1-(5-(4-cyclopropyl-3-fluorophenyl)-2,3- dihydro-1H-inden-1-yl)-3-methyl- azetidin-3-ol Chiral separation 2 |
| 5a | 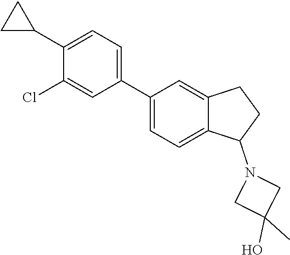 [SMILES: CC1(O)CN(C2CCc3cc(-c4ccc(C5CC5)c(Cl)c4)ccc32)C1] | 1-(5-(3-chloro-4-cyclopropylphenyl)-2,3- dihydro-1H-inden-1-yl)-3-methylazetidin- 3-ol Chiral separation 1 |
| 5b | 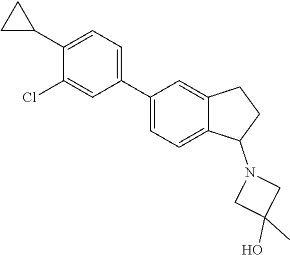 [SMILES: CC1(O)CN(C2CCc3cc(-c4ccc(C5CC5)c(Cl)c4)ccc32)C1] | 1-(5-(3-chloro-4-cyclopropylphenyl)-2,3- dihydro-1H-inden-1-yl)-3-methylazetidin- 3-ol Chiral separation 2 |
| 6 | 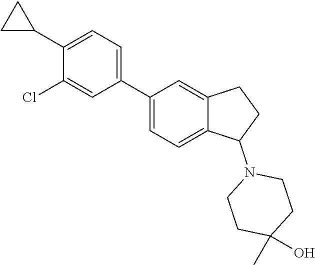 [SMILES: CC1(O)CCN(C2CCc3cc(-c4ccc(C5CC5)c(Cl)c4)ccc32)CC1] | 1-(5-(3-chloro-4-cyclopropylphenyl)-2,3- dihydro-1H-inden-1-yl)-4- methylpiperidin-4-ol |
| 7 | 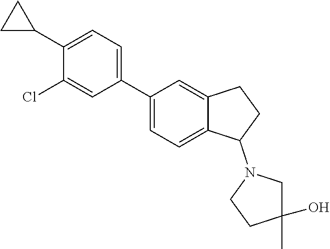 [SMILES: CC1(O)CCN(C2CCc3cc(-c4ccc(C5CC5)c(Cl)c4)ccc32)C1] | 1-(5-(3-chloro-4-cyclopropylphenyl)-2,3- dihydro-1H-inden-1-yl)-3- methylpyrrolidin-3-ol |
| 8 | 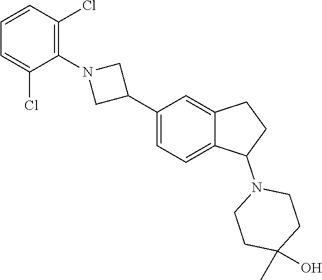 [SMILES: CC1(O)CCN(C2CCc3cc(C4CN(c5c(Cl)cccc5Cl)C4)ccc32)CC1] | 1-(5-(1-(2,6-dichlorophenyl)azetidin-3- yl)-2,3-dihydro-1H-inden-1-yl)-4-methyl- piperidin-4-ol |
| 9 | 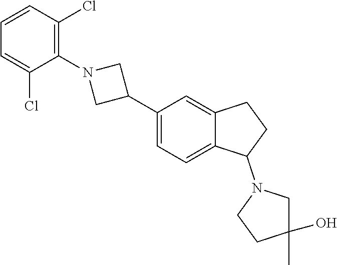 [SMILES: CC1(O)CCN(C2CCc3cc(C4CN(c5c(Cl)cccc5Cl)C4)ccc32)C1] | 1-(5-(1-(2,6-dichlorophenyl)azetidin-3- yl)-2,3-dihydro-1H-inden-1-yl)-3- methylpyrrolidin-3-ol |
| 10a | 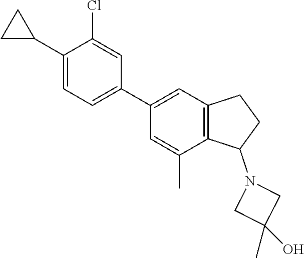 [SMILES: Cc1cc(-c2ccc(C3CC3)c(Cl)c2)cc2c1C(N1CC(C)(O)C1)CC2] | 1-(5-(3-chloro-4-cyclopropylphenyl)-7- methyl-2,3-dihydro-1H-inden-1-yl)-3- methylazetidin-3-ol Chiral separation 1 |
| 10b | 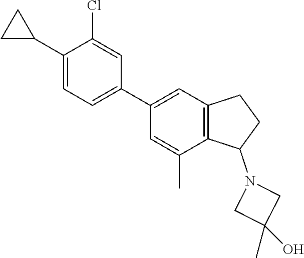 [SMILES: Cc1cc(-c2ccc(C3CC3)c(Cl)c2)cc2c1C(N1CC(C)(O)C1)CC2] | 1-(5-(3-chloro-4-cyclopropylphenyl)-7- methyl-2,3-dihydro-1H-inden-1-yl)-3- methylazetidin-3-ol Chiral separation 2 |
| 11 | 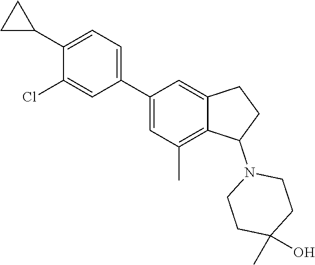 [SMILES: Cc1cc(-c2ccc(C3CC3)c(Cl)c2)cc2c1C(N1CCC(C)(O)CC1)CC2] | 1-(5-(3-chloro-4-cyclopropylphenyl)-7- methyl-2,3-dihydro-1H-inden-1-yl)-4- methylpiperidin-4-ol |
| 12 | 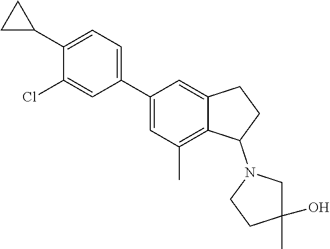 [SMILES: Cc1cc(-c2ccc(C3CC3)c(Cl)c2)cc2c1C(N1CCC(C)(O)C1)CC2] | 1-(5-(3-chloro-4-cyclopropylphenyl)-7- methyl-2,3-dihydro-1H-inden-1-yl)-3- methylpyrrolidin-3-ol |
| 13a | 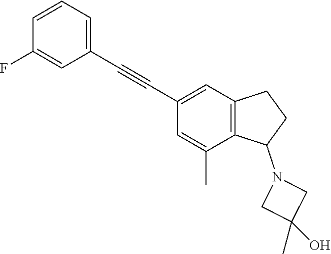 [SMILES: Cc1cc(C#Cc2cccc(F)c2)cc2c1C(N1CC(C)(O)C1)CC2] | (S)-1-(5-((3-fluorophenyl)ethynyl)-7- methyl-2,3-dihydro-1H-inden-1-yl)-3- methylazetidin-3-ol Chiral separation 1 |
| 13b | 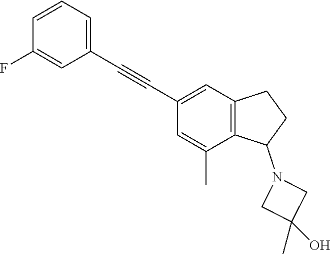 [SMILES: Cc1cc(C#Cc2cccc(F)c2)cc2c1C(N1CC(C)(O)C1)CC2] | (S)-1-(5-((3-fluorophenyl)ethynyl)-7- methyl-2,3-dihydro-1H-inden-1-yl)-3- methylazetidin-3-ol Chiral separation 2 |
| 14a | 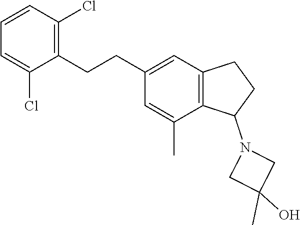 [SMILES: Cc1cc(CCc2c(Cl)cccc2Cl)cc2c1C(N1CC(C)(O)C1)CC2] | (S)-1-(5-(2,6-dichlorophenethyl)-7- methyl-2,3-dihydro-1H-inden-1-yl)-3- methylazetidin-3-ol Chiral separation 1 |
| 14b | 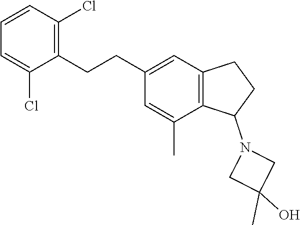 [SMILES: Cc1cc(CCc2c(Cl)cccc2Cl)cc2c1C(N1CC(C)(O)C1)CC2] | (S)-1-(5-(2,6-dichlorophenethyl)-7- methyl-2,3-dihydro-1H-inden-1-yl)-3- methylazetidin-3-ol Chiral separation 2 |
| 15a | 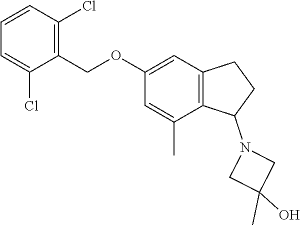 [SMILES: Cc1cc(OCc2c(Cl)cccc2Cl)cc2c1C(N1CC(C)(O)C1)CC2] | (S)-1-(5-((2,6-dichlorobenzyl)oxy)-7- methyl-2,3-dihydro-1H-inden-1-yl)-3- methylazetidin-3-ol Chiral separation 1 |
| 15b | 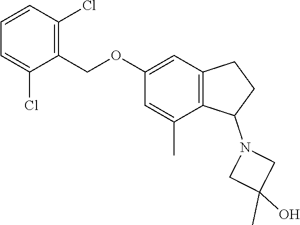 [SMILES: Cc1cc(OCc2c(Cl)cccc2Cl)cc2c1C(N1CC(C)(O)C1)CC2] | (S)-1-(5-((2,6-dichlorobenzyl)oxy)-7- methyl-2,3-dihydro-1H-inden-1-yl)-3- methylazetidin-3-ol Chiral separation 2 |
| 16a | 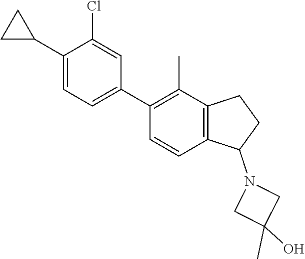 [SMILES: Cc1c(-c2ccc(C3CC3)c(Cl)c2)ccc2c1CCC2N1CC(C)(O)C1] | (S)-1-(5-(3-chloro-4-cyclopropylphenyl)- 4-methyl-2,3-dihydro-1H-inden-1-yl)-3- methylazetidin-3-ol Chiral separation 1 |
| 16b | 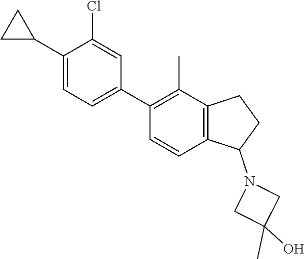 [SMILES: Cc1c(-c2ccc(C3CC3)c(Cl)c2)ccc2c1CCC2N1CC(C)(O)C1] | (S)-1-(5-(3-chloro-4-cyclopropylphenyl)- 4-methyl-2,3-dihydro-1H-inden-1-yl)-3- methylazetidin-3-ol Chiral separation 2 |
| 17a | 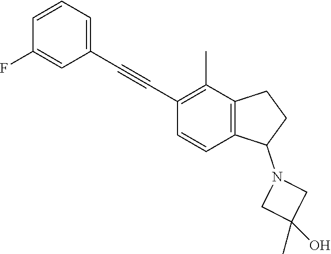 [SMILES: Cc1c(C#Cc2cccc(F)c2)ccc2c1CCC2N1CC(C)(O)C1] | (S)-1-(5-((3-fluorophenyl)ethynyl)-4- methyl-2,3-dihydro-1H-inden-1-yl)-3- methylazetidin-3-ol Chiral separation 1 |
| 17b | 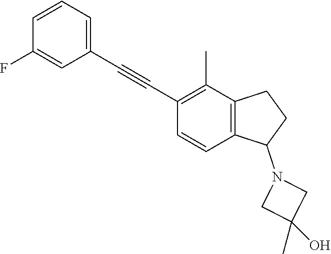 [SMILES: Cc1c(C#Cc2cccc(F)c2)ccc2c1CCC2N1CC(C)(O)C1] | (S)-1-(5-((3-fluorophenyl)ethynyl)-4- methyl-2,3-dihydro-1H-inden-1-yl)-3- methylazetidin-3-ol Chiral separation 2 |
| 18a | 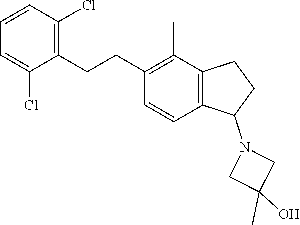 [SMILES: Cc1c(CCc2c(Cl)cccc2Cl)ccc2c1CCC2N1CC(C)(O)C1] | (S)-1-(5-(2,6-dichlorophenethyl)-4- methyl-2,3-dihydro-1H-inden-1-yl)-3- methylazetidin-3-ol Chiral separation 1 |
| 18b | 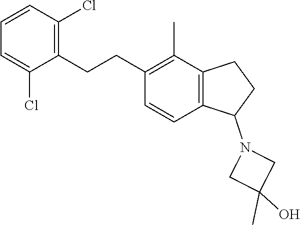 [SMILES: Cc1c(CCc2c(Cl)cccc2Cl)ccc2c1CCC2N1CC(C)(O)C1] | (S)-1-(5-(2,6-dichlorophenethyl)-4- methyl-2,3-dihydro-1H-inden-1-yl)-3- methylazetidin-3-ol Chiral separation 2 |
| 19a | 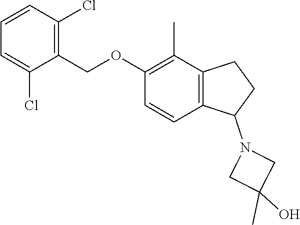 [SMILES: Cc1c(OCc2c(Cl)cccc2Cl)ccc2c1CCC2N1CC(C)(O)C1] | (S)-1-(5-((2,6-dichlorobenzyl)oxy)-4- methyl-2,3-dihydro-1H-inden-1-yl)-3- methylazetidin-3-ol Chiral separation 1 |
| 19b | 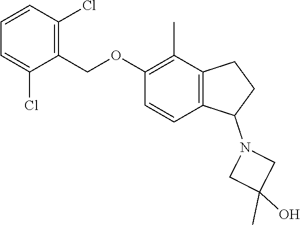 [SMILES: Cc1c(OCc2c(Cl)cccc2Cl)ccc2c1CCC2N1CC(C)(O)C1] | (S)-1-(5-((2,6-dichlorobenzyl)oxy)-4- methyl-2,3-dihydro-1H-inden-1-yl)-3- methylazetidin-3-ol Chiral separation 2 |
| 20a | 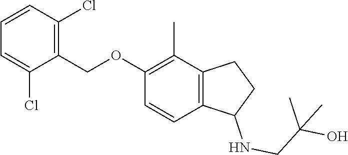 [SMILES: Cc1c(OCc2c(Cl)cccc2Cl)ccc2c1CCC2NCC(C)(C)O] | (S)-1-((5-((2,6-dichlorobenzyl)oxy)-4- methyl-2,3-dihydro-1H-inden-1- yl)amino)-2-methylpropan-2-ol Chiral separation 1 |
| 20b | 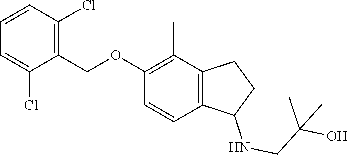 [SMILES: Cc1c(OCc2c(Cl)cccc2Cl)ccc2c1CCC2NCC(C)(C)O] | (S)-1-((5-((2,6-dichlorobenzyl)oxy)-4- methyl-2,3-dihydro-1H-inden-1- yl)amino)-2-methylpropan-2-ol Chiral separation 2 |
| 21a | 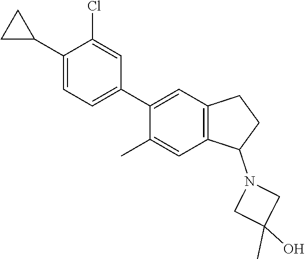 [SMILES: Cc1cc2c(cc1-c1ccc(C3CC3)c(Cl)c1)CCC2N1CC(C)(O)C1] | (S)-1-(5-(3-chloro-4-cyclopropylphenyl)- 6-methyl-2,3-dihydro-1H-inden-1-yl)-3- methylazetidin-3-ol Chiral separation 1 |
| 21b | 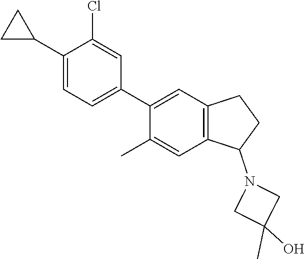 [SMILES: Cc1cc2c(cc1-c1ccc(C3CC3)c(Cl)c1)CCC2N1CC(C)(O)C1] | (S)-1-(5-(3-chloro-4-cyclopropylphenyl)- 6-methyl-2,3-dihydro-1H-inden-1-yl)-3- methylazetidin-3-ol Chiral separation 2 |
| 22a | 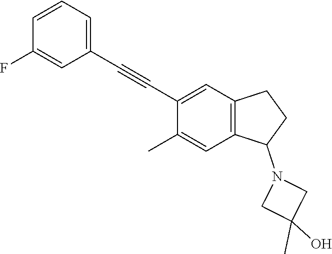 [SMILES: Cc1cc2c(cc1C#Cc1cccc(F)c1)CCC2N1CC(C)(O)C1] | (S)-1-(5-((3-fluorophenyl)ethynyl)-6- methyl-2,3-dihydro-1H-inden-1-yl)-3- methylazetidin-3-ol Chiral separation 1 |
| 22b | 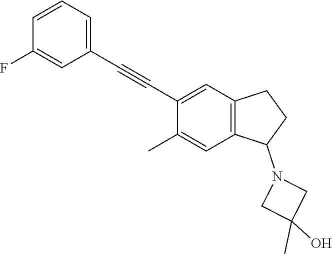 [SMILES: Cc1cc2c(cc1C#Cc1cccc(F)c1)CCC2N1CC(C)(O)C1] | (S)-1-(5-((3-fluorophenyl)ethynyl)-6- methyl-2,3-dihydro-1H-inden-1-yl)-3- methylazetidin-3-ol Chiral separation 2 |
| 23a | 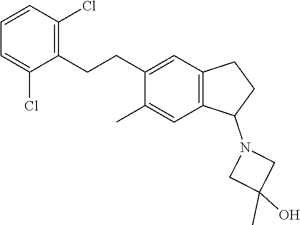 [SMILES: Cc1cc2c(cc1CCc1c(Cl)cccc1Cl)CCC2N1CC(C)(O)C1] | (S)-1-(5-(2,6-dichlorophenethyl)-6- methyl-2,3-dihydro-1H-inden-1-yl)-3- methylazetidin-3-ol Chiral separation 1 |
| 23b | 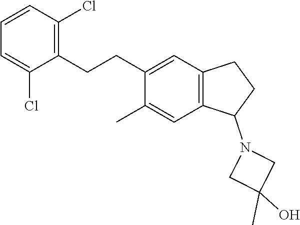 [SMILES: Cc1cc2c(cc1CCc1c(Cl)cccc1Cl)CCC2N1CC(C)(O)C1] | (S)-1-(5-(2,6-dichlorophenethyl)-6- methyl-2,3-dihydro-1H-inden-1-yl)-3- methylazetidin-3-ol Chiral separation 2 |
| 24a | 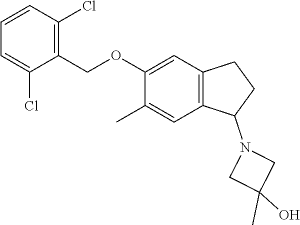 [SMILES: Cc1cc2c(cc1OCc1c(Cl)cccc1Cl)CCC2N1CC(C)(O)C1] | (S)-1-(5-((2,6-dichlorobenzyl)oxy)-6- methyl-2,3-dihydro-1H-inden-1-yl)-3- methylazetidin-3-ol Chiral separation 1 |
| 24b | 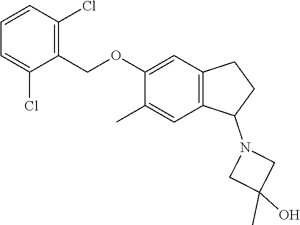 [SMILES: Cc1cc2c(cc1OCc1c(Cl)cccc1Cl)CCC2N1CC(C)(O)C1] | (S)-1-(5-((2,6-dichlorobenzyl)oxy)-6- methyl-2,3-dihydro-1H-inden-1-yl)-3- methylazetidin-3-ol Chiral separation 2 |
| 25a | 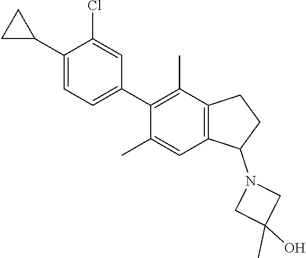 [SMILES: Cc1cc2c(c(C)c1-c1ccc(C3CC3)c(Cl)c1)CCC2N1CC(C)(O)C1] | (S)-1-(5-(3-chloro-4-cyclopropylphenyl)- 4,6-dimethyl-2,3-dihydro-1H-inden-1-yl)- 3-methylazetidin-3-ol Chiral separation 1 |
| 25b | 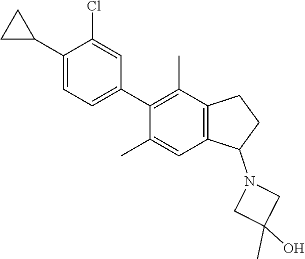 [SMILES: Cc1cc2c(c(C)c1-c1ccc(C3CC3)c(Cl)c1)CCC2N1CC(C)(O)C1] | (S)-1-(5-(3-chloro-4-cyclopropylphenyl)- 4,6-dimethyl-2,3-dihydro-1H-inden-1-yl)- 3-methylazetidin-3-ol Chiral separation 2 |
| 26a | 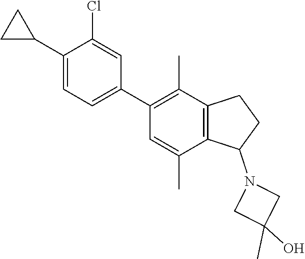 [SMILES: Cc1cc(-c2ccc(C3CC3)c(Cl)c2)c(C)c2c1C(N1CC(C)(O)C1)CC2] | (S)-1-(5-(3-chloro-4-cyclopropylphenyl)- 4,7-dimethyl-2,3-dihydro-1H-inden-1-yl)- 3-methylazetidin-3-ol Chiral separation 1 |
| 26b | 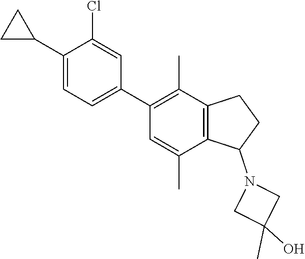 [SMILES: Cc1cc(-c2ccc(C3CC3)c(Cl)c2)c(C)c2c1C(N1CC(C)(O)C1)CC2] | (S)-1-(5-(3-chloro-4-cyclopropylphenyl)- 4,7-dimethyl-2,3-dihydro-1H-inden-1-yl)- 3-methylazetidin-3-ol Chiral separation 2 |
| 27a | 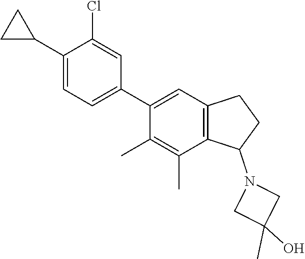 [SMILES: Cc1c(-c2ccc(C3CC3)c(Cl)c2)cc2c(c1C)C(N1CC(C)(O)C1)CC2] | (S)-1-(5-(3-chloro-4-cyclopropylphenyl)- 6,7-dimethyl-2,3-dihydro-1H-inden-1-yl)- 3-methylazetidin-3-ol Chiral separation 1 |
| 27b | 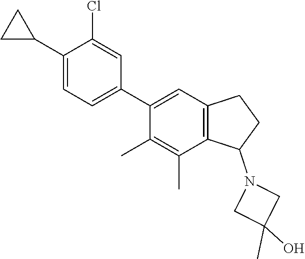 [SMILES: Cc1c(-c2ccc(C3CC3)c(Cl)c2)cc2c(c1C)C(N1CC(C)(O)C1)CC2] | (S)-1-(5-(3-chloro-4-cyclopropylphenyl)- 6,7-dimethyl-2,3-dihydro-1H-inden-1-yl)- 3-methylazetidin-3-ol Chiral separation 2 |
| 28 | 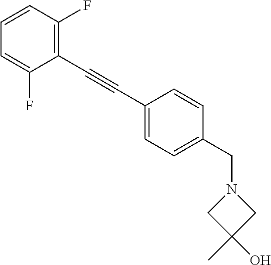 [SMILES: CC1(O)CN(Cc2ccc(C#Cc3c(F)cccc3F)cc2)C1] | 1-(4-((2,6-difluoro- phenyl)ethynyl)benzyl)-3-methylazetidin- 3-ol |
| 29 | 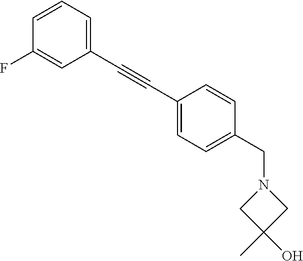 [SMILES: CC1(O)CN(Cc2ccc(C#Cc3cccc(F)c3)cc2)C1] | 1-(4-((3-fluorophenyl)-ethynyl)benzyl)-3- methylazetidin-3-ol |
| 30 | 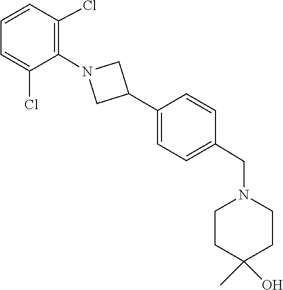 [SMILES: CC1(O)CCN(Cc2ccc(C3CN(c4c(Cl)cccc4Cl)C3)cc2)CC1] | 1-(4-(1-(2,6-dichlorophenyl)azetidin-3- yl)benzyl)-4-methylpiperidin-4-ol |
| 31 | 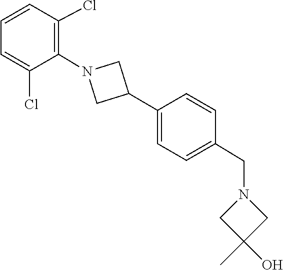 [SMILES: CC1(O)CN(Cc2ccc(C3CN(c4c(Cl)cccc4Cl)C3)cc2)C1] | 1-(4-(1-(2,6-dichlorophenyl)azetidin-3- yl)benzyl)-3-methylazetidin-3-ol |
| 32 | 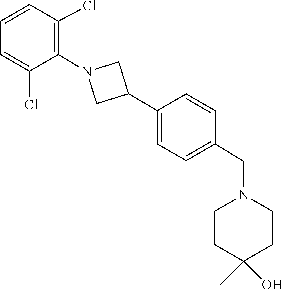 [SMILES: CC1(O)CCN(Cc2ccc(C3CN(c4c(Cl)cccc4Cl)C3)cc2)CC1] | 1-(4-(3-(2,6-dichlorophenyl)azetidin-1- yl)benzyl)-4-methylpiperidin-4-ol |
| 33 | 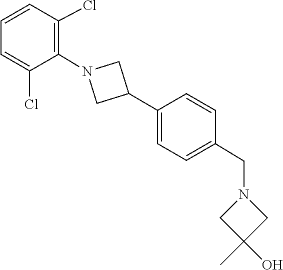 [SMILES: CC1(O)CN(Cc2ccc(C3CN(c4c(Cl)cccc4Cl)C3)cc2)C1] | 1-(4-(3-(2,6-dichlorophenyl)azetidin-1- yl)benzyl)-3-methylazetidin-3-ol |
| 34 | 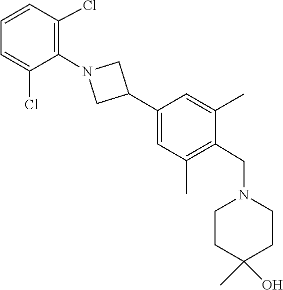 [SMILES: Cc1cc(C2CN(c3c(Cl)cccc3Cl)C2)cc(C)c1CN1CCC(C)(O)CC1] | 1-(4-(1-(2,6-dichlorophenyl)azetidin-3- yl)-2,6-dimethylbenzyl)-4- methylpiperidin-4-ol |
| 35 | 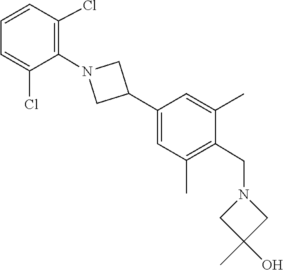 [SMILES: Cc1cc(C2CN(c3c(Cl)cccc3Cl)C2)cc(C)c1CN1CC(C)(O)C1] | 1-(4-((2,6- dichlorophenyl)ethynyl)benzyl)-3- methylazetidin-3-ol |
| 36 | 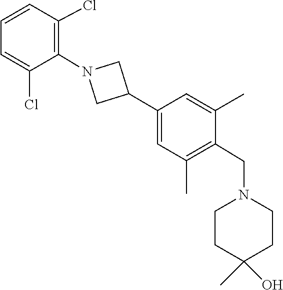 [SMILES: Cc1cc(C2CN(c3c(Cl)cccc3Cl)C2)cc(C)c1CN1CCC(C)(O)CC1] | 1-(4-(3-(2,6-dichlorophenyl)azetidin-1- yl)-2,6-dimethylbenzyl)-4- methylpiperidin-4-ol |
| 37 | 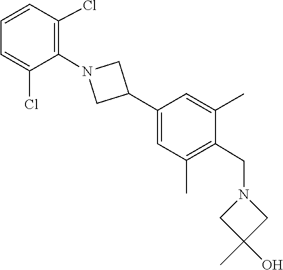 [SMILES: Cc1cc(C2CN(c3c(Cl)cccc3Cl)C2)cc(C)c1CN1CC(C)(O)C1] | 1-(4-(3-(2,6-dichlorophenyl)azetidin-1- yl)-2,6-dimethylbenzyl)-3-methylazetidin- 3-ol |
| 38 | 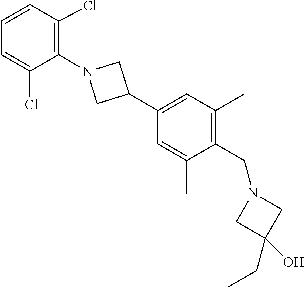 [SMILES: CCC1(O)CN(Cc2c(C)cc(C3CN(c4c(Cl)cccc4Cl)C3)cc2C)C1] | 1-(4-(1-(2,6-dichlorophenyl)azetidin-3- yl)-2,6-dimethylbenzyl)-3-ethylazetidin-3- ol |
| 39 | 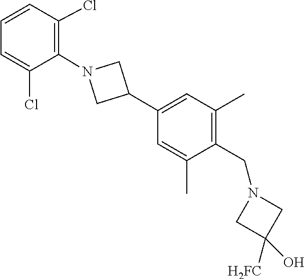 | 1-(4-(1-(2,6-dichlorophenyl)azetidin-3- yl)-2,6-dimethylbenzyl)-3- (fluoromethyl)azetidin-3-ol |
| 40 | 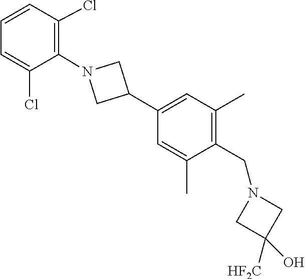 | 1-(4-(1-(2,6-dichlorophenyl)azetidin-3- yl)-2,6-dimethylbenzyl)-3- (difluoromethyl)azetidin-3-ol |
| 41 | 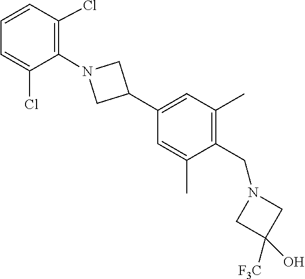 [SMILES: Cc1cc(C2CN(c3c(Cl)cccc3Cl)C2)cc(C)c1CN1CC(O)(C(F)(F)F)C1] | 1-(4-(1-(2,6-dichlorophenyl)azetidin-3- yl)-2,6-dimethylbenzyl)-3- (trifluoromethyl)azetidin-3-ol |
| 42 | 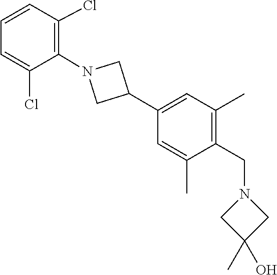 [SMILES: Cc1cc(C2CN(c3c(Cl)cccc3Cl)C2)cc(C)c1CN1CC(C)(O)C1] | 1-(4-(1-(2,6-difluorophenyl)azetidin-3- yl)-2,6-dimethylbenzyl)-3-methylazetidin- 3-ol |
| 43 | 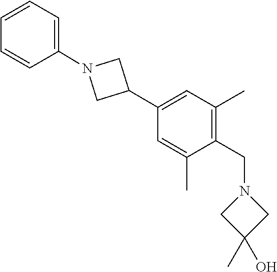 [SMILES: Cc1cc(C2CN(c3ccccc3)C2)cc(C)c1CN1CC(C)(O)C1] | 1-(2,6-dimethyl-4-(1-phenylazetidin-3- yl)benzyl)-3-methylazetidin-3-ol |
| 44 | 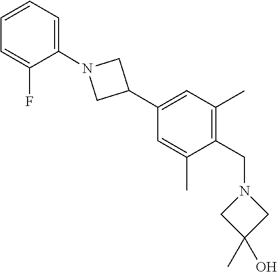 [SMILES: Cc1cc(C2CN(c3ccccc3F)C2)cc(C)c1CN1CC(C)(O)C1] | 1-(4-(1-(2-fluorophenyl)azetidin-3-yl)- 2,6-dimethylbenzyl)-3-methylazetidin-3- ol |
| 45 | 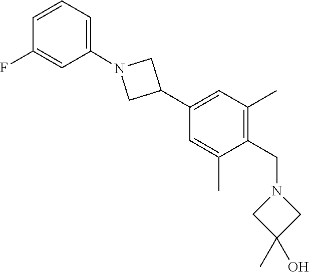 [SMILES: Cc1cc(C2CN(c3cccc(F)c3)C2)cc(C)c1CN1CC(C)(O)C1] | 1-(4-(1-(3-fluorophenyl)azetidin-3-yl)- 2,6-dimethylbenzyl)-3-methylazetidin-3- ol |
| 46 | 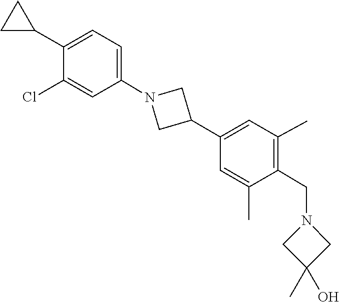 [SMILES: Cc1cc(C2CN(c3ccc(C4CC4)c(Cl)c3)C2)cc(C)c1CN1CC(C)(O)C1] | 1-(4-(1-(3-chloro-4- cyclopropylphenyl)azetidin-3-yl)-2,6- dimethylbenzyl)-3-methylazetidin-3-ol |
| 47 | 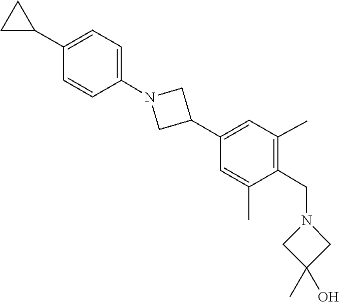 [SMILES: Cc1cc(C2CN(c3ccc(C4CC4)cc3)C2)cc(C)c1CN1CC(C)(O)C1] | 1-(4-(1-(4-cyclopropylphenyl)azetidin-3- yl)-2,6-dimethylbenzyl)-3-methylazetidin- 3-ol |
| 48 | 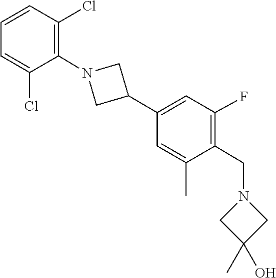 [SMILES: Cc1cc(C2CN(c3c(Cl)cccc3Cl)C2)cc(F)c1CN1CC(C)(O)C1] | 1-(4-(1-(2,6-dichlorophenyl)azetidin-3- yl)-2-fluoro-6-methylbenzyl)-3- methylazetidin-3-ol |
| 49 | 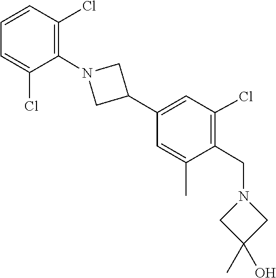 [SMILES: Cc1cc(C2CN(c3c(Cl)cccc3Cl)C2)cc(Cl)c1CN1CC(C)(O)C1] | 1-(2-chloro-4-(1-(2,6- dichlorophenyl)azetidin-3-yl)-6- methylbenzyl)-3-methylazetidin-3-ol |
| 50 | 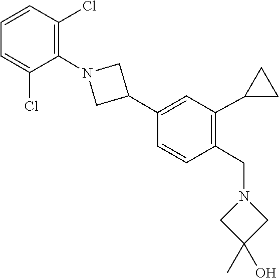 [SMILES: CC1(O)CN(Cc2ccc(C3CN(c4c(Cl)cccc4Cl)C3)cc2C2CC2)C1] | 1-(2-cyclopropyl-4-(1-(2,6- dichlorophenyl)azetidin-3-yl)benzyl)-3- methylazetidin-3-ol |
| 51 | 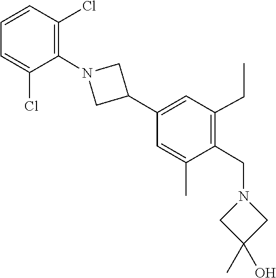 [SMILES: CCc1cc(C2CN(c3c(Cl)cccc3Cl)C2)cc(C)c1CN1CC(C)(O)C1] | 1-(4-(1-(2,6-dichlorophenyl)azetidin-3- yl)-2-ethyl-6-methylbenzyl)-3- methylazetidin-3-ol |
| 52 | 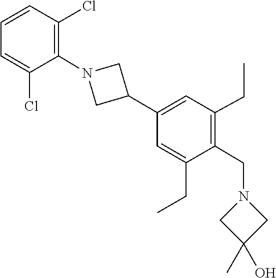 [SMILES: CCc1cc(C2CN(c3c(Cl)cccc3Cl)C2)cc(CC)c1CN1CC(C)(O)C1] | 1-(4-(1-(2,6-dichlorophenyl)azetidin-3- yl)-2,6-diethylbenzyl)-3-methylazetidin-3- ol |
| 53 | 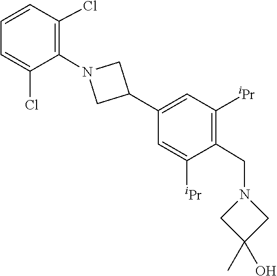 [SMILES: CC(C)c1cc(C2CN(c3c(Cl)cccc3Cl)C2)cc(C(C)C)c1CN1CC(C)(O)C1] | 1-(4-(1-(2,6-dichlorophenyl)azetidin-3- yl)-2,6-diisopropylbenzyl)-3- methylazetidin-3-ol |
| 54 | 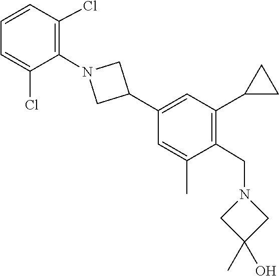 [SMILES: Cc1cc(C2CN(c3c(Cl)cccc3Cl)C2)cc(C2CC2)c1CN1CC(C)(O)C1] | 1-(2-cyclopropyl-4-(1-(2,6- dichlorophenyl)azetidin-3-yl)-6- methylbenzyl)-3-methylazetidin-3-ol |
| 55 | 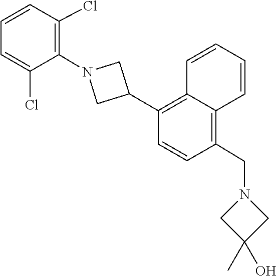 [SMILES: CC1(O)CN(Cc2ccc(C3CN(c4c(Cl)cccc4Cl)C3)c3ccccc23)C1] | 1-((4-(1-(2,6-dichlorophenyl)azetidin-3- yl)naphthalen-1-yl)methyl)-3- methylazetidin-3-ol |
| 56 | 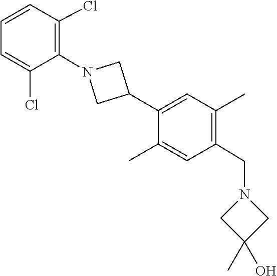 [SMILES: Cc1cc(C2CN(c3c(Cl)cccc3Cl)C2)c(C)cc1CN1CC(C)(O)C1] | 1-(4-(1-(2,6-dichlorophenyl)azetidin-3- yl)-2,5-dimethylbenzyl)-3-methylazetidin- 3-ol |
| 57 | 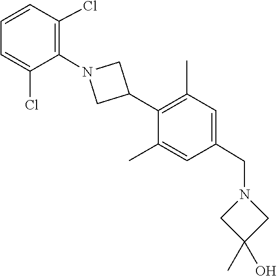 [SMILES: Cc1cc(CN2CC(C)(O)C2)cc(C)c1C1CN(c2c(Cl)cccc2Cl)C1] | 1-(4-(1-(2,6-dichlorophenyl)azetidin-3- yl)-3,5-dimethylbenzyl)-3-methylazetidin- 3-ol |
| 58 | 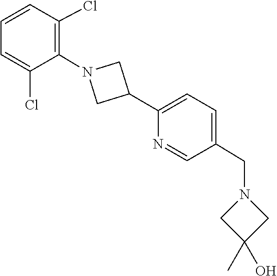 [SMILES: CC1(O)CN(Cc2ccc(C3CN(c4c(Cl)cccc4Cl)C3)nc2)C1] | 1-((6-(1-(2,6-dichlorophenyl)azetidin-3- yl)pyridin-3-yl)methyl)-3-methylazetidin- 3-ol |
| 59 | 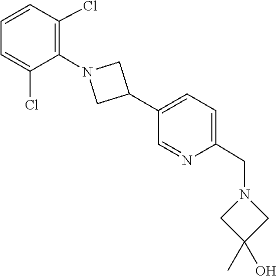 [SMILES: CC1(O)CN(Cc2ccc(C3CN(c4c(Cl)cccc4Cl)C3)cn2)C1] | 1-((5-(1-(2,6-dichlorophenyl)azetidin-3- yl)pyridin-2-yl)methyl)-3-methylazetidin- 3-ol |
| 60 | 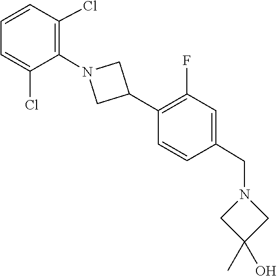 [SMILES: CC1(O)CN(Cc2ccc(C3CN(c4c(Cl)cccc4Cl)C3)c(F)c2)C1] | 1-(4-(1-(2,6-dichlorophenyl)azetidin-3- yl)-3-fluorobenzyl)-3-methylazetidin-3-ol |
| 61 | 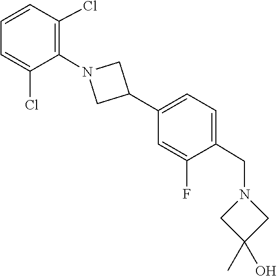 [SMILES: CC1(O)CN(Cc2ccc(C3CN(c4c(Cl)cccc4Cl)C3)cc2F)C1] | 1-(4-(1-(2,6-dichlorophenyl)azetidin-3- yl)-2-fluorobenzyl)-3-methylazetidin-3-ol |
| 62 | 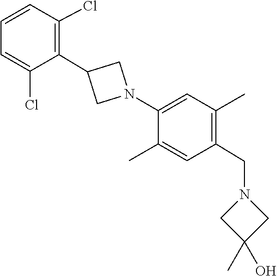 [SMILES: Cc1cc(N2CC(c3c(Cl)cccc3Cl)C2)c(C)cc1CN1CC(C)(O)C1] | 1-(4-(3-(2,6-dichlorophenyl)azetidin-1- yl)-2,5-dimethylbenzyl)-3-methylazetidin- 3-ol |
| 63 | 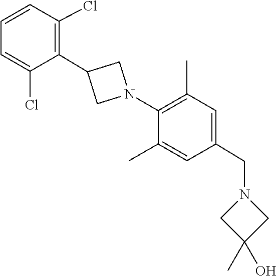 [SMILES: Cc1cc(CN2CC(C)(O)C2)cc(C)c1N1CC(c2c(Cl)cccc2Cl)C1] | 1-(4-(3-(2,6-dichlorophenyl)azetidin-1- yl)-3,5-dimethylbenzyl)-3-methylazetidin- 3-ol |
| 64 | 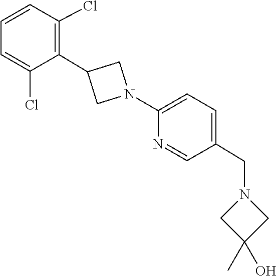 [SMILES: CC1(O)CN(Cc2ccc(N3CC(c4c(Cl)cccc4Cl)C3)nc2)C1] | 1-((6-(3-(2,6-dichlorophenyl)azetidin-1- yl)pyridin-3-yl)methyl)-3-methylazetidin- 3-ol |
| 65 | 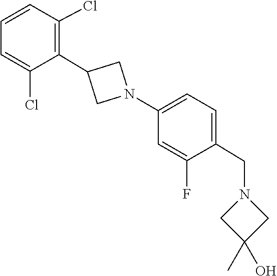 [SMILES: CC1(O)CN(Cc2ccc(N3CC(c4c(Cl)cccc4Cl)C3)cc2F)C1] | 1-(4-(3-(2,6-dichlorophenyl)azetidin-1- yl)-2-fluorobenzyl)-3-methylazetidin-3-ol |
| 66 | 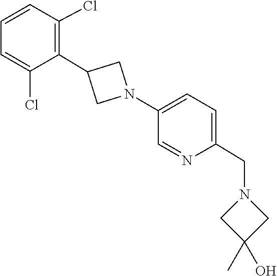 [SMILES: CC1(O)CN(Cc2ccc(N3CC(c4c(Cl)cccc4Cl)C3)cn2)C1] | 1-((5-(3-(2,6-dichlorophenyl)azetidin-1- yl)pyridin-2-yl)methyl)-3-methylazetidin- 3-ol |
| 67 | 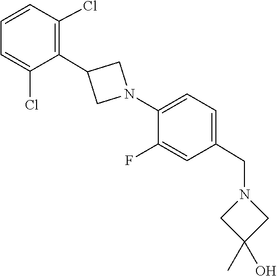 [SMILES: CC1(O)CN(Cc2ccc(N3CC(c4c(Cl)cccc4Cl)C3)c(F)c2)C1] | 1-(4-(3-(2,6-dichlorophenyl)azetidin-1- yl)-3-fluorobenzyl)-3-methylazetidin-3-ol |

or a pharmaceutically acceptable salt thereof.

It is understood that in the present description, combinations of substituents and/or variables of the depicted formulae are permissible only if such contributions result in stable compounds.

Furthermore, all compounds of Formula (I) that exist in free base or acid form can be converted to their pharmaceutically acceptable salts by treatment with the appropriate inorganic or organic base or acid by methods known to one skilled in the art. Salts of the compounds of Formula (I) can be converted to their free base or acid form by standard techniques.

### Methods of Synthesis

The compounds described herein can be made using conventional organic syntheses and commercially available starting materials, or the methods provided herein. By way of example and not limitation, compounds of Formula (I) can be prepared as outlined in Scheme 1, as well as in the examples set forth herein. It should be noted that one skilled in the art would know how to modify the procedures set forth in the illustrative schemes and examples to arrive at the desired products.

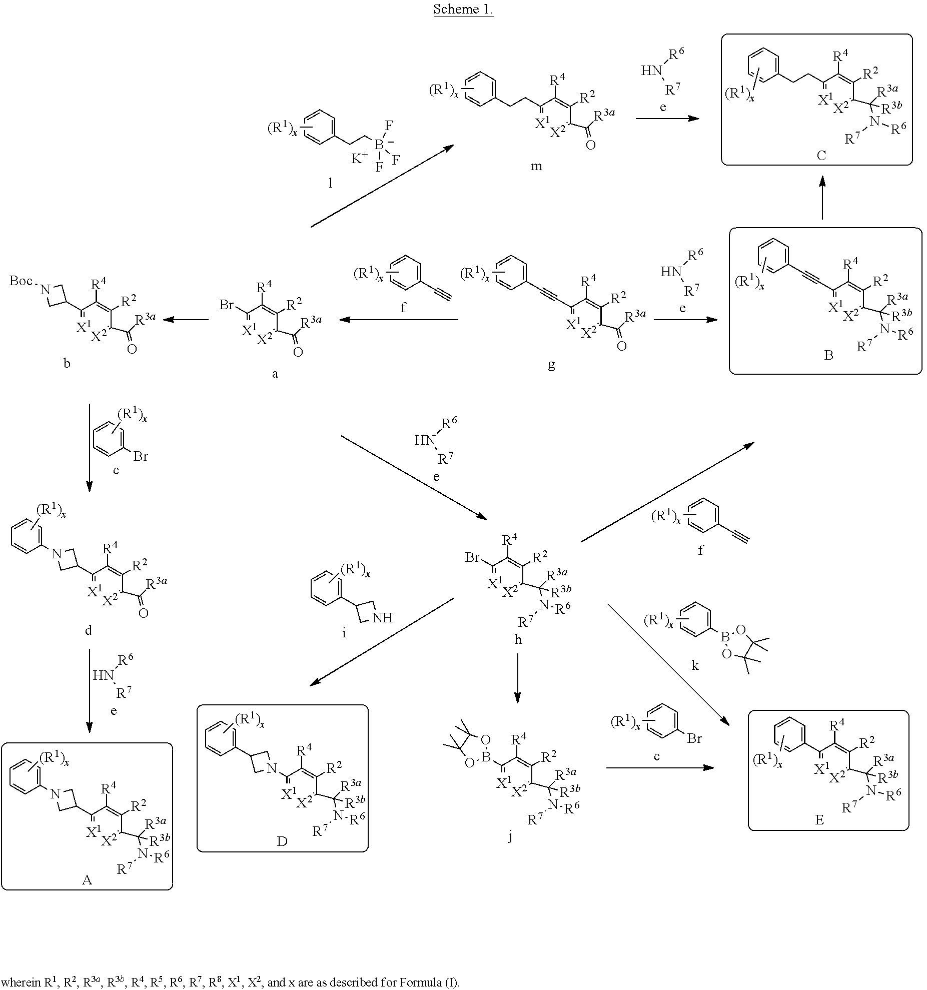

As outlined in Scheme 1, compounds of Formula A can be synthesized from a bromine-substituted ring a via coupling with a Boc-protected 3-iodo-azetidine to form intermediate b, which after deprotection is subsequently reacted with an aryl bromide c to form intermediate d. Subsequent Schiff's base reaction with intermediate d with an amine e provides the compound of Formula A.

As outlined in Scheme 1, compounds of Formula B can be synthesized from a bromine-substituted ring a via coupling with an aryl alkyne f to form intermediate g, which by subsequent Schiff's base reaction with an amine e forms the compound of Formula B. Alternatively, the bromine-substituted ring a can first undergo a Schiff's base reaction with the amine e to form intermediate h, which can then couple with the aryl alkyne f to form Formula B. Further hydrogenation of the compound of Formula B can provide the compound of Formula C. Compounds of the formula C can also be prepared by coupling trifluoroboratesalts I to intermediate a, followed by reductive amination with amine e.

As outlined in Scheme 1, compounds of Formula D can be synthesized by reacting a bromine-substituted ring a with an amine e to form intermediate h, which by subsequent coupling reaction with the amine of the azetidine-aryl i forms the compound of Formula D.

As outlined in Scheme 1, compounds of Formula E can be synthesized from Cintermediate h via two different routes. The first involves two step reaction, in which first a via a coupling reaction between intermediate h and a dioxaborolane compound forms intermediate j, which further couples with an aryl bromide c to form the compound of Formula E. The other involves a direct coupling reaction between intermediate h and a dioxaborolane aryl k to form a compound of Formula E.

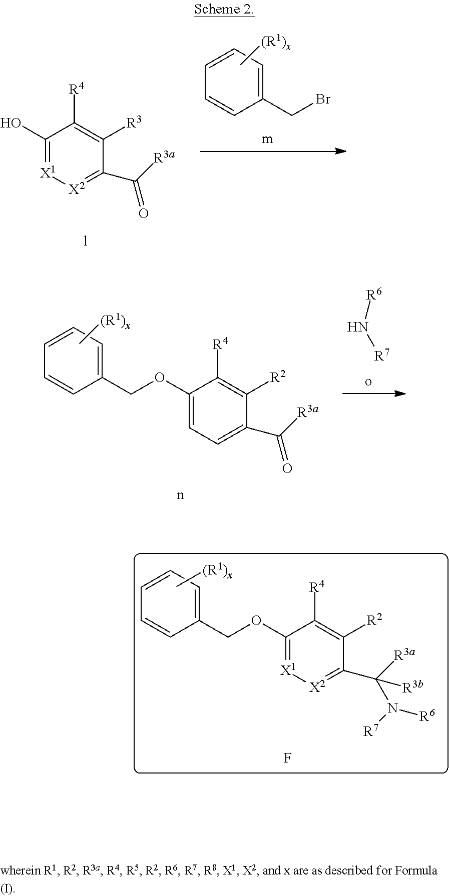

Scheme 2 shows a synthetic route to compounds of Formula F. Reaction of intermediate I with benzyl bromide m forms intermediate n, which is then coupled to amine o to form the compound of Formula F.

### Methods of Use

Embodiments of the present disclosure provide a method for modulating sphingosine 1-phosphate receptor 5 (S1P5) in a subject in need thereof, the method comprising administering to the subject an effective amount of a compound of Formula (I). Modulation (e.g., inhibition or activation) of S1P5 can be assessed and demonstrated by a wide variety of ways known in the art. Kits and commercially available assays can be utilized for determining whether and to what degree S1P5 has been modulated (e.g., inhibited or activated).

In one aspect, provided herein is a method of modulating S1P5 comprising contacting S1P5 with an effective amount of a compound of Formula (I) or any embodiment or variation thereof. In some embodiments, the compound of Formula (I) inhibits S1P5. In other embodiments, the compound of Formula (I) activates S1P5. In some embodiments, the compound of Formula (I) is an agonist of S1P5. In some embodiments, the compound of Formula (I) is an antagonist of S1P5.

In some embodiments, a compound of Formula (I) modulates the activity of S1P5 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) modulates the activity of S1P5 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In another aspect, provided herein is a method for treating a neurological disease in a subject in need thereof, comprising administering to the subject an effective amount of a compound of Formula (I). In some embodiments, provided herein is a method for preventing a neurological disease in a subject in need thereof, comprising administering to the subject an effective amount of a compound of Formula (I). Non-limiting examples of a neurological disease include Alzheimer's disease, multiple sclerosis (MS), amyotrophic lateral sclerosis (ALS), migraine, Bell's Palsy, ataxia, cerebral aneurysm, epilepsy, seizures, acute spinal cord injury, Guillain-Barre syndrome, meningitis, Niemann Pick disease, and Parkinson's disease. In some embodiments, the neurological disease is Alzheimer's disease or multiple sclerosis. In some embodiments, the neurological disease is Alzheimer's disease. In some embodiments, the neurological disease is multiple sclerosis.

In some embodiments, administering a compound of Formula (I) to a subject that is predisposed to a neurological disease prevents the subject from developing any symptoms of the neurological disease. In some embodiments, administering a compound of Formula (I) to a subject that is does not yet display symptoms of a neurological disease prevents the subject from developing any symptoms of the neurological disease. In some embodiments, administering a compound of Formula (I) to a subject in need thereof diminishes the extent of the neurological disease in the subject. In some embodiments, administering a compound of Formula (I) to a subject in need thereof stabilizes the neurological disease (prevents or delays the worsening of the neurological disease). In some embodiments, administering a compound of Formula (I) to a subject in need thereof delays the occurrence or recurrence of the neurological disease. In some embodiments, administering a compound of Formula (I) to a subject in need thereof slows the progression of the neurological disease. In some embodiments, administering a compound of Formula (I) to a subject in need thereof provides a partial remission of the neurological disease. In some embodiments, administering a compound of Formula (I) to a subject in need thereof provides a total remission of the neurological disease. In some embodiments, administering a compound of Formula (I) to a subject in need thereof decreases the dose of one or more other medications required to treat the neurological disease. In some embodiments, administering a compound of Formula (I) to a subject in need thereof enhances the effect of another medication used to treat the neurological disease. In some embodiments, administering a compound of Formula (I) to a subject in need thereof delays the progression of the neurological disease. In some embodiments, administering a compound of Formula (I) to a subject in need thereof increases the quality of life of the subject having a neurological disease. In some embodiments, administering a compound of Formula (I) to a subject in need thereof prolongs survival of a subject having a neurological disease.

In one aspect, provided herein is method of preventing a subject that is predisposed to a neurological disease from developing any symptoms of the neurological disease, the method comprising administering a compound of Formula (I) to the subject. In some embodiments, provided herein is a method of preventing a subject that does not yet display symptoms of a neurological disease from developing any symptoms of the neurological disease, the method comprising administering a compound of Formula (I) to the subject.

In some aspects, provided herein is a method of diminishing the extent of a neurological disease in a subject, the method comprising administering a compound of Formula (I) to the subject. In some embodiments, provided herein is a method of stabilizing a neurological disease in a subject, the method comprising administering a compound of Formula (I) to the subject. In some embodiments, the method prevents the worsening of the neurological disease. In some embodiments, the method delays the worsening of the neurological disease.

In another aspect, provided herein is a method of delaying the occurrence or recurrence of a neurological disease in a subject, the method comprising administering a compound of Formula (I) to the subject.

In some embodiments, provided herein is a method of slowing the progression of a neurological disease in a subject, the method comprising administering a compound of Formula (I) to the subject. In some embodiments, the method provides a partial remission of the neurological disease. In some embodiments, the method provides a total remission of the neurological disease.

In further aspects, provided herein is a method of decreasing the dose of one or more other medications required to treat a neurological disease in a subject, the method comprising administering a compound of Formula (I) to the subject. In some embodiments, provided herein is a method of enhancing the effect of another medication used to treat a neurological disease in a subject, the method comprising administering a compound of Formula (I) to the subject.

Also provided here is a method of delaying the progression of a neurological disease in a subject, the method comprising administering a compound of Formula (I) to the subject. In some embodiments, the method increases the quality of life of the subject having a neurological disease. In some embodiments, the method prolongs survival of the subject having a neurological disease.

In another aspect, provided herein is a method for treating neurological symptoms caused by a disease in a subject in need thereof, comprising administering to the subject an effective amount of a compound of Formula (I). In some embodiments, provided herein is a method for preventing neurological symptoms caused by a disease in a subject in need thereof, comprising administering to the subject an effective amount of a compound of Formula (I). In some embodiments, administering a compound of Formula (I) to a subject that is predisposed to a disease which causes neurological symptoms prevents the subject from developing any neurological symptoms. In some embodiments, administering a compound of Formula (I) to a subject that is does not yet display neurological symptoms of a disease which causes neurological symptoms prevents the subject from developing any neurological symptoms. In some embodiments, administering a compound of Formula (I) to a subject in need thereof diminishes the extent of the neurological symptoms caused by the disease in the subject. In some embodiments, administering a compound of Formula (I) to a subject in need thereof stabilizes the neurological symptoms of the disease (prevents or delays the worsening of the neurological symptoms). In some embodiments, administering a compound of Formula (I) to a subject in need thereof delays the occurrence or recurrence of the neurological symptoms caused by the disease. In some embodiments, administering a compound of Formula (I) to a subject in need thereof slows the progression of the neurological symptoms caused by the disease. In some embodiments, administering a compound of Formula (I) to a subject in need thereof provides a partial remission of the disease which causes neurological symptoms. In some embodiments, administering a compound of Formula (I) to a subject in need thereof provides a total remission of the disease which causes neurological symptoms. In some embodiments, administering a compound of Formula (I) to a subject in need thereof decreases the dose of one or more other medications required to treat the disease which causes neurological symptoms. In some embodiments, administering a compound of Formula (I) to a subject in need thereof enhances the effect of another medication used to treat the neurological symptoms of the disease. In some embodiments, administering a compound of Formula (I) to a subject in need thereof delays the progression of the disease which causes neurological symptoms. In some embodiments, administering a compound of Formula (I) to a subject in need thereof increases the quality of life of the subject having a disease which causes neurological symptoms. In some embodiments, administering a compound of Formula (I) to a subject in need thereof prolongs survival of a subject having a disease which causes neurological symptoms. In some embodiments, the disease is Niemann-Pick disease.

In some embodiments, compounds of Formula (I) are useful for treating a disorder selected from Alzheimer's disease, arthritis, rheumatoid arthritis, osteoarthritis, juvenile chronic arthritis, Lyme arthritis, psoriatic arthritis, reactive arthritis, and septic arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection (including but not limited to bone marrow and solid organ rejection), acute or chronic immune disease associated with organ transplantation, sarcoidosis, atherosclerosis, disseminated intravascular coagulation, Kawasaki's disease. Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acute transverse myelitis, Huntington's chorea, Parkinson's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinemia), dilated cardiomyopathy, infertility, female infertility, ovarian failure, premature ovarian failure, fibrotic lung disease, chronic wound healing, cryptogenic fibrosing alveolitis, post-inflammatory interstitial lung disease, fibrosis, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polytnyositis associated lung disease, Sjogren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjogren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and ThI Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma), and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aordic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, Burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, Epstein Barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallerrorden-Spatz disease, Hashimoto's thyroiditis, hay fever, heart transplant rejection, hemachromatosis, hemodialysis, hemolytic uremic syndrome/thrombolytic thrombocytopenia purpura, hemorrhage, hepatitis (A), His bundle arrythmias, HIV infection/HIV neuropathy, Hodgkin's disease, hyperkinetic movement disorders, hypersensitity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphedema, malaria, malignamt Lymphoma, malignant histiocytosis, malignant melanoma, meningitis, meningococcemia, metabolic/idiopathic, migraine headache, mitochondrial multisystem disorder, mixed connective tissue disease, monoclonal gammopathy, multiple myeloma, multiple systems degenerations (Mencel Dejerine-Thomas Shi-Drager and Machado-Joseph), myasthenia gravis, Mycobacterium avium intracellulare, Mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, Pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynaud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, acute pain, age-associated memory impairment (AAMI), anxiety attention deficit disorder, attention deficit disorder in general, attention deficit hyperactivity disorder (ADHD), bipolar disorder, cancer pain, central neuropathic pain syndromes, central post-stroke pain, chemotherapy-induced neuropathy, cognitive deficits and dysfunction in psychiatric disorders, cognitive deficits associated with aging and neurodegeneration, cognitive deficits associated with diabetes, cognitive deficits of schizophrenia, complex regional pain syndrome, declines in cognitive function in Alzheimer's and associated dementias, deficits in attention, dementia, dementia associated with Down's syndrome, dementia associated with Lewy bodies, depression in Cushing's syndrome, diminished CNS function associated with traumatic brain injury, diseases with deficits of memory, dizziness, drug abuse, epilepsy, HIV sensory neuropathy, Huntingdon's disease, hyperalgesia including neuropathic pain, inflammation and inflammatory disorders, inflammatory hyperalgesia, inflammatory pain, insulin resistance syndrome, jet lag, lack of circulation, learning, major depressive disorder, medullary thyroid carcinoma, Meniere's disease, metabolic syndrome, mild cognitive impairment, mood alteration, motion sickness, multiple sclerosis pain, narcolepsy, need for new blood vessel growth associated with vascularization of skin grafts and lack of circulation, need for new blood vessel growth associated with wound healing, neuropathic pain, neuropathy, neuropathy secondary to tumor infiltration, noninflammatory pain, obesity, obsessive compulsive disorder, painful diabetic neuropathy, panic disorder, Parkinson disease pain, pathological sleepiness, phantom limb pain, Pick's Disease, polycystic ovary syndrome, post traumatic stress disorder, post-herpetic neuralgia, post-mastectomy pain, post-surgical pain, psychotic depression, schizoaffective disorder, seizures, senile dementia, sepsis syndrome, sleep disorders, smoking cessation, spinal cord injury pain, steroid-induced acute psychosis, sub-categories of neuropathic pain including peripheral neuropathic pain syndromes, substance abuse including alcohol abuse, Syndrome X, Tourette's syndrome, treatment resistant depression, trigeminal neuralgia, type II diabetes, vertigo, and vestibular disorders.

### Pharmaceutical Compositions and Routes of Administration

The compounds provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The compounds disclosed herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds of Formula (I) in the pharmaceutical composition may be at a level that will exercise the desired effect; for example, about 0.005 mg/kg of a subject's body weight to about 10 mg/kg of a subject's body weight in unit dosage for both oral and parenteral administration.

The dose of a compound of Formula (I) to be administered to a subject is rather widely variable and can be subject to the judgment of a health-care practitioner. In general, the compounds disclosed herein can be administered one to four times a day in a dose of about 0.001 mg/kg of a subject's body weight to about 10 mg/kg of a subject's body weight, but the above dosage may be properly varied depending on the age, body weight and medical condition of the subject and the type of administration. In one embodiment, the dose is about 0.001 mg/kg of a subject's body weight to about 5 mg/kg of a subject's body weight, about 0.01 mg/kg of a subject's body weight to about 5 mg/kg of a subject's body weight, about 0.05 mg/kg of a subject's body weight to about 1 mg/kg of a subject's body weight, about 0.1 mg/kg of a subject's body weight to about 0.75 mg/kg of a subject's body weight or about 0.25 mg/kg of a subject's body weight to about 0.5 mg/kg of a subject's body weight. In one embodiment, one dose is given per day. In any given case, the amount of the compound of Formula (I) administered will depend on such factors as the solubility of the active component, the formulation used and the route of administration.

In some embodiments, a compound of Formula (I) is administered to a subject at a dose of about 0.01 mg/day to about 750 mg/day, about 0.1 mg/day to about 375 mg/day, about 0.1 mg/day to about 150 mg/day, about 0.1 mg/day to about 75 mg/day, about 0.1 mg/day to about 50 mg/day, about 0.1 mg/day to about 25 mg/day, or about 0.1 mg/day to about 10 mg/day.

In another embodiment, provided herein are unit dosage formulations that comprise between about 0.1 mg and 500 mg, about 1 mg and 250 mg, about 1 mg and about 100 mg, about 1 mg and about 50 mg, about 1 mg and about 25 mg, or between about 1 mg and about 10 mg of a compound of Formula (I).

In a particular embodiment, provided herein are unit dosage formulations comprising about 0.1 mg or 100 mg of a compound of Formula (I).

In another embodiment, provided herein are unit dosage formulations that comprise 0.5 mg, 1 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of a compound of Formula (I).

A compound of Formula (I) can be administered once, twice, three, four or more times daily. In a particular embodiment, doses of 100 mg or less are administered as a once daily dose and doses of more than 100 mg are administered twice daily in an amount equal to one half of the total daily dose.

A compound of Formula (I) can be administered orally for reasons of convenience. In one embodiment, when administered orally, a compound of Formula (I) is administered with a meal and water. In another embodiment, the compound of Formula (I) is dispersed in water or juice (e.g., apple juice or orange juice) or any other liquid and administered orally as a solution or a suspension.

The compounds disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a compound of Formula (I) without an additional carrier, excipient or vehicle.

In another embodiment, provided herein are compositions comprising an effective amount of a compound of Formula (I) and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of Formula (I) with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of Formula (I) as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound of Formula (I) can be delayed or prolonged by proper formulation. For example, a slowly soluble pellet of the compound of Formula (I) can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of Formula (I) in oily or emulsified vehicles that allow it to disperse slowly in the serum.

### Exemplary Embodiments

The present disclosure is further described by the following embodiments. The features of each of the embodiments are combinable with any of the other embodiments where appropriate and practical.

Embodiment 1. A compound of Formula (I):

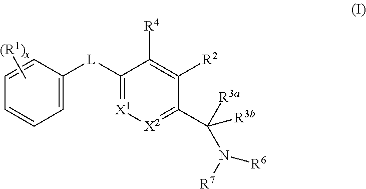

or a pharmaceutically acceptable salt thereof, wherein:
L is —C≡C—, —HC═CH—, —CH₂CH₂—, —CH₂O—,

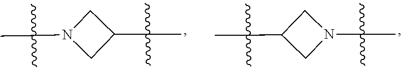 [SMILES: *C1CN(*)C1 ; *C1CN(*)C1]

or a bond;
each R¹ is independently halo, —CN, C₁-C₆ alkyl, C₁-C₆ haloalkyl, C₁-C₆ alkoxy, or C₃-C₆ cycloalkyl;
x is 0-5;
R² is H, halo, C₁-C₆ alkyl, C₃-C₆ cycloalkyl, or C₁-C₆ haloalkyl;
R^{3a} and R^{3b} are each H;
or R² and R^{3a} are taken together with the carbon atoms to which they are attached to form a fused cyclopentyl;
or R² and R⁴ are taken together with the carbon atoms to which they are attached to form a fused phenyl;
R⁴ is H, halo, —CN, C₁-C₆ alkyl, C₁-C₆ haloalkyl, C₁-C₆ alkoxy, or C₃-C₆ cycloalkyl;
X¹ and X² are independently N or CR⁵;
each R⁵ is independently H, halo, —CN, C₁-C₆ alkyl, C₁-C₆ haloalkyl, C₁-C₆ alkoxy, or C₃-C₆ cycloalkyl;
R⁶ is H;
R⁷ is C₁-C₆ alkyl-OH;
or R⁶ and R⁷ are taken together with the nitrogen atom to which they are attached to form a 4- to 6-membered heterocyclyl substituted with n R⁸ groups;
n is 1-5; and
each R⁸ is independently halo, —CN, C₁-C₆ alkyl, C₁-C₆ haloalkyl, C₁-C₆ alkoxy, or —OH, provided that at least one R⁸ is —OH.

Embodiment 2. The compound of embodiment 1, or a pharmaceutically acceptable salt thereof, wherein:
L is —C≡C—, —CH₂CH₂—, or —CH₂O—.

Embodiment 3. The compound of embodiment 1, or a pharmaceutically acceptable salt thereof, wherein:
L is

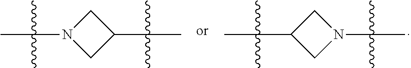 [SMILES: *C1CN(*)C1 ; *C1CN(*)C1]

Embodiment 4. The compound of embodiment 1, or a pharmaceutically acceptable salt thereof, wherein:
L is a bond.

Embodiment 5. The compound of any one of embodiments 1-4, or a pharmaceutically acceptable salt thereof, wherein:
each R¹ is independently halo, —CN, C₁-C₃ alkyl, C₁-C₃ haloalkyl, C₁-C₃ alkoxy, or C₃-C₆ cycloalkyl.

Embodiment 6. The compound of embodiment 5, or a pharmaceutically acceptable salt thereof, wherein:
each R¹ is independently F, C₁, or cyclopropyl.

Embodiment 7. The compound of any one of embodiments 1-6, or a pharmaceutically acceptable salt thereof, wherein:
x is 0, 1, or 2.

Embodiment 8. The compound of any one of embodiments 1-7, or a pharmaceutically acceptable salt thereof, wherein:

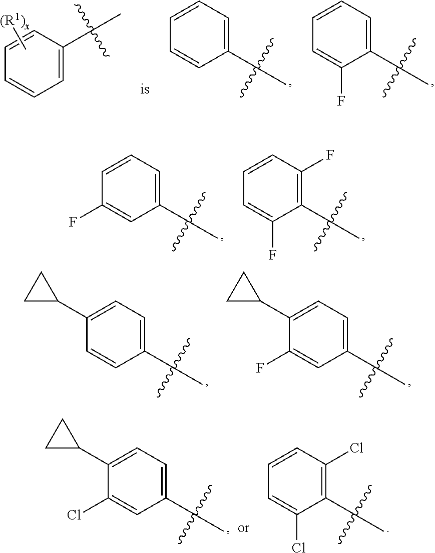

Embodiment 9. The compound of any one of embodiments 1-8, or a pharmaceutically acceptable salt thereof, wherein:
R² is H, halo, C₁-C₃ alkyl, C₃-C₆ cycloalkyl, or C₁-C₃ haloalkyl.

Embodiment 10. The compound of embodiment 9, or a pharmaceutically acceptable salt thereof, wherein:
R² is H, F, CI, —CH₃, —CH₂CH₃, —CH(CH₃)₂, or cyclopropyl.

Embodiment 11. The compound of any one of embodiments 1-8, or a pharmaceutically acceptable salt thereof, wherein:
R² and R^{3a} are taken together with the carbon atoms to which they are attached to form a fused cyclopentyl; and
R^{3b} is H.

Embodiment 12. The compound of any one of embodiments 1-10, or a pharmaceutically acceptable salt thereof, wherein:
R^{3a} and R^{3b} are each H.

Embodiment 13. The compound of any one of embodiments 1-8 and 12, or a pharmaceutically acceptable salt thereof, wherein:
R² and R⁴ are taken together with the carbon atoms to which they are attached to form a fused phenyl.

Embodiment 14. The compound of any one of embodiments 1-12, or a pharmaceutically acceptable salt thereof, wherein:
R⁴ is H, halo, —CN, C₁-C₃ alkyl, C₁-C₃ haloalkyl, C₁-C₃ alkoxy, or C₃-C₆ cycloalkyl.

Embodiment 15. The compound of embodiment 14, or a pharmaceutically acceptable salt thereof, wherein:
R⁴ is H, F, or —CH₃.

Embodiment 16. The compound of any one of embodiments 1-15, or a pharmaceutically acceptable salt thereof, wherein:
X¹ and X² are independently CR⁵.

Embodiment 17. The compound of any one of embodiments 1-15, or a pharmaceutically acceptable salt thereof, wherein:
X¹ is N; and
X² is CR⁵.

Embodiment 18. The compound of any one of embodiments 1-15, or a pharmaceutically acceptable salt thereof, wherein:
X¹ is CR⁵; and
X² is N.

Embodiment 19. The compound of any one of embodiments 1-18, or a pharmaceutically acceptable salt thereof, wherein:
each R⁵ is independently H, halo, —CN, C₁-C₃ alkyl, C₁-C₃ haloalkyl, C₁-C₃ alkoxy, or C₃-C₆ cycloalkyl.

Embodiment 20. The compound of embodiment 19, or a pharmaceutically acceptable salt thereof, wherein:
each R⁵ is independently H, F, —CH₃, —CH₂CH₃, or —CH(CH₃)₂.

Embodiment 21. The compound of any one of embodiments 1-20, or a pharmaceutically acceptable salt thereof, wherein:

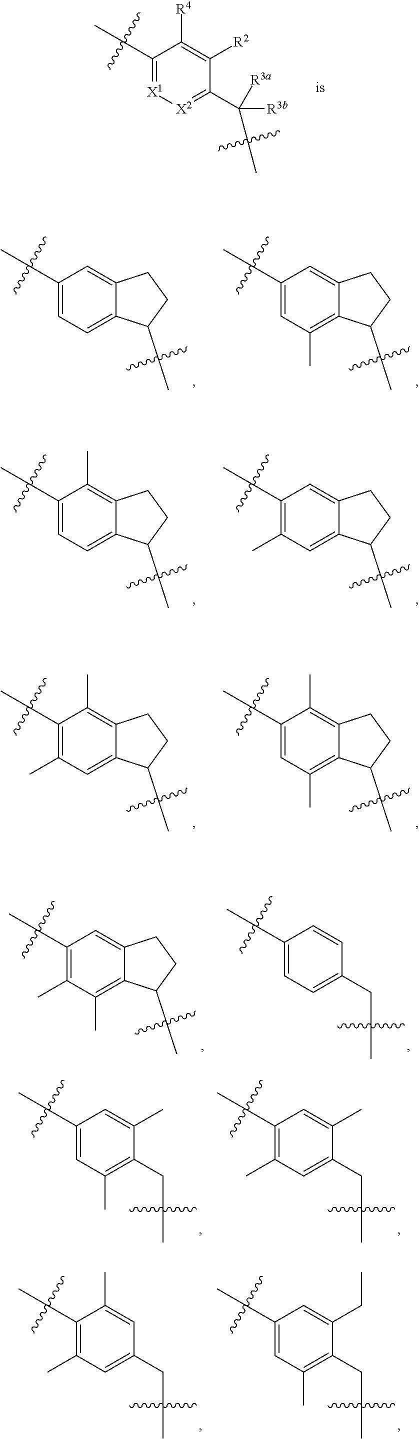

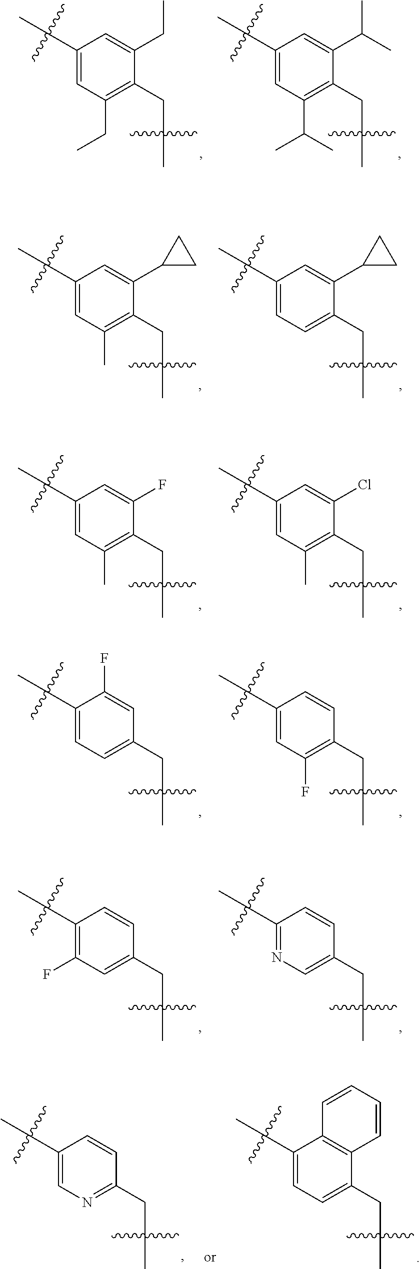

Embodiment 22. The compound of any one of embodiments 1-21, or a pharmaceutically acceptable salt thereof, wherein:
R⁶ is H; and
R⁷ is C₁-C₆ alkyl-OH.

Embodiment 23. The compound of embodiment 22, or a pharmaceutically acceptable salt thereof, wherein:
R⁶ is H; and
R⁷ is —CH₂C(OH)(CH₃)₂.

Embodiment 24. The compound of any one of embodiments 1-21, or a pharmaceutically acceptable salt thereof, wherein:
R⁶ and R⁷ are taken together with the nitrogen atom to which they are attached to form

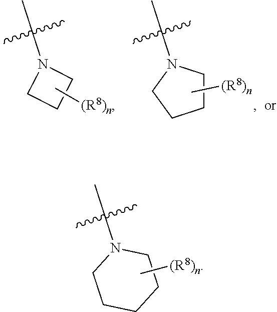

Embodiment 25. The compound of embodiment 24, or a pharmaceutically acceptable salt thereof, wherein:
each R⁸ is independently halo, —CN, C₁-C₃ alkyl, C₁-C₃ haloalkyl, C₁-C₃ alkoxy, or —OH.

Embodiment 26. The compound of embodiment 25, or a pharmaceutically acceptable salt thereof, wherein:
each R⁸ is independently-CH₃, —CH₂CH₃, —CFH₂, —CF₂H, —CF₃, or —OH.

Embodiment 27. The compound of any one of embodiments 1-21 and 24-26, or a pharmaceutically acceptable salt thereof, wherein:
n is 2.

Embodiment 28. The compound of embodiment 27, or a pharmaceutically acceptable salt thereof, wherein one R⁸ is —OH.

Embodiment 29. The compound of any one of embodiments 1-21 and 24-28, or a pharmaceutically acceptable salt thereof, wherein:

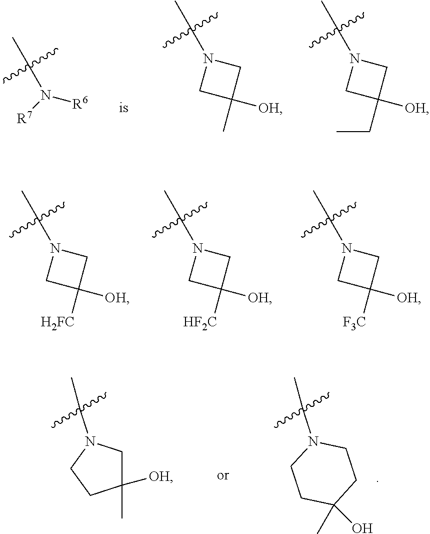

Embodiment 30. The compound of any one of embodiments 1-8, 11, and 14-29, or a pharmaceutically acceptable salt thereof, wherein the compound is of Formula (II):

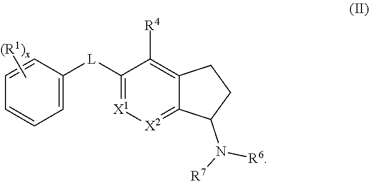

Embodiment 31. The compound of embodiment 30, or a pharmaceutically acceptable salt thereof, wherein the compound is of Formula (II-A) or (II-B):

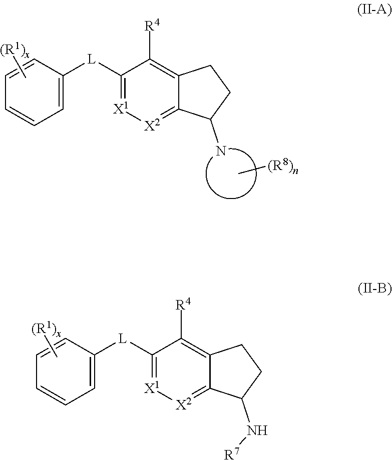

and

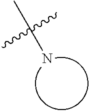 [SMILES: *N]

is a 4- to 6-membered heterocyclyl.

Embodiment 32. The compound of any one of embodiments 1-10 and 12-29, or a pharmaceutically acceptable salt thereof, wherein the compound is of Formula (III):

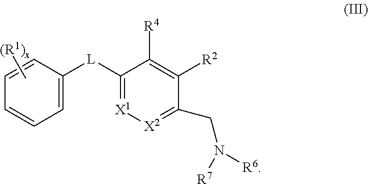

Embodiment 33. The compound of embodiment 32, or a pharmaceutically acceptable salt thereof, wherein the compound is of Formula (III-A) or (III-B):

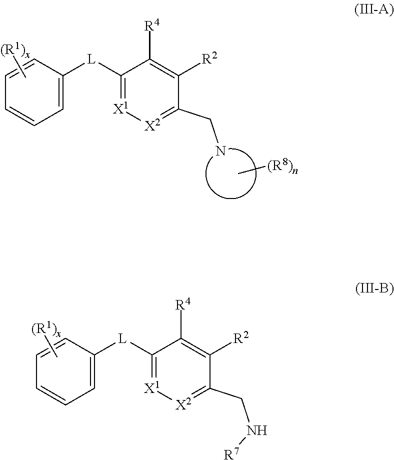

and

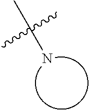 [SMILES: *N]

is a 4- to 6-membered heterocyclyl.

Embodiment 34. A compound selected from the compounds of Table 1 and pharmaceutically acceptable salts thereof.

Embodiment 35. A pharmaceutical composition comprising the compound of any one of embodiments 1-34, or a pharmaceutically acceptable salt thereof, and a pharmaceutically acceptable excipient.

Embodiment 36. A method of modulating sphingosine 1-phosphate receptor 5 (S1P5) comprising contacting S1P5 with an effective amount of the compound of any one of embodiments 1-34, or a pharmaceutically acceptable salt thereof, or the pharmaceutical composition of embodiment 35.

Embodiment 37. A method of treating a neurological disease in a subject in need thereof, comprising administering to the subject an effective amount of the compound of any one of embodiments 1-34, or a pharmaceutically acceptable salt thereof, or the pharmaceutical composition of embodiment 35.

Embodiment 38. The method of embodiment 37, wherein the neurological disease is Alzheimer's disease, multiple sclerosis, migraine, and amyotrophic lateral sclerosis.

### EXAMPLES

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

As used in certain of the chemical structures provided in the following Examples, designation of a particular atom with “or” indicates that the absolute stereochemistry of the indicated atom was not determined.

The following abbreviations may be relevant for the application.

### Abbreviations

| | |
|---|---|
| ACN/MeCN | acetonitrile |
| DCM | dichloromethane |
| DEA | diethanolamine |
| DMF | dimethylformamide |
| DMSO | dimethyl sulfoxide |
| e.e. | enantiomeric excess |
| EA | ethyl acetate |
| EDTA | ethylenediaminetetraacetic acid |
| equiv. | equivalents |
| ESI | electrospray ionization |
| EtOAc | ethyl acetate |
| EtOH | ethanol |
| h | hour(s) |
| HEPES | 4-(2-hydroxyethyl)-1-piperazineethanesulfonic acid |
| Hex | hexanes |
| HPLC | high performance liquid chromatography |
| IPA | isopropyl alcohol |
| LCMS | liquid chromatography mass spectrometry |
| MeOH/MeOD | Methanol/deuterated methanol |
| Me-THF | methyltetrahydrofuran |
| min | minute(s) |
| Pd(dppf)Cl₂ | [1,1′-bis(diphenylphosphino)ferrocene]dichloropalladium(II) |
| Pd₂(dba)₃ | tris(dibenzylideneacetone)dipalladium (0) |
| PE | petroleum ether |
| PPh₃ | triphenylphosphine |
| prep. | preparative |
| rt | room temperature or ambient temperature |
| RT | retention time |
| sat. | saturated |
| SFC | supercritical fluid chromatography |
| TBSOTf | tert-Butyldimethylsilyl trifluoromethanesulfonate |
| t-BuBrettphos | 2-(di-tert-butylphosphino)-2′,4′,6′-triisopropyl-3,6-dimethoxy- |
| | 1,1′-biphenyl |
| TFA | trifluoroacetic acid |
| THF | tetrahydrofuran |
| TLC | thin layer chromatography |

### Synthetic Examples

### Example S1. 1-(5-((2-fluoro-phenyl)ethynyl)-2,3-dihydro-1 H-inden-1-yl)-3-methylazetidin-3-ol (1a & 1b)

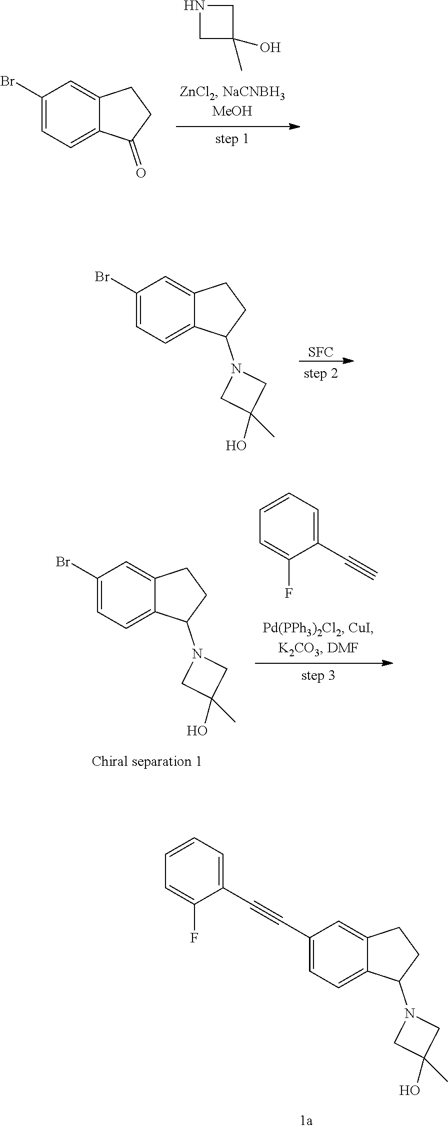

### Synthesis of 1-(5-bromoindan-1-yl)-3-methyl-azetidin-3-ol

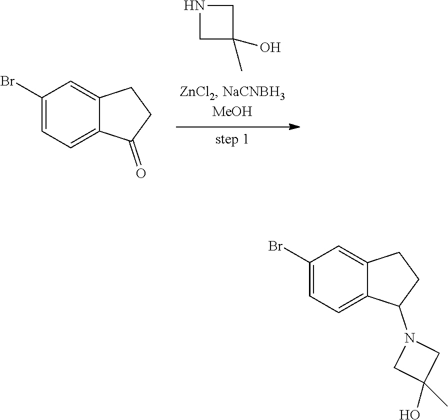

A mixture of NaCNBH₃ (4.76 g, 75.8 mmol, 4 equiv.) and ZnCl₂ (4 M in 4Me-THF, 28.5 mL, 113.8 mmol, 6 equiv.) in methanol (50 mL) was stirred for 30 min at room temperature. Then 5-bromoindan-1-one (4.0 g, 18.9 mmol, 1 equiv.) and 3-methylazetidin-3-ol (3.3 g, 37.9 mmol, 2 equiv.) were added in portions. The resulting mixture was stirred overnight at 60° C. LCMS showed the reaction was completed. The reaction was quenched by water (100 mL) and extracted with DCM (3×30 mL). The combined organic layers were concentrated under reduced pressure. The residue was purified by flash chromatography on silica gel (eluted with ethyl acetate/petroleum ether, 1:1) to afford 1-(5-bromoindan-1-yl)-3-methyl-azetidin-3-ol (5 g, 93%) as a yellow oil. LCMS (ESI, m/z): 282 [M+H]⁺.

### Chiral Separation of 1-(5-bromoindan-1-yl)-3-methyl-azetidin-3-ol

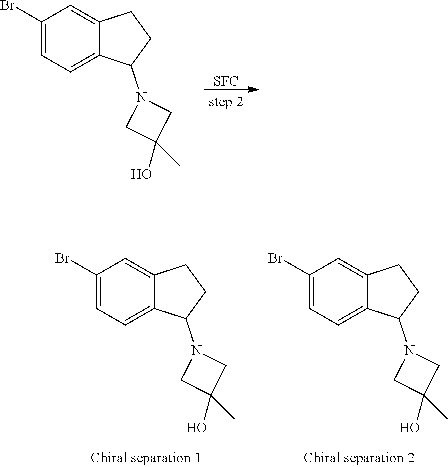

5 g of the racemic product was resolved by SFC(Column: Lux Cellulose-4, 3*25 cm, 5 μm; Mobile Phase A: CO₂, Mobile Phase B: IPA (0.2% DEA); Flow rate: 100 mL/min; Gradient: 30% B; 220 nm) to afford the first eluting peak (2.2 g, RT: 3.28 min) and the second eluting peak (2.1 g, RT: 4.07 min) both as light yellow oil.

### Synthesis of 1-[5-[2-(2-fluorophenyl)ethynyl]indan-1-yl]-3-methyl-azetidin-3-ol (1a)

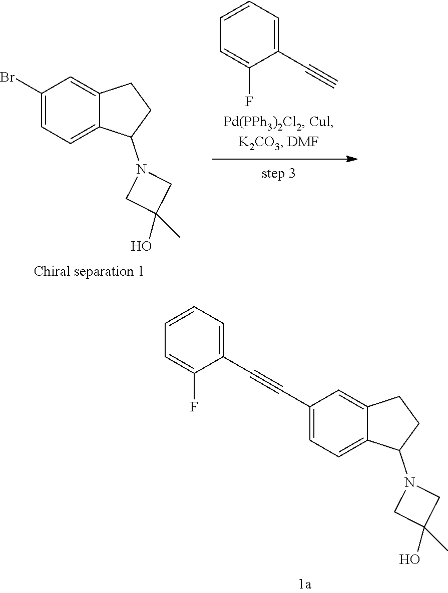

To a stirred solution of 1-(5-bromoindan-1-yl)-3-methyl-azetidin-3-ol (100 mg, 0.35 mmol, 1 equiv.) in DMF (2 mL) were added 1-ethynyl-2-fluoro-benzene (213 mg, 1.77 mmol, 5 equiv.), Pd(PPh₃)₂Cl₂ (25 mg, 0.04 mmol, 0.1 equiv.), CuI (14 mg, 0.07 mmol, 0.2 equiv.) and K₂CO₃ (143 mg, 1.06 mmol, 3 equiv.). The resulting mixture was stirred overnight at 80° C. LCMS showed the reaction was completed. The reaction mixture was filtered through Celite and the filtrate was concentrated under reduced pressure. The residue was purified by Prep-HPLC (Column: XBridge Shield RP18 OBD Column, 5 μm, 19*150 mm; Mobile Phase A: water (10 mM NH₄HCO₃+0.1% NH₃·H₂O), Mobile Phase B: ACN; Flow rate: 60 mL/min; Gradient: 40% B to 60% B in 7 min; 210/254 nm; RT: 6.52 min) to afford 1-[5-[2-(2-fluorophenyl)ethynyl]indan-1-yl]-3-methyl-azetidin-3-ol (25.6 mg, 22%) as a white solid.

LCMS (ESI, m/z): 322 [M+H]⁺. Analytic Conditions: column: EVO C18, 3.0*50 mm, 2.6 μm; mobile phase A: water (5 mM NH₄HCO₃), mobile phase B: acetonitrile; flow rate: 1.20 mL/min; gradient: 10% B to 95% B in 2.00 min, hold at 95% for 0.60 min, 95% B to 10% B in 0.15 min; 254 nm; RT: 1.569 min.

¹H NMR (400 MHZ, DMSO-d6) δ 7.64-7.60 (m, 1H), 7.50-7.45 (m, 1H), 7.42 (s, 1H), 7.37-7.25 (m, 4H), 5.16 (s, 1H), 3.84-3.81 (m, 1H), 3.19-3.17 (m, 1H), 3.13-3.09 (m, 2H), 2.96 (d, J=6.4 Hz, 1H), 2.91 (t, J=8.0 Hz, 1H), 2.79-2.72 (m, 1H), 2.09-2.00 (m, 1H), 1.87-1.80 (m, 1H), 1.32 (s, 3H).

### Synthesis of 1-[5-[2-(2-fluorophenyl)ethynyl]indan-1-yl]-3-methyl-azetidin-3-ol (1b)

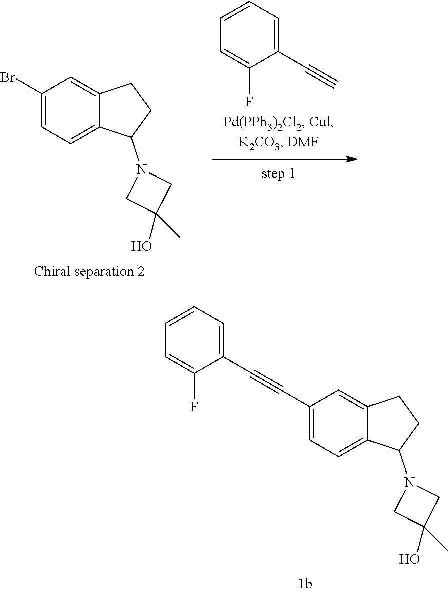

To a stirred solution of 1-(5-bromoindan-1-yl)-3-methyl-azetidin-3-ol (100 mg, 0.35 mmol, 1 equiv.) in DMF (2 mL) were added 1-ethynyl-2-fluoro-benzene (213 mg, 1.77 mmol, 5 equiv.), Pd(PPh₃)₂Cl₂ (25 mg, 0.04 mmol, 0.1 equiv.), CuI (14 mg, 0.07 mmol, 0.2 equiv.) and K₂CO₃ (143 mg, 1.06 mmol, 3 equiv.). The resulting mixture was stirred overnight at 80° C. LCMS showed the reaction was completed. The reaction mixture was filtered through Celite and the filtrate was concentrated under reduced pressure. The residue was purified by Prep-HPLC (Column: XBridge Shield RP18 OBD Column, 5 μm, 19*150 mm; Mobile Phase A: water (10 mM NH₄HCO₃+0.1% NH₃·H₂O), Mobile Phase B: ACN; Flow rate: 60 mL/min; Gradient: 40% B to 60% B in 7 min; 210/254 nm; RT: 6.52 min) to afford 1-[5-[2-(2-fluorophenyl)ethynyl]indan-1-yl]-3-methyl-azetidin-3-ol (25.3 mg, 22%) as a white solid.

LCMS (ESI, m/z): 322 [M+H]⁺. Analytic Conditions: column: EVO C18, 3.0*50 mm, 2.6 μm; mobile phase A: water (5 mM NH₄HCO₃), mobile phase B: acetonitrile; flow rate: 1.20 mL/min; gradient: 10% B to 95% B in 2.00 min, hold at 95% for 0.60 min, 95% B to 10% B in 0.15 min; 254 nm; RT: 1.569 min.

¹H NMR (400 MHZ, DMSO-d6) δ 7.64-7.60 (m, 1H), 7.50-7.45 (m, 1H), 7.42 (s, 1H), 7.36-7.25 (m, 4H), 5.16 (s, 1H), 3.84-3.81 (m, 1H), 3.19-3.17 (m, 1H), 3.13-3.09 (m, 2H), 2.96 (d, J=6.8 Hz, 1H), 2.91 (t, J=8.0 Hz, 1H), 2.79-2.72 (m, 1H), 2.09-2.00 (m, 1H), 1.87-1.79 (m, 1H), 1.32 (s, 3H)

### Example S2. 1-[5-[2-(3-fluorophenyl)ethynyl]-indan-1-yl]-3-methyl-azetidin-3-ol (2a & 2b)

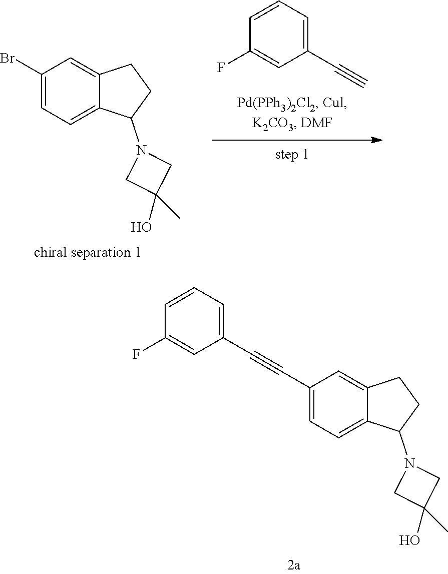

### Synthesis of 1-[5-[2-(3-fluorophenyl)ethynyl]indan-1-yl]-3-methyl-azetidin-3-ol (2a)

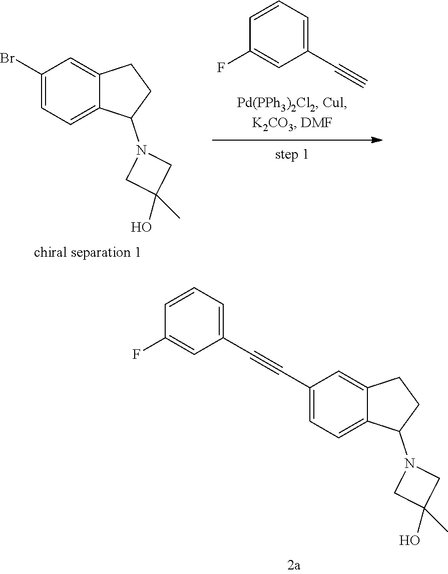

To a stirred solution of 1-(5-bromoindan-1-yl)-3-methyl-azetidin-3-ol (100 mg, 0.35 mmol, 1 equiv.) in DMF (2 mL) were added 1-ethynyl-3-fluoro-benzene (213 mg, 1.77 mmol, 5 equiv.), Pd(PPh₃)₂Cl₂ (25 mg, 0.04 mmol, 0.1 equiv.), CuI (14 mg, 0.07 mmol, 0.2 equiv.) and K₂CO₃ (143 mg, 1.06 mmol, 3 equiv.). The resulting mixture was stirred overnight at 80° C. LCMS showed the reaction was completed. The reaction mixture was filtered through Celite and the filtrate was concentrated under reduced pressure. The residue was purified by Prep-HPLC (Column: YMC-Actus Triart C18, 30*250 mm, 5 μm; Mobile Phase A: water (10 mM NH₄HCO₃+0.1% NH₃. H₂O), Mobile Phase B: ACN; Flow rate: 60 mL/min; Gradient: 45% B to 75% B in 7 min; 254/210 nm; RT: 6.42 min) to afford 1-[5-[2-(3-fluorophenyl)ethynyl]indan-1-yl]-3-methyl-azetidin-3-ol (94 mg, 82%) as a white solid.

LCMS (ESI, m/z): 322 [M+H]⁺. Analytic Conditions: column: Shim-pack XR-ODS, 3.0*50 mm, 2.2 μm; mobile phase A: water (0.05% TFA), mobile phase B: acetonitrile (0.05% TFA); flow rate: 1.20 mL/min; gradient: 5% B to 100% B in 2.00 min, hold at 100% for 0.70 min, 100% B to 5% B in 0.05 min; 254 nm; RT: 1.294 min.

¹H NMR (400 MHZ, DMSO-d₆) δ 7.50-7.44 (m, 1H), 7.42-7.38 (m, 3H), 7.37-7.34 (m, 1H), 7.30-7.25 (m, 2H), 5.16 (s, 1H), 3.84-3.81 (m, 1H), 3.19-3.17 (m, 1H), 3.13-3.09 (m, 2H), 2.96 (d, J=6.8 Hz, 1H), 2.91 (t, J=8.0 Hz, 1H), 2.79-2.72 (m, 1H), 2.09-2.00 (m, 1H), 1.87-1.80 (m, 1H), 1.32 (s, 3H).

### Synthesis of 1-[5-[2-(3-fluorophenyl)ethynyl]indan-1-yl]-3-methyl-azetidin-3-ol (2b)

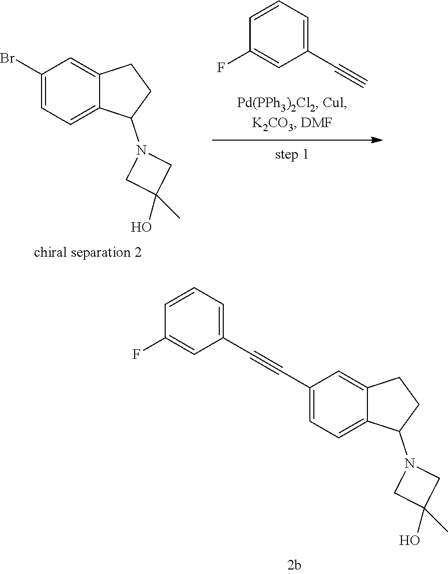

To a stirred solution of 1-(5-bromoindan-1-yl)-3-methyl-azetidin-3-ol (100 mg, 0.35 mmol, 1 equiv.) in DMF (2 mL) were added 1-ethynyl-3-fluoro-benzene (213 mg, 1.77 mmol, 5 equiv.), Pd(PPh₃)₂Cl₂ (25 mg, 0.04 mmol, 0.1 equiv.), CuI (14 mg, 0.07 mmol, 0.2 equiv.) and K₂CO₃ (143 mg, 1.06 mmol, 3 equiv.). The resulting mixture was stirred overnight at 80° C. LCMS showed the reaction was completed. The reaction mixture was filtered through Celite and the filtrate was concentrated under reduced pressure. The residue was purified by Prep-HPLC (Column: YMC-Actus Triart C18, 30*250 mm, 5 μm; Mobile Phase A: water (10 mM NH₄HCO₃+0.1% NH₃. H₂O), Mobile Phase B: ACN; Flow rate: 60 mL/min; Gradient: 45% B to 75% B in 7 min; 254/210 nm; RT: 6.32 min) to afford 1-[5-[2-(3-fluorophenyl)ethynyl]indan-1-yl]-3-methyl-azetidin-3-ol (85.9 mg, 74%) as a white solid.

LCMS (ESI, m/z): 322 [M+H]⁺. Analytic Conditions: column: Shim-pack XR-ODS, 3.0*50 mm, 2.2 μm; mobile phase A: water (0.05% TFA), mobile phase B: acetonitrile (0.05% TFA); flow rate: 1.20 mL/min; gradient: 5% B to 100% B in 2.00 min, hold at 100% for 0.70 min, 100% B to 5% B in 0.05 min; 254 nm; RT: 1.292 min.

¹H NMR (400 MHZ, DMSO-d₆) δ 7.50-7.44 (m, 1H), 7.42-7.37 (m, 3H), 7.36-7.34 (m, 1H), 7.30-7.25 (m, 2H), 5.17 (s, 1H), 3.84-3.81 (m, 1H), 3.19-3.17 (m, 1H), 3.14-3.09 (m, 2H), 2.96 (d, J=6.4 Hz, 1H), 2.91 (t, J=8.0 Hz, 1H), 2.79-2.72 (m, 1H), 2.09-2.00 (m, 1H), 1.87-1.79 (m, 1H), 1.32 (s, 3H).

### Example S3. 1-(5-((2,6-dichlorobenzyl)oxy)-2,3-dihydro-1H-inden-1-yl)-3-methylazetidin-3-ol (3a & 3b)

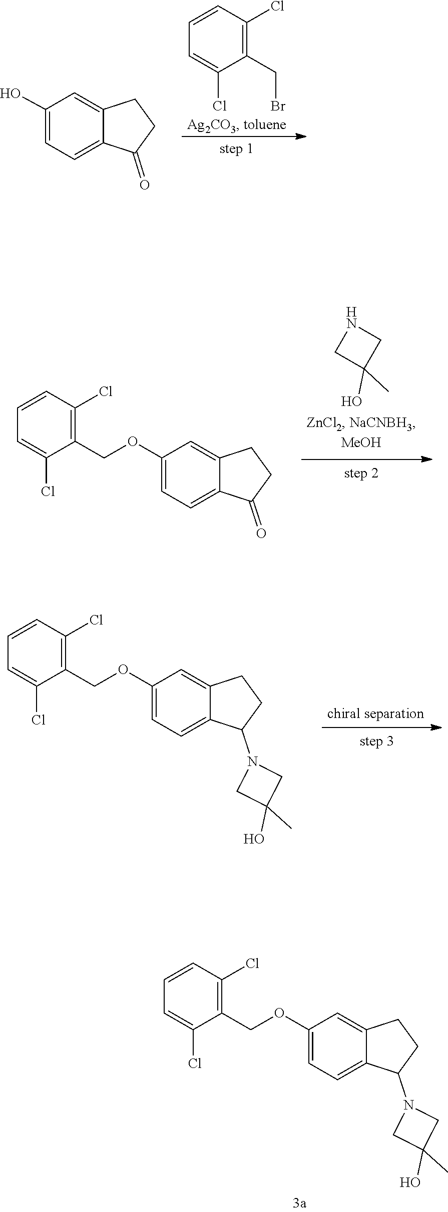

### Synthesis of 5-[(2,6-dichlorophenyl) methoxy]indan-1-one

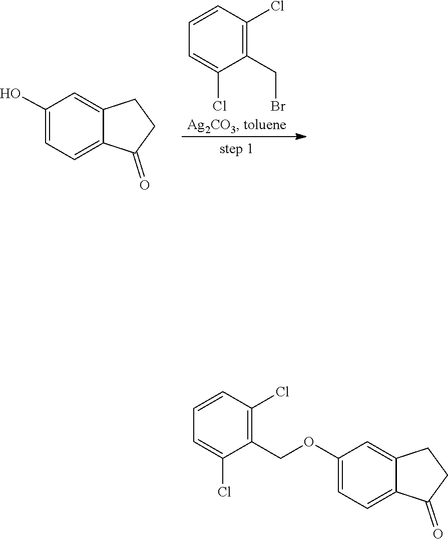 [SMILES: O=C1CCc2cc(O)ccc21 ; Clc1cccc(Cl)c1CBr ; O=C1CCc2cc(OCc3c(Cl)cccc3Cl)ccc21]

To a stirred solution of 5-hydroxyindan-1-one (2.0 g, 13.5 mmol, 1 equiv.) in toluene (15 mL) were added 2-(bromomethyl)-1,3-dichloro-benzene (6.5 g, 27.0 mmol, 2 equiv.) and Ag₂CO₃ (11.2 g, 40.5 mmol, 3 equiv.). The resulting mixture was stirred overnight at 110° C. LCMS showed the reaction was completed. The reaction mixture was filtered and the filtrate was concentrated under reduced pressure. The residue was purified by flash chromatography on silica gel (eluted with ethyl acetate/petroleum ether, 1:1) to afford 5-[(2,6-dichlorophenyl) methoxy]indan-1-one (775 mg, 19%) as a yellow oil. LCMS (ESI, m z): 307 [M+H]⁺.

### Synthesis of 1-(5-((2,6-dichlorobenzyl)oxy)-2,3-dihydro-1H-inden-1-yl)-3-methylazetidin-3-ol

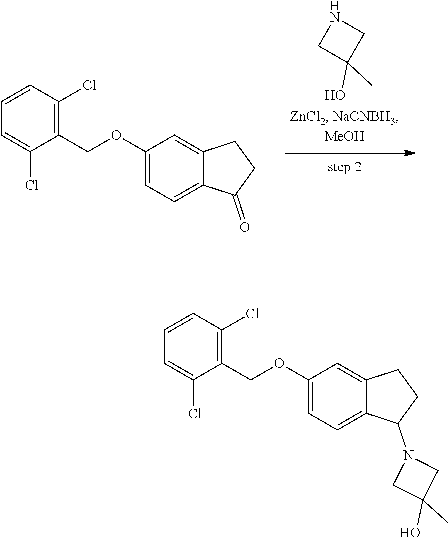

A mixture of NaCNBH₃ (82 mg, 1.30 mmol, 4 equiv.) and ZnCl₂ (4 M in 4Me-THF, 0.17 mL, 0.65 mmol, 2 equiv.) in methanol (2 mL) was stirred for 30 min at room temperature. Then 3-methylazetidin-3-ol (57 mg, 0.650 mmol, 2 equiv.) and 5-[(2,6-dichlorophenyl) methoxy]indan-1-one (100, mg, 0.33 mmol, 1 equiv.) were added. The resulting mixture was stirred overnight at 60° C. LCMS showed the reaction was completed. The reaction was quenched by water (20 mL) and extracted with DCM (3×10 mL). The combined organic layers were concentrated under reduced pressure. The residue was purified by Prep-HPLC(Column: XBridge Shield RP18 OBD Column, 5 μm, 19*150 mm; Mobile Phase A: water (10 mM NH+HCO₃+0.1% NH₃. H₂O), Mobile Phase B: ACN; Flow rate: 60 mL/min; Gradient: 35% B to 65% B in 7 min; 210/254 nm; RT: 6.59 min) to afford 1-(5-((2,6-dichlorobenzyl)oxy)-2,3-dihydro-1H-inden-1-yl)-3-methylazetidin-3-ol (100 mg, 80%) as a white solid. LCMS (ESI, m z): 378 [M+H]⁺.

### Chiral Separation of 1-(5-((2,6-dichlorobenzyl)oxy)-2,3-dihydro-1H-inden-1-yl)-3-methylazetidin-3-ol (3a)

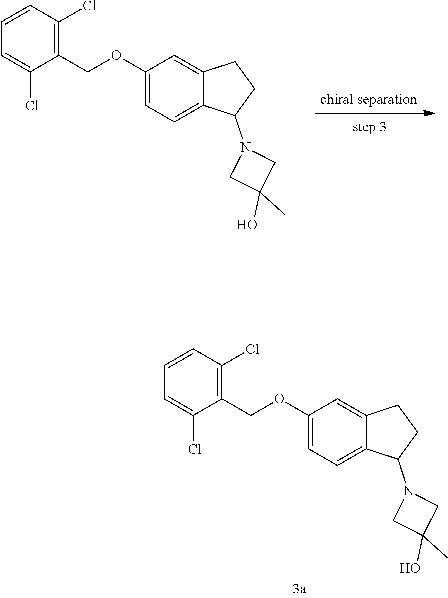 [SMILES: CC1(O)CN(C2CCc3cc(OCc4c(Cl)cccc4Cl)ccc32)C1 ; CC1(O)CN(C2CCc3cc(OCc4c(Cl)cccc4Cl)ccc32)C1]

100 mg of the racemic product was resolved by chiral-HPLC(Column: CHIRALPAK IG, 20*250 mm, 5 μm; Mobile Phase A: Hex (0.5% 2M NH₃-MeOH)-HPLC, Mobile Phase B: EtOH-HPLC; Flow rate: 20 mL/min; Gradient: 10% B to 10% B in 17 min; 220/254 nm) to afford the first eluting peak (43.3 mg, RT: 9.736 min) as a white solid.

LCMS (ESI, m/z): 378 [M+H]⁺. Analytic Conditions: column: HALO C18, 3.0*50 mm, 2.0 μm; mobile phase A: water (0.05% TFA), mobile phase B: acetonitrile (0.05% TFA); flow rate: 1.20 mL/min; gradient: 5% B to 100% B in 1.20 min, hold at 100% for 0.60 min, 100% B to 5% B in 0.03 min; 210 nm; RT: 0.916 min.

¹H NMR (400 MHZ, DMSO-d₆) δ 7.57-7.55 (m, 2H), 7.49-7.45 (m, 1H), 7.17 (d, J=8.0 Hz, 1H), 6.93 (d, J=2.4 Hz, 1H), 6.80 (dd, J=8.0, 2.4 Hz, 1H), 5.18 (s, 2H), 5.14 (s, 1H), 3.75-3.73 (m, 1H), 3.19-3.17 (m, 1H), 3.09-3.06 (m, 2H), 2.95-2.87 (m, 2H), 2.74-2.67 (m, 1H), 2.08-1.97 (m, 1H), 1.86-1.79 (m, 1H), 1.31 (s, 3H).

### Chiral Separation of 1-(5-((2,6-dichlorobenzyl)oxy)-2,3-dihydro-1H-inden-1-yl)-3-methylazetidin-3-ol (3b)

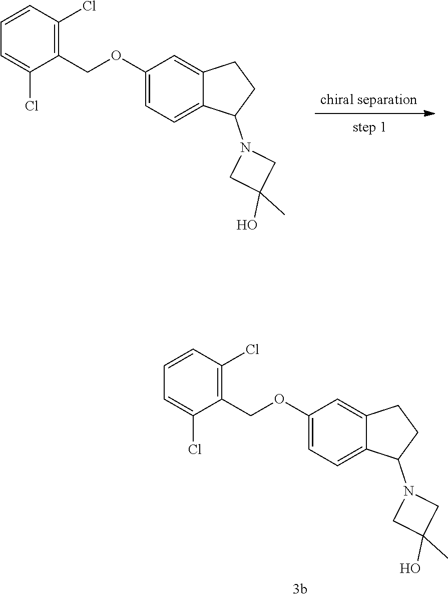 [SMILES: CC1(O)CN(C2CCc3cc(OCc4c(Cl)cccc4Cl)ccc32)C1 ; CC1(O)CN(C2CCc3cc(OCc4c(Cl)cccc4Cl)ccc32)C1]

100 mg of the racemic product was resolved by chiral-HPLC(Column: CHIRALPAK IG, 20*250 mm, 5 μm; Mobile Phase A: Hex (0.5% 2M NH₃-MeOH)-HPLC, Mobile Phase B: EtOH-HPLC; Flow rate: 20 mL/min; Gradient: 10% B to 10% B in 17 min; 220/254 nm) to afford the first eluting peak (45.8 mg, RT: 12.940 min) as a white solid.

LCMS (ESI, m/z): 378 [M+H]⁺. Analytic Conditions: column: HALO C18, 3.0*50 mm, 2.0 μm; mobile phase A: water (0.05% TFA), mobile phase B: acetonitrile (0.05% TFA); flow rate: 1.20 mL/min; gradient: 5% B to 100% B in 1.20 min, hold at 100% for 0.60 min, 100% B to 5% B in 0.03 min; 210 nm; RT: 0.911 min.

¹H NMR (400 MHZ, DMSO-d₆) δ 7.58-7.55 (m, 2H), 7.49-7.45 (m, 1H), 7.17 (d, J=8.0 Hz, 1H), 6.93 (d, J=2.4 Hz, 1H), 6.80 (dd, J=8.0, 2.4 Hz, 1H), 5.18 (s, 2H), 5.14 (s, 1H), 3.75-3.73 (m, 1H), 3.19-3.17 (m, 1H), 3.10-3.06 (m, 2H), 2.95-2.87 (m, 2H), 2.74-2.67 (m, 1H), 2.08-1.97 (m, 1H), 1.86-1.79 (m, 1H), 1.31 (s, 3H).

### Example S4. 1-(5-(4-cyclopropyl-3-fluorophenyl)-2,3-dihydro-1H-inden-1-yl)-3-methyl azetidin-3-ol (4a & 4b)

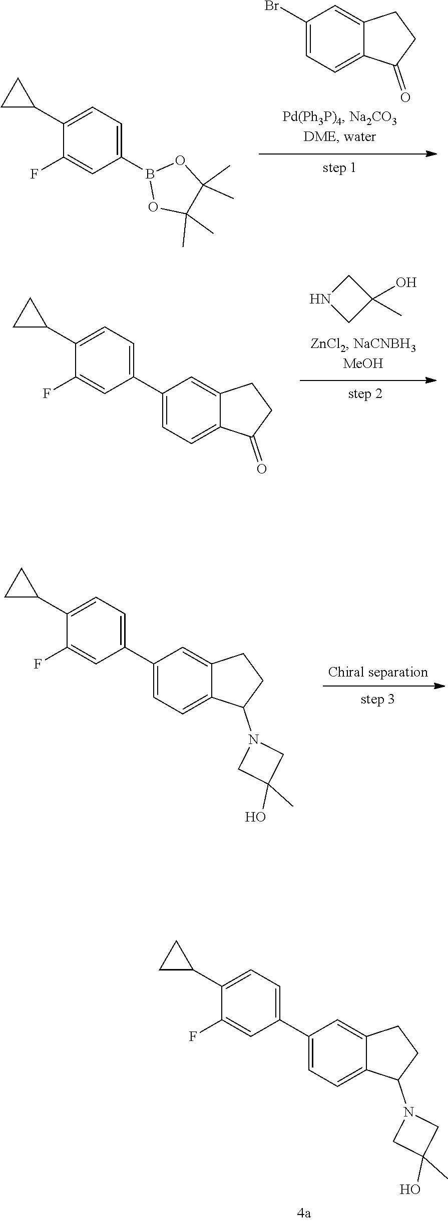

### Synthesis of 5-(3-fluoro-4-cyclopropyl-phenyl) indan-1-one

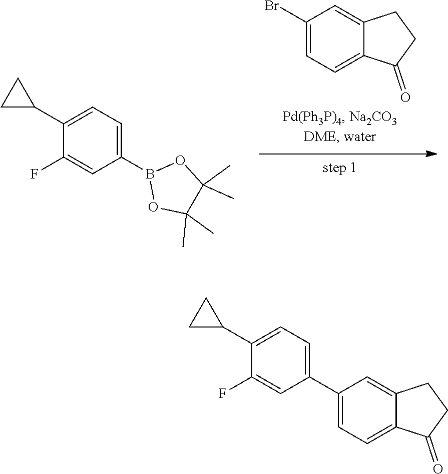 [SMILES: CC1(C)OB(c2ccc(C3CC3)c(F)c2)OC1(C)C ; O=C1CCc2cc(Br)ccc21 ; O=C1CCc2cc(-c3ccc(C4CC4)c(F)c3)ccc21]

A mixture of 5-bromoindan-1-one (500 mg, 2.37 mmol, 1 equiv.), 2-(3-fluoro-4-cyclopropyl-phenyl)-4,4,5,5-tetramethyl-1,3,2-dioxaborolane (681 mg, 2.60 mmol, 1.1 equiv.), Na2CO₃ (750 mg, 7.11 mmol, 3 equiv.) and Pd(PPh₃)₄ (274 mg, 0.24 mmol, 0.1 equiv.) in 1,2-dimethoxyethane (8 mL) and water (1.5 mL) was stirred for 3 h at 80° C. under nitrogen atmosphere. LCMS showed the reaction was completed. The reaction mixture was quenched with water (20 mL) and extracted with DCM (3*10 mL). The combined organic layers were concentrated under reduced pressure. The residue was purified by flash chromatography on silica gel (eluted with DCM/MeOH, 19:1) to afford 5-(3-fluoro-4-cyclopropyl-phenyl) indan-1-one (200 mg, 32%) as a yellow oil. LCMS (ESI, m/z): 267 [M+H]⁺.

### Synthesis of 1-[5-(3-chloro-4-fluoropropyl-phenyl) indan-1-yl]-3-methyl-azetidin-3-ol

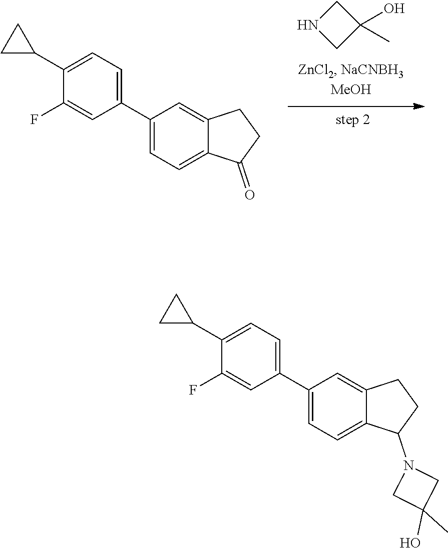

A mixture of NaCNBH₃ (133 mg, 2.12 mmol, 4 equiv.) and ZnCl₂ (2 M in 4Me-THF, 0.53 mL, 1.06 mmol, 2 equiv.) in methanol (3 mL) was stirred for 30 min at room temperature. Then 3-methylazetidin-3-ol (92 mg, 1.06 mmol, 2, equiv.) and 5-(3-fluoro-4-cyclopropyl-phenyl) indan-1-one (140 mg, 0.53 mmol, 1 equiv.) were added. The resulting mixture was stirred overnight at 60° C. LCMS showed the reaction was completed. The reaction was quenched by water (20 mL) and extracted with DCM (3×10 mL). The combined organic layers were concentrated under reduced pressure. The residue was purified by Prep-HPLC (Column: YMC-Actus Triart C18, 30*250, 5 μm; Mobile Phase A: water (10 mM NH₄HCO_{3+0.1}% NH₃ H₂O), Mobile Phase B: ACN; Flow rate: 60 mL/min; Gradient: 54% B to 84% B in 7 min; 254/210 nm; RT1:6.42 min) to afford 1-[5-(3-chloro-4-fluoropropyl-phenyl) indan-1-yl]-3-methyl-azetidin-3-ol (100 mg, 56%) as a white solid. LCMS (ESI, m z): 338 [M+H]⁺

### Chiral Separation of 1-[5-(3-chloro-4-fluoropropyl-phenyl) indan-1-yl]-3-methyl-azetidin-3-ol (4a)

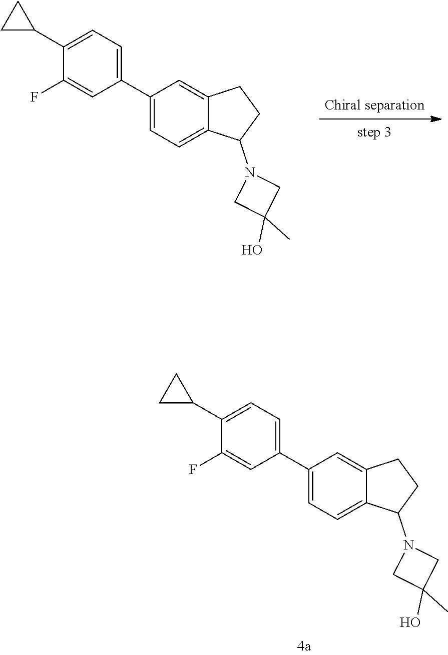 [SMILES: CC1(O)CN(C2CCc3cc(-c4ccc(C5CC5)c(F)c4)ccc32)C1 ; CC1(O)CN(C2CCc3cc(-c4ccc(C5CC5)c(F)c4)ccc32)C1]

The racemate was separated by chiral-HPLC(Column: CHIRALPAK IG, 3*25 cm, 5 μm; Mobile Phase A: Hex (0.5% 2M NH₃-MeOH)-HPLC, Mobile Phase B: IPA-HPLC; Flow rate: 45 mL/min; Gradient: 5% B to 5% B in 24 min; Wave Length: 220/254 nmn) to afford the first eluting peak (47.2 mg, Rt: 15.160 min) as a white solid.

LCMS (ESI, m/z): 338 [M+H]⁺. Analytic Conditions: column: EVO C18, 3.0*50 mm, 2.6 μm; mobile phase A: water (5 mM NH₄HCO₃), mobile phase B: acetonitrile; flow rate: 1.20 mL/min; gradient: 10% B to 95% B in 2.00 min, hold at 95% for 0.60 min, 95% B to 10% B in 0.15 min; 254 nm; RT: 1.690 min.

¹H NMR (400 MHZ, Methanol-d₄) δ7.49 (s, 1H), 7.43-7.38 (m, 2H), 7.32 (dd, J=8.0, 2.0 Hz, 1H), 7.27 (dd, J=12.0, 2.0 Hz, 1H), 7.01 (t, J=8.0 Hz, 1H), 4.04-4.01 (m, 1H), 3.48-3.42 (m, 2H), 3.40-3.38 (m, 1H), 3.25-3.23 (m, 1H), 3.17-3.09 (m, 1H), 2.92-2.85 (m, 1H), 2.30-2.21 (m, 1H), 2.15-2.08 (m, 1H), 1.98-1.91 (m, 1H), 1.48 (s, 3H), 1.05-1.00 (m, 2H), 0.79-0.75 (m, 2H).

### Chiral Separation of 1-[5-(3-chloro-4-fluoropropyl-phenyl) indan-1-yl]-3-methyl-azetidin-3-ol (4b)

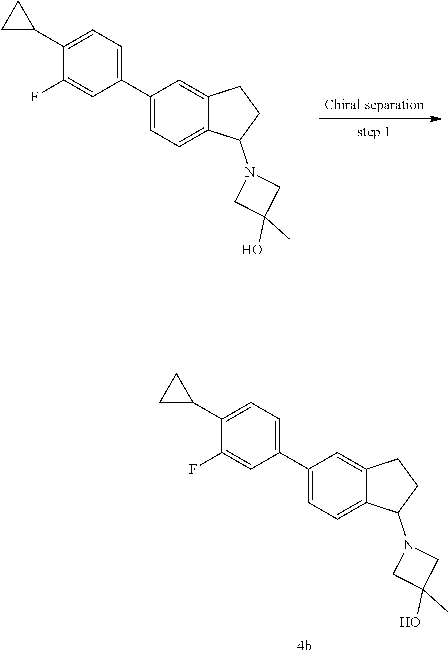 [SMILES: CC1(O)CN(C2CCc3cc(-c4ccc(C5CC5)c(F)c4)ccc32)C1 ; CC1(O)CN(C2CCc3cc(-c4ccc(C5CC5)c(F)c4)ccc32)C1]

The racemate was separated by chiral-HPLC(Column: CHIRALPAK IG, 3*25 cm, 5 μm; Mobile Phase A: Hex (0.5% 2M NH₃—MeOH)—HPLC, Mobile Phase B: IPA-HPLC; Flow rate: 45 mL/min; Gradient: 5% B to 5% B in 24 min; Wave Length: 220/254 nmn) to afford the first eluting peak (47.2 mg, Rt: 21.712 min) as a white solid.

LCMS (ESI, m/z): 338 [M+H]⁺. Analytic Conditions: column: EVO C18, 3.0*50 mm, 2.6 μm; mobile phase A: water (5 mM NH₄HCO₃), mobile phase B: acetonitrile; flow rate: 1.20 mL/min; gradient: 10% B to 95% B in 2.00 min, hold at 95% for 0.60 min, 95% B to 10% B in 0.15 min; 254 nm; RT: 1.687 min.

¹H NMR (400 MHZ, Methanol-d₄) δ7.49 (s, 1H), 7.43-7.37 (m, 2H), 7.31 (dd, J=8.0, 2.0 Hz, 1H), 7.27 (dd, J=12.0, 2.0 Hz, 1H), 7.00 (t, J=8.0 Hz, 1H), 4.03-4.00 (m, 1H), 3.47-3.42 (m, 2H), 3.39-3.37 (m, 1H), 3.24-3.22 (m, 1H), 3.17-3.09 (m, 1H), 2.91-2.84 (m, 1H), 2.29-2.20 (m, 1H), 2.15-2.08 (m, 1H), 1.97-1.90 (m, 1H), 1.48 (s, 3H), 1.04-0.99 (m, 2H), 0.78-0.75 (m, 2H).

### Example S5. 1-(5-(3-chloro-4-cyclopropylphenyl)-2,3-dihydro-1H-inden-1-yl)-3-methylazetidin-3-ol (5a & 5b)

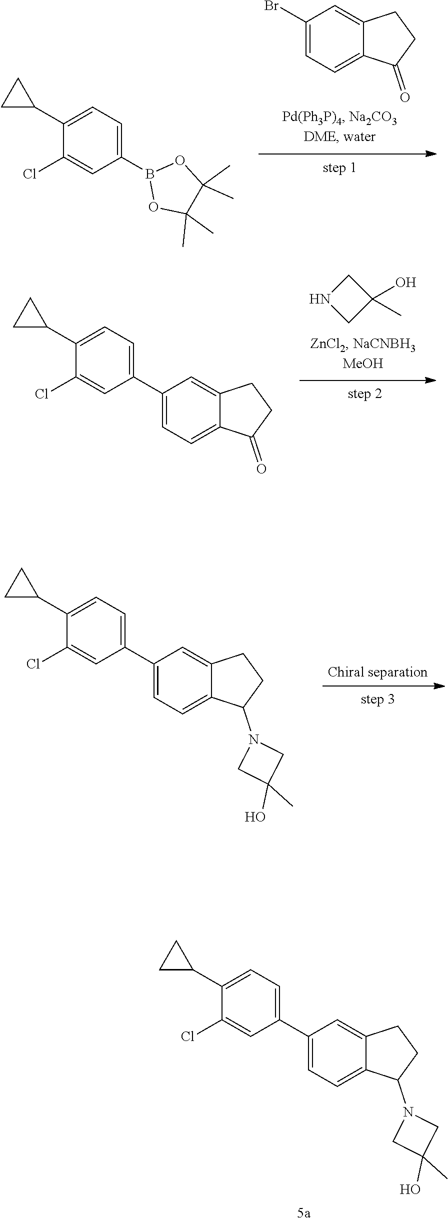

### Synthesis of 5-(3-chloro-4-cyclopropyl-phenyl) indan-1-one

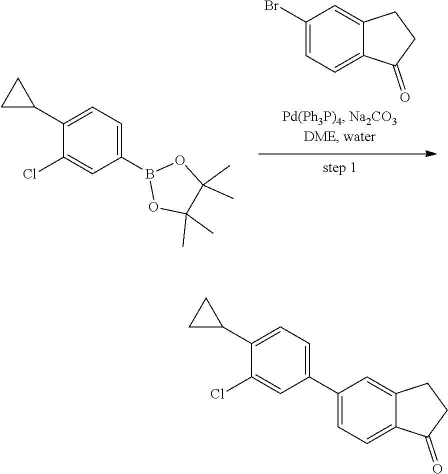 [SMILES: CC1(C)OB(c2ccc(C3CC3)c(Cl)c2)OC1(C)C ; O=C1CCc2cc(Br)ccc21 ; O=C1CCc2cc(-c3ccc(C4CC4)c(Cl)c3)ccc21]

A mixture of 5-bromoindan-1-one (500 mg, 2.37 mmol, 1 equiv.), 2-(3-chloro-4-cyclopropyl-phenyl)-4,4,5,5-tetramethyl-1,3,2-dioxaborolane (727 mg, 2.60 mmol, 1.1 equiv.), Na2CO₃ (750 mg, 7.11 mmol, 3 equiv.) and Pd(PPh₃)+ (274 mg, 0.24 mmol, 0.1 equiv.) in 1,2-dimethoxyethane (8 mL) and water (1.5 mL) was stirred for 3 h at 80° C. under nitrogen atmosphere. LCMS showed the reaction was completed. The reaction mixture was quenched with water (20 mL) and extracted with DCM (3*10 mL). The combined organic layers were concentrated under reduced pressure. The residue was purified by flash chromatography on silica gel (eluted with DCM/MeOH, 20:1) to afford 5-(3-chloro-4-cyclopropyl-phenyl) indan-1-one (350 mg, 52%) as a yellow oil. LCMS (ESI, m z): 283 [M+H]⁺.

### Synthesis of 1-[5-(3-chloro-4-cyclopropyl-phenyl) indan-1-yl]-3-methyl-azetidin-3-ol

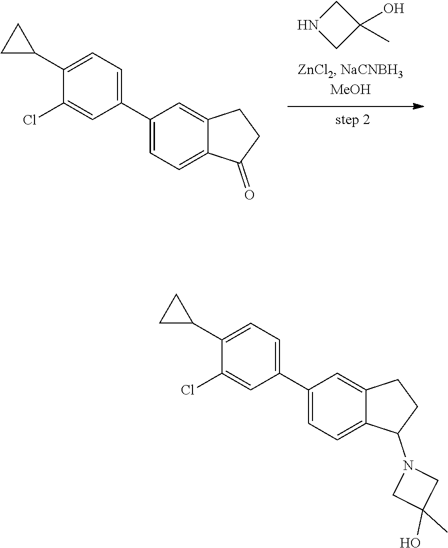

A mixture of NaCNBH₃ (133 mg, 2.12 mmol, 4 equiv.) and ZnCl₂ (2 M in 4Me-THF, 0.53 mL, 1.06 mmol, 2 equiv.) in methanol (3 mL) was stirred for 30 min at room temperature. Then 3-methylazetidin-3-ol (92 mg, 1.06 mmol, 2, equiv.) and 5-(3-chloro-4-cyclopropyl-phenyl) indan-1-one (150 mg, 0.53 mmol, 1 equiv.) were added. The resulting mixture was stirred overnight at 60° C. LCMS showed the reaction was completed. The reaction was quenched by water (20 mL) and extracted with DCM (3×10 mL). The combined organic layers were concentrated under reduced pressure. The residue was purified by Prep-HPLC (Column: SunFire Prep C18 OBD Column, 19×150 mm, 5 μm; Mobile Phase A: water (0.05% HCl), Mobile Phase B: ACN; Flow rate: 60 mL/min; Gradient: 20% B to 45% B in 10 min; 254/210 nm; Rt: 8.19 min) to afford 1-[5-(3-chloro-4-cyclopropyl-phenyl) indan-1-yl]-3-methyl-azetidin-3-ol (120 mg, 64%) as a yellow solid. LCMS (ESI, m z): 354 [M+H]⁺.

### Chiral Separation of 1-[5-(3-chloro-4-cyclopropyl-phenyl) indan-1-yl]-3-methyl-azetidin-3-ol (5a)

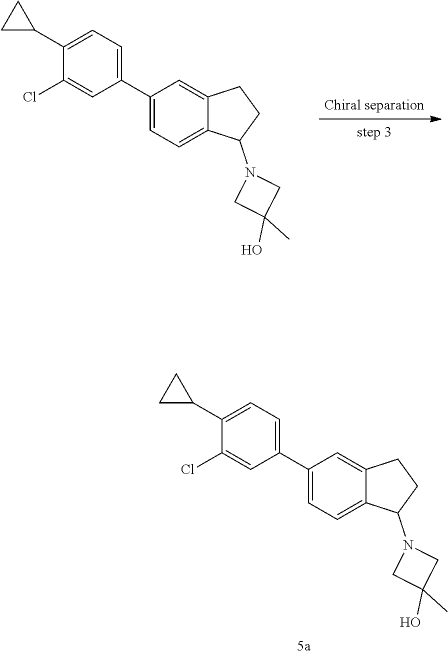 [SMILES: CC1(O)CN(C2CCc3cc(-c4ccc(C5CC5)c(Cl)c4)ccc32)C1 ; CC1(O)CN(C2CCc3cc(-c4ccc(C5CC5)c(Cl)c4)ccc32)C1]

The racemate was separated by SFC(Column: Lux 5 μm Cellulose-4, 3*25 cm, 5 μm; Mobile Phase A: CO₂, Mobile Phase B: IPA (0.5% 2M NH₃-MeOH); Flow rate: 60 mL/min; Gradient: 50% B; 220 nm) to afford the first eluting peak (29.0 mg, Rt: 5.59 min) as a yellow solid.

LCMS (ESI, m/z): 354 [M+H]⁺. Analytic Conditions: column: Shim-pack XR-ODS, 3.0*50 mm, 2.2 μm; mobile phase A: water (0.05% TFA), mobile phase B: acetonitrile (0.05% TFA); flow rate: 1.20 mL/min; gradient: 5% B to 100% B in 2.00 min, hold at 100% for 0.70 min, 100% B to 5% B in 0.05 min; 254 nm; RT: 1.645 min.

¹H NMR (300 MHZ, Methanol-d₄) δ7.64-7.61 (m, 3H), 7.57-7.53 (m, 1H), 7.48 (dd, J=8.1, 1.8 Hz, 1H), 7.07 (d, J=8.1 Hz, 1H), 4.96 (dd, J=7.8, 1.8 Hz, 1H), 4.31-4.16 (m, 3H), 4.08 (d, J=10.5 Hz, 1H), 3.29-3.21 (m, 1H), 3.10-3.00 (m, 1H), 2.64-2.51 (m, 1H), 2.29-2.19 (m, 2H), 1.56 (s, 3H), 1.10-1.03 (m, 2H), 0.77-0.71 (m, 2H).

### Chiral Separation of 1-[5-(3-chloro-4-cyclopropyl-phenyl) indan-1-yl]-3-methyl-azetidin-3-ol (5b)

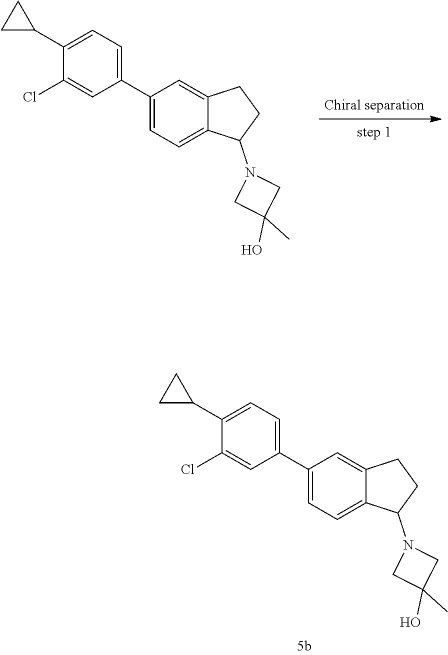 [SMILES: CC1(O)CN(C2CCc3cc(-c4ccc(C5CC5)c(Cl)c4)ccc32)C1 ; CC1(O)CN(C2CCc3cc(-c4ccc(C5CC5)c(Cl)c4)ccc32)C1]

The racemate was separated by SFC(Column: Lux 5 μm Cellulose-4, 3*25 cm, 5 μm; Mobile Phase A: CO₂, Mobile Phase B: IPA (0.5% 2M NH₃-MeOH); Flow rate: 60 mL/min; Gradient: 50% B; 220 nm) to afford the second eluting peak (33.8 mg, Rt: 8.34 min) as a yellow solid.

LCMS (ESI, m/z): 354 [M+H]⁺. Analytic Conditions: column: Shim-pack XR-ODS, 3.0*50 mm, 2.2 μm; mobile phase A: water (0.05% TFA), mobile phase B: acetonitrile (0.05% TFA); flow rate: 1.20 mL/min; gradient: 5% B to 100% B in 2.00 min, hold at 100% for 0.70 min, 100% B to 5% B in 0.05 min; 254 nm; RT: 1.652 min.

¹H NMR (300 MHz, Methanol-d₄) δ 7.64-7.61 (m, 3H), 7.57-7.54 (m, 1H), 7.48 (dd, J=8.1, 1.8 Hz, 1H), 7.08 (d, J=8.1 Hz, 1H), 4.96 (dd, J=7.8, 3.0 Hz, 1H), 4.31-4.16 (m, 3H), 4.08 (d, J=10.5 Hz, 1H), 3.29-3.21 (m, 1H), 3.10-3.01 (m, 1H), 2.64-2.51 (m, 1H), 2.28-2.19 (m, 2H), 1.56 (s, 3H), 1.10-1.03 (m, 2H), 0.77-0.72 (m, 2H).

### Example S6. 1-(5-(3-chloro-4-cyclopropylphenyl)-2,3-dihydro-1H-inden-1-yl)-4-methylpiperidin-4-ol (6)

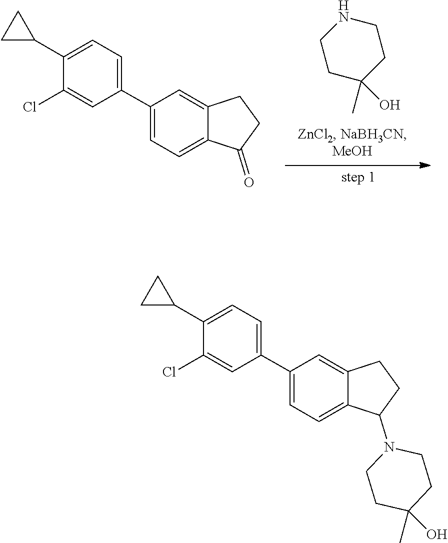

A mixture of NaCNBH₃ (90 mg, 1.40 mmol, 4 equiv.) and ZnCl₂ (4 M in 4Me-THF, 0.18 mL, 0.71 mmol, 2 equiv.) in methanol (5 mL) was stirred for 30 min at room temperature. Then 5-(3-chloro-4-cyclopropyl-phenyl) indan-1-one (100 mg, 0.35 mmol, 1 equiv.) and 4-methylpiperidin-4-ol (41 mg, 0.35 mmol, 1 equiv.) were added in portions. The resulting mixture was stirred overnight at 60° C. LCMS showed the reaction was completed. The reaction was quenched by water (20 mL) and extracted with DCM (3×10 mL). The combined organic layers were concentrated under reduced pressure. The residue was purified by prep-HPLC (Column: Xselect CSH OBD Column 30*150 mm 5 μm; Mobile Phase A: water (0.1% FA), Mobile Phase B: ACN; Flow rate: 60 mL/min; Gradient: 21% B to 45% B in 7 min; 254/220 nm; RT: 5.95 min) to afford 1-(5-(3-chloro-4-cyclopropylphenyl)-2,3-dihydro-1H-inden-1-yl)-4-methylpiperidin-4-ol (12.4 mg, 9%) as a white solid.

¹H NMR (400 MHZ, Chloroform-d) δ 7.60-7.59 (m, 1H), 7.45-7.38 (m, 4H), 6.99 (d, J=8.0 Hz, 1H), 4.38-4.35 (m, 1H), 3.15-3.07 (m, 2H), 2.93-2.57 (m, 4H), 2.52-2.43 (m, 1H), 2.28-2.14 (m, 3H), 1.98-1.85 (m, 3H), 1.37 (d, J=6.0 Hz, 3H), 1.09-1.00 (m, 2H), 0.77-0.742 (m, 2H).

LCMS (ESI, m/z): 382 [M+H]⁺. Analytic Conditions: column: HALO C18, 3.0*30 mm, 2.7 μm; mobile Phase A: water (0.05% TFA), mobile Phase B: acetonitrile (0.05% TFA); flow rate: 1.50 mL/min; gradient: 5% B to 100% B in 1.30 min, hold at 100% for 0.50 min, 100% B to 5% B in 0.03 min; 254 nm; RT: 0.998 min.

### Example S7. 1-(5-(3-chloro-4-cyclopropylphenyl)-2,3-dihydro-1H-inden-1-yl)-3-methylpyrrolidin-3-ol (7)

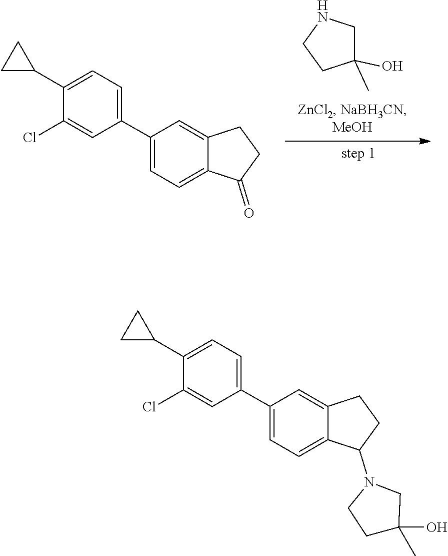

A mixture of NaCNBH₃ (91 mg, 1.41 mmol, 4 equiv.) and ZnCl₂ (2 M in 4Me-THF, 0.36 mL, 0.71 mmol, 2 equiv.) in methanol (5 mL) was stirred for 30 min at room temperature. Then 5-(3-chloro-4-cyclopropyl-phenyl) indan-1-one (100 mg, 0.35 mmol, 1 equiv.) and 3-methylpyrrolidin-3-ol (34 mg, 0.35 mmol, 1 equiv.) were added in portions. The resulting mixture was stirred overnight at 60° C. LCMS showed the reaction was completed. The reaction was quenched by water (20 mL) and extracted with DCM (3×10 mL). The combined organic layers were concentrated under reduced pressure. The residue was purified by prep-HPLC (Column: Sun Fire Prep C18 OBD Column, 19×150 mm, 5 μm; Mobile Phase A: water (0.1% FA), Mobile Phase B: ACN; Flow rate: 60 mL/min; Gradient: 12% B to 42% B in 8 min, hold at 42% B for 1 min; 254/210 nm; RT: 8.32 min) to afford 1-(5-(3-chloro-4-cyclopropylphenyl)-2,3-dihydro-1H-inden-1-yl)-3-methylpyrrolidin-3-ol (23.4 mg, 18%) as a yellow oil.

¹H NMR (400 MHZ, Chloroform-d) δ 7.59 (s, 1H), 7.54-7.50 (m, 2H), 7.38 (d, J=8.0 Hz, 1H), 7.25-7.22 (m, 1H), 7.03-7.01 (m, 1H), 5.03-4.93 (m, 1H), 3.31-3.11 (m, 5H), 2.70-2.46 (m, 3H), 2.29-2.23 (m, 1H), 1.40 (m, 3H), 1.28 (s, 2H), 1.11-1.06 (m, 2H), 0.78-0.74 (m, 2H).

LCMS (ESI, m/z): 368 [M+H]⁺. Analytic Conditions: column: HALO C18, 3.0*30 mm, 2.7 μm; mobile Phase A: water (0.05% TFA), mobile Phase B: acetonitrile (0.05% TFA); flow rate: 1.50 mL/min; gradient: 5% B to 100% B in 1.30 min, hold at 100% for 0.50 min, 100% B to 5% B in 0.03 min; 254 nm; RT: 0.993 min.

### Example S8. 1-(5-(1-(2,6-dichlorophenyl) azetidin-3-yl)-2,3-dihydro-1H-inden-1-yl)-4-methyl-piperidin-4-ol (8)

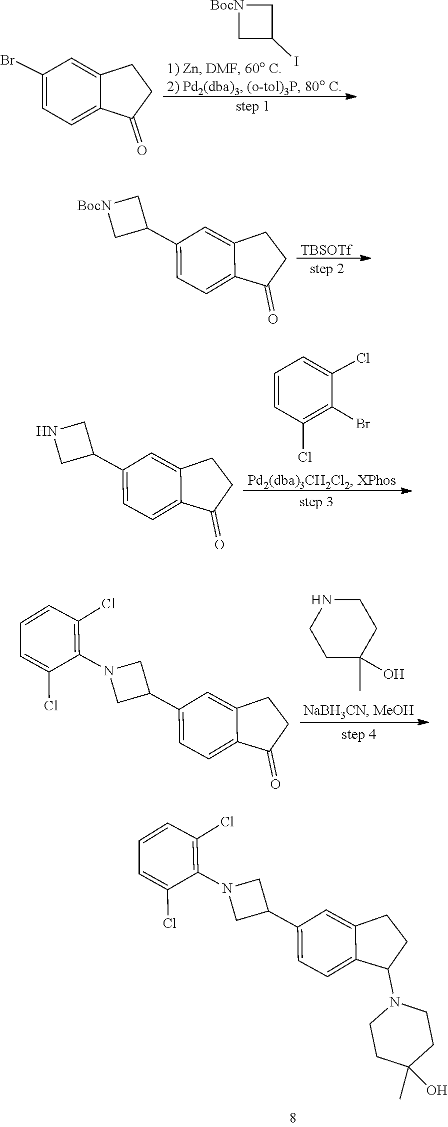

### Synthesis of tert-butyl 3-(1-oxoindan-5-yl) azetidine-1-carboxylate

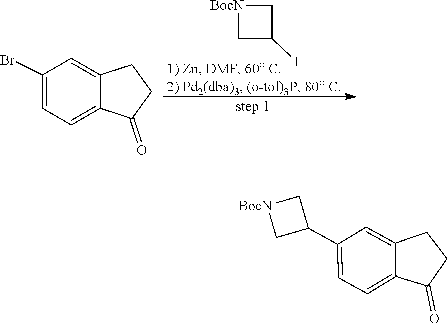 [SMILES: O=C1CCc2cc(Br)ccc21 ; CC(C)(C)OC(=O)N1CC(I)C1 ; CC(C)(C)OC(=O)N1CC(c2ccc3c(c2)CCC3=O)C1]

To a stirred solution of tert-butyl 3-iodoazetidine-1-carboxylate (60 g, 211 mmol, 4.00 equiv.) in DMF (900 mL) were added Zinc powder (22.5 g, 344 mmol, 7.00 equiv.). The mixture solution was stirred at 80° C. for 2 h. Then 5-bromoindan-1-one (10.5 g, 49.8 mmol, 1.00 equiv.), Pd2 (dba)₃ (4.5 g, 4.97 mmol, 0.10 equiv.) and tri-m-tolylphosphane (3.0 g, 9.95 mmol, 0.20 equiv.) were added. The resulting mixture was stirred overnight at 80° C. under nitrogen atmosphere. LCMS showed the reaction was completed. The reaction was filtered. The filtrated was diluted with water (2 L) and extracted with ethyl acetate (5× 400 mL). The organic layers were concentrated under vacuum. The residue was purified by flash column chromatography on silica gel (eluted with dichloromethane/methanol, 20/1) to afford tert-butyl 3-(1-oxoindan-5-yl) azetidine-1-carboxylate (4.5 g, 31%) as an off-white solid. LCMS (ESI, m/z): 288 [M+H]⁺.

### Synthesis of 5-(azetidin-3-yl)-2,3-dihydro-1H-inden-1-one

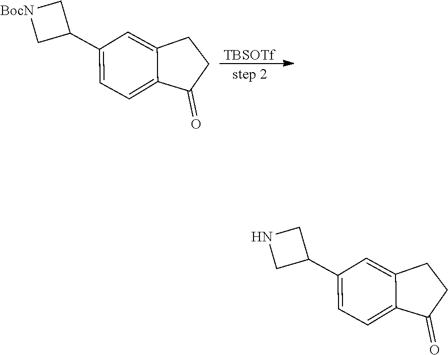 [SMILES: "TBSOTf" ; CC(C)(C)OC(=O)N1CC(c2ccc3c(c2)CCC3=O)C1 ; O=C1CCc2cc(C3CNC3)ccc21]

To a solution of tert-butyl 3-(1-oxo-2,3-dihydro-1H-inden-5-yl) azetidine-1-carboxylate (600 mg, 1.50 mmol, 1.00 equiv.) and in DCM (5 mL) was added TBSOTf (0.4 mL, 2.25 mmol, 1.50 equiv.). The resulting solution was stirred at room temperature for 1 h. TLC showed the reaction was completed. The reaction mixture was concentrated under reduced pressure. The crude product was purified by flash column on C18 (eluted with water/ACN, 5/95) to afford 5-(azetidin-3-yl)-2,3-dihydro-1H-inden-1-one (300 mg, 77%) as a white solid. LCMS (ESI, m z): 188 [M+H]⁺.

### Synthesis of 5-(1-(2,6-dichlorophenyl) azetidin-3-yl)-2,3-dihydro-1H-inden-1-one

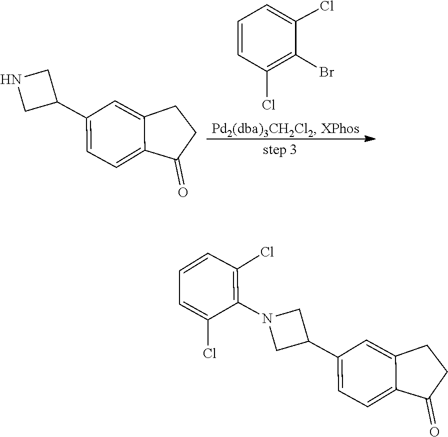 [SMILES: O=C1CCc2cc(C3CNC3)ccc21 ; Clc1cccc(Cl)c1Br ; O=C1CCc2cc(C3CN(c4c(Cl)cccc4Cl)C3)ccc21]

A solution of 5-(azetidin-3-yl)-2,3-dihydro-1H-inden-1-one (300 mg, 1.60 mmol, 1.00 equiv.), 2-bromo-1,3-dichlorobenzene (358 mg, 1.60 mmol, 1.00 equiv.), Pd2 (dba)₃CH₂Cl₂ (166 mg, 0.160 mmol, 0.10 equiv.), t-BuONa (470 mg, 4.80 mmol, 3.00 equiv.) and XPhos (152 mg, 3.200 mmol, 0.20 equiv.) in toluene (10 mL) was stirred at 90° C. for 2 h under N₂ atmosphere. LCMS showed the reaction was completed. The reaction mixture was concentrated and purified by flash column on silica gel (eluted with PE/EA, 1/1) to afford 5-(1-(2,6-dichlorophenyl) azetidin-3-yl)-2,3-dihydro-1H-inden-1-one (150 mg, 28%) as a yellow oil. LCMS (ESI, m z): 332 [M+H]⁺.

### Synthesis of 1-[5-[1-(2,6-dichlorophenyl) azetidin-3-yl]indan-1-yl]-4-methyl-piperidin-4-ol (8)

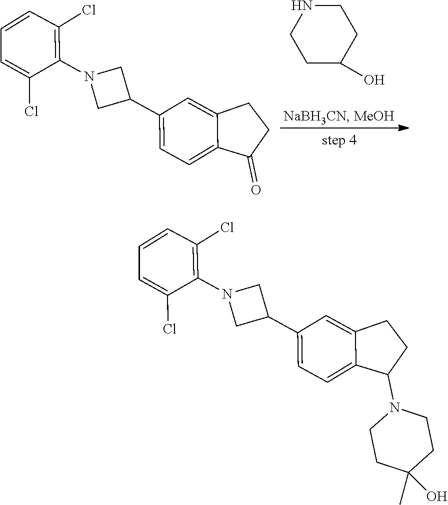 [SMILES: O=C1CCc2cc(C3CN(c4c(Cl)cccc4Cl)C3)ccc21 ; OC1CCNCC1 ; CC1(O)CCN(C2CCc3cc(C4CN(c5c(Cl)cccc5Cl)C4)ccc32)CC1]

To a stirred solution of 5-[1-(2,6-dichlorophenyl) azetidin-3-yl]indan-1-one (50 mg, 0.15 mmol, 1.00 equiv.) and 4-methylpiperidin-4-ol (34 mg, 0.30 mmol, 2.00 equiv.) in methanol (2 mL) were added NaBH₃CN (28 mg, 0.45 mmol, 3.00 equiv.) and ZnCl₂ (2 M in THF, 0.15 mL, 0.30 mmol, 2.00 equiv.). The mixture solution was stirred overnight at 80° C. LCMS showed the reaction was completed. The reaction was quenched with water (10 mL) and extracted with dichloromethane (2×10 mL). The organic layers were concentrated under vacuum. The residue was purified by prep-HPLC(Column: XBridge C18 OBD Prep Column, 5 μm, 19*250 mm; Mobile Phase A: water (10 mM NH₄HCO₃), Mobile Phase B: ACN; Flow rate: 25 mL/min; Gradient: 62% B to 75% B in 8 min; Wave Length: 254/220 nm; RT: 6.97 min) to afford 1-[5-[1-(2,6-dichlorophenyl) azetidin-3-yl]indan-1-yl]-4-methyl-piperidin-4-ol (26.9 mg, 40%) as an off-white solid.

LCMS (ESI, m/z): 431 [M+H]⁺. Analytic Conditions: column: HALO C18 Column 3.0*30 mm, 2.0 μm; mobile Phase A: water/0.05% TFA, mobile Phase B: acetonitrile/0.05% TFA; flow rate: 1.20 mL/min; gradient: 5% B to 100% B in 1.2 min, hold at 100% for 0.6 min, 100% B to 5% B in 0.03 min; 220 nm; RT: 0.893 min.

¹H NMR (300 MHZ, DMSO-d₆) δ 7.27-7.21 (m, 5H), 6.74 (t, J=8.1 Hz, 1H), 4.82 (t, J=8.1 Hz, 2H), 4.40-4.32 (m, 2H), 4.24 (t, J=7.2 Hz, 2H), 4.04 (s, 1H), 3.79-3.69 (m, 1H), 2.91-2.68 (m, 2H), 2.56-2.38 (m, 2H), 2.21-2.15 (m, 1H), 2.05-1.93 (m, 2H), 1.49-1.34 (m, 4H), 1.08 (s, 3H).

### Example S9. 1-(5-(1-(2,6-dichlorophenyl) azetidin-3-yl)-2,3-dihydro-1H-inden-1-yl)-3-methylpyrrolidin-3-ol (9)

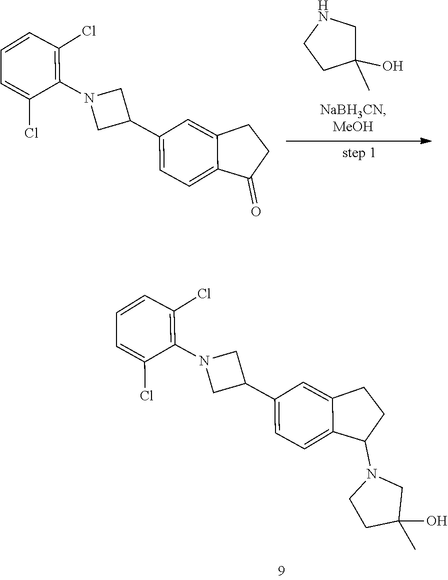

To a stirred solution of 5-[1-(2,6-dichlorophenyl) azetidin-3-yl]indan-1-one (60 mg, 0.18 mmol, 1.00 equiv.) and 3-methylpyrrolidin-3-ol (54 mg, 0.54 mmol, 3.00 equiv.) in methanol (2.0 mL) were added NaBH₃CN (34 mg, 0.54 mmol, 3.00 equiv.) and ZnCl₂ (2M in THF, 0.18 mL, 0.36 mmol, 2.00 equiv.). The mixture solution was stirred overnight at 80° C. LCMS showed the reaction was completed. The reaction was quenched with water (10 mL) and extracted with dichloromethane (2×10 mL). The organic layers were concentrated under vacuum. The residue was purified by prep-HPLC(Column: XBridge C18 OBD Prep Column, 5 μm, 19*250 mm; Mobile Phase A: water (10 mM NH+HCO₃), Mobile Phase B: ACN; Flow rate: 25 mL/min; Gradient: 60% B to 90% B in 9 min; Wave Length: 254/220 nm; RT: 8.12 min) to afford 1-[5-[1-(2,6-dichlorophenyl) azetidin-3-yl]indan-1-yl]-3-methyl-pyrrolidin-3-ol (66.1 mg, 86%) as an light-orange semi-solid.

LCMS (ESI, m/z): 417 [M+H]⁺. Analytic Conditions: column: HALO C18 Column 3.0*30 mm, 2.0 μm; mobile Phase A: water/0.05% TFA, mobile Phase B: acetonitrile/0.05% TFA; flow rate: 1.20 mL/min; gradient: 5% B to 65% B in 1.7 min, 65% B to 100% B in 0.3 min, hold at 100% for 0.6 min, 100% B to 5% B in 0.10 min; 254 nm; RT: 1.495 min.

¹H NMR (400 MHZ, DMSO-d₆) δ 7.29-7.18 (m, 5H), 6.75 (t, J=8.0 Hz, 1H), 4.82 (t, J=8.0 Hz, 2H), 4.46-4.44 (m, 1H), 4.34 (t, J=7.6 Hz, 2H), 4.10 (t, J=6.0 Hz, 1H), 3.78-3.70 (m, 1H), 2.95-2.88 (m, 1H), 2.77-2.67 (m, 2H), 2.63-2.54 (m, 1H), 2.52-2.45 (m, 1H), 2.09-2.00 (m, 2H), 1.73-1.60 (m, 2H), 1.22 (s, 3H).

### Example S10. 1-(5-(3-chloro-4-cyclopropylphenyl)-7-methyl-2,3-dihydro-1H-inden-1-yl)-3-methylazetidin-3-ol (10a & 10b)

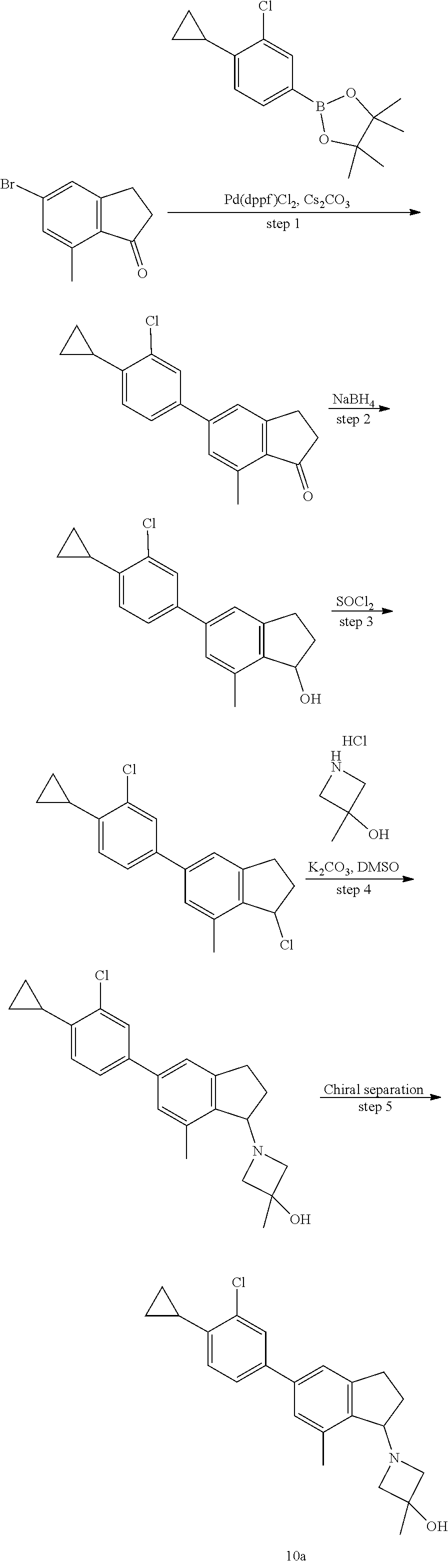

### Synthesis of 5-(3-chloro-4-cyclopropyl-phenyl)-7-methyl-indan-1-one

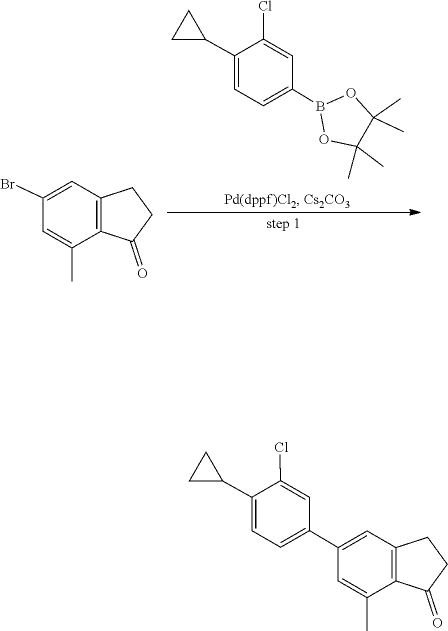 [SMILES: CC1(C)OB(c2ccc(C3CC3)c(Cl)c2)OC1(C)C ; Cc1cc(Br)cc2c1C(=O)CC2 ; Cc1cc(-c2ccc(C3CC3)c(Cl)c2)cc2c1C(=O)CC2]

To a stirred solution of 5-bromo-7-methyl-indan-1-one (500 mg, 2.22 mmol, 1.00 equiv.) and 2-(3-chloro-4-cyclopropyl-phenyl)-4,4,5,5-tetramethyl-1,3,2-dioxaborolane (804 mg, 2.89 mmol, 1.30 equiv.) in 1,4-dioxane (10 mL) and water (1.0 mL) were added Pd(dppf)Cl2 (162 mg, 0.22 mmol, 0.10 equiv.) and Cs₂CO₃ (2.2 g, 6.66 mmol, 3.00 equiv.). The resulting mixture was stirred at 80° C. for 4 h. LCMS showed the reaction was completed. The reaction was concentrated under vacuum. The residue was purified by flash column chromatography on silica gel (eluted with dichloromethane/methanol, 10/1) to afford 5-(3-chloro-4-cyclopropyl-phenyl)-7-methyl-indan-1-one (400 mg, 60%) as an off-white solid. LCMS (ESI, m/z): 297 [M+H]⁺.

### Synthesis of 5-(3-chloro-4-cyclopropyl-phenyl)-7-methyl-indan-1-ol

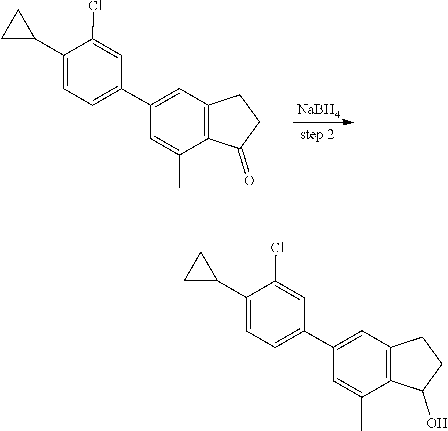

To a stirred solution of 5-(3-chloro-4-cyclopropyl-phenyl)-7-methyl-indan-1-one (500 mg, 1.68 mmol, 1.00 equiv.) in methanol (5 mL) was added NaBH₄ (191 mg, 5.05 mmol, 3.00 equiv.). The resulting mixture was stirred at 0° C. for 2 h. LCMS showed the reaction was completed. The reaction was quenched by water (20 mL) and extracted with ethyl acetate (3×10 mL). The combined organic layers were concentrated under vacuum. The residue was purified by flash column chromatography on C18 silica (eluted with water/acetonitrile, 2/3) to afford 5-(3-chloro-4-cyclopropyl-phenyl)-7-methyl-indan-1-ol (350 mg, 69%) as a light yellow oil. LCMS (ESI, m/z): 299 [M+H]⁺.

### Synthesis of 1-chloro-5-(3-chloro-4-cyclopropyl-phenyl)-7-methyl-indane

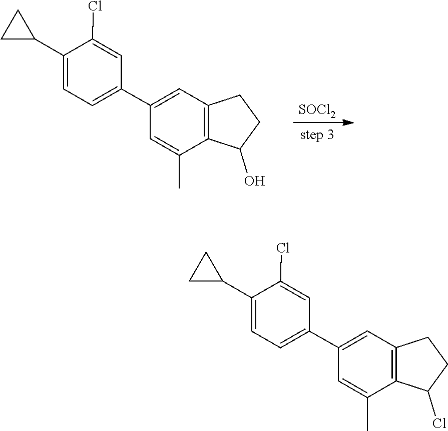

To a stirred solution of 5-(3-chloro-4-cyclopropyl-phenyl)-7-methyl-indan-1-ol (250 mg, 0.84 mmol, 1.00 equiv.) in DCM (5 mL) was added SOCI2 (0.3 mL, 4.180 mmol, 5.00 equiv.). The mixture was stirred at 0° C. for 1 h. TLC showed the reaction was completed. The reaction was concentrated under vaccum to afford 1-chloro-5-(3-chloro-4-cyclopropyl-phenyl)-7-methyl-indane as crude product, which was used in the next step directly without further purification. LCMS (ESI, m/z): 317 [M+H]⁺.

### Synthesis of 1-[5-(3-chloro-4-cyclopropyl-phenyl)-7-methyl-indan-1-yl]-3-methyl-azetidin-3-ol

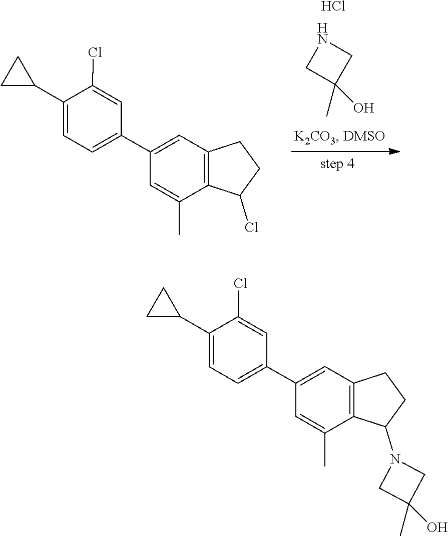

To a stirred solution of 1-chloro-5-(3-chloro-4-cyclopropyl-phenyl)-7-methyl-indane (250 mg, 0.79 mmol, 1.00 equiv.) and 3-methylazetidin-3-ol (102 mg, 1.18 mmol, 1.50 equiv.) in DMSO (5 mL) was added K₂CO₃ (326 mg, 2.360 mmol, 3.00 equiv.). The resulting mixture was stirred at 80° C. for 16 h. LCMS showed the reaction was completed. The reaction was diluted with water (30 mL) and extracted with ethyl acetate (3×15 mL). The combined organic layers were concentrated under vacuum. The residue was purified by flash column chromatography on C18 silica (eluted with water/acetonitrile, 1/3) to afford 1-[5-(3-chloro-4-cyclopropyl-phenyl)-7-methyl-indan-1-yl]-3-methyl-azetidin-3-ol (80 mg, 27%) as an off-white solid. LCMS (ESI, m/z): 368 [M+H]⁺.

### Chiral Separation of 1-[5-(3-chloro-4-cyclopropyl-phenyl)-7-methyl-indan-1-yl]-3-methyl-azetidin-3-ol (10a)

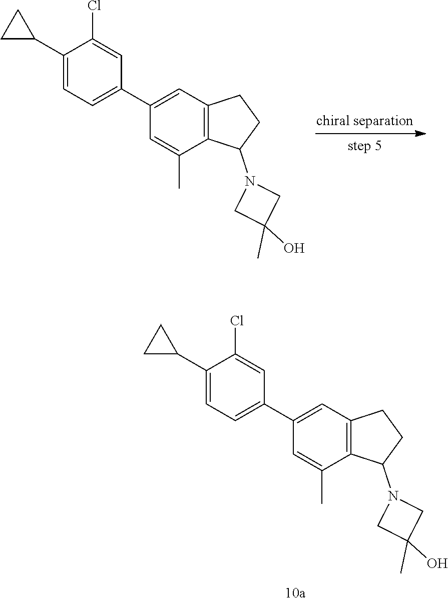 [SMILES: Cc1cc(-c2ccc(C3CC3)c(Cl)c2)cc2c1C(N1CC(C)(O)C1)CC2 ; Cc1cc(-c2ccc(C3CC3)c(Cl)c2)cc2c1C(N1CC(C)(O)C1)CC2]

The racemate (80 mg) was purified by Prep-Chiral HPLC(Column: Lux 5 μm Cellulose-2, 2.12*25 cm, 5 μm; Mobile Phase A: Hex (0.2% DEA)-HPLC, Mobile Phase B: EtOH-HPLC; Flow rate: 20 mL/min; Gradient: 2% B to 2% B in 25 min; 220/254 nm; RT1:15.924 min) to afford 1-[5-(3-chloro-4-cyclopropyl-phenyl)-7-methyl-indan-1-yl]-3-methyl-azetidin-3-ol (29.9 mg, 35%) as an off-white solid.

¹H NMR (300 MHz, DMSO-d₆) δ 7.65 (d, J=2.1 Hz, 1H), 7.50 (dd, J=8.1, 2.1 Hz, 1H), 7.31 (s, 1H), 7.23 (s, 1H), 7.07 (d, J=8.1 Hz, 1H), 5.11 (s, 1H), 4.08-4.04 (m, 1H), 3.12 (d, J=6.0 Hz, 1H), 3.06-2.91 (m, 4H), 2.80-2.71 (m, 1H), 2.42 (s, 3H), 2.22-2.10 (m, 2H), 1.96-1.84 (m, 1H), 1.32 (s, 3H), 1.07-1.00 (m, 2H), 0.77-0.72 (m, 2H).

LCMS (ESI, m/z): 368 [M+H]⁺. Analytic Conditions: column: L-column3 C18 Column 3.0*30 mm, 2.0 μm; mobile Phase A: Water/5 mM NH₄HCO₃, mobile Phase B: acetonitrile; flow rate: 1.5000 mL/min; gradient: 30% B to 80% B in 1.80 min, 80% B to 95% B in 0.5 min, hold at 95% for 0.5 min, 95% B to 10% B in 0.1 min; 254 nm; RT: 2.006 min.

### Chiral Separation of 1-[5-(3-chloro-4-cyclopropyl-phenyl)-7-methyl-indan-1-yl]-3-methyl-azetidin-3-ol (10b)

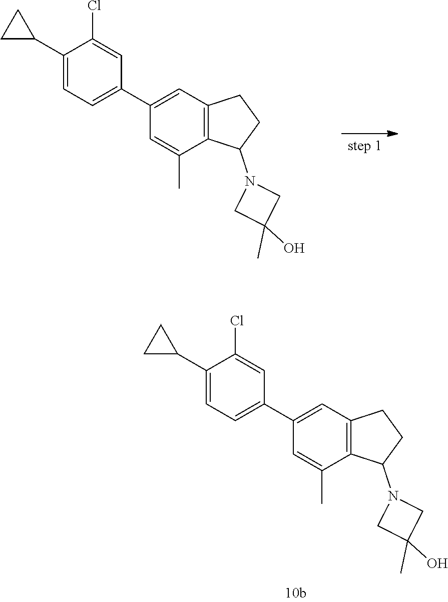 [SMILES: Cc1cc(-c2ccc(C3CC3)c(Cl)c2)cc2c1C(N1CC(C)(O)C1)CC2 ; Cc1cc(-c2ccc(C3CC3)c(Cl)c2)cc2c1C(N1CC(C)(O)C1)CC2]

The racemate (80 mg) was purified by Prep-Chiral HPLC(Column: Lux 5 μm Cellulose-2, 2.12*25 cm, 5 μm; Mobile Phase A: Hex (0.2% DEA)-HPLC, Mobile Phase B: EtOH-HPLC; Flow rate: 20 mL/min; Gradient: 2% B to 2% B in 25 min; 220/254 nm; RT2:22.478 min) to afford 1-[5-(3-chloro-4-cyclopropyl-phenyl)-7-methyl-indan-1-yl]-3-methyl-azetidin-3-ol (33.4 mg, 41%) as an off-white solid.

¹H NMR (300 MHz, DMSO-d₆) δ 7.66 (d, J=2.1 Hz, 1H), 7.50 (dd, J=8.1, 2.1 Hz, 1H), 7.31 (s, 1H), 7.24 (s, 1H), 7.07 (d, J=8.1 Hz, 1H), 5.11 (s, 1H), 4.06 (d, J=6.6 Hz, 1H), 3.12 (d, J=6.0 Hz, 1H), 3.06-2.94 (m, 4H), 2.80-2.72 (m, 1H), 2.42 (s, 3H), 2.22-2.10 (m, 2H), 1.96-1.84 (m, 1H), 1.32 (s, 3H), 1.07-1.00 (m, 2H), 0.77-0.72 (m, 2H).

LCMS (ESI, m/z): 368 [M+H]⁺. Analytic Conditions: column: EVO C18, 2.1*30 mm, 2.6 μm; mobile phase A: water (5 mM NH₄HCO₃), mobile phase B: acetonitrile; flow rate: 1.20 mL/min; gradient: 10% B to 95% B in 1.20 min, hold at 95% for 0.58 min, 95% B to 10% B in 0.05 min; 254 nm; RT: 1.157 min

### Example S11. 1-(5-(3-chloro-4-cyclopropylphenyl)-7-methyl-2,3-dihydro-1H-inden-1-yl)-4-methylpiperidin-4-ol (11)

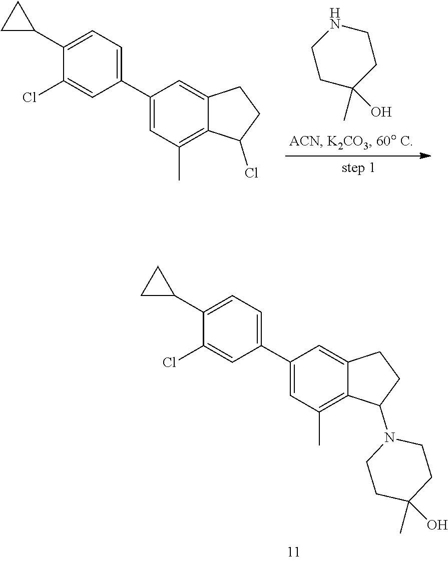

To a solution of 1-chloro-5-(3-chloro-4-cyclopropyl-phenyl)-7-methyl-indane (150 mg, 0.47 mmol, 1.00 equiv.) in MeCN (5 mL) were added 4-methylpiperidin-4-ol (82 mg, 0.71 mmol, 1.50 equiv.) and K₂CO₃ (196 mg, 1.42 mmol, 3.00 equiv.). The resulting mixture was stirred at 60° C. for 3 h. LCMS showed the reaction was completed. The reaction mixture was filtered and concentrated under reduced pressure. The reaction mixture was filtered and concentrated under reduced pressure. The residue was purified by prep-HPLC(Column: XSelect CSH Prep C18 OBD Column, 19*150 mm, 5 μm; Mobile Phase A: water (0.05% TFA), Mobile Phase B: ACN; Flow rate: 60 mL/min; Gradient: 23% B to 43% B in 10 min; 254/210 nm; RT: 9.65 min) to afford 1-[5-(3-chloro-4-cyclopropyl-phenyl)-7-methyl-indan-1-yl]-4-methyl-piperidin-4-ol (83.7 mg, 43%) as an off-white solid.

¹H NMR (400 MHZ, DMSO-d₆) δ 7.74 (d, J=2.0 Hz, 1H), 7.58 (dd, J=8.0, 2.0 Hz, 1H), 7.54 (m, 1H), 7.47 (m, 1H), 7.11 (d, J=8.0 Hz, 1H), 5.00-4.97 (m, 1H), 3.41-3.38 (m, 1H), 3.33-3.19 (m, 2H), 3.13-3.00 (m, 2H), 2.93-2.86 (m, 1H), 2.58-2.56 (m, 4H), 2.41-2.30 (m, 1H), 2.22-2.15 (m, 1H), 1.84-1.74 (m, 2H), 1.69-1.60 (m, 2H), 1.15 (s, 3H), 1.07-1.02 (m, 2H), 0.78-0.74 (m, 2H).

¹⁹F NMR (282 MHZ, DMSO-d₆) δ -74.202.

LCMS (ESI, m/z): 396 [M+H]⁺. Analytic Conditions: column: Titank C18, 3.0*50 mm, 3.0 μm; mobile Phase A: water/5 mM NH₄HCO₃, mobile Phase B: acetonitrile; flow rate: 1.50 mL/min; gradient: 80% B to 95% B in 1.80 min, hold at 95% for 0.80 min, 95% B to 10% B in 0.15 min; 254 nm; RT: 1.289 min,

### Example S12. 1-(5-(3-chloro-4-cyclopropylphenyl)-7-methyl-2,3-dihydro-1H-inden-1-yl)-3-methylpyrrolidin-3-ol (12)

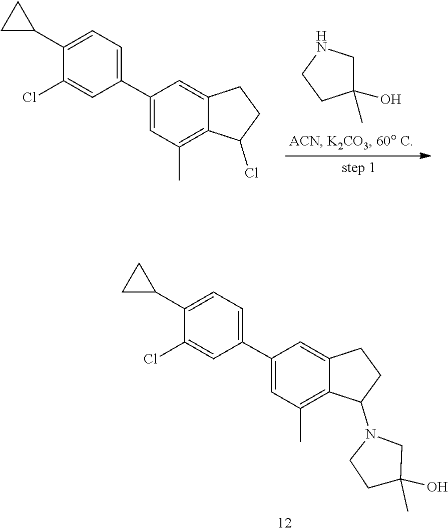

To a solution of 1-chloro-5-(3-chloro-4-cyclopropyl-phenyl)-7-methyl-indane (150 mg, 0.47 mmol, 1.00 equiv.) in MeCN (5 mL) were added 3-methylpyrrolidin-3-ol (58 mg, 0.58 mmol, 1.2 equiv.) and K₂CO₃ (196 mg, 1.42 mmol, 3.00 equiv.). The resulting mixture was stirred at 60° C. for 3 h. LCMS showed the reaction was completed. The reaction mixture was filtered and concentrated under reduced pressure. The reaction mixture was filtered and concentrated under reduced pressure. The residue was purified by prep-HPLC(Column: XSelect CSH Prep C18 OBD Column, 19*150 mm, 5 μm; Mobile Phase A: water (0.05% TFA), Mobile Phase B: ACN; Flow rate: 60 mL/min; Gradient: 23% B to 43% B in 10 min; 254/210 nm; RT: 9.65 min) to afford 1-[5-(3-chloro-4-cyclopropyl-phenyl)-7-methyl-indan-1-yl]-3-methyl-pyrrolidin-3-ol (54.1 mg, 29%) as an off-white solid.

¹H NMR (400 MHZ, DMSO-d₆) δ 7.73 (d, J=2.0 Hz, 1H), 7.57 (dd, J=8.0, 2.0 Hz, 1H), 7.52 (m, 1H), 7.45 (m, 1H), 7.10 (d, J=8.0 Hz, 1H), 5.14-4.97 (m, 1H), 3.43-3.37 (m, 3H), 3.28-3.22 (m, 1H), 3.14-3.03 (m, 1H), 2.97-2.79 (m, 1H), 2.60-2.57 (m, 4H), 2.44-2.33 (m, 1H), 2.22-2.15 (m, 1H), 2.12-1.80 (m, 2H), 1.38-1.33 (m, 3H), 1.08-1.02 (m, 2H), 0.78-0.73 (m, 2H). ¹⁹F NMR (282 MHZ, DMSO-d₆) δ -74.011.

LCMS (ESI, m/z): 382 [M+H]⁺. Analytic Conditions: column: Titank C18, 3.0*50 mm, 3.0 μm; mobile Phase A: water/5 mM NH₄HCO₃, mobile Phase B: acetonitrile; flow rate: 1.50 mL/min; gradient: 80% B to 95% B in 1.80 min, hold at 95% for 0.80 min, 95% B to 10% B in 0.15 min; 254 nm; RT: 1.079 min.

### Example S13. 1-(5-((3-fluorophenyl)ethynyl)-7-methyl-2,3-dihydro-1H-inden-1-yl)-3-methylazetidin-3-ol (13a & 13b)

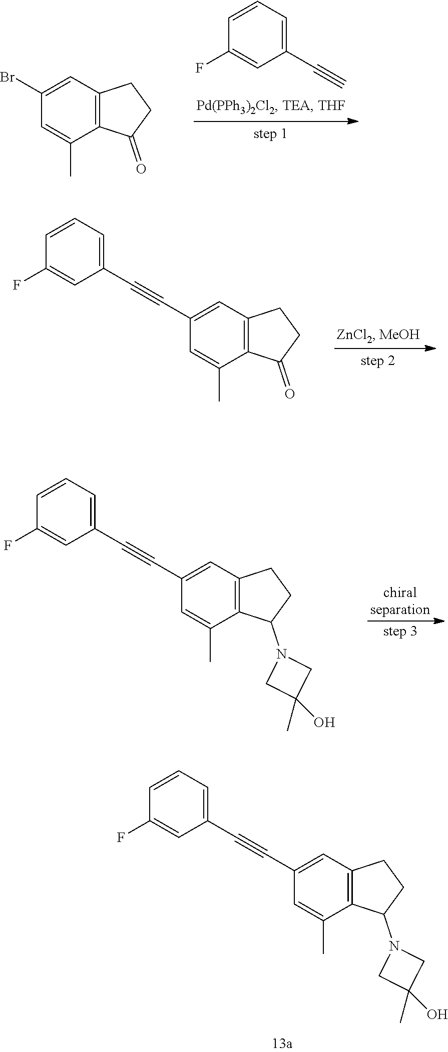

### Synthesis of 5-[2-(3-fluorophenyl)ethynyl]-7-methyl-indan-1-one

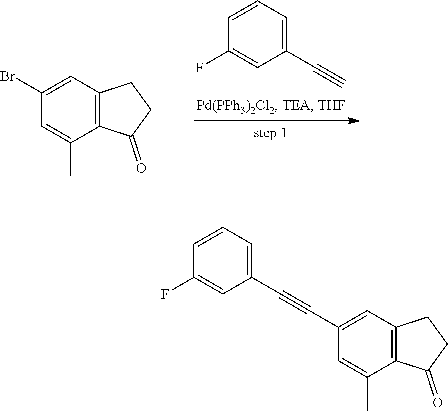

A solution of 1-ethynyl-3-fluoro-benzene (533 mg, 4.44 mmol, 2.00 equiv.), 5-bromo-7-methyl-indan-1-one (500 mg, 2.220 mmol, 1.00 equiv.), K2CO₃ (919 mg, 6.66 mmol, 3.00 equiv.), Cul (21 mg, 0.11 mmol, 0.05 equiv.) and Pd(PPh₃)₂Cl₂ (155 mg, 0.22 mmol, 0.10 equiv.) in DMF (10.0 mL) was stirred at 80° C. for 16 h. LCMS showed that the reaction was complete. The reaction mixture was concentrated under reduced pressure. The residue was purified by flash column chromatography on silica gel (eluted with PE/EtOAc, 3/1) to afford 5-[2-(3-fluorophenyl)ethynyl]-7-methyl-indan-1-one (500 mg, 85%) as an off-white solid. LCMS (ESI, m/z): 265 [M+H]⁺

### Synthesis of 1-[5-[2-(3-fluorophenyl)ethynyl]-7-methyl-indan-1-yl]-3-methyl-azetidin-3-ol

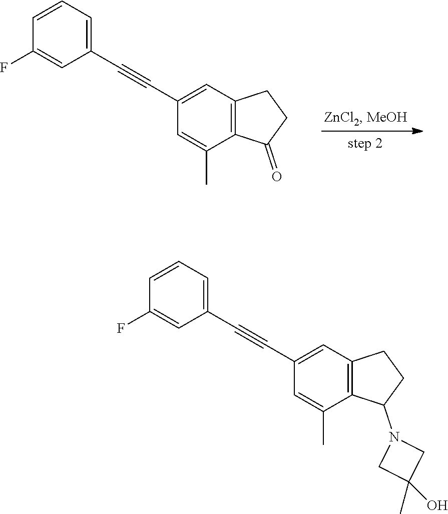 [SMILES: Cc1cc(C#Cc2cccc(F)c2)cc2c1C(=O)CC2 ; Cc1cc(C#Cc2cccc(F)c2)cc2c1C(N1CC(C)(O)C1)CC2]

A solution of 5-[2-(3-fluorophenyl)ethynyl]-7-methyl-indan-1-one (500 mg, 1.89 mmol, 1.00 equiv.), 3-methylazetidin-3-ol (164 mg, 1.89 mmol, 1.00 equiv.), ZnCl₂ (2 M in THF, 1.9 mL, 3.78 mmol, 2.00 equiv.) and NaBH₃CN (363 mg, 5.68 mmol, 3.00 equiv.) in methanol (10 mL) was stirred at 80° C. for 16 h. LCMS showed the reaction was completed. The reaction mixture was quenched with water (50 mL) and extracted with DCM (3*30 mL). The combined organic layers were concentrated under reduced pressure. The residue was purified by flash chromatography on silica gel (eluted with ethyl acetate/petroleum ether, 1:1) to afford 1-[5-[2-(3-fluorophenyl)ethynyl]-7-methyl-indan-1-yl]-3-methyl-azetidin-3-ol (80 mg, 12%) as an off-white solid. LCMS (ESI, m/z): 336 [M+H]⁺.

### Chiral Separation of 1-[5-[2-(3-fluorophenyl)ethynyl]-7-methyl-indan-1-yl]-3-methyl-azetidin-3-ol (13a)

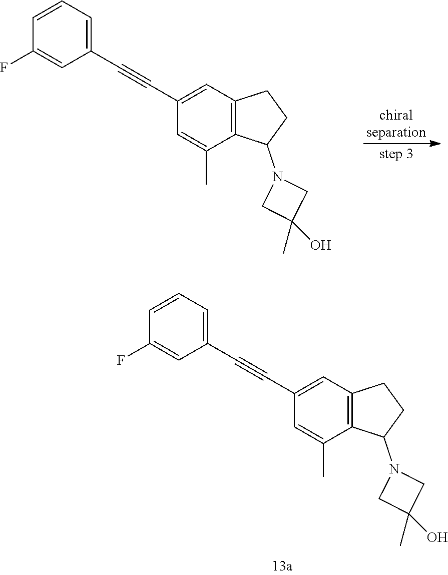 [SMILES: Cc1cc(C#Cc2cccc(F)c2)cc2c1C(N1CC(C)(O)C1)CC2 ; Cc1cc(C#Cc2cccc(F)c2)cc2c1C(N1CC(C)(O)C1)CC2]

The racemate (80 mg) was purified by Prep-Chiral (Column: Lux 5 μm Cellulose-2, 2.12*25 cm,5 μm; Mobile Phase A: Hex (0.1% DEA)-HPLC, Mobile Phase B: IPA-HPLC; Flow rate: 20 mL/min; Gradient: 98% B to 98% B in 25 min; 220/254 nm; RT1:17.904 min) to afford 1-[5-[2-(3-fluorophenyl)ethynyl]-7-methyl-indan-1-yl]-3-methyl-azetidin-3-ol (the first eluting peak, 17.7 mg, 21%) as an off-white solid.

¹H NMR (300 MHz, DMSO-d₆) δ 7.52-7.45 (m, 1H), 7.41-7.37 (m, 2H), 7.31-7.24 (m, 2H), 7.18 (s, 1H), 5.12 (s, 1H), 4.08 (d, J=6.6 Hz, 1H), 3.11 (d, J=6.3 Hz, 1H), 3.02 (d, J=6.3 Hz, 1H), 2.98-2.90 (m, 3H), 2.79-2.71 (m, 1H), 2.38 (s, 3H), 2.19-2.12 (m, 1H), 1.96-1.83 (m, 1H), 1.32 (s, 3H).

¹⁹F NMR (376 MHZ, DMSO-d₆) δ -112.515.

LCMS (ESI, m/z): 336 [M+H]⁺. Analytic Conditions: column: L-column3 C18 Column 3.0*30 mm, 2.0 μm; mobile Phase A: Water/5 mM NH₄HCO₃, mobile Phase B: acetonitrile; flow rate: 1.5000 mL/min; gradient: 30% B to 95% B in 2.19 min, hold at 95% for 0.6 min, 95% B to 10% B in 0.03 min; 254 nm; RT: 1.701 min.

### Chiral Separation of 1-[5-[2-(3-fluorophenyl)ethynyl]-7-methyl-indan-1-yl]-3-methyl-azetidin-3-ol (13b)

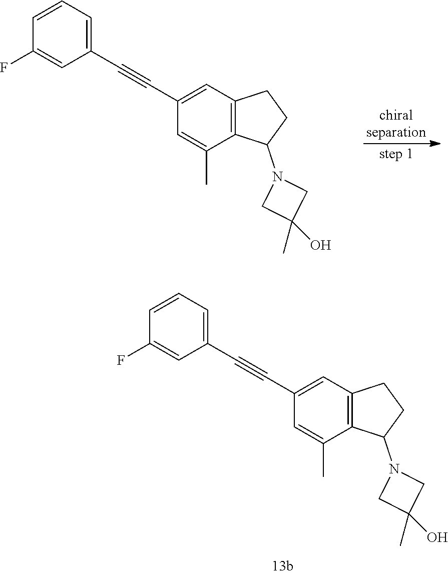 [SMILES: Cc1cc(C#Cc2cccc(F)c2)cc2c1C(N1CC(C)(O)C1)CC2 ; Cc1cc(C#Cc2cccc(F)c2)cc2c1C(N1CC(C)(O)C1)CC2]

The racemate (80 mg) was purified by Prep-Chiral (Column: Lux 5 μm Cellulose-2, 2.12*25 cm,5 μm; Mobile Phase A: Hex (0.1% DEA)-HPLC, Mobile Phase B: IPA-HPLC; Flow rate: 20 mL/min; Gradient: 98% B to 98% B in 25 min; 220/254 nm; RT1:17.904 min) to afford 1-[5-[2-(3-fluorophenyl)ethynyl]-7-methyl-indan-1-yl]-3-methyl-azetidin-3-ol (the second eluting peak, 21.9 mg, 27%) as an off-white solid.

¹H NMR (300 MHZ, DMSO-d₆) δ 7.52-7.45 (m, 1H), 7.41-7.37 (m, 2H), 7.31-7.24 (m, 2H), 7.18 (s, 1H), 5.12 (s, 1H), 4.08 (d, J=6.6 Hz, 1H), 3.10 (d, J=6.0 Hz, 1H), 3.02 (d, J=6.0 Hz, 1H), 2.98-2.90 (m, 3H), 2.79-2.71 (m, 1H), 2.38 (s, 3H), 2.19-2.12 (m, 1H), 1.96-1.83 (m, 1H), 1.32 (s, 3H).

¹⁹F NMR (376 MHz, DMSO-d₆) δ -112.173.

LCMS (ESI, m/z): 336 [M+H]⁺. Analytic Conditions: column: L-column3 C18 Column 3.0*30 mm, 2.0 μm; mobile phase A: Water/5 mM NH₄HCO₃, mobile phase B: acetonitrile; flow rate: 1.5000 mL/min; gradient: 30% B to 95% B in 2.19 min, hold at 95% for 0.6 min, 95% B to 10% B in 0.03 min; 254 nm; RT: 1.702 min.

### Example S14. 1-(5-(2,6-dichlorophenethyl)-7-methyl-2,3-dihydro-1H-inden-1-yl)-3-methylazetidin-3-ol (14a & 14b)

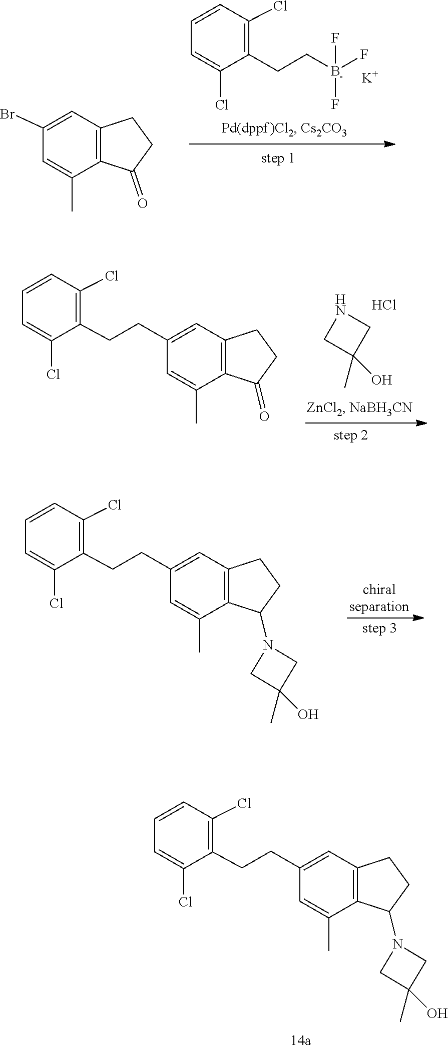

### Synthesis of 5-[2-(2,6-dichlorophenyl)ethyl]-7-methyl-indan-1-one

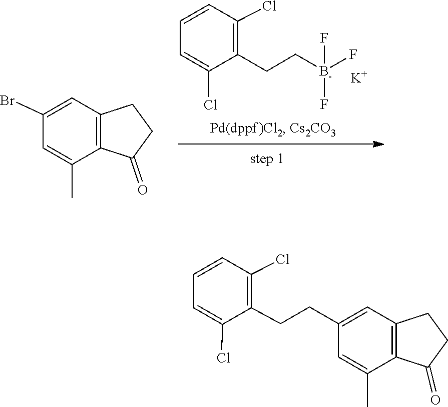

A solution of 5-bromo-7-methyl-indan-1-one (500 mg, 2.22 mmol, 1.00 equiv.), potassium 2-(2,6-dichlorophenyl)ethyl-trifluoro-boranuide (811 mg, 2.89 mmol, 1.30 equiv.) Pd(dppf)Cl₂ (162 mg, 0.22 mmol, 0.10 equiv.) and Cs₂CO₃ (2.1 mg, 6.66 mmol, 3.00 equiv.), in toluene (10 mL) was stirred at 80° C. for 2 h. LCMS showed the reaction was completed. The reaction was concentrated under reduced pressure. The residue was purified by flash column chromatography on silica gel (eluted with PE/EtOAc, 3:1) to get 5-[2-(2,6-dichlorophenyl)ethyl]-7-methyl-indan-1-one (500 mg, 70% yield) as an off-white solid. LCMS (ESI, m/z): 319 [M+H]⁺.

### Synthesis of 1-[5-[2-(2,6-dichlorophenyl)ethyl]-7-methyl-indan-1-yl]-3-methyl-azetidin-3-ol

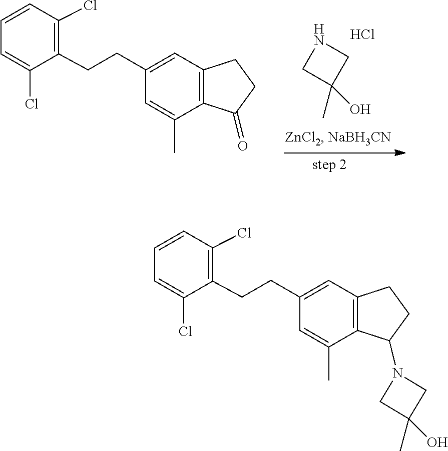

A solution of 5-[2-(2,6-dichlorophenyl)ethyl]-7-methyl-indan-1-one (500 mg, 1.57 mmol, 1.00 equiv.), 3-methylazetidin-3-ol (136 mg, 1.57 mmol, 1.00 equiv.), ZnCl₂ (2 M in THF, 1.5 mL, 3.13 mmol, 2.00 equiv.) and NaBH₃CN (300 mg, 4.70 mmol, 3.00 equiv.) in methanol (10 mL) was stirred at 80° C. for 16 h. LCMS showed the reaction was completed. The reaction mixture was quenched with water (50 mL) and extracted with DCM (3*20 mL). The combined organic layers were concentrated under reduced pressure. The residue was purified by flash column chromatography on silica gel (eluted with water/acetonitrile, 1:2) to 1-[5-[2-(2,6-dichlorophenyl)ethyl]-7-methyl-indan-1-yl]-3-methyl-azetidin-3-ol (100 mg, 16%) as an off-white solid. LCMS (ESI, m/z): 390 [M+H]⁺.

### Chiral Separation of 1-[5-[2-(2,6-dichlorophenyl)ethyl]-7-methyl-indan-1-yl]-3-methyl-azetidin-3-ol (14a)

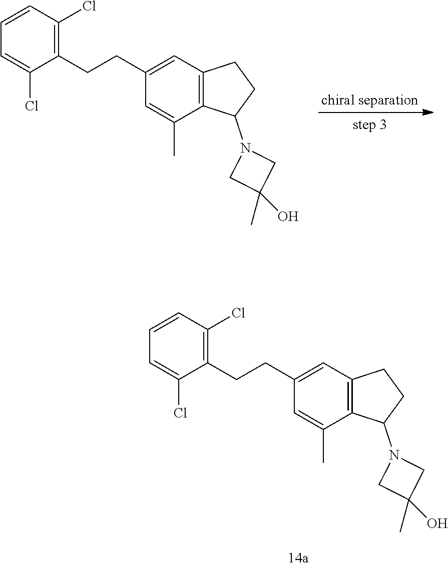 [SMILES: Cc1cc(CCc2c(Cl)cccc2Cl)cc2c1C(N1CC(C)(O)C1)CC2 ; Cc1cc(CCc2c(Cl)cccc2Cl)cc2c1C(N1CC(C)(O)C1)CC2]

The racemate (100 mg) was purified by Prep-Chiral HPLC(Column: Lux 5 μm Cellulose-2, 2.12*25 cm, 5 μm; Mobile Phase A: Hex (0.5% 2 M NH₃-MeOH)-HPLC, Mobile Phase B: EtOH-HPLC; Flow rate: 20 mL/min; Gradient: 98% B to 98% B in 15 min; 220/254 nm; RT1:10. 221 min) to afford the desired isomer 1-[5-[2-(2,6-dichlorophenyl)ethyl]-7-methyl-indan-1-yl]-3-methyl-azetidin-3-ol (the first eluting peak, 27.7 mg, 27%) as an off-white solid.

¹H NMR (300 MHz, DMSO-d₆) δ 7.50-7.47 (m, 2H), 7.33-7.27 (m, 1H), 6.94 (s, 1H), 6.84 (s, 1H), 5.10 (s, 1H), 4.02 (d, J=6.6 Hz, 1H), 3.13-3.07 (m, 3H), 3.02-2.90 (m, 4H), 2.73-2.65 (m, 3H), 2.35 (s, 3H), 2.15-2.08 (m, 1H), 1.93-1.80 (m, 1H), 1.31 (s, 3H).

LCMS (ESI, m/z): 390 [M+H]⁺. Analytic Conditions: column: HALO C18 Column 3.0*30 mm, 2.7 μm; mobile Phase A: Water/0.05% TFA, mobile Phase B: Acetonitrile/0.05% TFA; flow rate: 1.50 mL/min; gradient: 5% B to 100% B in 1.19 min, hold at 100% for 0.6 min, 100% B to 5% B in 0.03 min; 220 nm; RT: 0.994 min.

### Chiral Separation of 1-[5-[2-(2,6-dichlorophenyl)ethyl]-7-methyl-indan-1-yl]-3-methyl-azetidin-3-ol (14b)

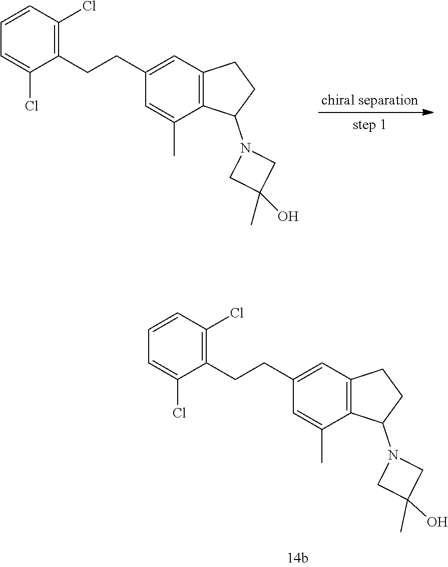 [SMILES: Cc1cc(CCc2c(Cl)cccc2Cl)cc2c1C(N1CC(C)(O)C1)CC2 ; Cc1cc(CCc2c(Cl)cccc2Cl)cc2c1C(N1CC(C)(O)C1)CC2]

The racemate (100 mg) was purified by Prep-Chiral HPLC(Column: Lux 5 μm Cellulose-2, 2.12*25 cm,5 μm; Mobile Phase A: Hex (0.5% 2 M NH₃-MeOH)-HPLC, Mobile Phase B: EtOH-HPLC; Flow rate: 20 mL/min; Gradient: 98% B to 98% B in 15 min; 220/254 nm; RT2:11. 608 min) to afford the desired isomer 1-[5-[2-(2,6-dichlorophenyl)ethyl]-7-methyl-indan-1-y1]-3-methyl-azetidin-3-ol (the second eluting peak, 27.5 mg, 27%) as an off-white solid.

¹H NMR (300 MHz, DMSO-d₆) δ 7.51-7.47 (m, 2H), 7.33-7.27 (m, 1H), 6.94 (s, 1H), 6.84 (s, 1H), 5.09 (s, 1H), 4.01 (d, J=6.6 Hz, 1H), 3.12-3.07 (m, 3H), 3.01-2.90 (m, 4H), 2.73-2.64 (m, 3H), 2.35 (s, 3H), 2.15-2.08 (m, 1H), 1.92-1.83 (m, 1H), 1.31 (s, 3H).

LCMS (ESI, m/z): 390 [M+H]⁺. Analytic Conditions: column: HALO C18 Column 3.0*30 mm, 2.7 μm; mobile Phase A: Water/0.05% TFA, mobile Phase B: Acetonitrile/0.05% TFA; flow rate: 1.5000 mL/min; gradient: 5% B to 100% B in 1.19 min, hold at 100% for 0.6 min, 100% B to 5% B in 0.03 min; 220 nm; RT: 0.994 min.

### Example S15. 1-(5-((2,6-dichlorobenzyl)oxy)-7-methyl-2,3-dihydro-1H-inden-1-yl)-3-methylazetidin-3-ol (15a & 15b)

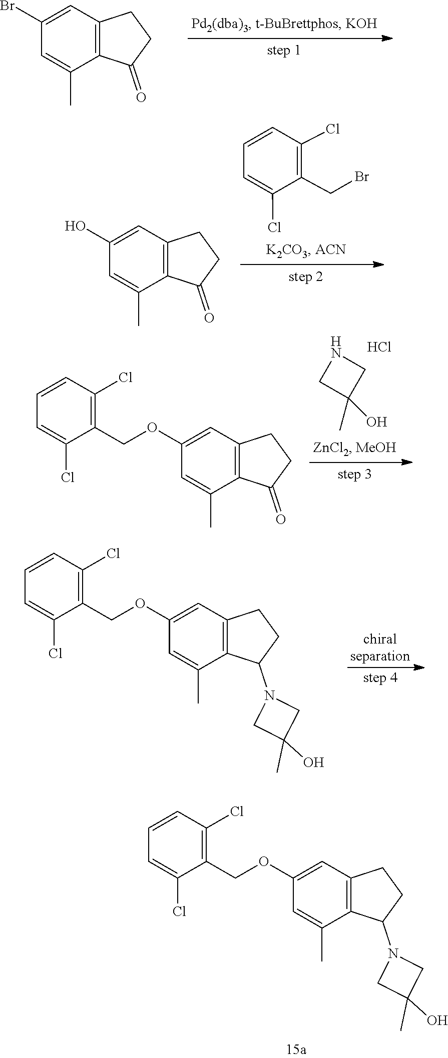

### Synthesis of 5-hydroxy-7-methyl-indan-1-one

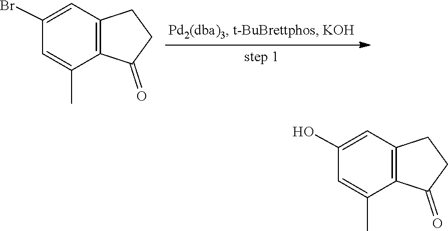

A solution of 5-bromo-7-methyl-indan-1-one (800 mg, 3.55 mmol, 1.00 equiv.) and KOH(597 mg, 10.660 mmol, 3.00 equiv.), Pd2 (dba)₃ CHC13 (367 mg, 0.36 mmol, 0.10 equiv.) and 1-BuBrettphos (344 mg, 0.71 mmol, 0.20 equiv.) in 1,4-dioxane (10 mL) and water (1 mL) was stirred at 80° C. for 16 h. LCMS showed the reaction was completed. The reaction was acidified to pH 4˜5 with 1M HCl. The reaction mixture was filtered and the filtration was concentrated under reduced pressure. The residue was purified by flash chromatography on C18 silica (eluted with water/acetonitrile, 2:3) to afford 5-hydroxy-7-methyl-indan-1-one (480 mg, 83%) as an off-white solid. LCMS (ESI, m/z): 163 [M+H]⁺.

### Synthesis of 5-[(2,6-dichlorophenyl) methoxy]-7-methyl-indan-1-one

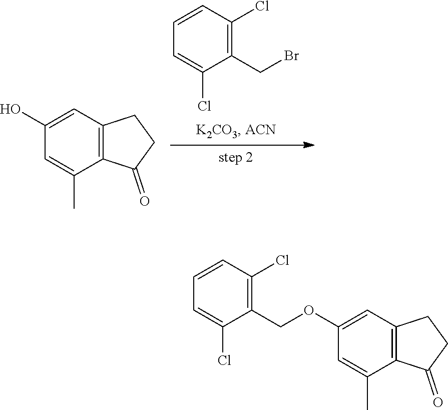 [SMILES: Clc1cccc(Cl)c1CBr ; Cc1cc(O)cc2c1C(=O)CC2 ; Cc1cc(OCc2c(Cl)cccc2Cl)cc2c1C(=O)CC2]

A solution of 5-hydroxy-7-methyl-indan-1-one (450 mg, 2.77 mmol, 1.00 equiv.), 2-(bromomethyl)-1,3-dichloro-benzene (732 mg, 3.05 mmol, 1.10 equiv.) and K₂CO₃ (1.1 mg, 8.32 mmol, 3.00 equiv.) in MeCN (10 mL) was stirred at 60° C. for 2 h. LCMS showed the reaction was completed. The reaction mixture was filtered and the filtrate was concentrated under reduced pressure. The residue was purified by flash chromatography on C18 silica (eluted with water/acetonitrile, 1:6) to afford 5-[(2,6-dichlorophenyl) methoxy]-7-methyl-indan-1-one (600 mg, 67% yield) as an off-white solid. LCMS (ESI, m/z): 321 [M+H]⁺.

### Synthesis of 1-[5-[(2,6-dichlorophenyl) methoxy]-7-methyl-indan-1-yl]-3-methyl-azetidin-3-ol

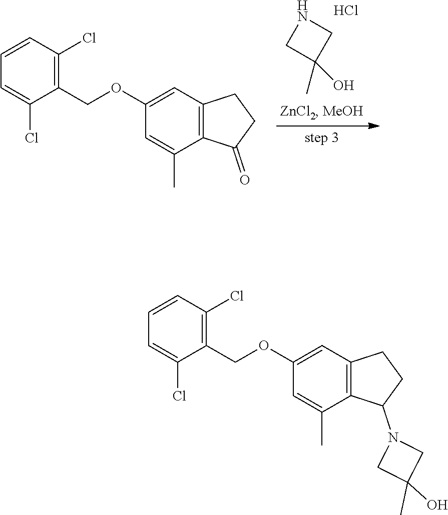

A solution of 5-[(2,6-dichlorophenyl) methoxy]-7-methyl-indan-1-one (600 mg, 1.87 mmol, 1.00 equiv.), 3-methylazetidin-3-ol (162 mg, 1.87 mmol, 1.00 equiv.), ZnCl₂ (2 M in THF, 1.8 mL, 3.74 mmol, 2.00 equiv.) and NaBH₃CN (358 mg, 5.60 mmol, 3.00 equiv.) in methanol (15 mL) was stirred at 80° C. for 16 h. LCMS showed the reaction was completed. The reaction mixture was quenched with water (50 mL) and extracted with DCM (3*20 mL). The combined organic layers were concentrated under reduced pressure. The residue was purified by flash chromatography on C18 silica (eluted with water/acetonitrile, 1:2) to afford 1-[5-[(2,6-dichlorophenyl) methoxy]-7-methyl-indan-1-yl]-3-methyl-azetidin-3-ol (150 mg, 20%) as an off-white solid. LCMS (ESI, m/z): 392 [M+H]⁺.

### Chiral Separation of 1-[5-[(2,6-dichlorophenyl) methoxy]-7-methyl-indan-1-yl]-3-methyl-azetidin-3-ol (15a)

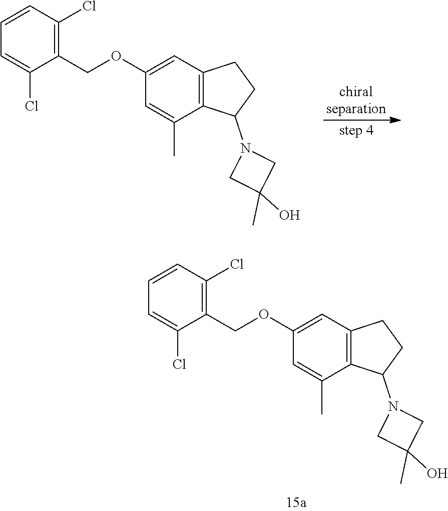

The racemate (120 mg) was purified by SFC(Column: Lux 5 μm Celluloes-3, 3*25 cm, 5 μm; Mobile Phase A: CO₂, Mobile Phase B: IPA (0.5% 2 M NH₃-MeOH); Flow rate: 80 mL/min; Gradient: 30% B; 220 nm; RT1:4.21 min) to give the desired isomer 1-[5-[(2,6-dichlorophenyl) methoxy]-7-methyl-indan-1-yl]-3-methyl-azetidin-3-ol (the first eluting peak, 33.1 mg, 27%) as an off-white solid.

¹H NMR (300 MHz, DMSO-d₆) δ 7.59-7.56 (m, 2H), 7.47 (dd, J=9.0, 6.6 Hz, 1H), 6.75 (d, J=2.4 Hz, 1H), 6.63 (d, J=2.4 Hz, 1H), 5.17 (s, 2H), 5.10 (s, 1H), 3.99 (d, J=6.3 Hz, 1H), 3.09 (d, J=6.0 Hz, 1H), 3.02-2.89 (m, 4H), 2.73-2.64 (m, 1H), 2.33 (s, 3H), 2.14-2.07 (m, 1H), 1.94-1.82 (m, 1H), 1.31 (s, 3H).

LCMS (ESI, m/z): 392 [M+H]⁺. Analytic Conditions: column: HALO C18 Column 3.0*30 mm, 2.7 μm; mobile Phase A: Water/0.05% TFA, mobile Phase B:

Acetonitrile/0.05% TFA; flow rate: 1.5000 mL/min; gradient: 5% B to 100% B in 1.19 min, hold at 100% for 0.6 min, 100% B to 5% B in 0.03 min; 220 nm; RT: 0.923 min.

### Chiral Separation of 1-[5-[(2,6-dichlorophenyl) methoxy]-7-methyl-indan-1-yl]-3-methyl-azetidin-3-ol (15b)

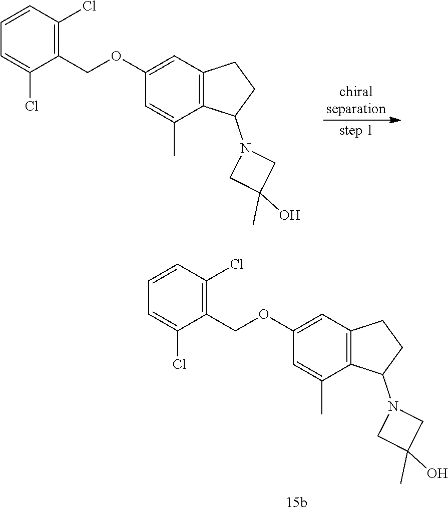

The racemate (120 mg) was purified by SFC(Column: Lux 5 μm Celluloes-3, 3*25 cm, 5 μm; Mobile Phase A: CO₂, Mobile Phase B: IPA (0.5% 2 M NH₃-MeOH); Flow rate: 80 mL/min;

Gradient: 30% B; 220 nm; RT2:4.98 min) to give the desired isomer 1-[5-[(2,6-dichlorophenyl) methoxy]-7-methyl-indan-1-yl]-3-methyl-azetidin-3-ol (the second eluting peak, 21.6 mg, 17%) as an off-white solid.

¹H NMR (300 MHz, DMSO-d₆) δ 7.59-7.56 (m, 2H), 7.47 (dd, J=9.3, 6.3 Hz, 1H), 6.75 (d, J=2.4 Hz, 1H), 6.63 (d, J=2.4 Hz, 1H), 5.17 (s, 2H), 5.09 (s, 1H), 3.99-3.95 (m, 1H), 3.09-3.05 (m, 1H), 3.01-2.90 (m, 4H), 2.73-2.64 (m, 1H), 2.33 (s, 3H), 2.14-2.07 (m, 1H), 1.94-1.82 (m, 1H), 1.31 (s, 3H).

LCMS (ESI, m/z): 392 [M+H]⁺. Analytic Conditions: column: HALO C18 Column 3.0*30 mm, 2.7 μm; mobile Phase A: Water/0.05% TFA, mobile Phase B:

Acetonitrile/0.05% TFA; flow rate: 1.5000 mL/min; gradient: 5% B to 100% B in 1.19 min, hold at 100% for 0.6 min, 100% B to 5% B in 0.03 min; 220 nm; RT: 0.931 min.

### Example S16. 1-(5-(3-chloro-4-cyclopropylphenyl)-4-methyl-2,3-dihydro-1H-inden-1-yl)-3-methylazetidin-3-ol (16a & 16b)

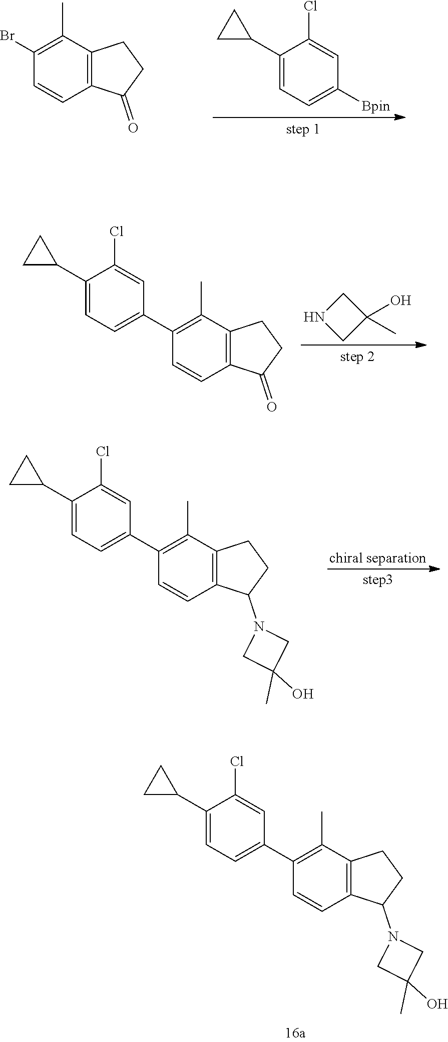

### Synthesis of 5-(3-chloro-4-cyclopropyl-phenyl)-4-methyl-indan-1-one

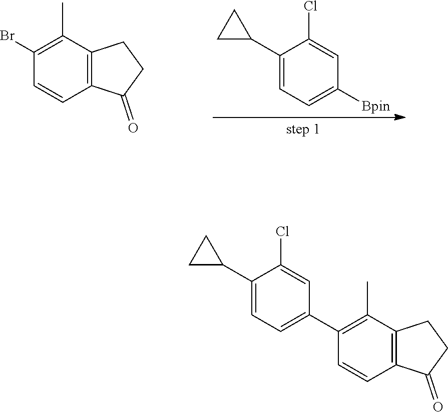

To a solution of 5-bromo-4-methyl-indan-1-one (600 mg, 2.67 mmol, 1.00 equiv.) in 1,4-dioxane (8 mL) and water (0.8 mL) were added 2-(3-chloro-4-cyclopropyl-phenyl)-4,4,5,5-tetramethyl-1,3,2-dioxaborolane (1.48 g, 5.33 mmol, 2.00 equiv.), t-BuONa (767 mg, 8.00 mmol, 3.00 equiv.) and Pd(dppf)Cl₂ (217 mg, 0.27 mmol, 0.10 equiv.). The reaction was stirred for 12 h at 90° C. LCMS showed the reaction was completed. The reaction was concentrated under vacuum. The residue was purified by flash column chromatography on silica gel (eluted with dichloromethane/methanol, 17/1) to afford 5-(3-chloro-4-cyclopropyl-phenyl)-4-methyl-indan-1-one (432 mg, 54%) as a yellow solid. LCMS (ESI, m z): 297 [M+H]⁺.

### Synthesis of 1-[5-(3-chloro-4-cyclopropyl-phenyl)-4-methyl-indan-1-yl]-3-methyl-azetidin-3-ol

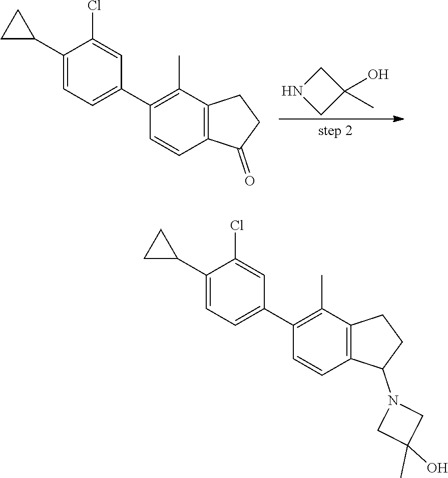

A mixture of 5-(3-chloro-4-cyclopropyl-phenyl)-4-methyl-indan-1-one (250 mg, 0.84 mmol, 1.00 equiv.), 3-methylazetidin-3-ol (109 mg, 1.26 mmol, 1.50 equiv.), ZnCl₂ (1.1 mL, 2M in 4Me-THF, 2.11 mmol, 2.50 equiv.) and NaBH₃CN (215 mg, 3.37 mmol, 4.00 equiv.) in methanol (8 mL) was stirred for 12 h at 60° C. LCMS showed the reaction was completed. The reaction was quenched by water (30 mL) and extracted with EtOAc (2×15 mL). The combined organic layers were concentrated under reduced pressure. The residue was purified by flash column chromatography on C18 silica (eluted with water/acetonitrile, 1/3) to afford 1-[5-(3-chloro-4-cyclopropyl-phenyl)-4-methyl-indan-1-yl]-3-methyl-azetidin-3-ol (165 mg, 53%) as a yellow solid. LCMS (ESI, m z): 368 [M+H]⁺.

### Chiral Separation of 1-[5-(3-chloro-4-cyclopropyl-phenyl)-4-methyl-indan-1-yl]-3-methyl-azetidin-3-ol (16a)

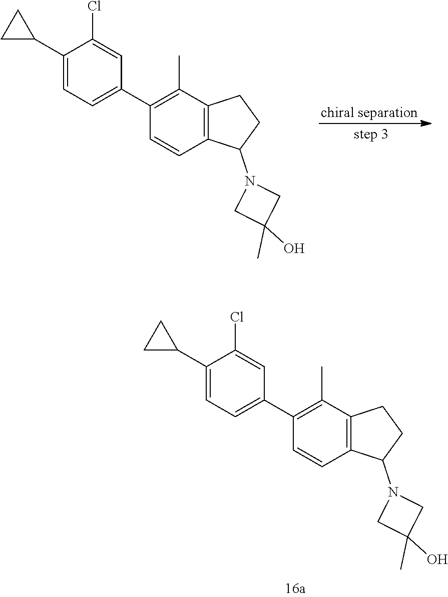 [SMILES: Cc1c(-c2ccc(C3CC3)c(Cl)c2)ccc2c1CCC2N1CC(C)(O)C1 ; Cc1c(-c2ccc(C3CC3)c(Cl)c2)ccc2c1CCC2N1CC(C)(O)C1]

The racemate (80 mg) was purified by Prep-Chiral HPLC(Column: Lux 5 μm Cellulose-2, 12*25 cm, 5 μm; Mobile Phase A: Hex (0.5% 2M NH₃-MeOH)-HPLC, Mobile Phase B: EtOH-HPLC; Flow rate: 20 mL/min; Gradient: 2% B to 2% B in 21 min; 254/220 nm; RT1:16. 824 min) to afford 1-[5-(3-chloro-4-cyclopropyl-phenyl)-4-methyl-indan-1-yl]-3-methyl-azetidin-3-ol (9.9 mg, 12%) as an off-white solid.

LCMS (ESI, m/z): 368 [M+H]⁺. Analytic Conditions: column: L-column3 C18 Column 3.0*30 mm, 2.0 μm; mobile Phase A: Water/5 mM NH₄HCO₃, mobile Phase B: acetonitrile; flow rate: 1.5000 mL/min; gradient: 30% B to 80% B in 1.80 min, 80% B to 95% B in 0.5 min, hold at 95% for 0.5 min, 95% B to 10% B in 0.1 min; 254 nm; RT: 1.771 min.

¹H NMR (400 MHZ, DMSO-d₆) δ 7.31 (d, J=2.0 Hz, 1H), 7.18-7.13 (m, 2H), 7.07 (d, J=8.0 Hz, 1H), 6.99 (d, J=7.6 Hz, 1H), 5.19 (s, 1H), 3.87 (s, 1H), 3.20-3.00 (m, 3H), 2.90-2.84 (m, 2H), 2.77-2.69 (m, 1H), 2.21-2.14 (m, 1H), 2.10 (s, 3H), 2.09-2.04 (m, 1H), 1.91-1.84 (m, 1H), 1.32 (s, 3H), 1.06-1.01 (m, 2H), 0.77-0.73 (m, 2H).

### Chiral Separation of 1-[5-(3-chloro-4-cyclopropyl-phenyl)-4-methyl-indan-1-yl]-3-methyl-azetidin-3-ol (16b)

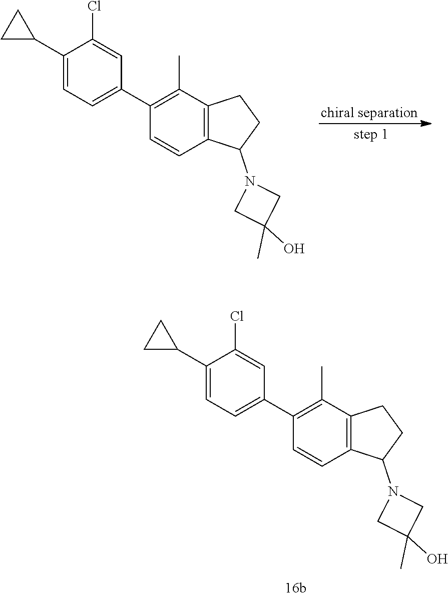 [SMILES: Cc1c(-c2ccc(C3CC3)c(Cl)c2)ccc2c1CCC2N1CC(C)(O)C1 ; Cc1c(-c2ccc(C3CC3)c(Cl)c2)ccc2c1CCC2N1CC(C)(O)C1]

The racemate (80 mg) was purified by Prep-Chiral HPLC(Column: Lux 5 μm Cellulose-2, 12*25 cm, 5 μm; Mobile Phase A: Hex (0.5% 2M NH₃-MeOH)-HPLC, Mobile Phase B: EtOH-HPLC; Flow rate: 20 mL/min; Gradient: 2% B to 2% B in 21 min; 254/220 nm; RT2:19. 055 min) to afford 1-[5-(3-chloro-4-cyclopropyl-phenyl)-4-methyl-indan-1-yl]-3-methyl-azetidin-3-ol (31 mg, 36%) as an off-white solid.

LCMS (ESI, m/z): 368 [M+H]⁺. Analytic Conditions: column: L-column3 C18 Column 4.6*100 mm, 3.0 μm; mobile Phase A: Water/5 mM NH₄HCO₃, mobile Phase B: acetonitrile; flow rate: 1.50 mL/min; gradient: 30% B to 80% B in 6.00 min, 80% B to 95% B in 2.00 min, 95% B to 10% B in 2.00 min; 254 nm; RT: 6.295 min.

¹H NMR (400 MHZ, DMSO-d₆) δ 7.31 (d, J=1.6 Hz, 1H), 7.17 (dd, J=8.0, 1.6 Hz, 1H), 7.12 (d, J=8.0 Hz, 1H), 7.07 (d, J=8.0 Hz, 1H), 6.98 (d, J=8.0 Hz, 1H), 5.14 (s, 1H), 3.83-3.80 (m, 1H), 3.21-3.17 (m, 1H), 3.09 (s, 2H), 2.94 (d, J=6.4 Hz, 1H), 2.91-2.83 (m, 1H), 2.75-2.67 (m, 1H), 2.21-2.15 (m, 1H), 2.10 (s, 3H), 2.07-2.00 (m, 1H), 1.89-1.82 (m, 1H), 1.32 (s, 3H), 1.06-0.99 (m, 2H), 0.77-0.73 (m, 2H).

### Example S17. 1-(5-((3-fluorophenyl)ethynyl)-4-methyl-2,3-dihydro-1H-inden-1-yl)-3-methylazetidin-3-ol (17a & 17b)

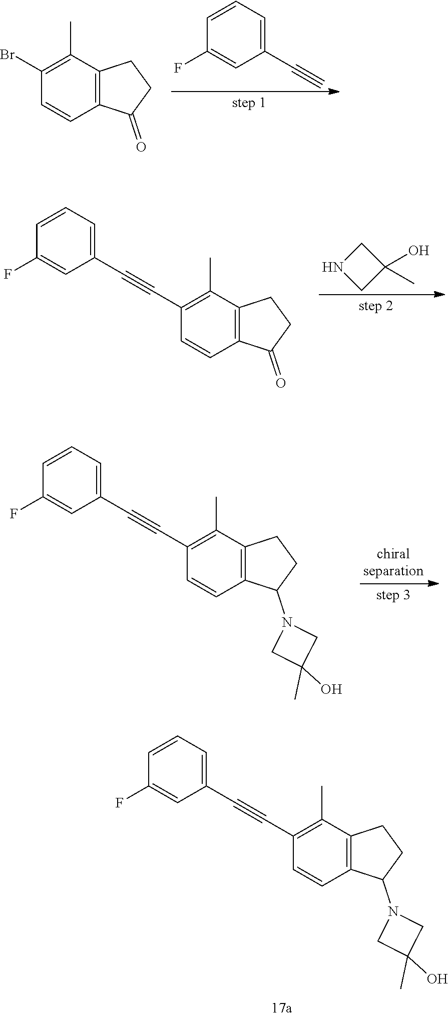

### Synthesis of 5-[2-(3-fluorophenyl)ethynyl]-4-methyl-indan-1-one

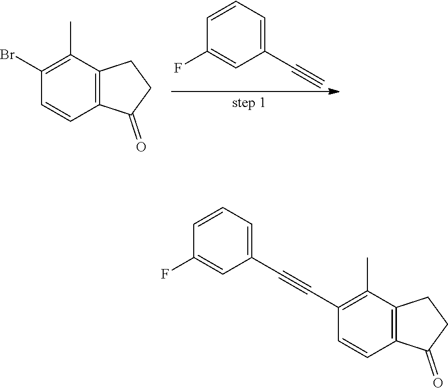

A solution of 5-bromo-4-methyl-indan-1-one (300 mg, 1.33 mmol, 1.00 equiv.), 1-ethynyl-3-fluoro-benzene (480 mg, 4.00 mmol, 3.00 equiv.), K2CO₃ (368 mg, 2.67 mmol, 2.00 equiv.), Pd(PPh₃)₂Cl₂ (93 mg, 0.13 mmol, 0.10 equiv.) and CuI (51 mg, 0.27 mmol, 0.20 equiv.), in DMF (3 mL) was stirred at 60° C. for 2 days under nitrogen atmosphere. LCMS showed the reaction was completed. The reaction mixture was concentrated under reduced pressure. The residue was purified by flash column chromatography on silica gel (eluted with PE/EtOAc, 1/1) to afford 5-[2-(3-fluorophenyl)ethynyl]-4-methyl-indan-1-one (300 mg, 85%) as a yellow solid. LCMS (ESI, m z): 265 [M+H]⁺.

### Synthesis of 1-[5-[2-(3-fluorophenyl)ethynyl]-4-methyl-indan-1-yl]-3-methyl-azetidin-3-ol

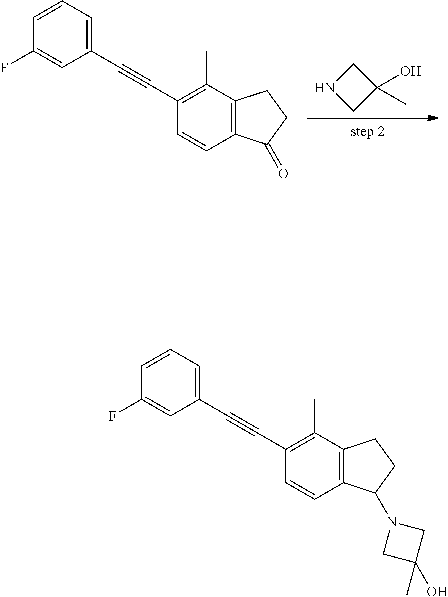

A solution of 5-[2-(3-fluorophenyl)ethynyl]-4-methyl-indan-1-one (300 mg, 1.14 mmol, 1.00 equiv.), 3-methylazetidin-3-ol (198 mg, 2.28 mmol, 2.00 equiv.), ZnCl₂ (1.14 mL, 2M in 4Me-THF, 2.27 mmol, 2.00 equiv.) and NaBH₃CN (291 mg, 4.54 mmol, 3.00 equiv.) in methanol (10 mL) was stirred at 80° C. for 3 h. LCMS showed the reaction was completed. The reaction mixture was quenched with water (50 mL) and extracted with DCM (3*30 mL). The combined organic layers were concentrated under reduced pressure. The residue was purified by flash chromatography on silica gel (eluted with ethyl acetate/petroleum ether, 2:3) to afford 1-[5-[2-(3-fluorophenyl)ethynyl]-4-methyl-indan-1-yl]-3-methyl-azetidin-3-ol (350 mg, 92%) as a yellow solid. LCMS (ESI, m z): 356 [M+H]⁺

### Chiral Separation of 1-[5-[2-(3-fluorophenyl)ethynyl]-4-methyl-indan-1-yl]-3-methyl-azetidin-3-ol (17a)

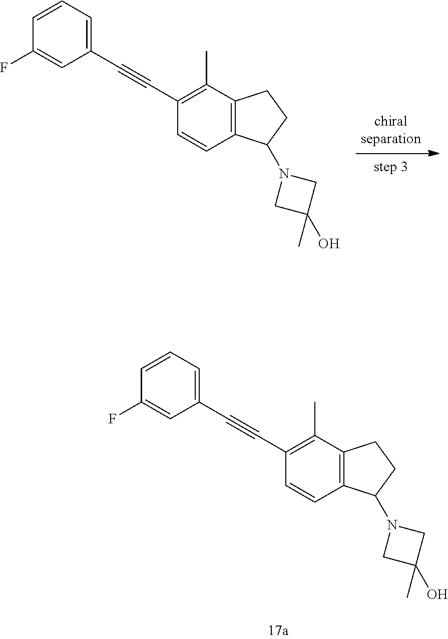 [SMILES: Cc1c(C#Cc2cccc(F)c2)ccc2c1CCC2N1CC(C)(O)C1 ; Cc1c(C#Cc2cccc(F)c2)ccc2c1CCC2N1CC(C)(O)C1]

The racemate (350 mg) was separated by chairl-HPLC(Column: Lux 5 μm Cellulose-2, 3*15 cm, 5 μm; Mobile Phase A: HEX (0.5% 2M NH₃-MeOH)-HPLC, Mobile Phase B: EtOH-HPLC; Flow rate: 20 mL/min; Gradient: 5% B to 5% B in 71 min; Wave Length: 220/254 nm; RT1:13.238 min; RT2:27.446 min) to afford the isomers. The first eluting peak compound was purified by prep-HPLC(Column: Sunfire prep C18 column, 30*150 mm, 5 μm; Mobile Phase A: water (0.1% FA), Mobile Phase B: ACN; Flow rate: 60 mL/min; Gradient: 15% B to 35% B in 10 min, hold at 35% B for 2 min; Wave Length: 254/220 nm; RT: 10.38 min) to afford the desired isomer 1-[5-[2-(3-fluorophenyl)ethynyl]-4-methyl-indan-1-yl]-3-methyl-azetidin-3-ol (71.7 mg, 20.3%) as a white solid.

¹H NMR (400 MHZ, DMSO-d₆) δ 8.17 (s, 1H, HFA), 7.51-7.45 (m, 1H), 7.43-7.39 (m, 2H), 7.34 (d, J=8.0 Hz, 1H), 7.30-7.25 (m, 1H), 7.13 (d, J=8.0 Hz, 1H), 5.18 (br, 1H), 3.87-3.85 (m, 1H), 3.22-3.20 (m, 1H), 3.14-3.10 (m, 2H), 2.97 (d, J=6.4 Hz, 1H), 2.92-2.84 (m, 1H), 2.77-2.70 (m, 1H), 2.38 (s, 3H), 2.10-2.01 (m, 1H), 1.89-1.81 (m, 1H), 1.32 (s, 3H).

LCMS (ESI, m/z): 336 [M+H]⁺. Analytic Conditions: column: HALO C18, 3.0*30 mm, 2.7 μm; mobile phase A: water (0.05% TFA), mobile phase B: acetonitrile (0.05% TFA); flow rate: 1.20 mL/min; gradient: 5% B to 100% B in 1.20 min, hold at 100% for 0.60 min, 100% B to 5% B in 0.03 min; 254 nm; RT: 0.921 min.

### Chiral Separation of 1-[5-[2-(3-fluorophenyl)ethynyl]-4-methyl-indan-1-yl]-3-methyl-azetidin-3-ol (17b)

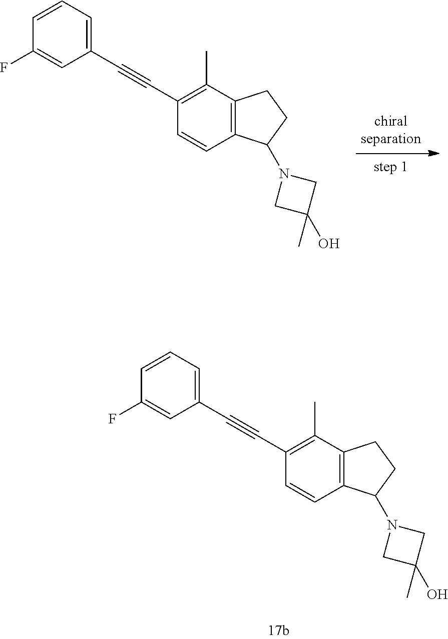 [SMILES: Cc1c(C#Cc2cccc(F)c2)ccc2c1CCC2N1CC(C)(O)C1 ; Cc1c(C#Cc2cccc(F)c2)ccc2c1CCC2N1CC(C)(O)C1]

The racemate (350 mg) was separated by chairl-HPLC(Column: Lux 5 μm Cellulose-2, 3*15 cm, 5 μm; Mobile Phase A: HEX (0.5% 2M NH₃-MeOH)-HPLC, Mobile Phase B: EtOH-HPLC; Flow rate: 20 mL/min; Gradient: 5% B to 5% B in 71 min; Wave Length: 220/254 nm; RT1:13.238 min; RT2:27.446 min) to afford the desired isomer 1-[5-[2-(3-fluorophenyl)ethynyl]-4-methyl-indan-1-yl]-3-methyl-azetidin-3-ol (the second eluting peak, 118.6 mg, 33.6%) as a white solid.

¹H NMR (400 MHz, DMSO-d₆) δ 7.51-7.45 (m, 1H), 7.43-7.39 (m, 2H), 7.34 (d, J=7.6 Hz, 1H), 7.30-7.25 (m, 1H), 7.13 (d, J=7.6 Hz, 1H), 5.14 (s, 1H), 3.84-3.82 (m, 1H), 3.18-3.17 (m, 1H), 3.11-3.07 (m, 2H), 2.95 (d, J=6.8 Hz, 1H), 2.92-2.83 (m, 1H), 2.77-2.70 (m, 1H), 2.38 (s, 3H), 2.09-2.00 (m, 1H), 1.88-1.81 (m, 1H), 1.32 (s, 3H).

LCMS (ESI, m/z): 336 [M+H]⁺. Analytic Conditions: column: HALO C18, 3.0*30 mm, 2.7 μm; mobile phase A: water (0.05% TFA), mobile phase B: acetonitrile (0.05% TFA); flow rate: 1.20 mL/min; gradient: 5% B to 100% B in 1.20 min, hold at 100% for 0.60 min, 100% B to 5% B in 0.03 min; 254 nm; RT: 0.922 min.

### Example S18. 1-(5-(2,6-dichlorophenethyl)-4-methyl-2,3-dihydro-1H-inden-1-yl)-3-methylazetidin-3-ol (18a & 18b)

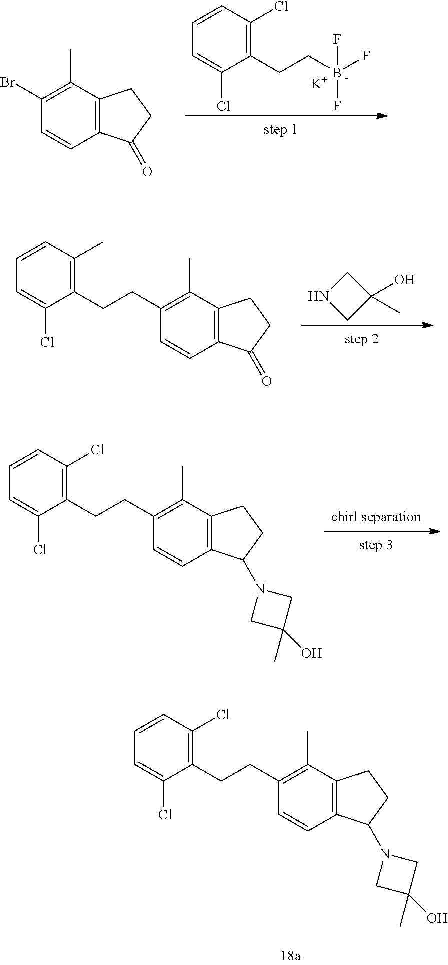

### Synthesis of 5-[2-(2,6-dichlorophenyl)ethyl]-4-methyl-indan-1-one

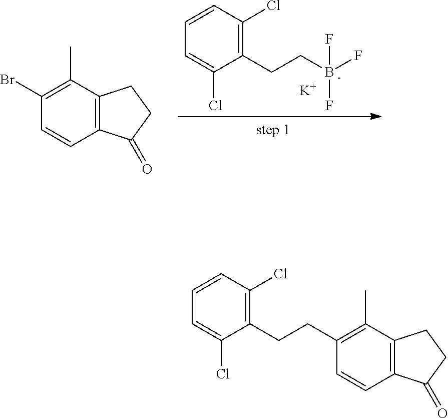

A solution of 5-bromo-4-methyl-indan-1-one (300 mg, 1.33 mmol, 1.00 equiv.), potassium 2-(2,6-dichlorophenyl)ethyl-trifluoro-boranuide (1.1 g, 4.00 mmol, 3.00 equiv.), Cs₂CO₃ (1.3 g, 4.00 mmol, 3.00 equiv.) and Pd(dppf)Cl2 (98 mg, 0.13 mmol, 0.10 equiv.), in toluene (5 mL) and water (0.5 mL) was stirred at 90° C. for 3 h under nitrogen atmosphere. LCMS showed the reaction was completed. The reaction was concentrated under reduced pressure. The residue was purified by flash column chromatography on silica gel (eluted with PE/EtOAc, 1:1) to get 5-[2-(2,6-dichlorophenyl)ethyl]-4-methyl-indan-1-one (400 mg, 94%) as a yellow solid. LCMS (ESI, m/z): 319 [M+H]⁺.

### Synthesis of 1-[5-[2-(2,6-dichlorophenyl)ethyl]-4-methyl-indan-1-yl]-3-methyl-azetidin-3-ol

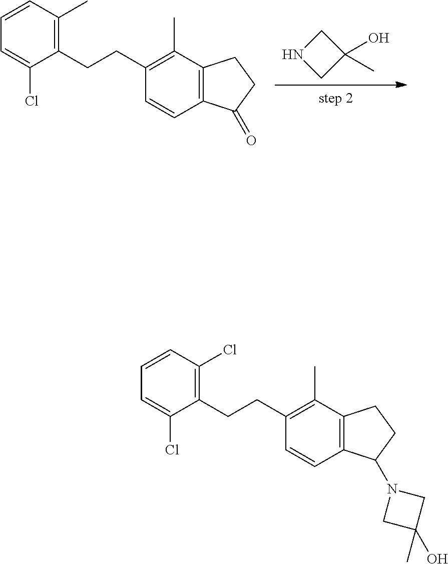

A solution of 5-[2-(2,6-dichlorophenyl)ethyl]-4-methyl-indan-1-one (400 mg, 1.25 mmol, 1.00 equiv.), 3-methylazetidin-3-ol (218 mg, 2.51 mmol, 2.00 equiv.), ZnCl₂ (1.25 mL, 2M in 4Me-THF, 2.51 mmol, 2.00 equiv.) and NaBH₃CN (321 mg, 5.01 mmol, 4.00 equiv.), in methanol (5 mL) was stirred at 80° C. for 3 h. LCMS showed the reaction was completed. The reaction mixture was quenched with water (50 mL) and extracted with DCM (3*20 mL). The combined organic layers were concentrated under reduced pressure. The residue was purified by prep-HPLC (Column: XBridge Prep C18 OBD Column, 30*100 mm, 5 μm; Mobile Phase A: water (10 mM NH₄HCO₃+0.1% NH₃ H₂O), Mobile Phase B: ACN; Flow rate: 60 mL/min; Gradient: 65% B to 85% B in 7 min; 254/220 nm; RT: 4.68 min) to get 1-[5-[2-(2,6-dichlorophenyl)ethyl]-4-methyl-indan-1-yl]-3-methyl-azetidin-3-ol (390 mg, 80%) as white solid. LCMS (ESI, m z): 390 [M+H]⁺

### Chiral Separation of 1-[5-[2-(2,6-dichlorophenyl)ethyl]-4-methyl-indan-1-yl]-3-methyl-azetidin-3-ol (18a)

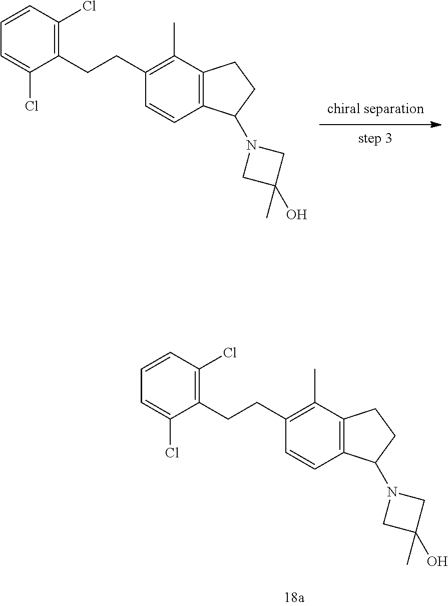 [SMILES: Cc1c(CCc2c(Cl)cccc2Cl)ccc2c1CCC2N1CC(C)(O)C1 ; Cc1c(CCc2c(Cl)cccc2Cl)ccc2c1CCC2N1CC(C)(O)C1]

The racemate (390 mg) was separated by chiral-HPLC(Column: Lux 5 μm Cellulose-4, 2.12*25 cm, 5 μm; Mobile Phase A: Hex (0.5% 2M NH₃-MeOH)-HPLC, Mobile Phase B: EtOH-HPLC; Flow rate: 20 mL/min; Gradient: 98% B to 98% B in 16 min; Wave Length: 220/254 nm; RT1 (min): 11.46; RT2 (min): 14.04) to get the desired isomer (first eluting peak, 66 mg, 16.3%) as a white solid.

¹H NMR (400 MHZ, DMSO-d₆) δ 7.49 (d, J=8.0 Hz, 2H), 7.30 (t, J=8.0 Hz, 1H), 7.03 (d, J=7.6 Hz, 1H), 6.99 (d, J=7.6 Hz, 1H), 5.13 (s, 1H), 3.78-3.75 (m, 1H), 3.18-3.16 (m, 1H), 3.06-3.00 (m, 4H), 2.92-2.80 (m, 2H), 2.77-2.66 (m, 3H), 2.25 (s, 3H), 2.04-1.95 (m, 1H), 1.85-1.78 (m, 1H), 1.30 (s, 3H).

LCMS (ESI, m/z): 390 [M+H]⁺. Analytic Conditions: column: HALO C18, 3.0*30 mm, 2.7 μm; mobile phase A: water (0.05% TFA), mobile phase B: acetonitrile (0.05% TFA); flow rate: 1.20 mL/min; gradient: 5% B to 100% B in 1.20 min, hold at 100% for 0.60 min, 100% B to 5% B in 0.03 min; 210 nm; RT: 1.003 min.

### Chiral Separation of 1-[5-[2-(2,6-dichlorophenyl)ethyl]-4-methyl-indan-1-yl]-3-methyl-azetidin-3-ol (18b)

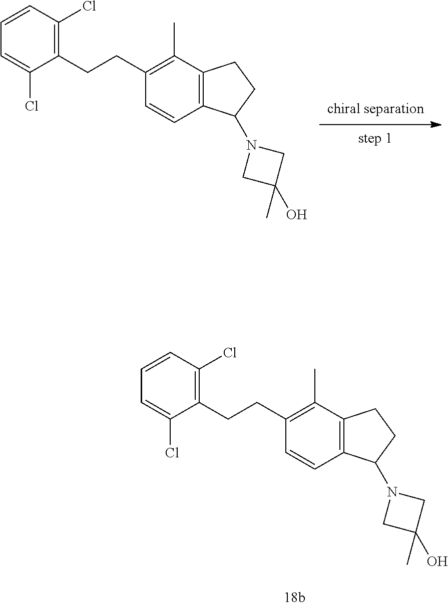 [SMILES: Cc1c(CCc2c(Cl)cccc2Cl)ccc2c1CCC2N1CC(C)(O)C1 ; Cc1c(CCc2c(Cl)cccc2Cl)ccc2c1CCC2N1CC(C)(O)C1]

The racemate (390 mg) was separated by chiral-HPLC(Column: Lux 5 μm Cellulose-4, 2.12*25 cm, 5 μm; Mobile Phase A: Hex (0.5% 2M NH₃-MeOH)-HPLC, Mobile Phase B: EtOH-HPLC; Flow rate: 20 mL/min; Gradient: 98% B to 98% B in 16 min; Wave Length: 220/254 nm; RT1 (min): 11.46; RT2 (min): 14.04) to get the desired isomer (second eluting peak, 65.9 mg, 16.7%) as a white solid.

¹H NMR (400 MHZ, DMSO-d₆) δ 7.48 (d, J=8.0 Hz, 2H), 7.29 (t, J=8.0 Hz, 1H), 7.02 (d, J=8.0 Hz, 1H), 6.98 (d, J=8.0 Hz, 1H), 5.19 (s, 1H), 3.77-3.74 (m, 1H), 3.18-3.16 (m, 1H), 3.06-3.00 (m, 4H), 2.92-2.80 (m, 2H), 2.76-2.66 (m, 3H), 2.24 (s, 3H), 2.04-1.95 (m, 1H), 1.84-1.78 (m, 1H), 1.30 (s, 3H).

LCMS (ESI, m/z): 390 [M+H]⁺. Analytic Conditions: column: HALO C18, 3.0*30 mm, 2.7 μm; mobile phase A: water (0.05% TFA), mobile phase B: acetonitrile (0.05% TFA); flow rate: 1.20 mL/min; gradient: 5% B to 100% B in 1.20 min, hold at 100% for 0.60 min, 100% B to 5% B in 0.03 min; 210 nm; RT: 0.990 min.

### Example S19. 1-(5-((2,6-dichlorobenzyl)oxy)-4-methyl-2,3-dihydro-1H-inden-1-yl)-3-methylazetidin-3-ol (19a & 19b)

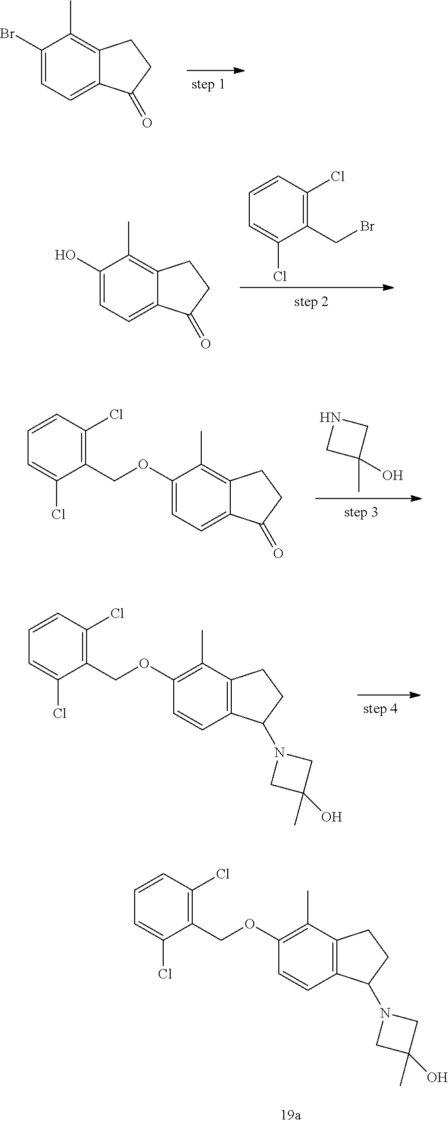

### Synthesis of 5-hydroxy-4-methyl-indan-1-one

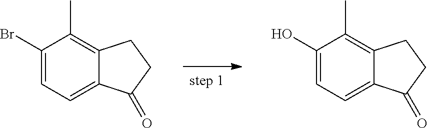

A solution of 5-bromo-4-methyl-indan-1-one (500 mg, 2.22 mmol, 1.00 equiv.), KOH(373 mg, 6.66 mmol, 3.00 equiv.). Pd2 (dba)₃ (203 mg, 0.22 mmol, 0.10 equiv.) and t-BuBrettPhos (215 mg, 0.44 mmol, 0.20 equiv.) in 1,4-dioxane (4 mL) and water (0.4 mL) was stirred for 12 h at 80° C. under nitrogen atmosphere. LCMS showed the reaction was completed. The reaction was acidified to pH 4˜5 with 1M HCl. The reaction mixture was filtered and the filtration was concentrated under reduced pressure. The residue was purified by flash chromatography on C18 silica (eluted with water/acetonitrile, 2:3) to afford 5-hydroxy-4-methyl-indan-1-one (284 mg, 78%) as a yellow solid. LCMS (ESI, m z): 163 [M+H]⁺.

### Synthesis of 5-[(2,6-dichlorophenyl) methoxy]-4-methyl-indan-1-one

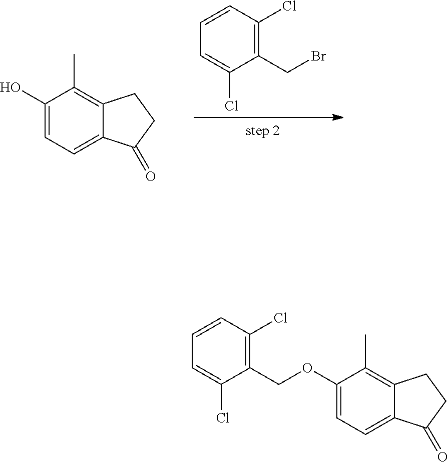 [SMILES: Cc1c(O)ccc2c1CCC2=O ; Clc1cccc(Cl)c1CBr ; Cc1c(OCc2c(Cl)cccc2Cl)ccc2c1CCC2=O]

To a solution of 5-hydroxy-4-methyl-indan-1-one (284 mg, 1.75 mmol, 1.00 equiv.) in MeCN (5 mL) was added 2-(bromomethyl)-1,3-dichloro-benzene (630 mg, 2.63 mmol, 1.50 equiv.) and K2CO₃ (724 mg, 5.25 mmol, 3.00 equiv.). The reaction was stirred for 12 h at 60° C. LCMS showed the reaction was completed. The reaction mixture was filtered and the filtrate was concentrated under reduced pressure. The residue was purified by flash chromatography on C18 silica (eluted with water/acetonitrile, 1:3) to afford 5-[(2,6-dichlorophenyl) methoxy]-4-methyl-indan-1-one (300 mg, 53%) as a yellow solid. LCMS (ESI, m z): 321 [M+H]⁺.

### Synthesis of 1-[5-[(2,6-dichlorophenyl) methoxy]-4-methyl-indan-1-yl]-3-methyl-azetidin-3-ol

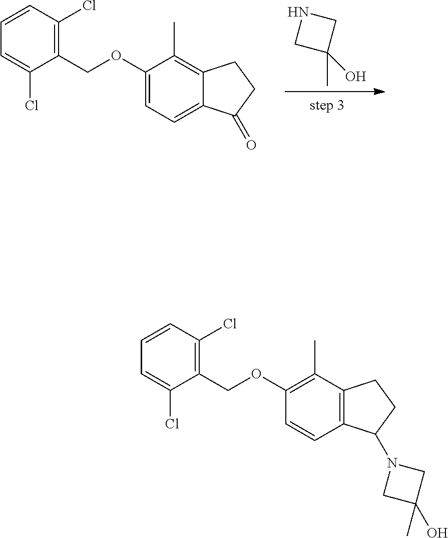

A solution of 5-[(2,6-dichlorophenyl) methoxy]-4-methyl-indan-1-one (300 mg, 0.93 mmol, 1.00 equiv.), 3-methylazetidin-3-ol (162 mg, 1.87 mmol, 2.00 equiv.), ZnCl₂ (1.2 mL, 2M in 4Me-THF, 2.33 mmol, 2.50 equiv.) and NaBH₃CN (239 mg, 3.74 mmol, 4.00 equiv.) in methanol (5 mL) was stirred for 12 h 60° C. LCMS showed the reaction was completed. The reaction mixture was quenched with water (50 mL) and extracted with DCM (3*20 mL). The combined organic layers were concentrated under reduced pressure. The residue was purified by flash chromatography on C18 silica (eluted with water/acetonitrile, 1:1) to afford 1-[5-[(2,6-dichlorophenyl) methoxy]-4-methyl-indan-1-yl]-3-methyl-azetidin-3-ol (300 mg, 81%) as a yellow solid. LCMS (ESI, m/z): 392 [M+H]⁺.

### Synthesis of 1-[5-[(2,6-dichlorophenyl) methoxy]-4-methyl-indan-1-yl]-3-methyl-azetidin-3-ol (19a)

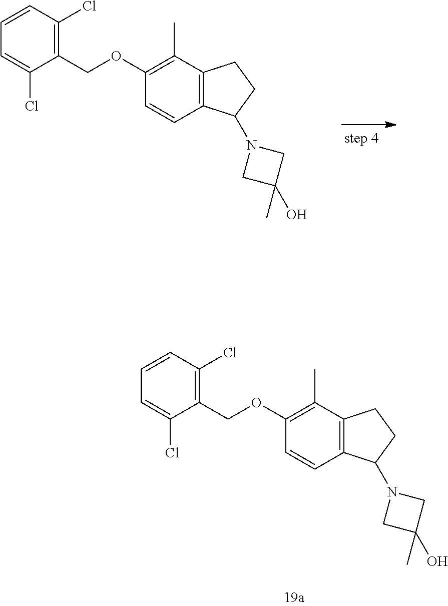 [SMILES: Cc1c(OCc2c(Cl)cccc2Cl)ccc2c1CCC2N1CC(C)(O)C1 ; Cc1c(OCc2c(Cl)cccc2Cl)ccc2c1CCC2N1CC(C)(O)C1]

The racemate (150 mg) was purified by Prep-Chiral HPLC(Column: Lux 5 μm Cellulose-2, 2.12*25 cm, 5 μm; Mobile Phase A: HEX (0.5% 2M NH₃-MeOH)-HPLC, Mobile Phase B: IPA-HPLC; Flow rate: 20 mL/min; Gradient: 90% B to 90% B in 15 min; 220/254 nm; RT1:11. 889 min) to afford 1-[5-[(2,6-dichlorophenyl) methoxy]-4-methyl-indan-1-yl]-3-methyl-azetidin-3-ol (37.7 mg, 24%) as an off-white solid.

¹H NMR (300 MHz, DMSO-d₆) δ 7.60-7.57 (m, 2H), 7.48 (dd, J=9.0, 6.9 Hz, 1H), 7.08 (d, J=8.1 Hz, 1H), 6.98 (d, J=8.1 Hz, 1H), 5.20 (s, 2H), 5.15 (s, 1H), 3.79-3.75 (m, 1H), 3.23-3.20 (m, 1H), 3.10-3.08 (m, 2H), 2.96-2.94 (m, 1H), 2.87-2.76 (m, 1H), 2.71-2.62 (m, 1H), 2.08-2.02 (m, 1H), 2.00 (s, 3H), 1.87-1.80 (m, 1H), 1.32 (s, 3H).

LCMS (ESI, m/z): 392 [M+H]⁺. Analytic Conditions: column: L-column3 C18 Column 3.0*30 mm, 2.0 μm; mobile Phase A: Water/5 mM NH₄HCO₃, mobile Phase B: acetonitrile; flow rate: 1.50 mL/min; gradient: 30% B to 80% B in 1.80 min, 80% B to 95% B in 0.5 min, hold at 95% for 0.5 min, 95% B to 10% B in 0.1 min; 210 nm; RT: 1.461 min.

### Synthesis of 1-[5-[(2,6-dichlorophenyl) methoxy]-4-methyl-indan-1-yl]-3-methyl-azetidin-3-ol (19b)

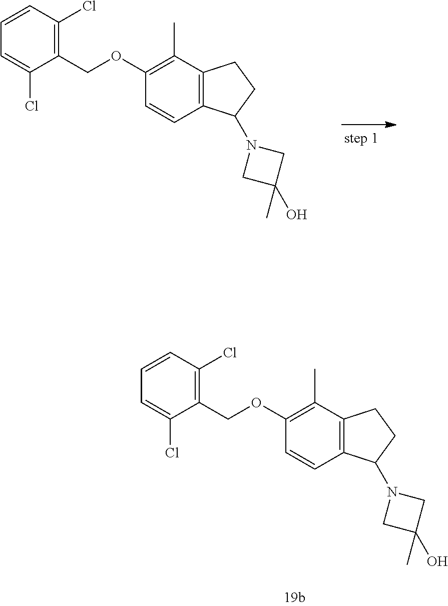 [SMILES: Cc1c(OCc2c(Cl)cccc2Cl)ccc2c1CCC2N1CC(C)(O)C1 ; Cc1c(OCc2c(Cl)cccc2Cl)ccc2c1CCC2N1CC(C)(O)C1]

The racemate (150 mg) was purified by Prep-Chiral HPLC(Column: Lux 5 μm Cellulose-2, 2.12*25 cm, 5 μm; Mobile Phase A: HEX (0.5% 2M NH₃-MeOH)-HPLC, Mobile Phase B: IPA-HPLC; Flow rate: 20 mL/min; Gradient: 90% B to 90% B in 15 min; 220/254 nm; RT1:13. 499 min) to afford 1-[5-[(2,6-dichlorophenyl) methoxy]-4-methyl-indan-1-yl]-3-methyl-azetidin-3-ol (36.1 mg, 23%) as an off-white solid.

¹H NMR (300 MHZ, DMSO-d₆) δ 7.60-7.55 (m, 2H), 7.46 (dd, J=9.0, 6.9 Hz, 1H), 7.08 (d, J=8.1 Hz, 1H), 6.97 (d, J=8.1 Hz, 1H), 5.19 (s, 2H), 3.80-3.77 (m, 1H), 3.24-3.21 (m, 1H), 3.11-3.08 (m, 2H), 2.97-2.95 (m, 1H), 2.87-2.76 (m, 1H), 2.70-2.61 (m, 1H), 2.09-2.02 (m, 1H), 2.00 (s, 3H), 1.88-1.78 (m, 1H), 1.33 (s, 3H).

LCMS (ESI, m/z): 392 [M+H]⁺. Analytic Conditions: column: L-column3 C18 Column 3.0*30 mm, 2.0 μm; mobile Phase A: Water/5 mM NH₄HCO₃, mobile Phase B: acetonitrile; flow rate: 1.50 mL/min; gradient: 30% B to 80% B in 1.80 min, 80% B to 95% B in 0.5 min, hold at 95% for 0.5 min, 95% B to 10% B in 0.1 min; 210 nm; RT: 1.462 min.

### Example S20. 1-((5-((2,6-dichlorobenzyl)oxy)-4-methyl-2,3-dihydro-1H-inden-1-yl)amino)-2-methylpropan-2-ol (20a & 20b)

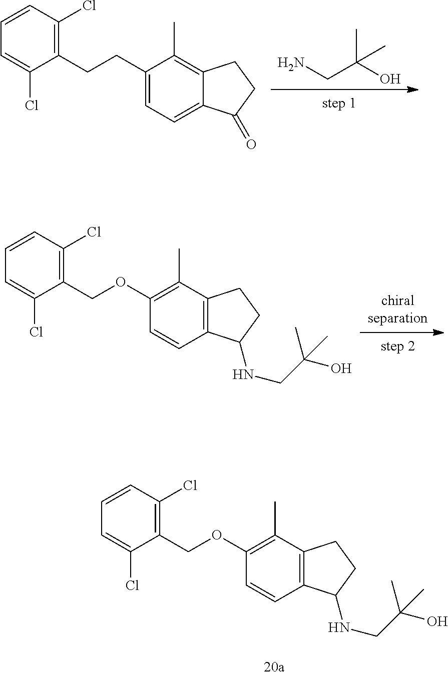

### Synthesis of 1-((5-((2,6-dichlorobenzyl)oxy)-4-methyl-2,3-dihydro-1H-inden-1-yl)amino)-2-methylpropan-2-ol

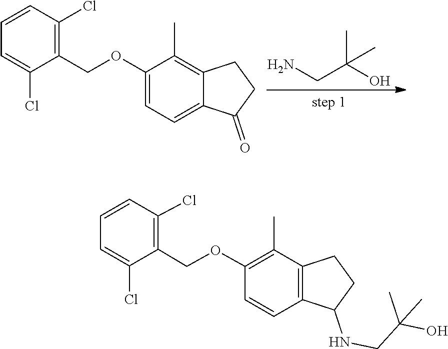

A solution of 5-[(2,6-dichlorophenyl) methoxy]-4-methyl-indan-1-one (120 mg, 0.37 mmol, 1.00 equiv.), 1-amino-2-methyl-propan-2-ol (66 mg, 0.75 mmol, 2.00 equiv.), NaHB₃CN (94 mg, 1.49 mmol, 4.00 equiv.) and ZnCl₂ (2M in 4Me-THF, 0.37 mL, 0.75 mmol, 2.00 equiv.) in methanol (5 mL) was stirred at 80° C. for 15 h. LCMS showed the reaction was completed. The reaction mixture was quenched with water (40 mL) and extracted with DCM (3*20 mL). The combined organic layers were concentrated under reduced pressure. The residue was purified by flash chromatography on C18 silica (eluted with PE/EtOAc, 1:3) to afford 1-((5-((2,6-dichlorobenzyl)oxy)-4-methyl-2,3-dihydro-1H-inden-1-yl)amino)-2-methylpropan-2-ol (120 mg, 81%) as a yellow oil. LCMS (ESI, m z): 394 [M+H]⁺.

### Chiral Separation of 1-((5-((2,6-dichlorobenzyl)oxy)-4-methyl-2,3-dihydro-1H-inden-1-yl)amino)-2-methylpropan-2-ol (20a)

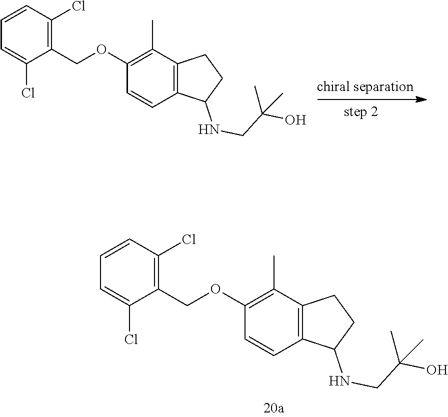 [SMILES: Cc1c(OCc2c(Cl)cccc2Cl)ccc2c1CCC2NCC(C)(C)O ; Cc1c(OCc2c(Cl)cccc2Cl)ccc2c1CCC2NCC(C)(C)O]

The racemate (120 mg) was separated by chiral-HPLC(Column: XBridge Prep C18 OBD Column, 30*100 mm, 5 μm; Mobile Phase A: water (10 mM NH₄HCO₃), Mobile Phase B: ACN; Flow rate: 60 mL/min; Gradient: 48% B to 73% B in 9 min, 73% B; Wave Length: 254/220 nm; RT: 8.85 min) to afford the desired isomer 1-((5-((2,6-dichlorobenzyl)oxy)-4-methyl-2,3-dihydro-1H-inden-1-yl)amino)-2-methylpropan-2-ol (the first eluting peak, 31.1 mg, 25%, 100% e.e.) as a yellow oil.

¹H NMR (400 MHZ, Methanol-d₄) δ7.48 (d, J=8.8 Hz, 1H), 7.47 (d, J=7.6 Hz, 1H), 7.37 (dd, J=8.8, 7.6 Hz, 1H), 7.22 (d, J=8.0 Hz, 1H), 7.01 (d, J=8.0 Hz, 1H), 5.30 (s, 2H), 4.29 (t, J=6.4 Hz, 1H), 3.02-2.95 (m, 1H), 2.80-2.72 (m, 1H), 2.64 (d, J=2.8 Hz, 2H), 2.44-2.36 (m, 1H), 2.09 (s, 3H), 1.98-1.91 (m, 1H), 1.25 (s, 3H), 1.24 (s, 3H). LCMS (ESI, m/z): 394 [M+H]⁺. Analytic Conditions: column: HALO C18 Column 3.0*30 mm, 2.7 μm; mobile Phase A: Water/0.05% TFA, mobile Phase B: Acetonitrile/0.05% TFA; flow rate: 1.50 mL/min; gradient: 5% B to 100% B in 1.19 min, hold at 100% for 0.6 min, 100% B to 5% B in 0.03 min; 210 nm; RT: 0.962 min.

### Chiral Separation of 1-((5-((2,6-dichlorobenzyl)oxy)-4-methyl-2,3-dihydro-1H-inden-1-yl)amino)-2-methylpropan-2-ol (20b)

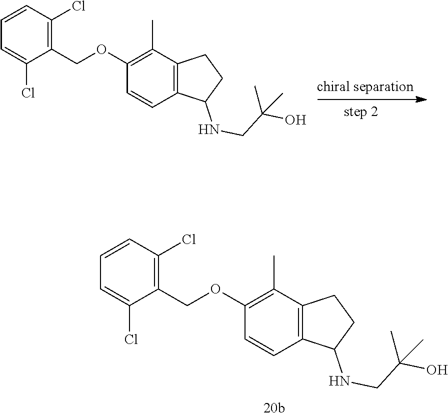 [SMILES: Cc1c(OCc2c(Cl)cccc2Cl)ccc2c1CCC2NCC(C)(C)O ; Cc1c(OCc2c(Cl)cccc2Cl)ccc2c1CCC2NCC(C)(C)O]

The racemate (120 mg) was separated by chiral-HPLC(Column: XBridge Prep C18 OBD Column, 30*100 mm, 5 μm; Mobile Phase A: water (10 mM NH+HCO₃), Mobile Phase B: ACN; Flow rate: 60 mL/min; Gradient: 48% B to 73% B in 9 min, 73% B; Wave Length: 254/220 nm; RT: 8.85 min) to afford the desired isomer 1-((5-((2,6-dichlorobenzyl)oxy)-4-methyl-2,3-dihydro-1H-inden-1-yl)amino)-2-methylpropan-2-ol (the second eluting peak, 25.4 mg, 20%, 99% e.e.) as a yellow oil.

¹H NMR (400 MHZ, Methanol-d₄) δ7.48 (d, J=8.8 Hz, 1H), 7.47 (d, J=7.6 Hz, 1H), 7.37 (dd, J=8.8, 7.6 Hz, 1H), 7.22 (d, J=8.0 Hz, 1H), 7.01 (d, J=8.0 Hz, 1H), 5.29 (s, 2H), 4.28 (t, J=6.4 Hz, 1H), 3.02-2.94 (m, 1H), 2.79-2.71 (m, 1H), 2.64 (d, J=2.0 Hz, 2H), 2.44-2.35 (m, 1H), 2.09 (s, 3H), 1.98-1.89 (m, 1H), 1.25 (s, 3H), 1.24 (s, 3H).

LCMS (ESI, m/z): 394 [M+H]⁺. Analytic Conditions: column: HALO C18 Column 3.0*30 mm, 2.7 μm; mobile Phase A: Water/0.05% TFA, mobile Phase B: Acetonitrile/0.05% TFA; flow rate: 1.50 mL/min; gradient: 5% B to 100% B in 1.19 min, hold at 100% for 0.6 min, 100% B to 5% B in 0.03 min; 210 nm; RT: 0.964 min.

### Example S21. 1-(5-(3-chloro-4-cyclopropylphenyl)-6-methyl-2,3-dihydro-1H-inden-1-yl)-3-methylazetidin-3-ol (21a & 21b)

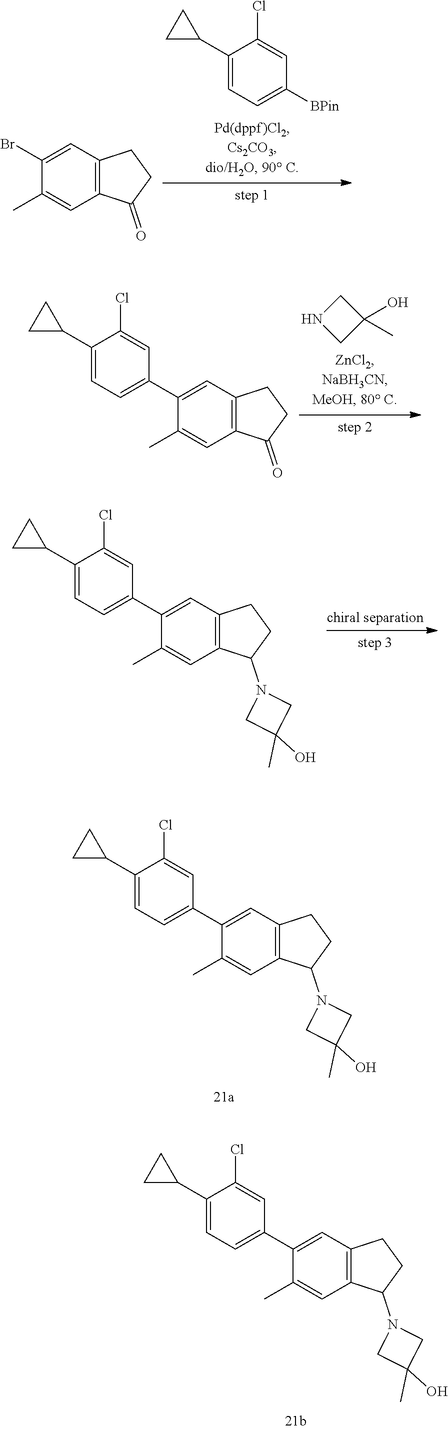

### Synthesis of 5-(3-chloro-4-cyclopropyl-phenyl)-6-methyl-indan-1-one

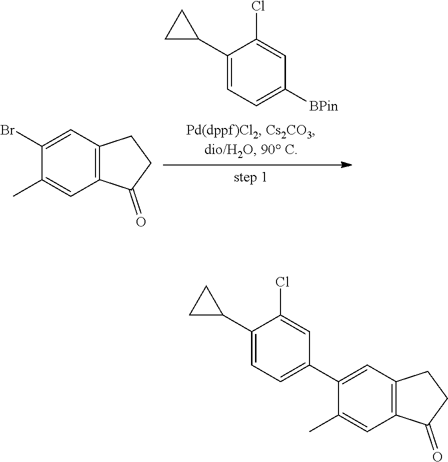

To a stirred solution of 5-bromo-6-methyl-indan-1-one (300 mg, 1.33 mmol, 1.00 equiv.) and 2-(3-chloro-4-cyclopropyl-phenyl)-4,4,5,5-tetramethyl-1,3,2-dioxaborolane (557 mg, 2.00 mmol, 1.50 equiv.) in 1,4-dioxane (2.5 mL) and water (0.25 mL) were added Pd(dppf)Cl2 (109 mg, 0.13 mmol, 0.10 equiv.) and Cs₂CO₃ (1303 mg, 4.00 mmol, 3.00 equiv.). The resulting mixture was stirred at 90° C. overnight. LCMS showed the reaction was completed. The reaction was concentrated under vacuum. The residue was purified by flash column chromatography on silica gel (eluted with PE: EtOAc=5:1) to afford 5-(3-chloro-4-cyclopropyl-phenyl)-6-methyl-indan-1-one (305 mg, 77.1%) as an orange oil. LCMS (ESI, m z): 297 [M+H]⁺.

### Synthesis of 1-[5-(3-chloro-4-cyclopropyl-phenyl)-6-methyl-indan-1-yl]-3-methyl-azetidin-3-ol

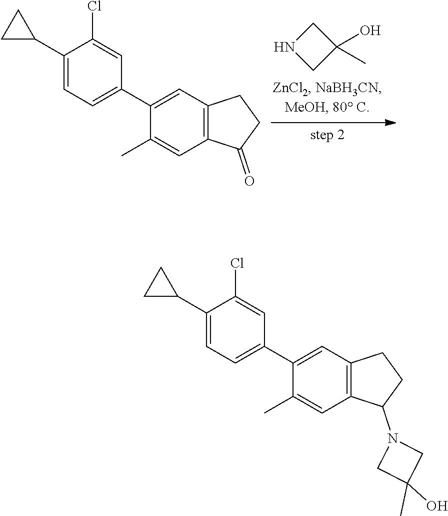

To a stirred solution of 5-(3-chloro-4-cyclopropyl-phenyl)-6-methyl-indan-1-one (300 mg, 1.01 mmol, 1.00 equiv.) and 3-methylazetidin-3-ol (176 mg, 2.02 mmol, 2.00 equiv.) in methanol (3 mL) were added ZnCl₂ (2.0 M in THF, 1 mL, 2.02 mmol, 2.00 equiv.) and NaBH₃CN (259 mg, 4.04 mmol, 4.00 equiv.). The resulting mixture was stirred 3 h at 80° C. LCMS showed the reaction was completed. The reaction was quenched by water (30 mL) and extracted with EtOAc (2×15 mL). The combined organic layers were concentrated under reduced pressure. The residue was purified by prep-HPLC(Column: YMC-Actus Triart C18 ExRS, 30*250, 5 μm; Mobile Phase A: water (10 mmol/L NH₄HCO₃+0.1% NH₃ H₂O), Mobile Phase B: ACN; Flow rate: 60 mL/min; Gradient: 65% B to 95% B in 7 min; 254/220 nm; RT: 6.32 min) to afford 1-[5-(3-chloro-4-cyclopropyl-phenyl)-6-methyl-indan-1-yl]-3-methyl-azetidin-3-ol (175 mg, 47.1%) as a white solid. LCMS (ESI, m z): 368 [M+H]⁺.

### Chiral Separation of 1-[5-(3-chloro-4-cyclopropyl-phenyl)-6-methyl-indan-1-yl]-3-methyl-azetidin-3-ol (21a)

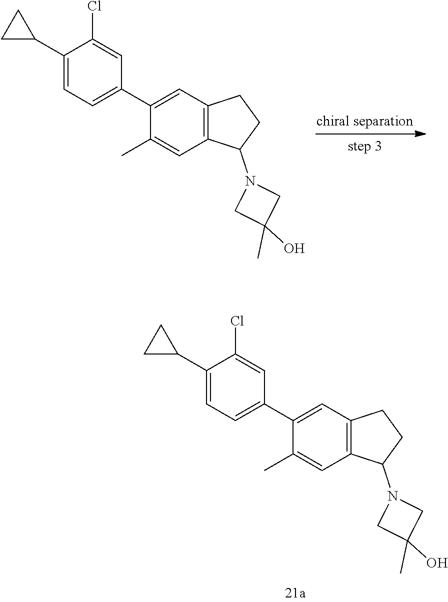 [SMILES: Cc1cc2c(cc1-c1ccc(C3CC3)c(Cl)c1)CCC2N1CC(C)(O)C1 ; Cc1cc2c(cc1-c1ccc(C3CC3)c(Cl)c1)CCC2N1CC(C)(O)C1]

The mixture of the isomers (175 mg) was separated by chiral-HPLC(Column: Lux 5 μm Cellulose-2, 2.12*25 cm, 5 μm; Mobile Phase A: Hex (0.5% 2 M NH₃-MeOH)-HPLC, Mobile Phase B: IPA-HPLC; Flow rate: 20 mL/min; Gradient: 5% B to 5% B in 22 min; Wave Length: 220/254 nm; RT1:9.35 min; RT2:17.19 min) to get the desired isomer (66.3 mg, 37.5%, 98.4% ee) as a white solid.

¹H NMR (400 MHZ, DMSO-d₆) δ 7.32 (d, J=1.6 Hz, 1H), 7.18 (dd, J=8.0, 1.6 Hz, 1H), 7.15 (s, 1H), 7.06 (d, J=8.0 Hz, 1H), 7.03 (s, 1H), 5.14 (s, 1H), 3.80-3.77 (m, 1H), 3.21-3.19 (m, 1H), 3.16-3.09 (m, 2H), 2.96-2.93 (m, 1H), 2.91-2.85 (m, 1H), 2.74-2.70 (m, 1H), 2.18 (s, 3H), 2.17-2.15 (m, 1H), 2.05-1.98 (m, 1H), 1.86-1.80 (m, 1H), 1.33 (s, 3H), 1.06-1.01 (m, 2H), 0.77-0.73 (m, 2H).

LCMS (ESI, m/z): 368 [M+H]⁺. Analytic Conditions: column: EVO C18, 2.1*30 mm, 2.6 μm; mobile phase A: water (5 mM NH₄HCO₃), mobile phase B: acetonitrile; flow rate: 1.20 mL/min; gradient: 10% B to 95% B in 1.20 min, hold at 95% for 0.58 min, 95% B to 10% B in 0.05 min; 254 nm; RT: 1.073 min.

### Chiral Separation of 1-[5-(3-chloro-4-cyclopropyl-phenyl)-6-methyl-indan-1-yl]-3-methyl-azetidin-3-ol (21b)

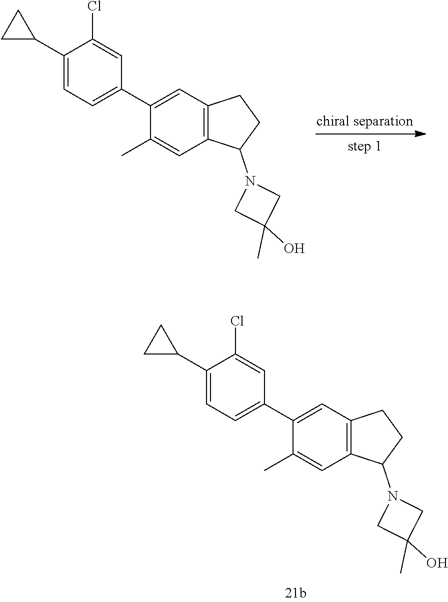 [SMILES: Cc1cc2c(cc1-c1ccc(C3CC3)c(Cl)c1)CCC2N1CC(C)(O)C1 ; Cc1cc2c(cc1-c1ccc(C3CC3)c(Cl)c1)CCC2N1CC(C)(O)C1]

The mixture of the isomers (175 mg) was separated by chiral-HPLC(Column: Lux 5 μm Cellulose-2, 2.12*25 cm, 5 μm; Mobile Phase A: Hex (0.5% 2 M NH₃-MeOH)-HPLC, Mobile Phase B: IPA-HPLC; Flow rate: 20 mL/min; Gradient: 5% B to 5% B in 22 min; Wave Length: 220/254 nm; RT1:9.35 min; RT2:17.19 min) to get the desired isomer (58.3 mg, 32.9%, 99.1% ee) as a white solid.

¹H NMR (400 MHZ, DMSO-d₆) δ 7.32 (d, J=1.6 Hz, 1H), 7.18 (dd, J=8.0, 1.6 Hz, 1H), 7.15 (s, 1H), 7.06 (d, J=8.0 Hz, 1H), 7.02 (s, 1H), 5.14 (s, 1H), 3.80-3.77 (m, 1H), 3.21-3.18 (m, 1H), 3.16-3.09 (m, 2H), 2.96-2.93 (m, 1H), 2.91-2.85 (m, 1H), 2.74-2.71 (m, 1H), 2.18 (s, 3H), 2.17-2.15 (m, 1H), 2.05-1.98 (m, 1H), 1.86-1.79 (m, 1H), 1.33 (s, 3H), 1.06-1.01 (m, 2H), 0.77-0.73 (m, 2H).

LCMS (ESI, m/z): 368 [M+H]⁺. Analytic Conditions: column: EVO C18, 2.1*30 mm, 2.6 μm; mobile phase A: water (5 mM NH₄HCO₃), mobile phase B: acetonitrile; flow rate: 1.20 mL/min; gradient: 10% B to 95% B in 1.20 min, hold at 95% for 0.58 min, 95% B to 10% B in 0.05 min; 254 nm; RT: 1.070 min.

### Example S22. 1-(5-((3-fluorophenyl)ethynyl)-6-methyl-2,3-dihydro-1H-inden-1-yl)-3-methylazetidin-3-ol (22a & 22b)

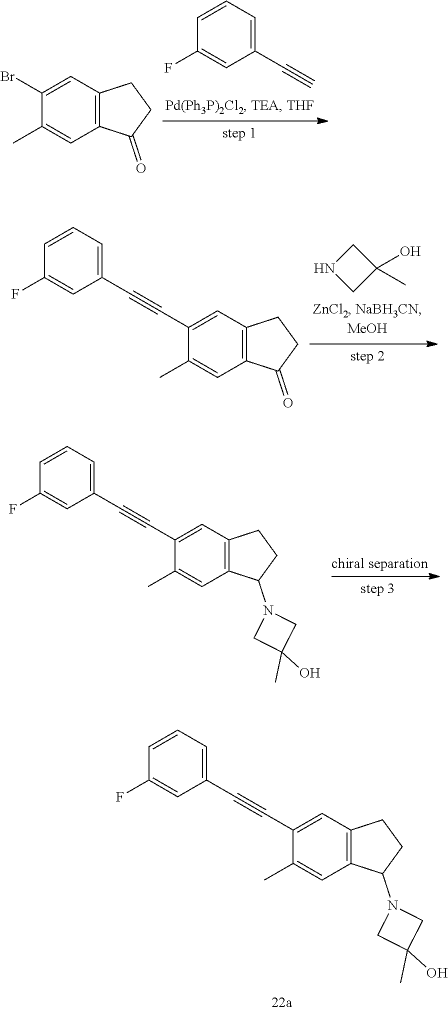

### Synthesis of 5-[2-(3-fluorophenyl)ethynyl]-6-methyl-indan-1-one

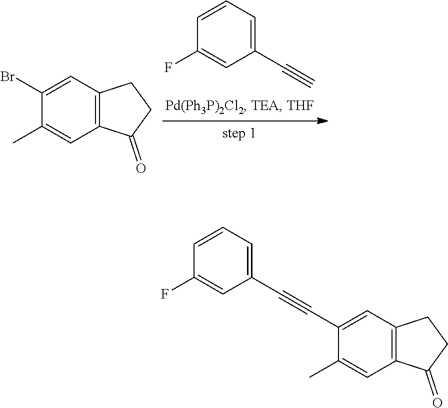

A solution of 5-bromo-6-methyl-indan-1-one (400 mg, 1.78 mmol, 1.00 equiv.), 1-ethynyl-3-fluoro-benzene (427 mg, 3.55 mmol, 2.00 equiv.), Pd(Ph3P)₂Cl₂ (125 mg, 0.18 mmol, 0.10 equiv.) and triethylamine (0.5 mL, 3.55 mmol, 2.00 equiv.) in THF (5 mL) was stirred at 60° C. under nitrogen atmosphere overnight. LCMS showed that the reaction was complete. The reaction mixture was concentrated under reduced pressure. The residue was purified by flash column chromatography on silica gel (eluted with PE/EtOAc, 10/1) to afford 5-[2-(3-fluorophenyl)ethynyl]-6-methyl-indan-1-one (460 mg, 98%) as a light-yellow solid. LCMS (ESI, m/z): 265 [M+H]⁺.

### Synthesis of 1-[5-[2-(3-fluorophenyl)ethynyl]-6-methyl-indan-1-yl]-3-methyl-azetidin-3-ol

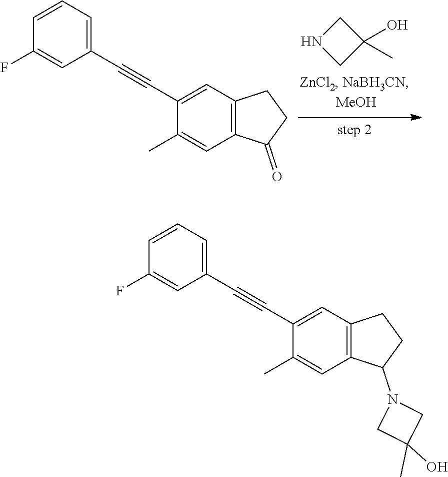

A solution of 5-[2-(3-fluorophenyl)ethynyl]-6-methyl-indan-1-one (460 mg, 1.74 mmol, 1.00 equiv.), 3-methylazetidin-3-ol (303 mg, 3.48 mmol, 2.00 equiv.), ZnCl₂ (2.0 M in THF, 1.7 mL, 3.48 mmol, 2.00 equiv.) and NaBH₃CN (446 mg, 6.96 mmol, 4.00 equiv.) in methanol (5 mL) was stirred at 80° C. overnight. LCMS showed the reaction was completed. The reaction mixture was quenched with water (50 mL) and extracted with DCM (3*30 mL). The combined organic layers were concentrated under reduced pressure. The residue was purified by prep-HPLC (Column: YMC-Actus Triart C18 ExRS, 30*250 mm, 5 μm; Mobile Phase A: water (10 mM NH₄HCO₃+0.1% NH₃. H₂O), Mobile Phase B: ACN; Flow rate: 60 mL/min; Gradient: 45% B to 45% B in 7 min, 45% B to 75% B in 11 min; 254/220 nm; RT: 7.53 min) to give 1-[5-[2-(3-fluorophenyl)ethynyl]-6-methyl-indan-1-yl]-3-methyl-azetidin-3-ol (380 mg, 65.1%) as a yellow oil. LCMS (ESI, m z): 336 [M+H]⁺.

### Chiral Separation of 1-[5-[2-(3-fluorophenyl)ethynyl]-6-methyl-indan-1-yl]-3-methyl-azetidin-3-ol (22a)

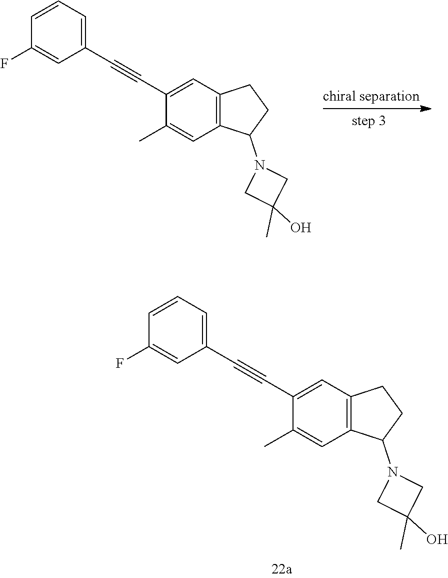 [SMILES: Cc1cc2c(cc1C#Cc1cccc(F)c1)CCC2N1CC(C)(O)C1 ; Cc1cc2c(cc1C#Cc1cccc(F)c1)CCC2N1CC(C)(O)C1]

The racemate (380 mg) was separated by chiral-HPLC(Column: CHIRALPAK IG, 2*25 cm, 5 μm; Mobile Phase A: Hex (0.2% DEA)-HPLC, Mobile Phase B: EtOH-HPLC; Flow rate: 20 mL/min; Gradient: 3% B to 3% B in 21 min; Wave Length: 220/254 nm; RT1:13.944 min; RT2:16.488 min) to afford the desired isomer 1-[5-[2-(3-fluorophenyl)ethynyl]-6-methyl-indan-1-yl]-3-methyl-azetidin-3-ol (the first eluting peak, 108.5 mg, 28%, 100% e.e.) as a white solid.

¹H NMR (400 MHZ, Methanol-d₄) δ7.44-7.38 (m, 2H), 7.35-7.33 (m, 1H), 7.27-7.23 (m, 2H), 7.15-7.10 (m, 1H), 4.00-3.97 (m, 1H), 3.47-3.41 (m, 2H), 3.37 (d, J=8.0 Hz, 1H), 3.22 (d, J=8.0 Hz, 1H), 3.09-3.01 (m, 1H), 2.84-2.77 (m, 1H), 2.50 (s, 3H), 2.26-2.17 (m, 1H), 1.95-1.87 (m, 1H), 1.48 (s, 3H).

LCMS (ESI, m/z): 336 [M+H]⁺. Analytic Conditions: column: HALO C18, 3.0*30 mm, 2.7 μm; mobile phase A: water (0.05% TFA), mobile phase B: acetonitrile (0.05% TFA);

flow rate: 1.20 mL/min; gradient: 5% B to 100% B in 1.20 min, hold at 100% for 0.60 min, 100% B to 5% B in 0.03 min; 254 nm; RT: 0.927 min.

### Chiral Separation of 1-[5-[2-(3-fluorophenyl)ethynyl]-6-methyl-indan-1-yl]-3-methyl-azetidin-3-ol (22b)

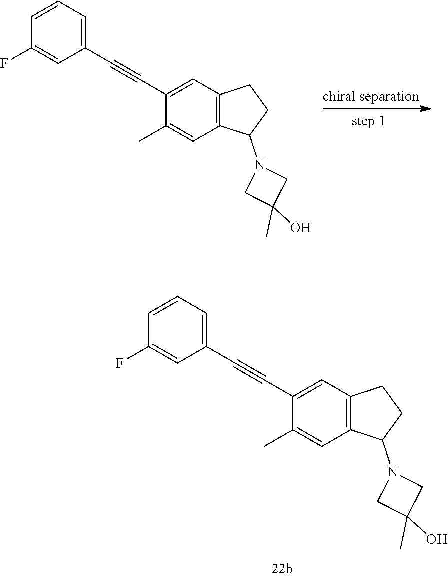 [SMILES: Cc1cc2c(cc1C#Cc1cccc(F)c1)CCC2N1CC(C)(O)C1 ; Cc1cc2c(cc1C#Cc1cccc(F)c1)CCC2N1CC(C)(O)C1]

The racemate (380 mg) was separated by chiral-HPLC(Column: CHIRALPAK IG, 2*25 cm, 5 μm; Mobile Phase A: Hex (0.2% DEA)-HPLC, Mobile Phase B: EtOH-HPLC; Flow rate: 20 mL/min; Gradient: 3% B to 3% B in 21 min; Wave Length: 220/254 nm; RT1:13.944 min; RT2:16.488 min) to afford the desired isomer 1-[5-[2-(3-fluorophenyl)ethynyl]-6-methyl-indan-1-yl]-3-methyl-azetidin-3-ol (the second eluting peak, 94.7 mg, 24.6%, 99.7% e.e.) as a white solid.

¹H NMR (400 MHZ, Methanol-d₄) δ7.44-7.38 (m, 2H), 7.36-7.33 (m, 1H), 7.27-7.24 (m, 2H), 7.16-7.10 (m, 1H), 4.01-3.98 (m, 1H), 3.47-3.42 (m, 2H), 3.38 (d, J=8.0 Hz, 1H), 3.23 (d, J=8.0 Hz, 1H), 3.09-3.01 (m, 1H), 2.85-2.77 (m, 1H), 2.50 (s, 3H), 2.27-2.18 (m, 1H), 1.95-1.88 (m, 1H), 1.48 (s, 3H).

LCMS (ESI, m/z): 336 [M+H]⁺. Analytic Conditions: column: L-column3 C18 Column 3.0*30 mm, 2.0 μm; mobile Phase A: Water/5 mM NH₄HCO₃, mobile Phase B: acetonitrile; flow rate: 1.5000 mL/min; gradient: 40% B to 80% B in 2.00 min, 80% B to 95% B in 0.25 min, hold at 95% for 0.55 min, 95% B to 10% B in 0.05 min; 254 nm; RT: 1.318 min. Example S23. 1-(5-(2,6-dichlorophenethyl)-6-methyl-2,3-dihydro-1H-inden-1-yl)-3-methylazetidin-3-ol (23a & 23b)

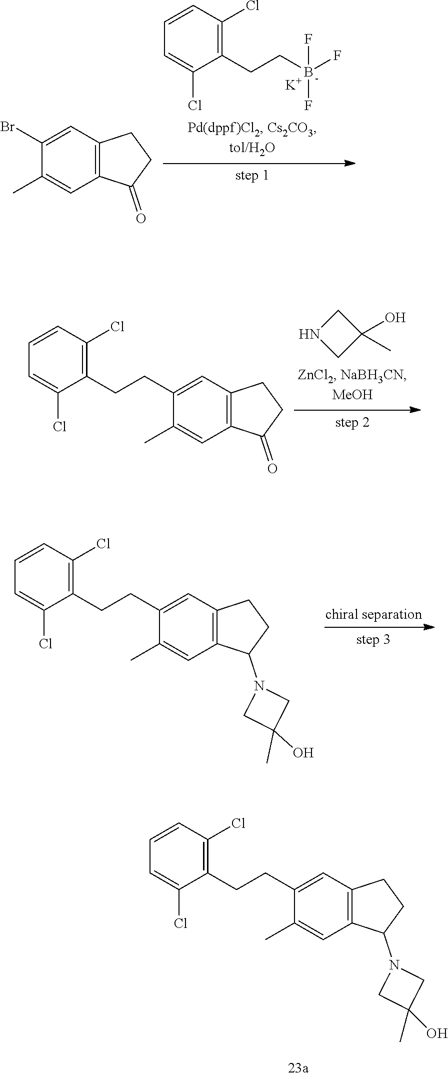

### Synthesis of 5-[2-(2,6-dichlorophenyl)ethyl]-6-methyl-indan-1-one

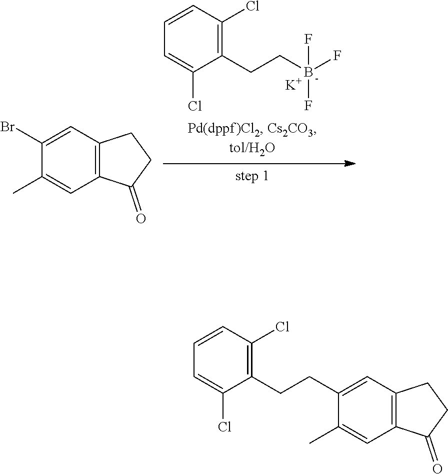

A mixture of 5-bromo-6-methyl-indan-1-one (300 mg, 1.33 mmol, 1.00 equiv.), potassium 2-(2,6-dichlorophenyl)ethyl-trifluoro-boranuide (749 mg, 2.67 mmol, 2.00 equiv.), Cs₂CO₃ (1.3 g, 4 mmol, 3.00 equiv.) and Pd(dppf)Cl2 (98 mg, 0.13 mmol, 0.10 equiv.) in toluene (5 mL) and water (1 mL) was stirred at 90° C. overnight. LCMS showed that the reaction was complete. The reaction was concentrated under reduced pressure. The residue was purified by flash column chromatography on silica gel (eluted with PE/EtOAc, 10:1) to get 5-[2-(2,6-dichlorophenyl)ethyl]-6-methyl-indan-1-one (400 mg, 94.0%) as an off-white solid. LCMS (ESI, m z): 319 [M+H]⁺.

### Synthesis of 1-[5-[2-(2,6-dichlorophenyl)ethyl]-6-methyl-indan-1-yl]-3-methyl-azetidin-3-ol

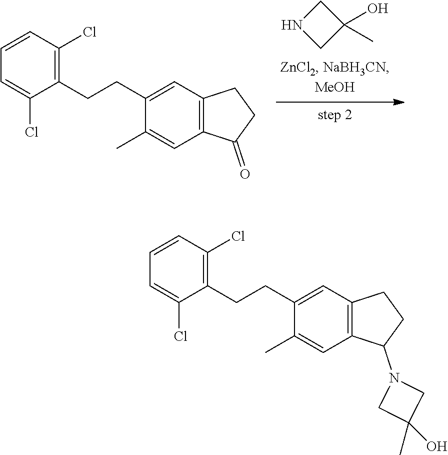

A solution of ZnCl₂ (2M in 4Me-THF, 1.26 mL, 2.51 mmol, 2.00 equiv.), NaBH₃CN (320.8 mg, 5.01 mmol, 4.00 equiv.), 5-[2-(2,6-dichlorophenyl)ethyl]-6-methyl-indan-1-one (400 mg, 1.25 mmol, 1.00 equiv.) and 3-methylazetidin-3-ol (218 mg, 2.51 mmol, 2.00 equiv.) in methanol (3 mL) was stirred at 80° C. overnight. LCMS showed that the reaction was complete. The reaction mixture was quenched with water (50 mL) and extracted with DCM (3*20 mL). The combined organic layers were concentrated under reduced pressure. The residue was purified by prep-HPLC(Column: XBridge Prep OBD C18 Column, 30×150 mm 5 μm; Mobile Phase A: water (10 mmol/L NH₄HCO₃+0.1% NH₃. H₂O), Mobile Phase B: ACN; Flow rate: 60 mL/min; Gradient: 53% B to 73% B in 7 min; 254/210 nm; RT: 6.3 min) to give 1-[5-[2-(2,6-dichlorophenyl)ethyl]-6-methyl-indan-1-yl]-3-methyl-azetidin-3-ol (192 mg, 39.3%) as a white solid. LCMS (ESI, m z): 390 [M+H]⁺.

### Chiral Separation of 1-[5-[2-(2,6-dichlorophenyl)ethyl]-6-methyl-indan-1-yl]-3-methyl-azetidin-3-ol (23a)

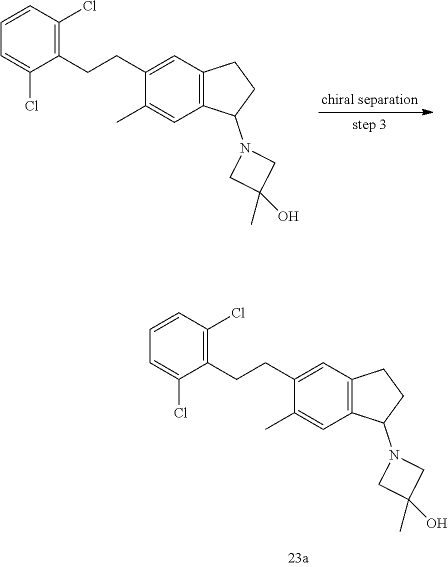 [SMILES: Cc1cc2c(cc1CCc1c(Cl)cccc1Cl)CCC2N1CC(C)(O)C1 ; Cc1cc2c(cc1CCc1c(Cl)cccc1Cl)CCC2N1CC(C)(O)C1]

The racemate (190 mg) was separated by chiral-HPLC(Column: Lux 5 μm Cellulose-2, 2.12*25 cm,5 μm; Mobile Phase A: HEX (0.5% 2M NH₃-MeOH)-HPLC, Mobile Phase B: EtOH-HPLC; Flow rate: 20 mL/min; Gradient: 95% B to 95% B in 13 min; 220/254 nm; RT1:8. 824 min; RT2:11.342 min) to afford the desired isomer 1-[5-[2-(2,6-dichlorophenyl)ethyl]-6-methyl-indan-1-yl]-3-methyl-azetidin-3-ol (25 mg, 12.9%, 99.7% e.e.) as a white solid.

¹H NMR (400 MHZ, Methanol-d₄) δ7.39 (d, J=8.0 Hz, 2H), 7.21 (t, J=8.0 Hz, 1H), 7.14 (s, 1H), 7.08 (s, 1H), 3.99-3.96 (m, 1H), 3.48-3.37 (m, 3H), 3.23 (d, J=7.6 Hz, 1H), 3.16-3.11 (m, 2H), 3.06-2.99 (m, 1H), 2.87-2.83 (m, 2H), 2.81-1.73 (m, 1H), 2.41 (s, 3H), 2.25-2.16 (m, 1H), 1.93-1.85 (m, 1H), 1.48 (s, 3H).

LCMS (ESI, m/z): 390 [M+H]⁺. Analytic Conditions: column: HALO C18, 3.0*30 mm, 2.7 μm; mobile phase A: water (0.05% TFA), mobile phase B: acetonitrile (0.05% TFA); flow rate: 1.20 mL/min; gradient: 5% B to 100% B in 1.20 min, hold at 100% for 0.60 min, 100% B to 5% B in 0.03 min; 220 nm; RT: 1.000 min.

### Chiral Separation of 1-[5-[2-(2,6-dichlorophenyl)ethyl]-6-methyl-indan-1-yl]-3-methyl-azetidin-3-ol (23b)

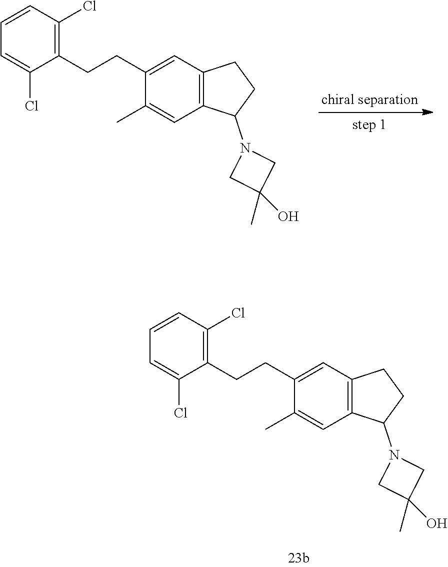 [SMILES: Cc1cc2c(cc1CCc1c(Cl)cccc1Cl)CCC2N1CC(C)(O)C1 ; Cc1cc2c(cc1CCc1c(Cl)cccc1Cl)CCC2N1CC(C)(O)C1]

The racemate (190 mg) was separated by chiral-HPLC(Column: Lux 5 μm Cellulose-2, 2.12*25 cm,5 μm; Mobile Phase A: HEX (0.5% 2M NH₃-MeOH)-HPLC, Mobile Phase B: EtOH-HPLC; Flow rate: 20 mL/min; Gradient: 95% B to 95% B in 13 min; 220/254 nm; RT1:8. 824 min; RT2:11.342 min) to afford the desired isomer 1-[5-[2-(2,6-dichlorophenyl)ethyl]-6-methyl-indan-1-yl]-3-methyl-azetidin-3-ol (31.3 mg, 16.3%, 98.9% e.e.) as a white solid.

¹H NMR (400 MHZ, Methanol-d₄) δ7.39 (d, J=8.0 Hz, 2H), 7.21 (t, J=8.0 Hz, 1H), 7.15 (s, 1H), 7.09 (s, 1H), 4.06-4.03 (m, 1H), 3.53-3.43 (m, 3H), 3.29 (d, J=8.0 Hz, 1H), 3.16-3.12 (m, 2H), 3.06-2.99 (m, 1H), 2.87-2.82 (m, 2H), 2.80-1.75 (m, 1H), 2.41 (s, 3H), 2.27-2.18 (m, 1H), 1.95-1.87 (m, 1H), 1.48 (s, 3H).

LCMS (ESI, m/z): 390 [M+H]⁺. Analytic Conditions: column: HALO C18, 3.0*30 mm, 2.7 μm; mobile phase A: water (0.05% TFA), mobile phase B: acetonitrile (0.05% TFA); flow rate: 1.20 mL/min; gradient: 5% B to 100% B in 1.20 min, hold at 100% for 0.60 min, 100% B to 5% B in 0.03 min; 220 nm; RT: 0.995 min.

### Example S24. 1-(5-((2,6-dichlorobenzyl)oxy)-6-methyl-2,3-dihydro-1H-inden-1-yl)-3-methylazetidin-3-ol (24a & 24b)

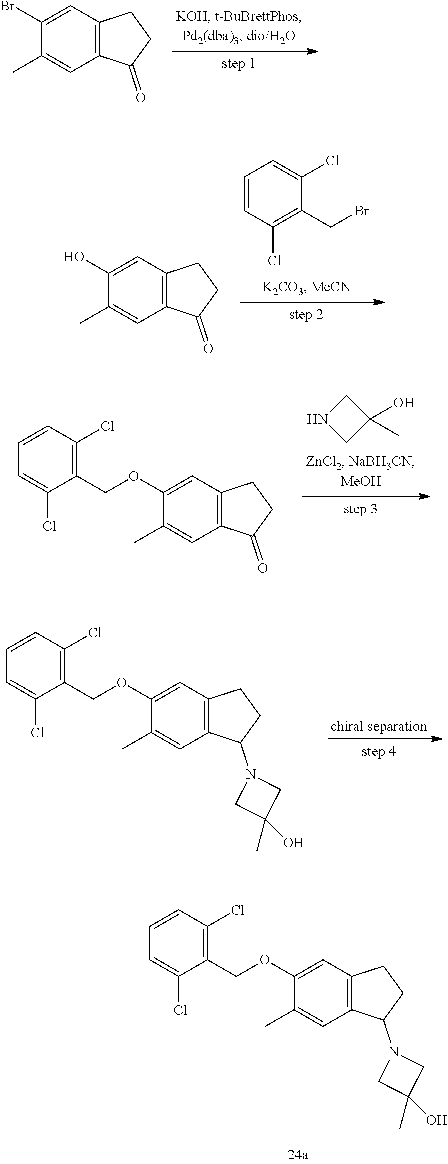

### Synthesis of 5-hydroxy-6-methyl-indan-1-one

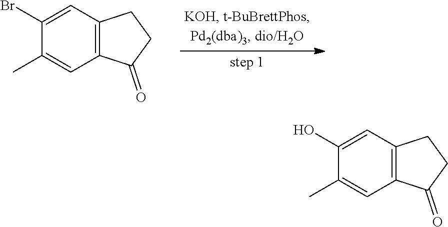

A solution of 5-bromo-6-methyl-indan-1-one (300 mg, 1.33 mmol, 1.00 equiv.), KOH(224 mg, 4 mmol, 3.00 equiv.), t-BuBrettPhos (129 mg, 0.27 mmol, 0.20 equiv.) and Pd2 (dba)₃ (122.1 mg, 0.13 mmol, 0.10 equiv.) in 1,4-dioxane (5 mL) and water (1 mL) was stirred at 80° C. overnight. LCMS showed that the reaction was complete. The reaction was acidified to pH 4˜5 with 1M HCl. The reaction mixture was filtered and the filtration was concentrated under reduced pressure. The residue was purified by flash chromatography on C18 silica (eluted with water/acetonitrile, 2:3) to afford 5-hydroxy-6-methyl-indan-1-one (175 mg, 81%) as a white solid. LCMS (ESI, m z): 163 [M+H]⁺.

### Synthesis of 5-[(2,6-dichlorophenyl) methoxy]-6-methyl-indan-1-one

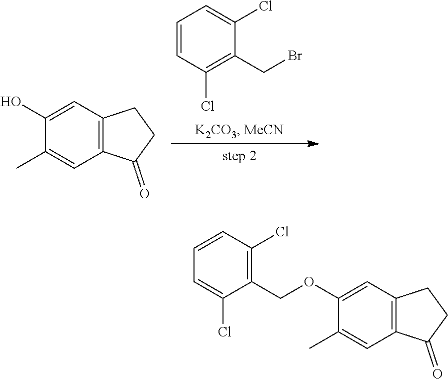 [SMILES: Cc1cc2c(cc1O)CCC2=O ; Clc1cccc(Cl)c1CBr ; Cc1cc2c(cc1OCc1c(Cl)cccc1Cl)CCC2=O]

A mixture of 5-hydroxy-6-methyl-indan-1-one (175 mg, 1.08 mmol, 1.00 equiv.), 2-(bromomethyl)-1,3-dichloro-benzene (388 mg, 1.62 mmol, 1.50 equiv.) and K₂CO₃ (447 mg, 3.24 mmol, 3.00 equiv.) in MeCN (4 mL) was stirred at 60° C. overnight. LCMS showed the reaction was completed. The reaction mixture was filtered and the filtrate was concentrated under reduced pressure. The residue was purified by flash chromatography on C18 silica (eluted with water/acetonitrile, 1:3) to afford 5-[(2,6-dichlorophenyl) methoxy]-6-methyl-indan-1-one (234 mg, 67.5%) as a white solid. LCMS (ESI, m z): 321 [M+H]⁺.

### Synthesis of 1-[5-[(2,6-dichlorophenyl) methoxy]-6-methyl-indan-1-yl]-3-methyl-azetidin-3-ol

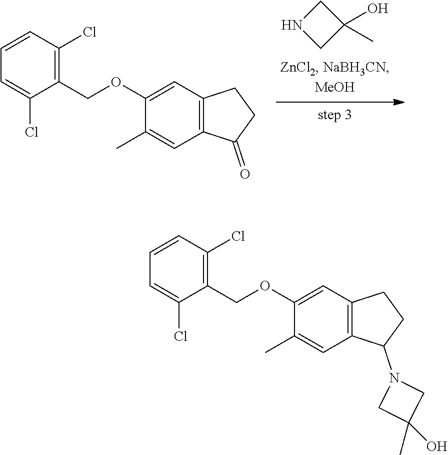

A solution of ZnCl₂ (2.0 M in THF, 0.65 mL, 1.31 mmol, 2.00 equiv.), NaBH₃CN (167 mg, 2.62 mmol, 4.00 equiv.), 5-[(2,6-dichlorophenyl) methoxy]-6-methyl-indan-1-one (210 mg, 0.65 mmol, 1.00 equiv.) and 3-methylazetidin-3-ol (114 mg, 1.31 mmol, 2.00 equiv.) in methanol (5 mL) was stirred at 80° C. overnight. LCMS showed that the reaction was complete. The reaction mixture was quenched with water (50 mL) and extracted with DCM (3*20 mL). The combined organic layers were concentrated under reduced pressure. The residue was purified by flash chromatography on C18 silica (eluted with water/acetonitrile, 1:1) to afford 1-[5-[(2,6-dichlorophenyl) methoxy]-6-methyl-indan-1-yl]-3-methyl-azetidin-3-ol (110 mg, 42.9%) as a light-yellow solid. LCMS (ESI, m z): 336 [M+H]⁺.

### Chiral Separation of 1-[5-[(2,6-dichlorophenyl) methoxy]-6-methyl-indan-1-yl]-3-methyl-azetidin-3-ol (24a)

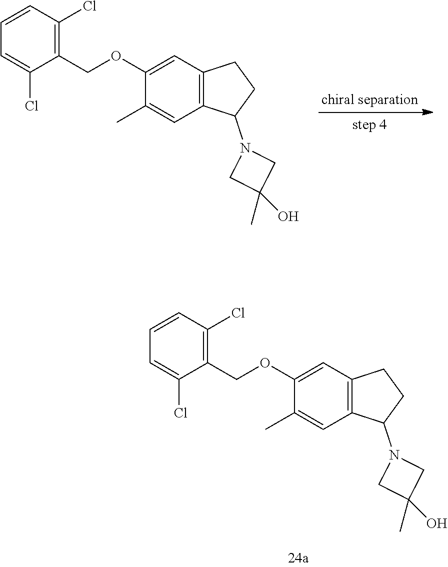 [SMILES: Cc1cc2c(cc1OCc1c(Cl)cccc1Cl)CCC2N1CC(C)(O)C1 ; Cc1cc2c(cc1OCc1c(Cl)cccc1Cl)CCC2N1CC(C)(O)C1]

The racemate (110 mg) was separated by chiral-HPLC(Column: CHIRALPAK IA, 2*25 cm,5 μm; Mobile Phase A: Hex (0.5% 2M NH₃-MeOH)-HPLC, Mobile Phase B: EtOH-HPLC; Flow rate: 20 mL/min; Gradient: 2% B to 2 B % in 19 min; 220/254 nm; RT1:10.127 min; RT2:11.859 min) to afford the desired isomer 1-[5-[(2,6-dichlorophenyl) methoxy]-6-methyl-indan-1-yl]-3-methyl-azetidin-3-ol (the first eluting peak, 37.3 mg, 32.9%, 97.9% e.e.) as a white solid.

¹H NMR (400 MHZ, Methanol-d₄) δ7.49-7.46 (m, 2H), 7.37 (dd, J=9.2, 7.6 Hz, 1H), 7.12 (s, 1H), 7.03 (s, 1H), 5.30 (s, 2H), 4.04-4.01 (m, 1H), 3.51-3.48 (m, 2H), 3.43 (d, J=8.0 Hz, 1H), 3.29 (d, J=8.0 Hz, 1H), 3.12-3.06 (m, 1H), 2.88-2.81 (m, 1H), 2.30-2.21 (m, 1H), 2.13 (s, 3H), 1.97-1.90 (m, 1H), 1.48 (s, 3H).

LCMS (ESI, m/z): 392 [M+H]⁺. Analytic Conditions: column: HALO C18, 3.0*30 mm, 2.7 μm; mobile phase A: water (0.05% TFA), mobile phase B: acetonitrile (0.05% TFA); flow rate: 1.20 mL/min; gradient: 5% B to 100% B in 1.30 min, hold at 100% for 0.50 min, 100% B to 5% B in 0.03 min; 220 nm; RT: 0.955 min.

### Chiral Separation of 1-[5-[(2,6-dichlorophenyl) methoxy]-6-methyl-indan-1-yl]-3-methyl-azetidin-3-ol (24b)

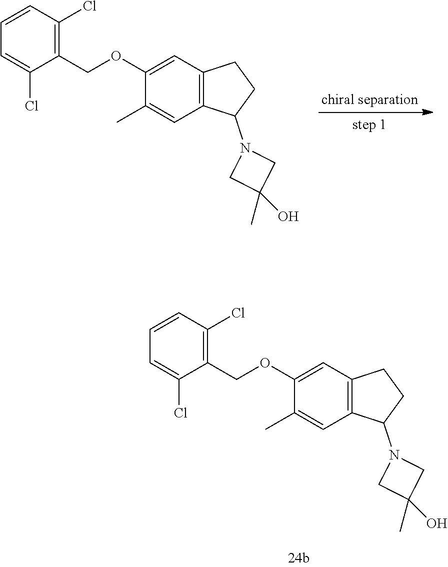 [SMILES: Cc1cc2c(cc1OCc1c(Cl)cccc1Cl)CCC2N1CC(C)(O)C1 ; Cc1cc2c(cc1OCc1c(Cl)cccc1Cl)CCC2N1CC(C)(O)C1]

The racemate (110 mg) was separated by chiral-HPLC(Column: CHIRALPAK IA, 2*25 cm,5 μm; Mobile Phase A: Hex (0.5% 2M NH₃-MeOH)-HPLC, Mobile Phase B: EtOH-HPLC; Flow rate: 20 mL/min; Gradient: 2% B to 2 B % in 19 min; 220/254 nm; RT1:10.127 min; RT2:11.859 min) to afford the desired isomer 1-[5-[(2,6-dichlorophenyl) methoxy]-6-methyl-indan-1-yl]-3-methyl-azetidin-3-ol (the second eluting peak, 21.7 mg, 19.5%, 97.6% e.e.) as a white solid.

¹H NMR (400 MHZ, Methanol-d₄) δ7.49-7.46 (m, 2H), 7.37 (dd, J=9.2, 7.6 Hz, 1H), 7.12 (s, 1H), 7.03 (s, 1H), 5.30 (s, 2H), 4.01-3.98 (m, 1H), 3.48-3.45 (m, 2H), 3.39 (d, J=8.0 Hz, 1H), 3.25 (d, J=8.0 Hz, 1H), 3.13-3.06 (m, 1H), 2.87-2.80 (m, 1H), 2.29-2.20 (m, 1H), 2.13 (s, 3H), 1.96-1.89 (m, 1H), 1.48 (s, 3H).

LCMS (ESI, m/z): 392 [M+H]⁺. Analytic Conditions: column: HALO C18, 3.0*30 mm, 2.7 μm; mobile phase A: water (0.05% TFA), mobile phase B: acetonitrile (0.05% TFA); flow rate: 1.20 mL/min; gradient: 5% B to 100% B in 1.30 min, hold at 100% for 0.50 min, 100% B to 5% B in 0.03 min; 210 nm; RT: 0.950 min.

### Example S25. 1-(5-(3-chloro-4-cyclopropylphenyl)-4,6-dimethyl-2,3-dihydro-1H-inden-1-yl)-3-methylazetidin-3-ol (25a & 25b)

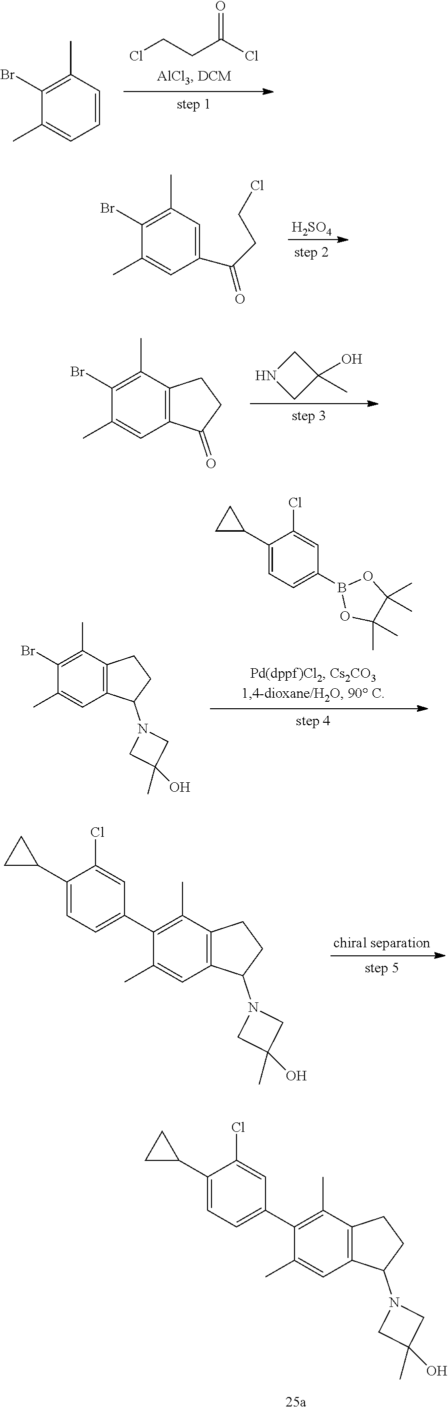

### Synthesis of 1-(4-bromo-3,5-dimethyl-phenyl)-3-chloro-propan-1-one

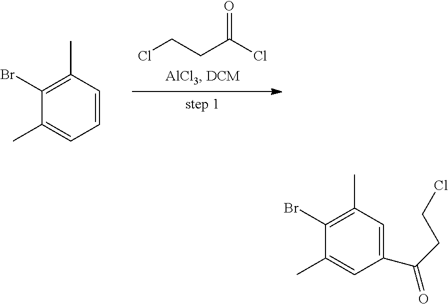

To a stirred solution of 3-chloropropanoyl chloride (1.37 g, 10.8 mmol, 1.00 equiv.) and AlCl3 (2.0 g, 15.2 mmol, 1.50 equiv.) in DCM (40 mL) was added the solution of 2-bromo-1,3-dimethyl-benzene (2 g, 10.8 mmol, 1.00 equiv.) in DCM (4 mL) dropwise at 0° C. The resulting mixture was then allowed to stir at room temperature for 12 h. The reaction mixture was quenched with ice water (40 mL) and conc. HCl (5 mL) and was stirred for 15 min. Then the mixture was extracted with DCM (3*20 mL). The combined organic lawyers were concentrated under reduced pressure to afford a mixture of isomers including 1-(4-bromo-3,5-dimethyl-phenyl)-3-chloro-propan-1-one as crude product, which was used directly for the next step without further purification. LCMS (ESI, m z): 275 [M+H]⁺.

### Synthesis of 5-bromo-4,6-dimethyl-indan-1-one

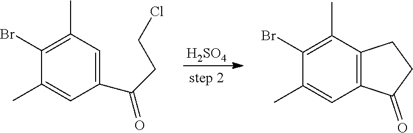

A solution of isomer mixture (including 1-(4-bromo-3,5-dimethyl-phenyl)-3-chloro-propan-1-one)(1.3 g, 4.72 mmol, 1.00 equiv.) in conc. H₂SO₄ (5 mL) was stirred at 90° C. for 1 h. Then the reaction mixture was quenched with ice water (50 mL) and was extracted with ethyl acetate (3*20 mL). The combined organic layers were concentrated under reduced pressure. The residue was purified by flash column chromatography on silica gel (eluted with PE: EtOAc=12:1) to afford a mixture of isomers of 5-bromo-4,6-dimethyl-indan-1-one (800 mg, 70%) as white solid, which was separated by achiral-SFC(Column: Green Sep Naphthyl, 3*25 cm,5 μm; Mobile Phase A: CO₂, Mobile Phase B: IPA (0.5% 2M NH₃-MeOH); Flow rate: 80 mL/min; Gradient: isocratic 30% B; Wave Length: 254 nm; RT1 (min): 3.63; RT2 (min): 4.18) to afford the desired isomer 5-bromo-4,6-dimethyl-indan-1-one (400 mg, 50%, 99% e.e.) as a white solid LCMS (ESI, m, z): 239 [M+H]⁺.

### Synthesis of 1-(5-bromo-4,6-dimethyl-indan-1-yl)-3-methyl-azetidin-3-ol

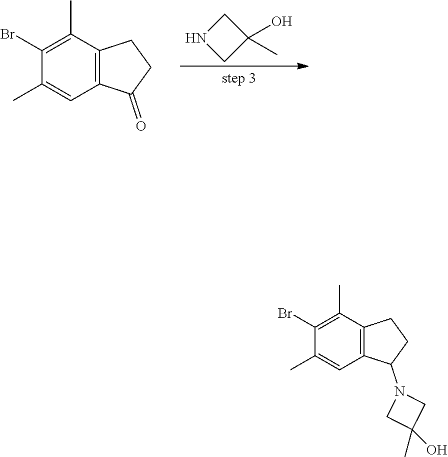

A solution of 5-bromo-4,6-dimethyl-indan-1-one (400 mg, 1.67 mmol, 1.00 equiv.), 3-methylazetidin-3-ol (145 mg, 1.67 mmol, 1.00 equiv.), ZnCl₂ (2M in 4Me-THF, 1.6 mL, 3.35 mmol, 2.00 equiv.) and NaH3BCN (421 mg, 6.69 mmol, 4.00 equiv.) in methanol (10 mL) was placed in a 25 ml round-bottom flask. The resulting solution was stirred at 60° C. for 15h. LCMS showed the reaction was completed. The reaction was quenched by water (60 mL) and extracted with EtOAc (2×20 mL). The combined organic layers were concentrated under reduced pressure. The residue was purified by flash chromatography on silica gel (eluted with ethyl acetate/petroleum ether, 2:3) to afford 1-(5-bromo-4,6-dimethyl-indan-1-yl)-3-methyl-azetidin-3-ol (200 mg, 39%) as a yellow oil. LCMS (ESI, m z): 310 [M+H]⁺.

### Synthesis of 1-(5-(3-chloro-4-cyclopropylphenyl)-4,6-dimethyl-2,3-dihydro-1H-inden-1-yl)-3-methylazetidin-3-ol

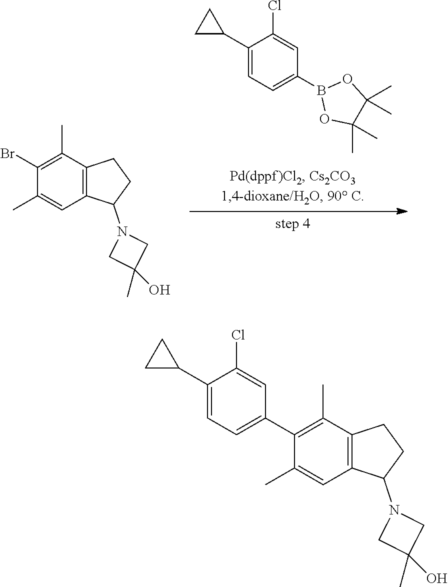 [SMILES: Cc1cc2c(c(C)c1Br)CCC2N1CC(C)(O)C1 ; CC1(C)OB(c2ccc(C3CC3)c(Cl)c2)OC1(C)C ; Cc1cc2c(c(C)c1-c1ccc(C3CC3)c(Cl)c1)CCC2N1CC(C)(O)C1]

A solution of 1-(5-bromo-4,6-dimethyl-indan-1-yl)-3-methyl-azetidin-3-ol (190 mg, 0.61 mmol, 1.00 equiv.), 2-(3-chloro-4-cyclopropyl-phenyl)-4,4,5,5-tetramethyl-1,3,2-dioxaborolane (170 mg, 0.61 mmol. 1.00 equiv.), Cs₂CO₃ (598 mg, 1.84 mmol, 3.00 equiv.) and Pd(dppf)Cl₂ (44 mg, 0.060 mmol, 0.10 equiv.) in 1,4-dioxane (10 mL) and water (1 mL) was was stirred at 90° C. for 4 h under N₂ atmosphere. LCMS showed the reaction was completed. The mixture was concentrated under reduced pressure. The residue was purified by flash chromatography on silica gel (eluted with ethyl acetate/petroleum ether, 1:1) to afford 1-(5-(3-chloro-4-cyclopropylphenyl)-4,6-dimethyl-2,3-dihydro-1H-inden-1-yl)-3-methylazetidin-3-ol (200 mg, 85%) as a yellow oil. LCMS (ESI, m z): 382 [M+H]⁺.

### Chiral Separation of 1-(5-(3-chloro-4-cyclopropylphenyl)-4,6-dimethyl-2,3-dihydro-1H-inden-1-yl)-3-methylazetidin-3-ol (25a)

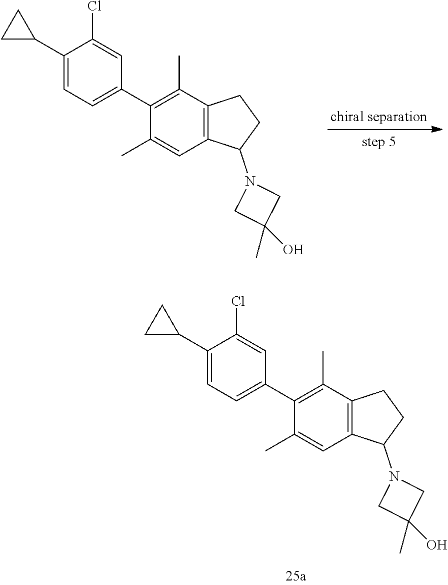 [SMILES: Cc1cc2c(c(C)c1-c1ccc(C3CC3)c(Cl)c1)CCC2N1CC(C)(O)C1 ; Cc1cc2c(c(C)c1-c1ccc(C3CC3)c(Cl)c1)CCC2N1CC(C)(O)C1]

The racemate (200 mg) was separated by chiral-HPLC(Column: Lux 5 μm Cellulose-2, 2.12*25 cm, 5 μm; Mobile Phase A: Hex (0.5% 2M NH₃-MeOH)-HPLC, Mobile Phase B: EtOH-HPLC; Flow rate: 20 mL/min; Gradient: 98% B to 98% B in 25 min; 220/254 nm; RT1:15.662 min; RT2:21.512 min) to afford the desired isomer (first eluting peak, 12.3 mg, 6%, 100% e.e.) as a white solid.

¹H NMR (400 MHZ, Methanol-d₄) δ7.09-7.04 (m, 3H), 6.95-6.89 (m, 1H), 4.01-3.98 (m, 1H), 3.47-3.43 (m, 2H), 3.38-3.35 (m, 1H), 3.24-3.21 (m, 1H), 3.03-2.95 (m, 1H), 2.81-2.74 (m, 1H), 2.29-2.18 (m, 2H), 1.99 (s, 3H), 1.97-1.94 (m, 1H), 1.92 (s, 3H), 1.48 (d, J=2.4 Hz, 3H), 1.09-1.04 (m, 2H), 0.78-0.74 (m, 2H).

LCMS (ESI, m/z): 382 [M+H]⁺. Analytic Conditions: column: HALO C18, 3.0*30 mm, 2.7 μm; mobile phase A: water (0.05% TFA), mobile phase B: acetonitrile (0.05% TFA); flow rate: 1.20 mL/min; gradient: 5% B to 70% B in 1.70 min, 70% B to 95% B in 0.30 min, hold at 100% for 0.60 min, 100% B to 5% B in 0.10 min; 220 nm; RT: 1.545 min.

### Chiral Separation of 1-(5-(3-chloro-4-cyclopropylphenyl)-4,6-dimethyl-2,3-dihydro-1H-inden-1-yl)-3-methylazetidin-3-ol (25b)

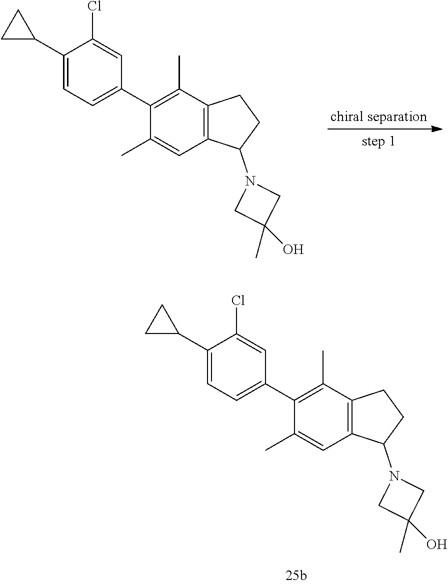 [SMILES: Cc1cc2c(c(C)c1-c1ccc(C3CC3)c(Cl)c1)CCC2N1CC(C)(O)C1 ; Cc1cc2c(c(C)c1-c1ccc(C3CC3)c(Cl)c1)CCC2N1CC(C)(O)C1]

The racemate (200 mg) was separated by chiral-HPLC(Column: Lux 5 μm Cellulose-2, 2.12*25 cm, 5 μm; Mobile Phase A: Hex (0.5% 2M NH₃-MeOH)-HPLC, Mobile Phase B: EtOH-HPLC; Flow rate: 20 mL/min; Gradient: 98% B to 98% B in 25 min; 220/254 nm; RT1:15.662 min; RT2:21.512 min) to afford the desired isomer (first eluting peak, 14 mg, 7%, 99% e.e.) as a white solid.

¹H NMR (400 MHZ, Methanol-d₄) δ7.10-7.05 (m, 3H), 6.95-6.90 (m, 1H), 4.02-3.99 (m, 1H), 3.46-3.43 (m, 2H), 3.39-3.37 (m, 1H), 3.24-3.21 (m, 1H), 3.03-2.95 (m, 1H), 2.81-2.74 (m, 1H), 2.29-2.18 (m, 2H), 2.00 (s, 3H), 1.97-1.94 (m, 1H), 1.92 (s, 3H), 1.48 (d, J=2.0 Hz, 3H), 1.09-1.04 (m, 2H), 0.78-0.74 (m, 2H).

LCMS (ESI, m/z): 382 [M+H]⁺. Analytic Conditions: column: HALO C18, 3.0*30 mm, 2.7 μm; mobile phase A: water (0.05% TFA), mobile phase B: acetonitrile (0.05% TFA); flow rate: 1.20 mL/min; gradient: 5% B to 100% B in 1.20 min, hold at 100% for 0.60 min, 100% B to 5% B in 0.03 min; 220 nm; RT: 1.033 min.

### Example S26. 1-(5-(3-chloro-4-cyclopropylphenyl)-4,7-dimethyl-2,3-dihydro-1H-inden-1-yl)-3-methylazetidin-3-ol (26a & 26b)

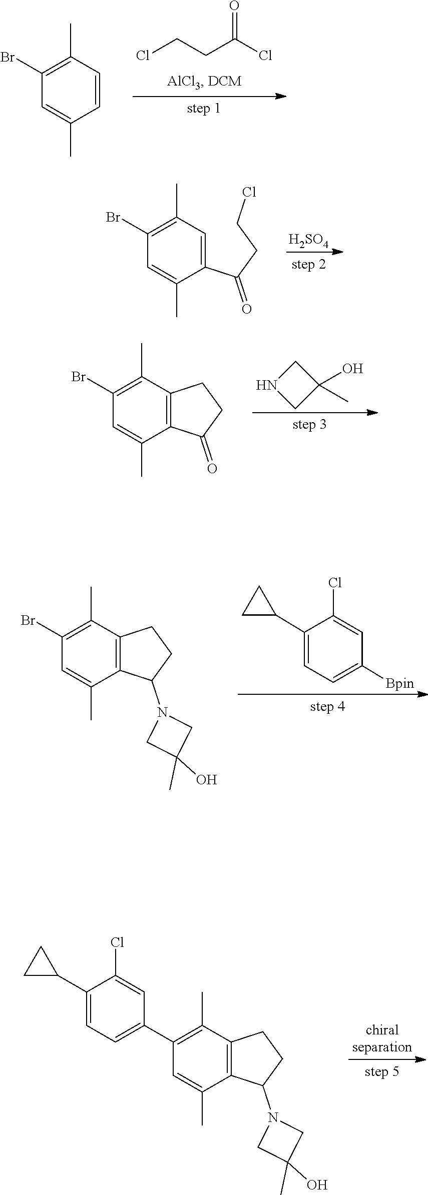

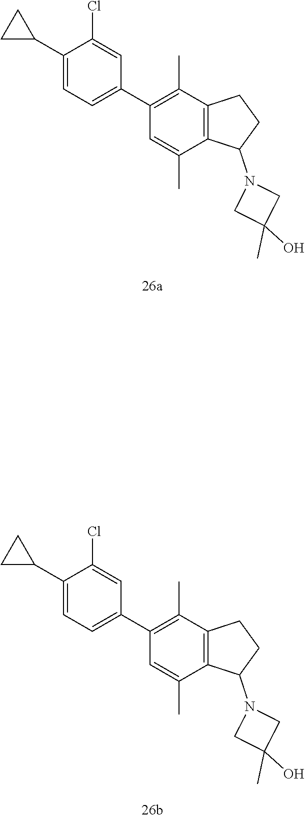 [SMILES: Cc1cc(-c2ccc(C3CC3)c(Cl)c2)c(C)c2c1C(N1CC(C)(O)C1)CC2 ; Cc1cc(-c2ccc(C3CC3)c(Cl)c2)c(C)c2c1C(N1CC(C)(O)C1)CC2]

### Synthesis of 1-(4-bromo-2,5-dimethylphenyl)-3-chloropropan-1-one

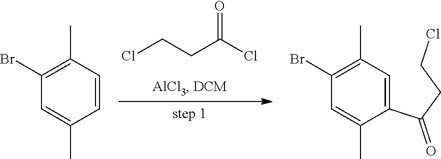

To the stirred solution of AlCl3 (2.2 g, 16.2 mmol, 1.5 equiv.) and 3-chloropropanoyl chloride (1.7 g, 13.0 mmol, 1.2 equiv.) in DCM (20 mL) was added the solution of 2-bromo-1,4-dimethyl-benzene (2 g, 10.8 mmol, 1 equiv.) in DCM (20 mL) dropwise at 0° C. The resulting mixture was then allowed to stir at room temperature for 12 h. The reaction mixture was quenched with ice water (40 mL) and conc. HCl (5 mL) and was stirred for 15 min. Then the mixture was extracted with ethyl acetate (3*50 mL). The combined organic lawyers were concentrated under reduced pressure to afford 1-(4-bromo-2,5-dimethylphenyl)-3-chloropropan-1-one as crude product, which was used directly for the next step without further purification. LCMS (ESI, m z): 275 [M+H]⁺.

### Synthesis of 5-bromo-4,7-dimethyl-indan-1-one

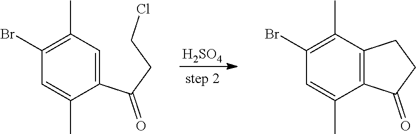

A solution of 1-(4-bromo-2,5-dimethylphenyl)-3-chloropropan-1-one (2.8 g, 10.8 mmol, 1 equiv.) in conc. H₂SO₄ (8 mL) was stirred at 90° C. for 1 h. Then the reaction mixture was quenched with ice water (50 mL) and was extracted with ethyl acetate (3*40 mL). The combined organic layers were concentrated under reduced pressure. The residue was purified by flash column chromatography on silica gel (eluted with PE: EtOAc=12:1) to afford 5-bromo-4,7-dimethyl-indan-1-one (2 g, 76.7%) as yellow solid. LCMS (ESI, m z): 239 [M+H]⁺.

### Synthesis of 1-(5-bromo-4,7-dimethyl-indan-1-yl)-3-methyl-azetidin-3-ol

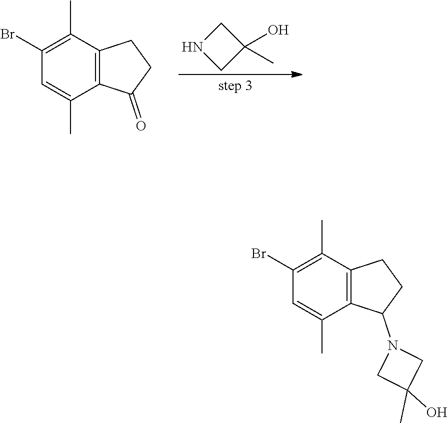

A solution of NaCNBH₃ (1.27 g, 33.46 mmol, 4 equiv.) and ZnCl₂ (4.2 mL, 2M in 4Me-THF, 8.36 mmol, 1 equiv.) in methanol (20 mL) was stirred at room temperature for 0.5 h. Then 5-bromo-4,7-dimethyl-indan-1-one (2 g, 8.36 mmol, 1 equiv.) and 3-methylazetidin-3-ol (1.46 g, 16.7 mmol, 2 equiv.) were added. The resulting mixture was stirred at 60° C. for 12 h. LCMS showed the reaction was completed. The reaction was quenched by water (100 mL) and extracted with EtOAc (2×30 mL). The combined organic layers were concentrated under reduced pressure. The residue was purified by flash chromatography on silica gel (eluted with ethyl acetate/petroleum ether, 1:1) to afford 1-(5-bromo-4,7-dimethyl-indan-1-yl)-3-methyl-azetidin-3-ol (500 mg, 19.2%) as a yellow oil. LCMS (ESI, m z): 310 [M+H]⁺.

### Synthesis of 1-[5-(3-chloro-4-cyclopropyl-phenyl)-4,7-dimethyl-indan-1-yl]-3-methyl-azetidin-3-ol

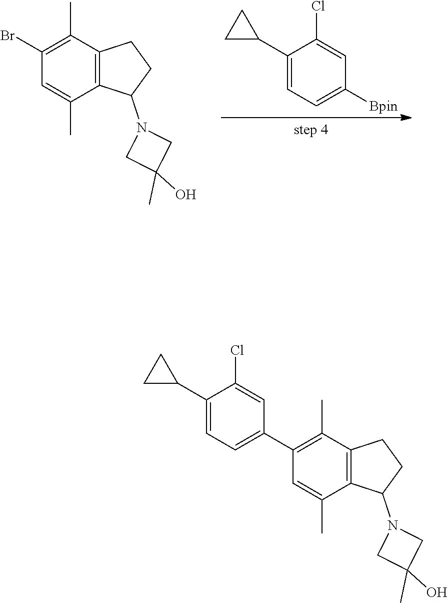

A mixture of 1-(5-bromo-4,7-dimethyl-indan-1-yl)-3-methyl-azetidin-3-ol (500, mg, 1.61 mmol, 1 equiv.), 2-(3-chloro-4-cyclopropyl-phenyl)-4,4,5,5-tetramethyl-1,3,2-dioxaborolane (898 mg, 3.22 mmol, 2 equiv.), Pd(dppf)Cl2 (118 mg, 0.16 mmol, 0.1 equiv.) and Cs₂CO₃ (1.57 g, 4.84 mmol, 3 equiv.) in 1,4-dioxane (5 mL) and water (0.5 mL) was stirred at 90° C. overnight. LCMS showed the reaction was completed. The mixture was concentrated under reduced pressure. The residue was purified by flash chromatography on silica gel (eluted with ethyl acetate/petroleum ether, 1:1) to afford 1-[5-(3-chloro-4-cyclopropyl-phenyl)-4,7-dimethyl-indan-1-yl]-3-methyl-azetidin-3-ol (300 mg, 48.7%) as a yellow oil. LCMS (ESI, m z): 382 [M+H]⁺.

### Chiral Separation of 1-[5-(3-chloro-4-cyclopropyl-phenyl)-4,7-dimethyl-indan-1-yl]-3-methyl-azetidin-3-ol (26a)

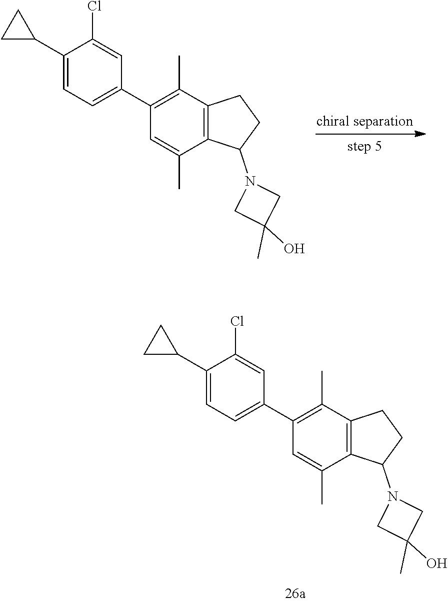 [SMILES: Cc1cc(-c2ccc(C3CC3)c(Cl)c2)c(C)c2c1C(N1CC(C)(O)C1)CC2 ; Cc1cc(-c2ccc(C3CC3)c(Cl)c2)c(C)c2c1C(N1CC(C)(O)C1)CC2]

The racemate (300 mg) was purified chiral HPLC(Column: Lux 5 μm Celluloes-3, 2.12*25 cm, 5 μm; Mobile Phase A: Hex (0.5% 2M NH₃-MeOH)-HPLC, Mobile Phase B: EtOH-HPLC; Flow rate: 20 mL/min; Gradient: 2% B to 2% B in 20 min; 220/254 nm; RT1:6.271 min; RT2:13.587 min) to afford the enantiomers. The first eluting peak enantiomer was purified by Prep-HPLC(Column: XBridge Prep OBD C18 Column, 30*150 mm 5 μm; Mobile Phase A: water (10 mM NH₄HCO₃), Mobile Phase B: ACN; Flow rate: 60 mL/min; Gradient: 65% B to 95% B in 7 min; 254/210 nm; RT1:5.68 min) to afford 1-[5-(3-chloro-4-cyclopropyl-phenyl)-4,7-dimethyl-indan-1-yl]-3-methyl-azetidin-3-ol (67.6 mg, 22.3%) as a white solid.

¹H NMR (300 MHz, Methanol-d₄) δ 7.24 (d, J=1.5 Hz, 1H), 7.10 (dd, J=7.8, 1.5 Hz, 1H), 7.03 (d, J=7.8 Hz, 1H), 6.83 (s, 1H), 4.14-4.11 (m, 1H), 3.30-3.27 (m, 2H), 3.24-3.21 (m, 1H), 3.16 (d, J=7.2 Hz, 1H), 3.10-2.99 (m, 1H), 2.80-2.72 (m, 1H), 2.40 (s, 3H), 2.28-2.12 (m, 2H), 2.10 (s, 3H), 2.07-1.98 (m, 1H), 1.45 (s, 3H), 1.08-1.01 (m, 2H), 0.76-0.71 (m, 2H).

LCMS (ESI, m/z): 382 [M+H]⁺. Analytic Conditions: column: YMCMeteoricCore C18 BIO, 2.1*30 mm, 2.7 μm; mobile phase A: water (5 mM NH₄HCO₃), mobile phase B: acetonitrile; flow rate: 1.20 mL/min; gradient: 10% B to 95% B in 1.20 min, hold at 95% for 0.58 min, 95% B to 10% B in 0.05 min; 254 nm; RT: 1.186 min.

### Chiral Separation of 1-[5-(3-chloro-4-cyclopropyl-phenyl)-4,7-dimethyl-indan-1-yl]-3-methyl-azetidin-3-ol (26b)

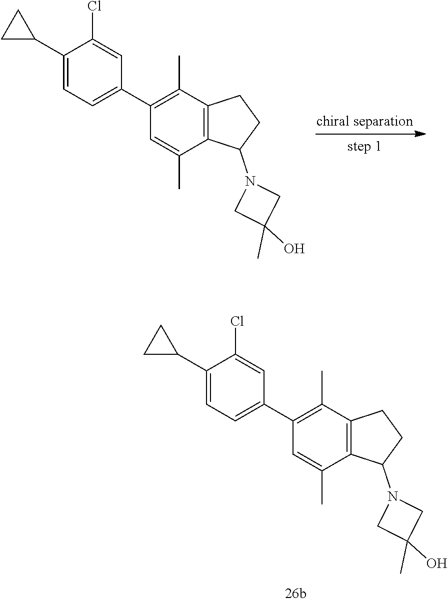 [SMILES: Cc1cc(-c2ccc(C3CC3)c(Cl)c2)c(C)c2c1C(N1CC(C)(O)C1)CC2 ; Cc1cc(-c2ccc(C3CC3)c(Cl)c2)c(C)c2c1C(N1CC(C)(O)C1)CC2]

The racemate (300 mg) was purified chiral HPLC(Column: Lux 5 μm Celluloes-3, 2.12*25 cm, 5 μm; Mobile Phase A: Hex (0.5% 2M NH₃-MeOH)-HPLC, Mobile Phase B: EtOH-HPLC; Flow rate: 20 mL/min; Gradient: 2% B to 2% B in 20 min; 220/254 nm; RT1:6.271 min; RT2:13.587 min) to afford the enantiomers. The second eluting peak enantiomer was purified by Prep-HPLC(Column: XBridge Prep OBD C18 Column, 30*150 mm 5 μm; Mobile Phase A: water (10 mM NH₄HCO₃), Mobile Phase B: ACN; Flow rate: 60 mL/min; Gradient: 65% B to 95% B in 7 min; 254/210 nm; RT1:8.32 min) to afford 1-[5-(3-chloro-4-cyclopropyl-phenyl)-4,7-dimethyl-indan-1-yl]-3-methyl-azetidin-3-ol (63.4 mg, 21.0%) as a white solid.

¹H NMR (300 MHZ, Methanol-d₄) δ7.24 (d, J=1.5 Hz, 1H), 7.10 (dd, J=7.8, 1.5 Hz, 1H), 7.03 (d, J=7.8 Hz, 1H), 6.83 (s, 1H), 4.14-4.11 (m, 1H), 3.30-3.27 (m, 2H), 3.24-3.21 (m, 1H), 3.16 (d, J=6.9 Hz, 1H), 3.10-2.99 (m, 1H), 2.80-2.72 (m, 1H), 2.41 (s, 3H), 2.28-2.12 (m, 2H), 2.11 (s, 3H), 2.07-1.97 (m, 1H), 1.45 (s, 3H), 1.08-1.02 (m, 2H), 0.76-0.71 (m, 2H).

LCMS (ESI, m/z): 382 [M+H]⁺. Analytic Conditions: column: HALO C18, 3.0*30 mm, 2.0 μm; mobile phase A: water (0.05% TFA), mobile phase B: acetonitrile (0.05% TFA); flow rate: 1.20 mL/min; gradient: 5% B to 100% B in 1.20 min, hold at 100% for 0.60 min, 100% B to 5% B in 0.03 min; 254 nm; RT: 1.045 min.

### Example S27. 1-(5-(3-chloro-4-cyclopropylphenyl)-6,7-dimethyl-2,3-dihydro-1H-inden-1-yl)-3-methylazetidin-3-ol (27a & 27b)

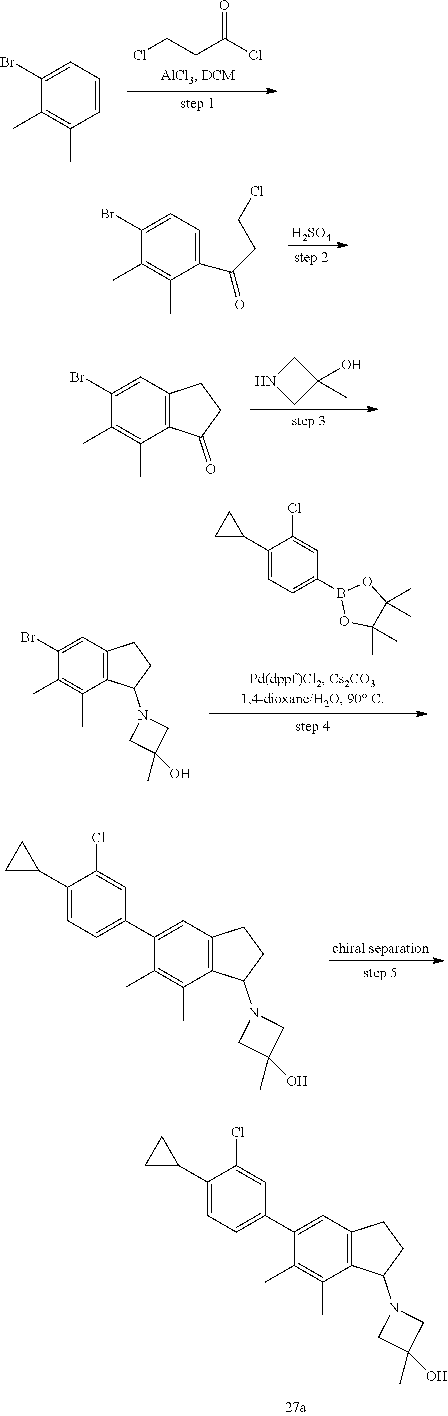

### Synthesis of 1-(4-bromo-2,3-dimethyl-phenyl)-3-chloro-propan-1-one

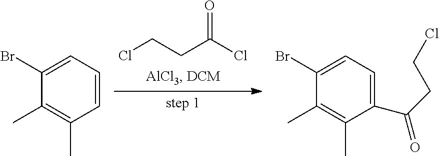

To a stirred solution of 3-chloropropanoyl chloride (3.4 g, 27.0 mmol, 1.00 equiv.) and AlCl₃ (5.1 g, 37.9 mmol, 1.50 equiv.) in DCM (80 mL) was added the solution of 1-bromo-2,3-dimethyl-benzene (5 g, 27.0 mmol, 1.00 equiv.) in DCM (8 mL) dropwise at 0° C. The resulting mixture was then allowed to stir at room temperature for 12 h. The reaction mixture was quenched with ice water (40 mL) and conc. HCl (5 mL) and was stirred for 15 min. Then the mixture was extracted with DCM (3*20 mL). The combined organic lawyers were concentrated under reduced pressure to afford a mixture of isomers including 1-(4-bromo-2,3-dimethyl-phenyl)-3-chloro-propan-1-one as crude product, which was used directly for the next step without further purification. LCMS (ESI, m z): 275 [M+H]⁺.

### Synthesis of 5-bromo-6,7-dimethyl-2,3-dihydro-1H-inden-1-one

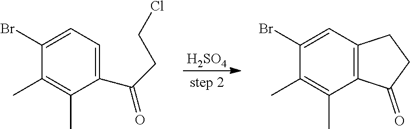

A solution of isomer mixture (including 1-(4-bromo-2,3-dimethyl-phenyl)-3-chloro-propan-1-one)(2.6 g, 9.44 mmol, 1.00 equiv.) in conc. H₂SO₄ (10 mL) was stirred at 90° C. for 1 h. Then the reaction mixture was quenched with ice water (50 mL) and was extracted with ethyl acetate (3*40 mL). The combined organic layers were concentrated under reduced pressure. The residue was purified by flash column chromatography on silica gel (eluted with PE: EtOAc=12:1) to afford a mixture of isomers (1.2 g, 55%) as white solid, which separated by achiral-SFC (Column: Green Sep Naphthyl, 3*25 cm, 5 μm; Mobile Phase A: CO₂, Mobile Phase B: IPA (0.5% 2M NH₃-MeOH); Flow rate: 70 mL/min; Gradient: isocratic 15% B; Wave Length: 254 nm; RT1 (min): 5.72; RT2 (min): 6.23; Sample Solvent: DCM-HPLC; Injection Volume: 1 mL; Number Of Runs: 40) to afford the desired isomer 5-bromo-6,7-dimethyl-2,3-dihydro-1H-inden-1-one (530 mg, 44%, 99% e.e.) as a white solid LCMS (ESI, m z): 239 [M+H]⁺.

### Synthesis of 1-(5-bromo-4,6-dimethyl-indan-1-yl)-3-methyl-azetidin-3-ol

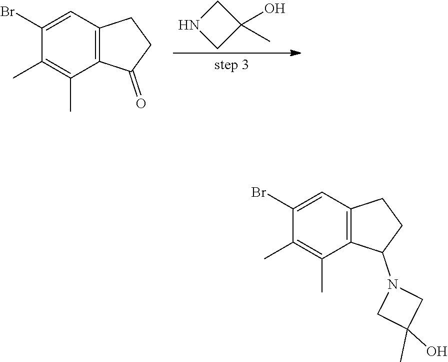

A solution of 5-bromo-6,7-dimethyl-2,3-dihydro-1H-inden-1-one (530 mg, 1.71 mmol, 1.00 equiv.), 3-methylazetidin-3-ol (158 mg, 1.71 mmol, 1.00 equiv.), ZnCl₂ (2M in 4Me-THF, 1.8 mL, 3.42 mmol, 2.00 equiv.) and NaH3BCN (430 mg, 6.84 mmol, 4.00 equiv.) in methanol (10 mL) was was stirred at 60° C. for 15 h. LCMS showed the reaction was completed. The reaction was quenched by water (60 mL) and extracted with EtOAc (2×20 mL). The combined organic layers were concentrated under reduced pressure. The residue was purified by flash chromatography on silica gel (eluted with ethyl acetate/petroleum ether, 2:3) to afford 1-(5-bromo-4,6-dimethyl-indan-1-yl)-3-methyl-azetidin-3-ol (120 mg, 17%) as a yellow oil. LCMS (ESI, m z): 310 [M+H]⁺.

### Synthesis of 1-(5-(3-chloro-4-cyclopropylphenyl)-6,7-dimethyl-2,3-dihydro-1H-inden-1-yl)-3-methylazetidin-3-ol

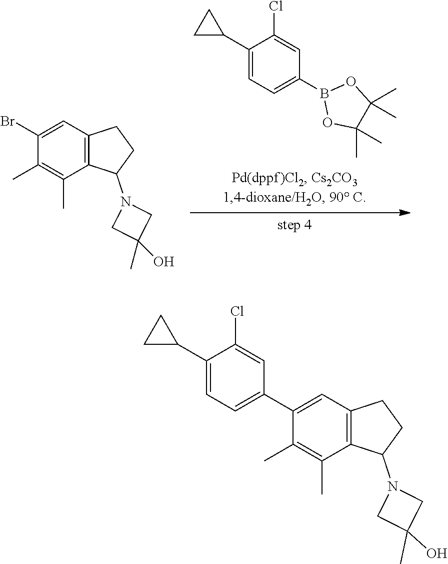 [SMILES: Cc1c(Br)cc2c(c1C)C(N1CC(C)(O)C1)CC2 ; CC1(C)OB(c2ccc(C3CC3)c(Cl)c2)OC1(C)C ; Cc1c(-c2ccc(C3CC3)c(Cl)c2)cc2c(c1C)C(N1CC(C)(O)C1)CC2]

A solution of 1-(5-bromo-4,6-dimethyl-indan-1-yl)-3-methyl-azetidin-3-ol (120 mg, 0.39 mmol, 1.00 equiv.), 2-(3-chloro-4-cyclopropyl-phenyl)-4,4,5,5-tetramethyl-1,3,2-dioxaborolane (170 mg, 0.39 mmol. 1.00 equiv.), Cs₂CO₃ (598 mg, 1.46 mmol, 3.00 equiv.) and Pd(dppf)Cl₂ (44 mg, 0.04 mmol, 0.10 equiv.) in 1,4-dioxane (10 mL) and water (1 mL) was stirred at 90° C. for 4 h under N₂ atmosphere. LCMS showed the reaction was completed. The mixture was concentrated under reduced pressure. The residue was purified by flash chromatography on silica gel (eluted with ethyl acetate/petroleum ether, 1:1) to afford 1-(5-(3-chloro-4-cyclopropylphenyl)-6,7-dimethyl-2,3-dihydro-1H-inden-1-yl)-3-methylazetidin-3-ol (60 mg, 85%) as a yellow oil. LCMS (ESI, m z): 382 [M+H]⁺.

### Chiral Separation of 1-(5-(3-chloro-4-cyclopropylphenyl)-6,7-dimethyl-2,3-dihydro-1H-inden-1-yl)-3-methylazetidin-3-ol (27a)

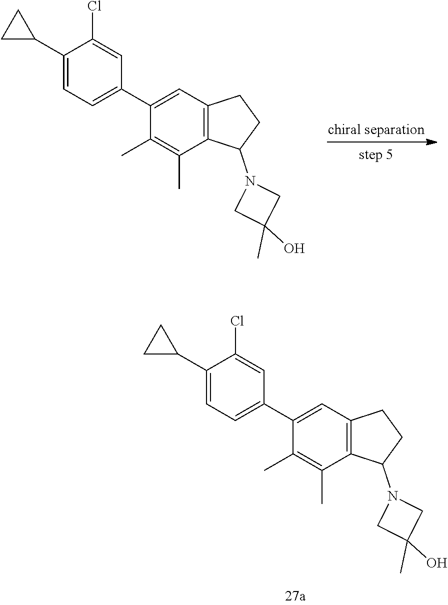 [SMILES: Cc1c(-c2ccc(C3CC3)c(Cl)c2)cc2c(c1C)C(N1CC(C)(O)C1)CC2 ; Cc1c(-c2ccc(C3CC3)c(Cl)c2)cc2c(c1C)C(N1CC(C)(O)C1)CC2]

The racemate (60 mg) was separated by chiral-HPLC(Column: CHIRALPAK IG-3. 4.6*50 cm, 3 μm; Mobile Phase A: Hex (0.1% DEA)-HPLC, Mobile Phase B: EtOH-HPLC; Flow rate: 20 mL/min; Gradient: 98% B to 98% B in 25 min; 220/254 nm; RT1:15.662 min; RT2:21.512 min) to afford the desired isomer (first eluting peak, 10.0 mg, 16%, 99% e.e.) as a white solid.

¹H NMR (400 MHZ, Methanol-d₄) δ7.23 (d, J=1.6 Hz, 1H), 7.09 (dd, J=8.0, 1.6 Hz, 1H), 7.04 (d, J=8.0 Hz, 1H), 6.92 (s, 1H), 4.22 (d, J=6.4 Hz, 1H), 3.30-3.27 (m, 2H), 3.24-3.23 (m, 1H), 3.19 (d, J=7.6 Hz, 1H), 3.16-3.09 (m, 1H), 2.78-2.72 (m, 1H), 2.40 (s, 3H), 2.28-2.21 (m, 1H), 2.17-2.13 (m, 1H), 2.12 (s, 3H), 2.07-2.02 (m, 1H), 1.45 (s, 3H), 1.08-1.03 (m, 2H), 0.77-0.72 (m, 2H).

LCMS (ESI, m/z): 382 [M+H]⁺. Analytic Conditions: column: HALO C18, 3.0*30 mm, 2.7 μm; mobile phase A: water (0.05% TFA), mobile phase B: acetonitrile (0.05% TFA); flow rate: 1.20 mL/min; gradient: 5% B to 100% B in 1.20 min, hold at 100% for 0.60 min, 100% B to 5% B in 0.03 min; 254 nm; RT: 1.027 min.

### Chiral Separation of 1-(5-(3-chloro-4-cyclopropylphenyl)-6,7-dimethyl-2,3-dihydro-1H-inden-1-yl)-3-methylazetidin-3-ol (27b)

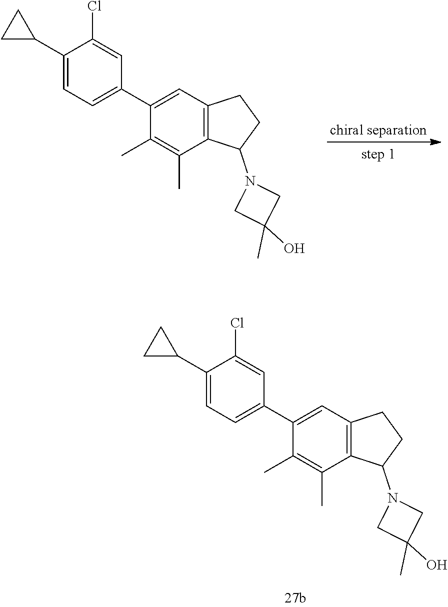 [SMILES: Cc1c(-c2ccc(C3CC3)c(Cl)c2)cc2c(c1C)C(N1CC(C)(O)C1)CC2 ; Cc1c(-c2ccc(C3CC3)c(Cl)c2)cc2c(c1C)C(N1CC(C)(O)C1)CC2]

The racemate (60 mg) was separated by chiral-HPLC(Column: CHIRALPAK IG-3. 4.6*50 cm, 3 μm; Mobile Phase A: Hex (0.1% DEA)-HPLC, Mobile Phase B: EtOH-HPLC; Flow rate: 20 mL/min; Gradient: 98% B to 98% B in 25 min; 220/254 nm; RT1:15.662 min; RT2:21.512 min) to afford the desired isomer (second eluting peak, 8.1 mg, 13%, 99% e.e.) as a white solid.

¹H NMR (400 MHZ, Methanol-d₄) δ7.23 (d, J=1.6 Hz, 1H), 7.09 (dd, J=8.0, 1.6 Hz, 1H), 7.03 (d, J=8.0 Hz, 1H), 6.92 (s, 1H), 4.22 (d, J=6.4 Hz, 1H), 3.32-3.30 (m, 2H), 3.25-3.23 (m, 1H), 3.19 (d, J=7.6 Hz, 1H), 3.13-3.09 (m, 1H), 2.78-2.72 (m, 1H), 2.40 (s, 3H), 2.28-2.21 (m, 1H), 2.16-2.13 (m, 1H), 2.12 (s, 3H), 2.08-2.02 (m, 1H), 1.45 (s, 3H), 1.08-1.03 (m, 2H), 0.77-0.73 (m, 2H).

LCMS (ESI, m/z): 382 [M+H]⁺. Analytic Conditions: column: HALO C18, 3.0*30 mm, 2.7 μm; mobile phase A: water (0.05% TFA), mobile phase B: acetonitrile (0.05% TFA); flow rate: 1.20 mL/min; gradient: 5% B to 100% B in 1.20 min, hold at 100% for 0.60 min, 100% B to 5% B in 0.03 min; 254 nm; RT: 1.024 min.

### Example S28. 1-(4-((2,6-difluoro-phenyl)ethynyl)benzyl)-3-methylazetidin-3-ol (28)

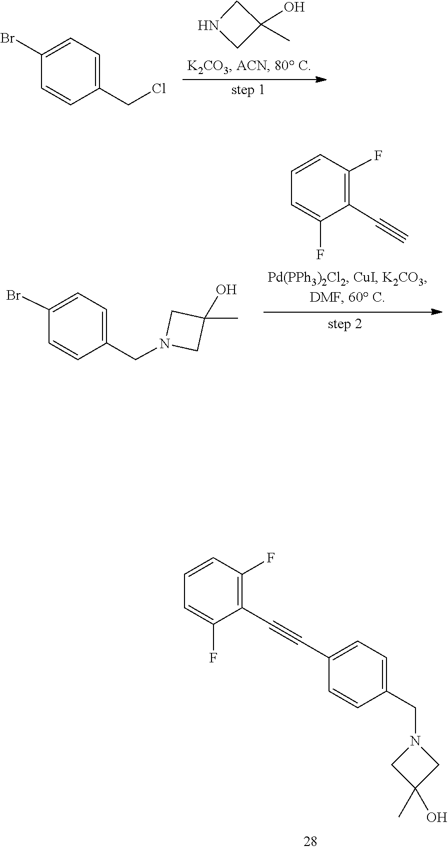

### Synthesis of 1-[(4-bromophenyl)methyl]-3-methyl-azetidin-3-ol

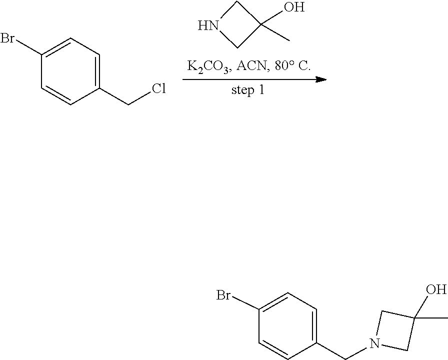

To a stirred solution of 1-bromo-4-(chloromethyl)benzene (1.0 g, 4.87 mmol, 1.00 equiv.) in MeCN (10 mL) were added 3-methylazetidin-3-ol (847 mg, 9.73 mmol, 2.00 equiv.), K2CO₃ (2.1 g, 14.6 mmol, 3.00 equiv.). The reaction was stirred at 80° C. for 3 h. LCMS showed the reaction was completed. The reaction was filtered and the filtrate was concentrated under reduced pressure. The residue was purified by flash column chromatography on C18 silica (eluted with water/acetonitrile, 1/2) to afford 1-[(4-bromophenyl)methyl]-3-methyl-azetidin-3-ol (1.0 g, 80%) as an off-white solid. LCMS (ESI, m z): 256/258 [M+H]⁺.

### Synthesis of 1-[4-[2-(2,6-difluorophenyl)ethynyl|phenyl]methyl]-3-methyl-azetidin-3-ol (28)

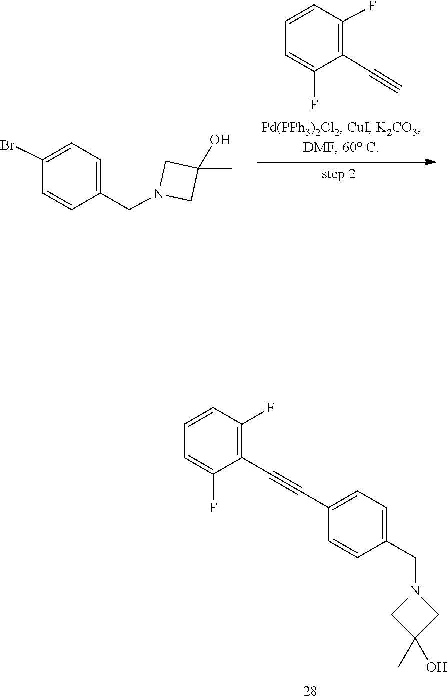

To a stirred solution of 1-[(4-bromophenyl)methyl]-3-methyl-azetidin-3-ol (100 mg, 0.39 mmol, 1.00 equiv.) in DMF (6 mL) were added 2-ethynyl-1,3-difluoro-benzene (108 mg, 0.78 mmol, 2.00 equiv.), Pd(PPh₃)₂Cl₂ (29 mg, 0.04 mmol, 0.10 equiv.), K2CO₃ (162 mg, 1.17 mmol, 3.00 equiv.) and CuI (4 mg, 0.02 mmol, 0.05 equiv.). The reaction was stirred at 60° C. for 3 h. LCMS showed the reaction was completed. The reaction was filtered through Celite and the filtrate was concentrated under reduced pressure. The residue was purified by prep-HPLC (Column: XBridge Prep C18 OBD Column, 19*150 mm 5 μm; Mobile Phase A: water (10 mM NH₄HCO₃), Mobile Phase B: ACN; Flow rate: 25 mL/min; Gradient: 47% B to 67% B in 7 min; 254/210 nm) to afford 1-[[4-[2-(2,6-difluorophenyl)ethynyl]phenyl]methyl]-3-methyl-azetidin-3-ol (50.8 mg, 41%) as an off-white solid.

LCMS (ESI, m/z): 314 [M+H]⁺. Analytic Conditions: column: Shim-pack Scepter C18 3.0*50 mm, 3.0 μm; mobile phase A: water (0.04% NH₃ H₂O), mobile phase B: acetonitrile; flow rate: 1.50 mL/min; gradient: 10% B to 95% B in 2.00 min, hold at 95% for 0.60 min, 95% B to 10% B in 0.20 min; 254 nm; RT: 1.632 min.

¹H NMR (400 MHZ, DMSO-d₆) δ 10.11 (s, 1H), 7.70-7.53 (m, 5H), 7.28 (t, J=8.4 Hz, 2H), 6.10 (s, 1H), 4.46-4.40 (m, 2H), 4.02-3.89 (m, 4H), 1.43 (s, 3H).

### Example S29. 1-(4-((3-fluorophenyl)-ethynyl)benzyl)-3-methylazetidin-3-ol (29)

### Synthesis of 1-[[4-[2-(3-fluorophenyl)ethynyl|phenyl|methyl]-3-methyl-azetidin-3-ol (29)

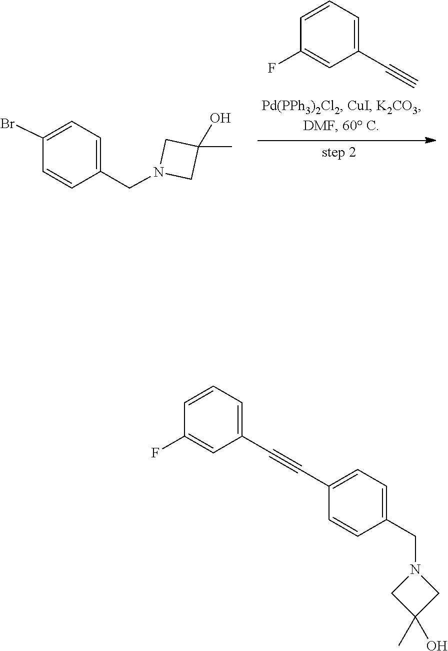

To a stirred solution of 1-[(4-bromophenyl)methyl]-3-methyl-azetidin-3-ol (100 mg, 0.39 mmol, 1.00 equiv.) in DMF (5 mL) were added 1-ethynyl-3-fluoro-benzene (94 mg, 0.78 mmol, 2.00 equiv.), Pd(PPh₃)₂Cl₂ (29 mg, 0.04 mmol, 0.10 equiv.), K2CO₃ (162 mg, 1.17 mmol, 3.00 equiv.) and CuI (4 mg, 0.02 mmol, 0.05 equiv.). The resulting mixture was stirred at 60° C. for 3 h. LCMS showed the reaction was completed. The reaction was filtered through Celite and the filtrate was concentrated under reduced pressure. The residue was purified by prep-HPLC(Column: XBridge Prep C18 OBD Column, 19*150 mm 5 μm; Mobile Phase A: water (10 mM NH₄HCO₃), Mobile Phase B: ACN; Flow rate: 25 mL/min; Gradient: 47% B to 67% B in 7 min; 254/210 nm) to afford 1-[4-[2-(3-fluorophenyl)ethynyl]phenyl]methyl]-3-methyl-azetidin-3-ol (48.1 mg, 41%) as an off-white solid.

LCMS (ESI, m/z): 296 [M+H]⁺. Analytic Conditions: column: Shim-pack Scepter C18 3.0*50 mm, 3.0 μm; mobile phase A: water (0.04% NH₃ H₂O), mobile phase B: Acetonitrile; flow rate: 1.50 mL/min; gradient: 10% B to 95% B in 2.00 min, hold at 95% for 0.60 min, 95% B to 10% B in 0.20 min; 254 nm; RT: 1.673 min.

¹H NMR (400 MHZ, DMSO-d₆) δ 10.35 (s, 1H), 7.65 (d, J=8.0 Hz, 2H), 7.54 (d, J=8.0 Hz, 2H), 7.51-7.47 (m, 1H), 7.45-7.41 (m, 2H), 7.34-7.29 (m, 1H), 6.16 (br, 1H), 4.43 (s, 2H), 4.13-3.86 (m, 4H), 1.43 (s, 3H).

### Example S30. 1-(4-(1-(2,6-dichlorophenyl) azetidin-3-yl)benzyl)-4-methylpiperidin-4-ol (30)

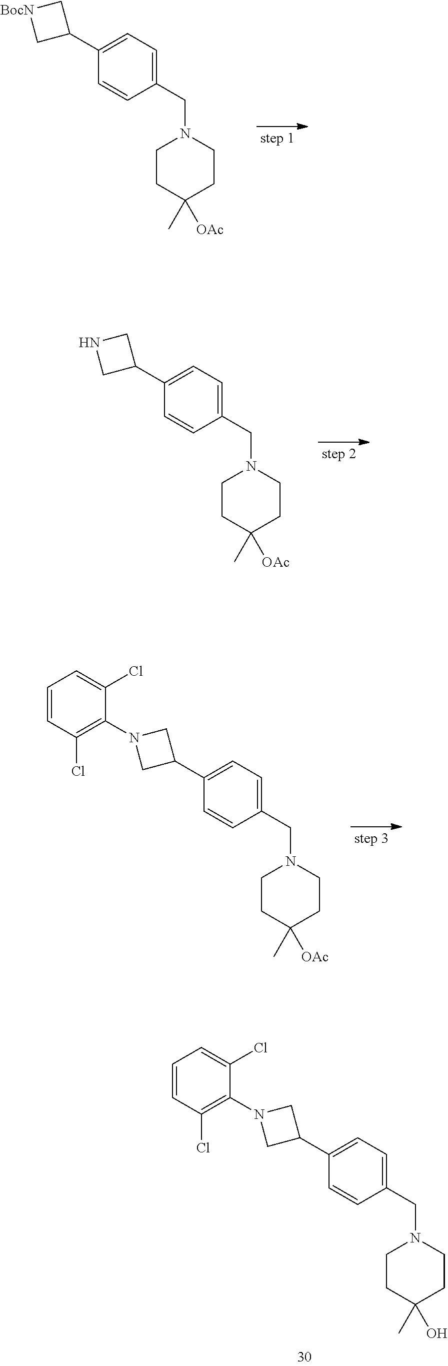 [SMILES: CC(=O)OC1(C)CCN(Cc2ccc(C3CN(C(=O)OC(C)(C)C)C3)cc2)CC1 ; CC(=O)OC1(C)CCN(Cc2ccc(C3CNC3)cc2)CC1 ; CC(=O)OC1(C)CCN(Cc2ccc(C3CN(c4c(Cl)cccc4Cl)C3)cc2)CC1 ; CC1(O)CCN(Cc2ccc(C3CN(c4c(Cl)cccc4Cl)C3)cc2)CC1]

### Synthesis of [1-[[4-(azetidin-3-yl)phenyl]methyl]-4-methyl-4-piperidyl]acetate

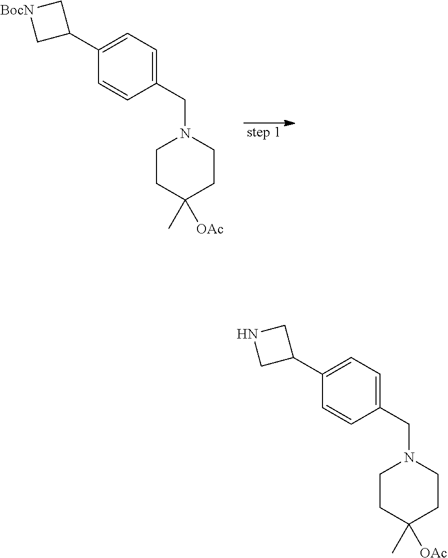

A solution of tert-butyl 3-[4-[(4-acetoxy-4-methyl-1-piperidyl)methyl]phenyl]azetidine-1-carboxylate (1.2 g, 2.98 mmol, 1.00 equiv.) and TBSOTf (2 mL, 11.29 mmol, 3.79 equiv.) in methanol (3 mL) was stirred at room temperature for 2 h. LCMS showed that the reaction was complete. The reaction mixture was concentrated under reduced pressure. The residue was purified by Flash column chromatography on C18 silica (eluted with water (0.05% TFA)/MeCN, 2/1) to afford [1-[4-(azetidin-3-yl)phenyl]methyl]-4-methyl-4-piperidyl]acetate (800 mg, 88.7%) as a yellow oil. LCMS (ESI, m z): 303 [M+H]⁺.

### Synthesis of [1-[[4-[1-(2,6-dichlorophenyl) azetidin-3-yl]phenyl]methyl]-4-methyl-4-piperidyl]acetate

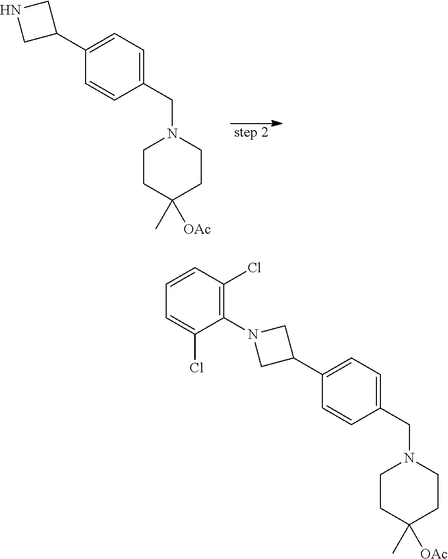

A mixture of [1-[[4-(azetidin-3-yl)phenyl]methyl]-4-methyl-4-piperidyl]acetate (750 mg, 2.48 mmol, 1.00 equiv.), 2-bromo-1,3-dichloro-benzene (1.1 g, 4.96 mmol, 2.00 equiv.), BrettPhos Pd G3 (225 mg, 0.25 mmol, 0.1 equiv.), K2CO₃ (1.0 g, 7.44 mmol, 3.00 equiv.) in tert-butanol (5 mL) was stirred at 80° C. under nitrogen atmosphere overnight. LCMS showed that the reaction was complete. The reaction mixture was concentrated under reduced pressure. The residue was purified by Flash column chromatography on C18 silica (eluted with water (0.05% TFA)/MeCN, 1/3) to afford [1-[4-[1-(2,6-dichlorophenyl) azetidin-3-yl]phenyl]methyl]-4-methyl-4-piperidyl]acetate (450 mg, 40.6%) as a yellow oil. LCMS (ESI, m z): 447 [M+H]⁺.

### Synthesis of 1-[4-[1-(2,6-dichlorophenyl) azetidin-3-yl]phenyl]methyl]-4-methyl-piperidin-4-ol (30)

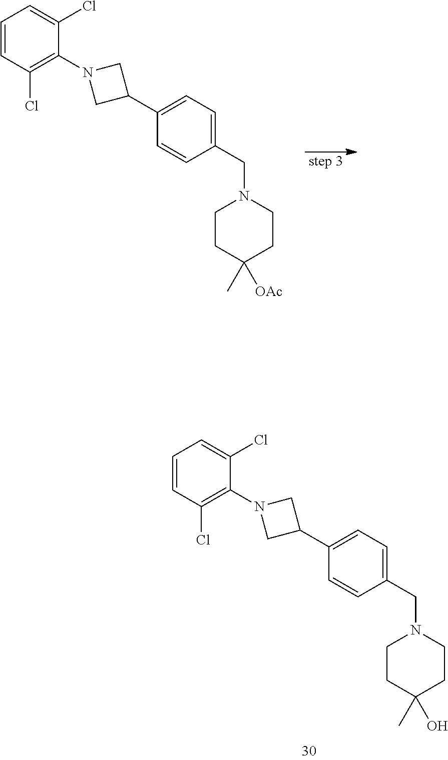 [SMILES: CC(=O)OC1(C)CCN(Cc2ccc(C3CN(c4c(Cl)cccc4Cl)C3)cc2)CC1 ; "step 3" ; CC1(O)CCN(Cc2ccc(C3CN(c4c(Cl)cccc4Cl)C3)cc2)CC1]

To a stirred solution of [1-[[4-[1-(2,6-dichlorophenyl) azetidin-3-yl]phenyl]methyl]-4-methyl-4-piperidyl]acetate (450 mg, 1.01 mmol, 1.00 equiv.) in methanol (2 mL) was added MeONa (2 mL, 10.5 mmol, 30% w in MeOH) dropwise. The resulting reaction was stirred at room temperature for 4h. LCMS showed that the reaction was complete. The reaction mixture was concentrated under reduced pressure. The residue was purified by prep-HPLC(Column: XBridge Prep OBD C18 Column, 30*150 mm, 5 μm; Mobile Phase A: water (10 mmol/L NH₄HCO₃), Mobile Phase B: ACN; Flow rate: 60 mL/min; Gradient: 56% B to 67% B in 10 min; Wave Length: 254/220 nm; RT (min): 9) to afford 1-[[4-[1-(2,6-dichlorophenyl) azetidin-3-yl]phenyl]methyl]-4-methyl-piperidin-4-ol (95.0 mg, 23.2%) as an orange oil.

LCMS (ESI, m/z): 405 [M+H]⁺. Analytic Conditions: column: HALO C18, 3.0*30 mm, 2.7 μm; mobile phase A: water (0.05% TFA), mobile phase B: acetonitrile (0.05% TFA); flow rate: 1.20 mL/min; gradient: 5% B to 70% B in 1.70 min, 70% B to 95% B in 0.30 min, hold at 95% for 0.60 min, 95% B to 5% B in 0.10 min; 254 nm; RT: 1.427 min.

¹H NMR (400 MHZ, Methanol-d₄) δ7.42 (d, J=8.0 Hz, 2H), 7.35 (d, J=8.0 Hz, 2H), 7.19 (d, J=8.0 Hz, 2H), 6.72 (t, J=8.0 Hz, 1H), 4.92-4.89 (m, 2H), 4.44-4.40 (m, 2H), 3.80-3.73 (m, 1H), 3.58 (s, 2H), 2.59-2.49 (m, 4H), 1.68-1.59 (m, 4H), 1.22 (s, 3H).

### Example S31. 1-(4-(1-(2,6-dichlorophenyl) azetidin-3-yl)benzyl)-3-methylazetidin-3-ol (31)

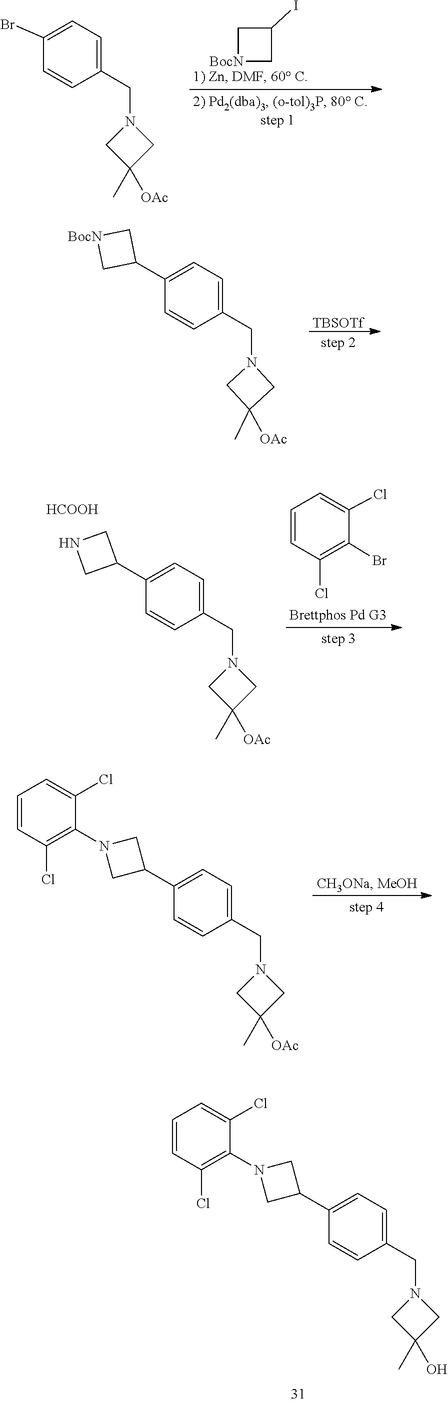

### Synthesis of tert-butyl 3-[4-[(3-acetoxy-3-methyl-azetidin-1-yl)methyl]phenyl]azetidine-1-carboxylate

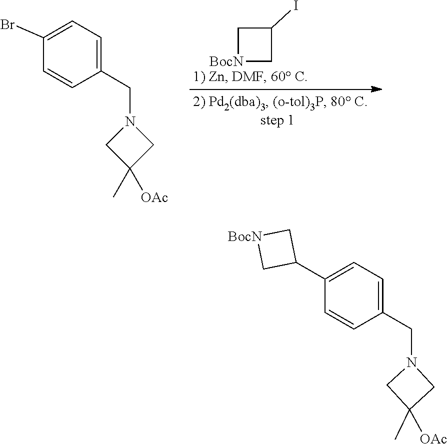 [SMILES: CC(=O)OC1(C)CN(Cc2ccc(Br)cc2)C1 ; CC(C)(C)OC(=O)N1CC(I)C1 ; CC(=O)OC1(C)CN(Cc2ccc(C3CN(C(=O)OC(C)(C)C)C3)cc2)C1]

A solution of tert-butyl 3-iodoazetidine-1-carboxylate (1.1 g, 4.02 mmol, 4.00 equiv.) and Zn (460 mg, 7.04 mmol, 7.00 equiv.) in DMF (15 mL) was stirred at 80° C. for 2 h. Then [1-[(4-bromophenyl)methyl]-3-methyl-azetidin-3-yl]acetate (300 mg, 1.01 mmol, 1.00 equiv.), Pd2 (dba)₃ (92 mg, 0.10 mmol, 0.10 equiv.) and tri-meta-tolylphosphane (61 mg, 0.20 mmol, 0.20 equiv.) were added. The resulting mixture was stirred at 80° C. for 16 h. LCMS showed the reaction was complete. The reaction mixture was filtered through Celite; the filter cake was washed with MeCN (3*10 mL). The filtrate was concentrated under reduced pressure. The residue was purified by Flash column chromatography on C18 silica (eluted with water (5 mM NH₄HCO₃)/MeCN, 1/6) to afford tert-butyl 3-[4-[(3-acetoxy-3-methyl-azetidin-1-yl)methyl]phenyl]azetidine-1-carboxylate (200 mg, 53%) as an off-white solid. LCMS (ESI, m/z): 375 [M+H]⁺.

### Synthesis of [1-[[4-(azetidin-3-yl)phenyl]methyl]-3-methyl-azetidin-3-yl]acetate

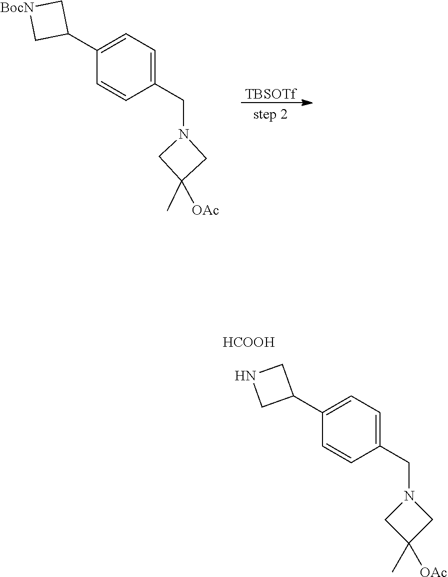 [SMILES: "TBSOTf" ; CC(=O)OC1(C)CN(Cc2ccc(C3CN(C(=O)OC(C)(C)C)C3)cc2)C1 ; "HCOOH" ; CC(=O)OC1(C)CN(Cc2ccc(C3CNC3)cc2)C1]

To a stirred solution of tert-butyl 3-[4-[(3-acetoxy-3-methyl-azetidin-1-yl)methyl]phenyl]azetidine-1-carboxylate (200 mg, 0.53 mmol, 1.00 equiv.) in DCM (4 mL) were added TBSOTf (0.3 mL, 1.60 mmol, 3.00 equiv.). The reaction was stirred at room temperature for 30 min. LCMS showed the reaction was complete. The reaction mixture was concentrated under reduced pressure. The residue was purified by Flash column chromatography on C18 silica (eluted with water (0.05% FA)/MeCN, 7/3) to afford [1-[4-(azetidin-3-yl)phenyl]methyl]-3-methyl-azetidin-3-yl]acetate (140 mg, 95% yield) as an off-white solid. LCMS (ESI, m/z): 275 [M+H]⁺.

### Synthesis of [1-[[4-[1-(2,6-dichlorophenyl) azetidin-3-yl]phenyl]methyl]-3-methyl-azetidin-3-yl]acetate

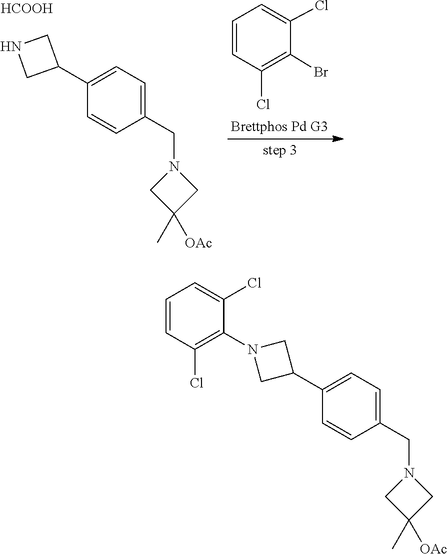

A solution of [1-[4-(azetidin-3-yl)phenyl]methyl]-3-methyl-azetidin-3-yl]acetate (120 mg, 0.44 mmol, 1.00 equiv.), 2-bromo-1,3-dichloro-benzene (148 mg, 0.66 mmol, 1.50 equiv.), BrettPhos Pd G3 (39 mg, 0.04 mmol, 0.10 equiv.) and K₂CO₃ (181 mg, 1.31 mmol, 3.00 equiv.) in tert-butanol (5 mL) was stirred at 80° C. for 2 h. LCMS showed the reaction was completed. The reaction mixture was concentrated under reduced pressure. The residue was purified by Flash column chromatography on C18 silica (eluted with water (0.05% FA)/MeCN, 3/7) to afford [1-[4-[1-(2,6-dichlorophenyl) azetidin-3-yl]phenyl]methyl]-3-methyl-azetidin-3-yl]acetate (60 mg, 32% yield) as an off-white oil. LCMS (ESI, m/z): 419 [M+H]⁺.

### Synthesis of 1-[4-[1-(2,6-dichlorophenyl) azetidin-3-yl]phenyl]methyl]-3-methyl-azetidin-3-ol (31)

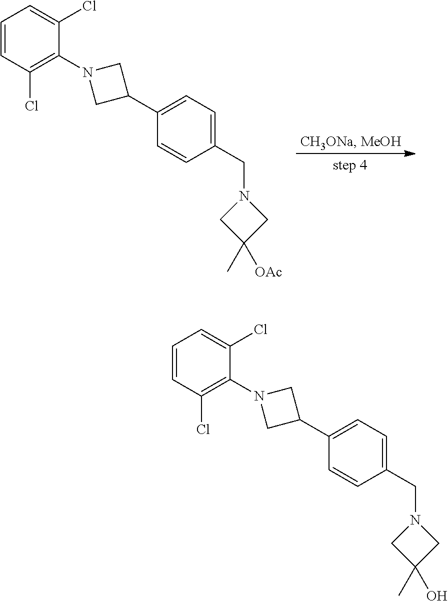

To a stirred solution of [1-[[4-[1-(2,6-dichlorophenyl) azetidin-3-yl]phenyl]methyl]-3-methyl-azetidin-3-yl]acetate (60 mg, 0.14 mmol, 1.00 equiv.) in methanol (2 mL) was added MeONa (30% in MeOH, 1.0 mL, 0.28 mmol, 2.00 equiv.). The mixture solution was stirred at 25° C. for 16 h. LCMS showed the reaction was completed The reaction mixture was concentrated under reduced pressure. The residue was purified by prep-HPLC(Column: Sunfire prep C18 column, 30*150 mm, 5 μm; Mobile Phase A: water (0.1% FA), Mobile Phase B: ACN; Flow rate: 60 mL/min; Gradient: 15% B to 45% B in 7 min; Wave Length: 254/220 nm; RT1 (min): 6.12) to afford 1-[[4-[1-(2,6-dichlorophenyl) azetidin-3-yl]phenyl]methyl]-3-methyl-azetidin-3-ol (33.4 mg, 61%) as an off-white solid.

¹H NMR (400 MHZ, Methanol-d₄) δ8.50 (s, 1H, HFA), 7.55 (d, J=7.6 Hz, 2H), 7.45 (d, J=7.6 Hz, 2H), 7.20 (d, J=8.0 Hz, 2H), 6.73 (t, J=8.0 Hz, 1H), 4.91 (t, J=8.0 Hz, 2H), 4.44-4.41 (m, 2H), 4.26 (s, 2H), 3.95 (d, J=10.4 Hz, 2H), 3.84-3.75 (m, 3H), 1.52 (s, 3H).

LCMS (ESI, m/z): 377 [M+H]⁺. Analytic Conditions: column: HALO C18 Column 3.0*30 mm, 2.0 μm; mobile Phase A: water/0.05% TFA, mobile Phase B: acetonitrile/0.05% TFA; flow rate: 1.20 mL/min; gradient: 5% B to 60% B in 1.8 min, 60% B to 100% B in 0.15 min, hold at 100% for 0.7 min, 100% B to 5% B in 0.15 min; 254 nm; RT: 1.538 min.

### Example S32. 1-(4-(3-(2,6-dichlorophenyl) azetidin-1-yl)benzyl)-4-methylpiperidin-4-ol (32)

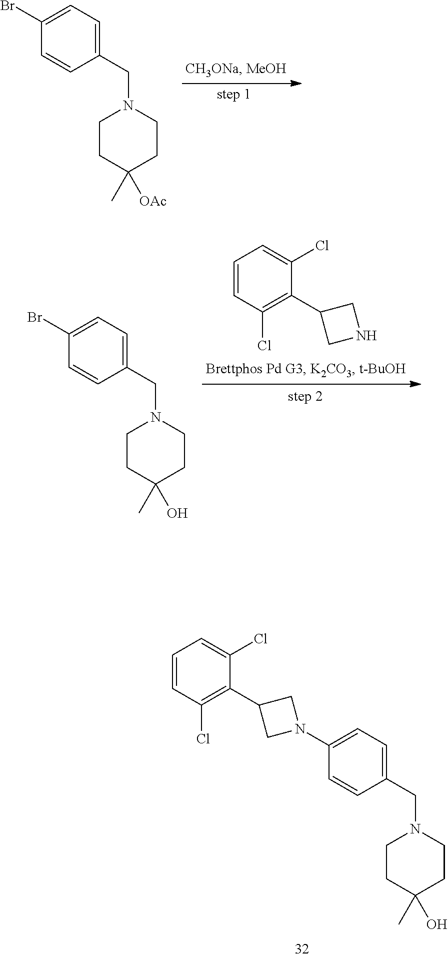 [SMILES: CC(=O)OC1(C)CCN(Cc2ccc(Br)cc2)CC1 ; CC1(O)CCN(Cc2ccc(Br)cc2)CC1 ; Clc1cccc(Cl)c1C1CNC1 ; CC1(O)CCN(Cc2ccc(N3CC(c4c(Cl)cccc4Cl)C3)cc2)CC1]

### Synthesis of 1-[(4-bromophenyl)methyl]-4-methyl-piperidin-4-ol

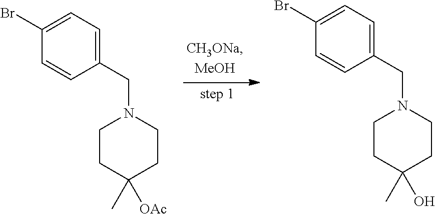 [SMILES: CC(=O)OC1(C)CCN(Cc2ccc(Br)cc2)CC1 ; CC1(O)CCN(Cc2ccc(Br)cc2)CC1]

To a stirred solution of [1-[(4-bromophenyl)methyl]-4-methyl-4-piperidyl]acetate (600 mg, 1.84 mmol, 1.00 equiv.) in methanol (5 mL) were added CH₃ONa (2M in MeOH, 0.9 mL, 1.84 mmol, 1.00 equiv.). The mixture solution was stirred at 25° C. for 16 h. LCMS showed the reaction was completed. The resulting solution was diluted with water (10 mL) and extracted with ethyl acetate (3×10 mL). The organic layers were concentrated under vacuum. The residue was purified by Flash column chromatography on C18 silica (eluted with water (5 mM NH₄HCO₃)/MeCN, 1/3) to afford 1-[(4-bromophenyl)methyl]-4-methyl-piperidin-4-ol (300 mg, 57% yield) as an off-white solid. LCMS (ESI, m/z): 284 [M+H]⁺

### Synthesis of 1-[4-[3-(2,6-dichlorophenyl) azetidin-1-yl]phenyl]methyl]-4-methyl-piperidin-4-ol (32)

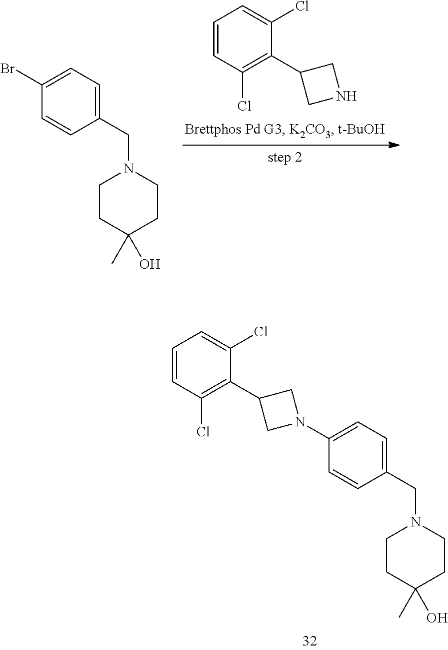 [SMILES: CC1(O)CCN(Cc2ccc(Br)cc2)CC1 ; Clc1cccc(Cl)c1C1CNC1 ; CC1(O)CCN(Cc2ccc(N3CC(c4c(Cl)cccc4Cl)C3)cc2)CC1]

To a stirred solution of 1-[(4-bromophenyl)methyl]-4-methyl-piperidin-4-ol (200 mg, 0.70 mmol, 1.00 equiv.) and 3-(2,6-dichlorophenyl) azetidine (142 mg, 0.70 mmol, 1.00 equiv.) in tert-butanol (4 mL) were added BrettPhos Pd G3 (63 mg, 0.07 mmol, 1.00 equiv.) and K₂CO₃ (291 mg, 2.11 mmol, 1.00 equiv.). The resulting mixture was stirred at 80° C. for 16 h. LCMS showed the reaction was completed. The reaction mixture was concentrated under reduced pressure. The residue was purified by prep-HPLC(Column: Sunfire prep C18 column, 30*150 mm, 5 μm; Mobile Phase A: water (0.1% FA), Mobile Phase B: ACN; Flow rate: 60 mL/min; Gradient: 16% B to 40% B in 7 min; Wave Length: 254/220 nm; RT1:5.7 min) to afford 1-[[4-[3-(2,6-dichlorophenyl) azetidin-1-yl]phenyl]methyl]-4-methyl-piperidin-4-ol (14.6 mg, 5%) as an off-white solid.

¹H NMR (400 MHZ, Methanol-d₄) δ8.54 (s, 1H, HFA), 7.39 (d, J=8.0 Hz, 2H), 7.33 (d, J=8.0 Hz, 2H), 7.23 (t, J=8.4 Hz, 1H), 6.62 (d, J=8.4 Hz, 2H), 4.78-4.69 (m, 1H), 4.50 (t, J=8.0 Hz, 2H), 4.24 (t, J=8.0 Hz, 2H), 4.17 (s, 2H), 3.24-3.18 (m, 4H), 1.82-1.78 (m, 4H), 1.29 (s, 3H).

LCMS (ESI, m/z): 405 [M+H]⁺. Analytic Conditions: column: HALO C18 Column 3.0*30 mm, 2.0 μm; mobile Phase A: water/0.05% TFA, mobile Phase B: acetonitrile/0.05% TFA; flow rate: 1.20 mL/min; gradient: 5% B to 60% B in 1.80 min, 60% B to 100% B in 0.15 min, hold at 100% for 0.70 min, 100% B to 5% B in 0.15 min; 254 nm; RT: 1.540 min.

### Example S33. 1-(4-(3-(2,6-dichlorophenyl) azetidin-1-yl)benzyl)-3-methylazetidin-3-ol (33)

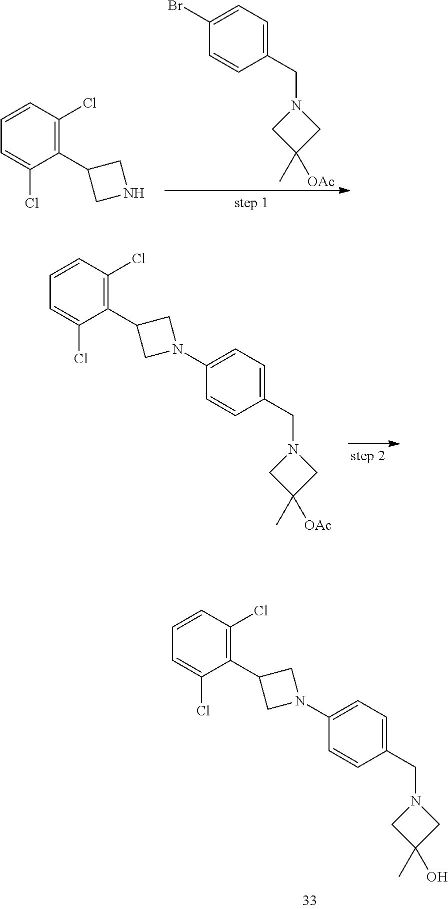 [SMILES: Clc1cccc(Cl)c1C1CNC1 ; CC(=O)OC1(C)CN(Cc2ccc(Br)cc2)C1 ; CC(=O)OC1(C)CN(Cc2ccc(N3CC(c4c(Cl)cccc4Cl)C3)cc2)C1 ; CC1(O)CN(Cc2ccc(N3CC(c4c(Cl)cccc4Cl)C3)cc2)C1]

### Synthesis of [1-[[4-[3-(2,6-dichlorophenyl) azetidin-1-yl]phenyl]methyl]-3-methyl-azetidin-3-yl]acetate

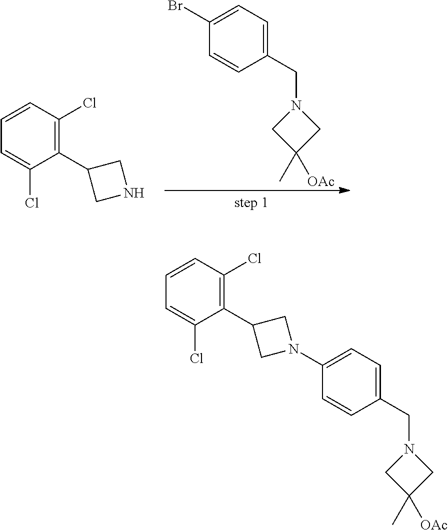 [SMILES: Clc1cccc(Cl)c1C1CNC1 ; CC(=O)OC1(C)CN(Cc2ccc(Br)cc2)C1 ; CC(=O)OC1(C)CN(Cc2ccc(N3CC(c4c(Cl)cccc4Cl)C3)cc2)C1]

To a stirred solution of 3-(2,6-dichlorophenyl) azetidine (150 mg, 0.74 mmol, 1.00 equiv.) in tert-butanol (5 mL) were added [1-[(4-bromophenyl)methyl]-3-methyl-azetidin-3-yl]acetate (443 mg, 1.48 mmol, 2.00 equiv.), BrettPhos Pd G3 (67 mg, 0.07 mmol, 0.10 equiv.) and K₂CO₃ (512 mg, 3.710 mmol, 5.00 equiv.). The reaction was stirred at 90° C. for 3 h. LCMS showed the reaction was completed. The reaction mixture was concentrated under reduced pressure. The residue was purified by Flash column chromatography on C18 silica (eluted with water (5 mM NH₄HCO₃)/MeCN, 1/3) to afford [1-[[4-[3-(2,6-dichlorophenyl) azetidin-1-yl]phenyl]methyl]-3-methyl-azetidin-3-yl]acetate (50 mg, 16%) as an off white solid. LCMS (ESI, m/z): 419 [M+H]⁺

### Synthesis of 1-[4-[3-(2,6-dichlorophenyl) azetidin-1-yl]phenyl]methyl]-3-methyl-azetidin-3-ol (33)

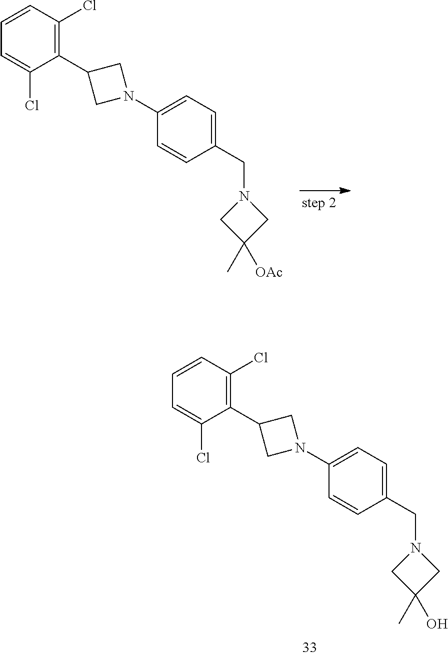 [SMILES: CC(=O)OC1(C)CN(Cc2ccc(N3CC(c4c(Cl)cccc4Cl)C3)cc2)C1 ; CC1(O)CN(Cc2ccc(N3CC(c4c(Cl)cccc4Cl)C3)cc2)C1]

To a stirred solution of [1-[[4-[3-(2,6-dichlorophenyl) azetidin-1-yl]phenyl]methyl]-3-methyl-azetidin-3-yl]acetate (50 mg, 0.12 mmol, 1.00 equiv.) in methanol (3 mL) were added MeONa (2M in MeOH, 0.12 mL, 0.24 mmol, 2.00 equv). The reaction was stirred at room temperature for 16 h. LCMS showed the reaction was completed. The resulting solution was diluted with water (10 mL) and extracted with ethyl acetate (3×10 mL). The organic layers were concentrated under vacuum. The residue was purified by prep-HPLC(Column: XBridge Prep C18 OBD Column, 19×150 mm 5 μm; Mobile Phase A: water (10 mM NH₄HCO₃), Mobile Phase B: ACN; Flow rate: 25 mL/min; Gradient: 47% B to 67% B in 7 min; 254/210 nm) to afford 1-[4-[3-(2,6-dichlorophenyl) azetidin-1-yl]phenyl]methyl]-3-methyl-azetidin-3-ol (29.8 mg, 66%) as an off white solid.

¹H NMR (300 MHZ, DMSO-d₆) δ 8.25 (s, 1H), 7.47 (d, J=7.8 Hz, 2H), 7.30 (t, J=7.8 Hz, 1H), 7.09 (d, J=7.8 Hz, 2H), 6.47 (d, J=7.8 Hz, 2H), 4.59-4.54 (m, 1H), 4.43 (t, J=7.8 Hz, 2H), 3.99 (t, J=7.8 Hz, 2H), 3.49 (s, 2H), 3.16 (d, J=6.6 Hz, 2H), 2.90 (d, J=6.6 Hz, 2H), 1.34 (s, 3H).

LCMS (ESI, m/z): 377 [M+H]⁺. Analytic Conditions: column: HALO C18 3.0*30 mm, 2.0 μm; mobile Phase A: water (0.05% TFA), mobile Phase B: acetonitrile (0.05% TFA); flow rate: 1.50 mL/min; gradient: 5% B to 60% B in 1.80 min, 60% B to 100% B in 0.15 min, hold at 100% for 0.40 min, 100% B to 5% B in 0.70 min; 254 nm; RT: 1.505 min.

### Example S34. 1-(4-(1-(2,6-dichlorophenyl) azetidin-3-yl)-2,6-dimethylbenzyl)-4-methylpiperidin-4-ol (34)

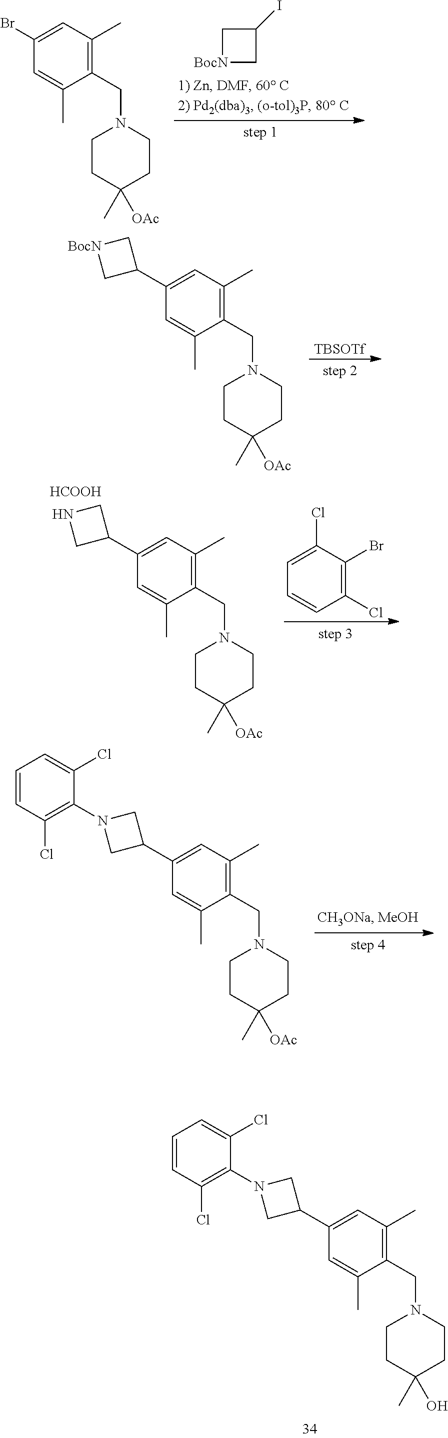

### Synthesis of tert-butyl 3-[4-[(4-acetoxy-4-methyl-1-piperidyl)methyl]-3,5-dimethyl-phenyl]azetidine-1-carboxylate

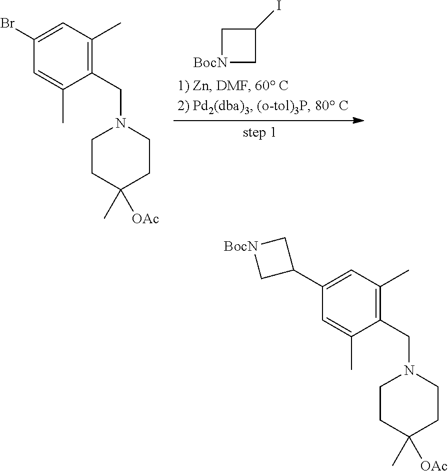 [SMILES: CC(=O)OC1(C)CCN(Cc2c(C)cc(Br)cc2C)CC1 ; CC(C)(C)OC(=O)N1CC(I)C1 ; CC(=O)OC1(C)CCN(Cc2c(C)cc(C3CN(C(=O)OC(C)(C)C)C3)cc2C)CC1]

A solution of tert-butyl 3-iodoazetidine-1-carboxylate (1.9 g, 6.77 mmol, 5.00 equiv.) and Zn (775 mg, 11.9 mmol, 7.00 equiv.) in DMF (30 mL) was stirred at 60° C. for 2 h. Then [1-[(4-bromo-2,6-dimethyl-phenyl)methyl]-4-methyl-4-piperidyl]acetate (600 mg, 1.69 mmol, 1.00 equiv.), Pd2 (dba)₃ (155 mg, 0.17 mmol, 0.10 equiv.) and tri-meta-tolylphosphane (102 mg, 0.34 mmol, 0.20 equiv.) were added. The resulting mixture was stirred at 80° C. for 16 h. LCMS showed the reaction was completed. The reaction mixture was filtered through Celite; the filter cake was washed with MeCN (3*10 mL). The filtrate was concentrated under reduced pressure. The residue was purified by Flash column chromatography on C18 silica (eluted with water (5 mM NH+HCO₃)/MeCN, 1/6) to afford tert-butyl 3-[4-[(4-acetoxy-4-methyl-1-piperidyl)methyl]-3,5-dimethyl-phenyl]azetidine-1-carboxylate (700 mg, 95%) as an off-white solid. LCMS (ESI, m/z): 431 [M+H]⁺.

### Synthesis of [1-[[4-(azetidin-3-yl)-2,6-dimethyl-phenyl]methyl]-4-methyl-4-piperidyl]acetate

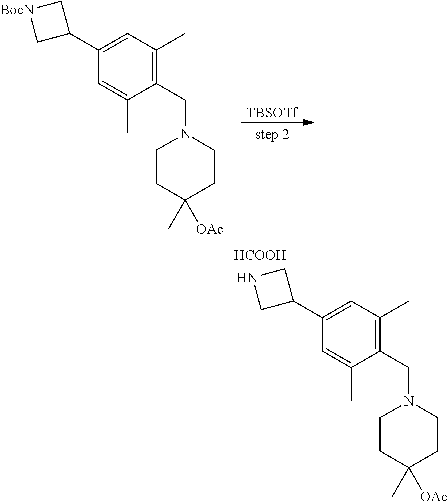 [SMILES: "TBSOTf" ; CC(=O)OC1(C)CCN(Cc2c(C)cc(C3CN(C(=O)OC(C)(C)C)C3)cc2C)CC1 ; "HCOOH" ; CC(=O)OC1(C)CCN(Cc2c(C)cc(C3CNC3)cc2C)CC1]

To a stirred solution of tert-butyl 3-[4-[(4-acetoxy-4-methyl-1-piperidyl)methyl]-3,5-dimethyl-phenyl]azetidine-1-carboxylate (700 mg, 1.63 mmol, 1.00 equiv.) in DCM (10 mL) was added TBSOTf (0.8 mL, 4.880 mmol, 3.00 equiv.). The mixture solution was stirred at 0° C. for 2 h. LCMS showed the reaction was completed. The reaction mixture was concentrated under reduced pressure. The residue was purified by Flash column chromatography on C18 silica (eluted with water (0.05% FA)/MeCN, 9/1) to afford [1-[4-(azetidin-3-yl)-2,6-dimethyl-phenyl]methyl]-4-methyl-4-piperidyl]acetate (500 mg, 93% yield) as an off-white solid. LCMS (ESI, m/z): 331 [M+H]⁺

### Synthesis of [1-[[4-[1-(2,6-dichlorophenyl) azetidin-3-yl]-2,6-dimethyl-phenyl methyl]-4-methyl-4-piperidyl]acetate

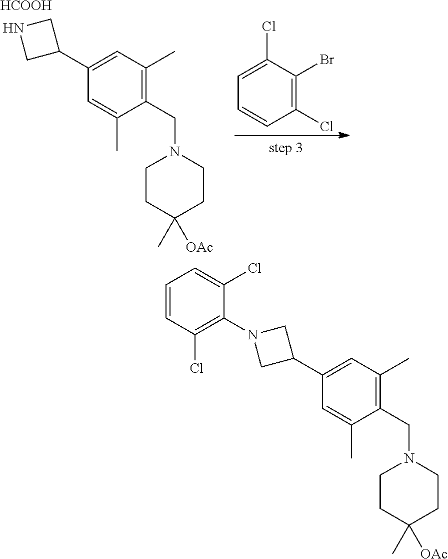

To a stirred solution of [1-[4-(azetidin-3-yl)-2,6-dimethyl-phenyl]methyl]-4-methyl-4-piperidyl]acetate (300 mg, 0.91 mmol, 1.00 equiv.) and 2-bromo-1,3-dichloro-benzene (307 mg, 1.36 mmol, 1.50 equiv.) in tert-butanol (5 mL) were added BrettPhos Pd G3 (82 mg, 0.09 mmol, 0.10 equiv.) and K₂CO₃ (375 mg, 2.72 mmol, 3.00 equiv.). The mixture solution was stirred at 80° C. for 16 h. LCMS showed the reaction was completed. The reaction mixture was concentrated under reduced pressure. The residue was purified by Flash column chromatography on C18 silica (eluted with water (0.05% FA)/MeCN, 3/7) to afford [1-[[4-[1-(2,6-dichlorophenyl) azetidin-3-yl]-2,6-dimethyl-phenyl]methyl]-4-methyl-4-piperidyl]acetate (120 mg, 27% yield) as an off-white solid. LCMS (ESI, m/z): 475 [M+H]⁺.

### Synthesis of 1-[[4-[1-(2,6-dichlorophenyl) azetidin-3-yl]-2,6-dimethyl-phenyl]methyl]-4-methyl-piperidin-4-ol (34)

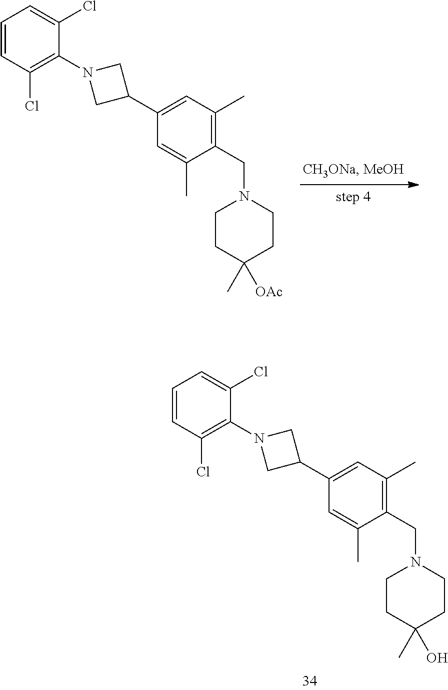 [SMILES: CC(=O)OC1(C)CCN(Cc2c(C)cc(C3CN(c4c(Cl)cccc4Cl)C3)cc2C)CC1 ; Cc1cc(C2CN(c3c(Cl)cccc3Cl)C2)cc(C)c1CN1CCC(C)(O)CC1]

To a stirred solution of [1-[[4-[1-(2,6-dichlorophenyl) azetidin-3-yl]-2,6-dimethyl-phenyl]methyl]-4-methyl-4-piperidyl]acetate (120 mg, 0.25 mmol, 1.00 equiv.) in methanol (1 mL) was added CH₃ONa (1M in MeOH, 0.3 mL, 0.25 mmol, 1.00 equiv.). The mixture solution was stirred at 25° C. for 16 h. LCMS showed the reaction was completed. The resulting solution was diluted with water (10 mL) and extracted with ethyl acetate (3×10 mL). The organic layers were concentrated under vacuum. The residue was purified by prep-HPLC(Column: XBridge Prep OBD C18 Column, 30*150 mm, 5 μm; Mobile Phase A: water (10 mmol/L NH₄HCO₃), Mobile Phase B: ACN; Flow rate: 60 mL/min; Gradient: 72% B to 90% B in 8 min; Wave Length: 254/220 nm; RT: 7.3 min) to afford 1-[[4-[1-(2,6-dichlorophenyl) azetidin-3-yl]-2,6-dimethyl-phenyl]methyl]-4-methyl-piperidin-4-ol (38.2 mg, 34%) as an off-white solid. ¹H NMR (400 MHZ, DMSO-d₆) δ 7.23 (d, J=8.0 Hz, 2H), 7.02 (s, 2H), 6.74 (t, J=8.0 Hz, 1H), 4.80 (t, J=8.0 Hz, 2H), 4.33 (t, J=7.2 Hz, 2H), 4.08 (s, 1H), 3.71-3.63 (m, 1H), 3.38 (s, 2H), 2.43-2.35 (m, 3H), 2.33 (s, 6H), 2.32-2.29 (m, 1H)1.44-1.32 (m, 4H), 1.08 (s, 3H).

LCMS (ESI, m/z): 433 [M+H]⁺. Analytic Conditions: column: HALO C18 Column 2.0*30 mm, 2.0 μm; mobile Phase A: water/0.05% TFA, mobile Phase B: acetonitrile/0.05% TFA; flow rate: 1.20 mL/min; gradient: 5% B to 100% B in 1.20 min, hold at 100% for 0.60 min, 100% B to 5% B in 0.03 min; 254 nm; RT: 0.917 min.

### Example S35. 1-(4-((2,6-dichlorophenyl)ethynyl)benzyl)-3-methylazetidin-3-ol (35)

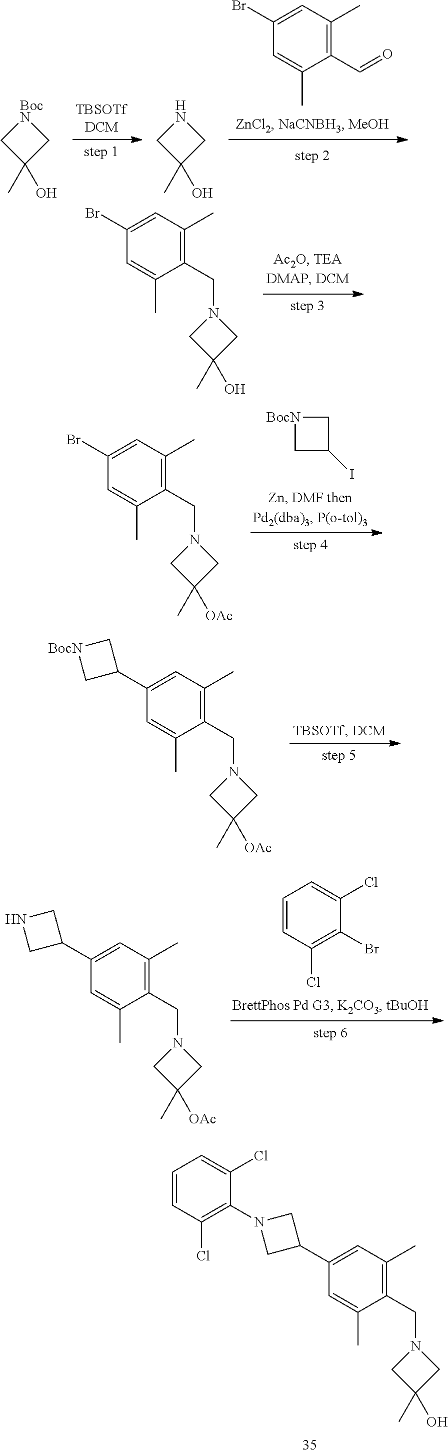

### Synthesis of 3-methylazetidin-3-ol

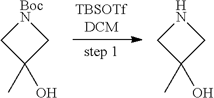

To a stirred solution/mixture of tert-butyl 3-hydroxy-3-methylazetidine-1-carboxylate (3.00 g, 16.0 mmol, 1.00 equiv.) in DCM (10 mL) was added TBSOTf (4.24 g, 16.0 mmol, 1.00 equiv.) dropwise at room temperature. The resulting mixture was stirred for 1 h at room temperature. LCMS showed the reaction was complete. The resulting mixture was concentrated under reduced pressure. The crude product 3-methylazetidin-3-ol (1.2 g) was used in the next step directly without further purification.

### Synthesis of 1-[(4-bromo-2,6-dimethylphenyl)methyl]-3-methylazetidin-3-ol

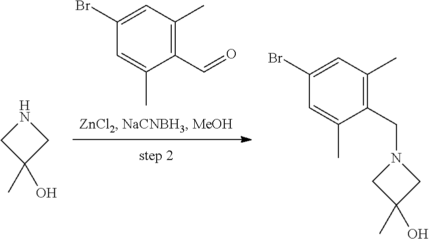

To a stirred solution of 3-methylazetidin-3-ol (1.00 g, 11.5 mmol, 1.00 equiv.) and 4-bromo-2,6-dimethylbenzaldehyde (2.45 g, 11.5 mmol, 1.00 equiv.) in MeOH(10 mL) were added NaBH₃CN (0.72 g, 11.5 mmol, 1.00 equiv.) and ZnCl₂ (5.8 mL, 2.0M in 2Me-THF, 11.5 mmol, 1.00 equiv.) in portions at room temperature. The resulting mixture was stirred overnight at 60° C. under nitrogen atmosphere. LCMS showed the reaction was complete. The reaction mixture was filtered through Celite; the filter cake was washed with MeOH(3*10 mL). The filtrate was concentrated under reduced pressure. The residue was purified by Flash column chromatography on C18 silica (eluted with water (5 mM NH+HCO₃)/MeCN, 3/7) to afford 1-[(4-bromo-2,6-dimethylphenyl)methyl]-3-methylazetidin-3-ol (1.8 g, 55.2%) as a light yellow oil. LCMS (ESI, m z): 284 [M+H]⁺.

### Synthesis of [1-[(4-bromo-2,6-dimethyl-phenyl)methyl]-3-methyl-azetidin-3-yl]acetate

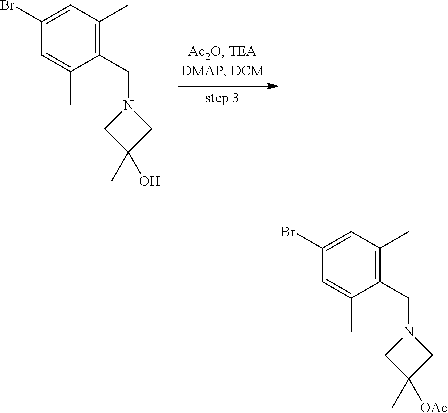 [SMILES: Cc1cc(Br)cc(C)c1CN1CC(C)(O)C1 ; CC(=O)OC1(C)CN(Cc2c(C)cc(Br)cc2C)C1]

To a stirred solution of 1-[(4-bromo-2,6-dimethyl-phenyl)methyl]-3-methyl-azetidin-3-ol (820 mg, 2.89 mmol, 1.00 equiv.) in DCM (10 mL) were added TEA (1.0 mL, 5.77 mmol, 2.00 equiv.), AczO(1.1 mL, 11.54 mmol, 4.00 equiv.) and DMAP (35 mg, 0.29 mmol, 0.10 equiv.). The reaction was stirred at room temperature overnight. LCMS showed the reaction was complete. The reaction mixture was concentrated under reduced pressure. The residue was purified by Flash column chromatography on C18 silica (eluted with water (5 mM NH₄HCO₃)/MeCN, 1/9) to afford [1-[(4-bromo-2,6-dimethyl-phenyl)methyl]-3-methyl-azetidin-3-yl]acetate (830 mg, 88.0%) as an off white solid. LCMS (ESI, m z): 326 [M+H]⁺.

### Synthesis of tert-butyl 3-[4-[(3-acetoxy-3-methyl-azetidin-1-yl)methyl]-3,5-dimethyl-phenyl]azetidine-1-carboxylate

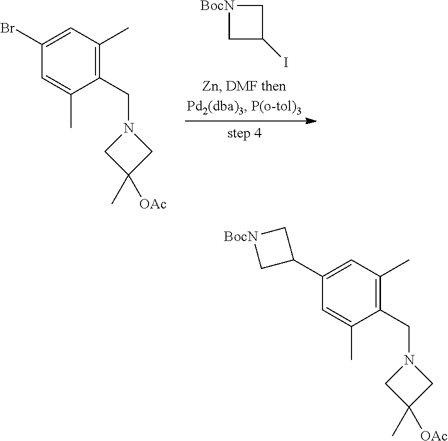 [SMILES: CC(=O)OC1(C)CN(Cc2c(C)cc(Br)cc2C)C1 ; CC(C)(C)OC(=O)N1CC(I)C1 ; CC(=O)OC1(C)CN(Cc2c(C)cc(C3CN(C(=O)OC(C)(C)C)C3)cc2C)C1]

A mixture of [1-[(4-bromo-2,6-dimethyl-phenyl)methyl]-3-methyl-azetidin-3-yl]acetate (430 mg, 1.32 mmol, 1.00 equiv.) and Zinc powder (603 mg, 9.23 mmol, 7.00 equiv.) in DMF (3 mL) was stirred at 60° C. for 2 h. Then Pd2 (dba)₃ (121 mg, 0.13 mmol, 0.10 equiv.) and tri (o-tolyl)phosphine (39 mg, 0.13 mmol, 0.10 equiv.) were added at room temperature. The resulting mixture was stirred at 80° C. for 3 h. LCMS showed the reaction was complete. The reaction mixture was filtered through Celite; the filter cake was washed with MeCN (3*10 mL). The filtrate was concentrated under reduced pressure. The residue was purified by Flash column chromatography on C18 silica (eluted with water (5 mM NH₄HCO₃)/MeCN, 1/6) to afford tert-butyl 3-[4-[(3-acetoxy-3-methyl-azetidin-1-yl)methyl]-3,5-dimethyl-phenyl]azetidine-1-carboxylate (210 mg, 39%) as an off-white solid. LCMS (ESI, m z): 403 [M+H]⁺.

### Synthesis of [1-[[4-(azetidin-3-yl)-2,6-dimethyl-phenyl]methyl]-3-methyl-azetidin-3-yl]acetate

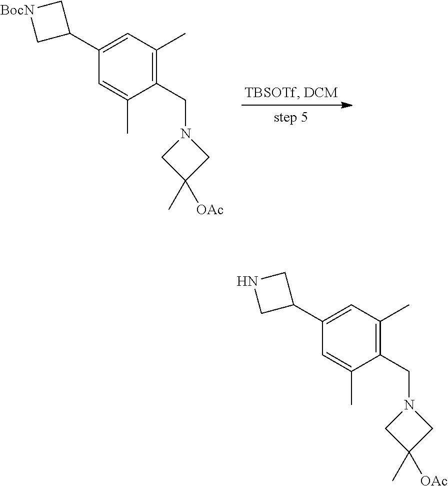 [SMILES: CC(=O)OC1(C)CN(Cc2c(C)cc(C3CN(C(=O)OC(C)(C)C)C3)cc2C)C1 ; CC(=O)OC1(C)CN(Cc2c(C)cc(C3CNC3)cc2C)C1]

To a stirred solution of tert-butyl 3-[4-[(3-acetoxy-3-methyl-azetidin-1-yl)methyl]-3,5-dimethyl-phenyl]azetidine-1-carboxylate (210 mg, 0.52 mmol, 1.00 equiv.) in DCM (2 mL) was added TBSOTf (137 mg, 0.52 mmol, 1.00 equiv.). The reaction was stirred at room temperature for 30 min. LCMS showed the reaction was complete. The reaction mixture was concentrated under reduced pressure. The residue was purified by Flash column chromatography on C18 silica (eluted with water (0.05% FA)/MeCN, 6/1) to afford [1-[4-(azetidin-3-yl)-2,6-dimethyl-phenyl]methyl]-3-methyl-azetidin-3-yl]acetate (150 mg, 95%) as an light yellow oil. LCMS (ESI, m z): 303 [M+H]⁺.

### Synthesis of 1-[[4-[1-(2,6-dichlorophenyl) azetidin-3-yl]-2,6-dimethyl-phenyl]methyl]-3-methyl-azetidin-3-ol (35)

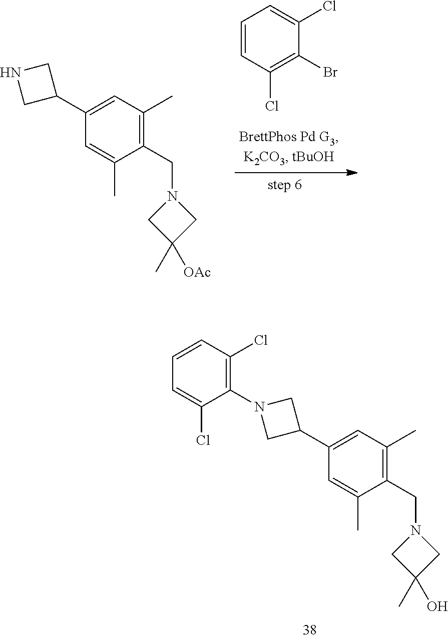 [SMILES: CC(=O)OC1(C)CN(Cc2c(C)cc(C3CNC3)cc2C)C1 ; Clc1cccc(Cl)c1Br ; Cc1cc(C2CN(c3c(Cl)cccc3Cl)C2)cc(C)c1CN1CC(C)(O)C1]

To a stirred solution of [1-[4-(azetidin-3-yl)-2,6-dimethyl-phenyl]methyl]-3-methyl-azetidin-3-yl]acetate (150 mg, 0.50 mmol, 1.00 equiv.) in tert-butanol (3 mL) were added BrettPhos Pd G3 (45 mg, 0.05 mmol, 0.10 equiv.), K₂CO₃ (342 mg, 2.48 mmol, 5.00 equiv.). The reaction was stirred at 60° C. for 3 h. LCMS showed the reaction was complete. The reaction mixture was concentrated under reduced pressure. The residue was purified by prep-HPLC(Column: XBridge Prep OBD C18 Column, 30*150 mm, 5 μm; Mobile Phase A: water (10 mM NH₄HCO₃), Mobile Phase B: ACN; Flow rate: 60 mL/min; Gradient: 68% B to 85% B in 9 min; Wavelength: 254/220 nm; RT: 8.22 min) to afford 1-[[4-[1-(2,6-dichlorophenyl) azetidin-3-yl]-2,6-dimethyl-phenyl]methyl]-3-methyl-azetidin-3-ol (16.9 mg, 8%) as an off white solid.

LCMS (ESI, m/z): 405 [M+H]⁺. Analytic Conditions: column: Shim-pack Scepter C18 3.0*50 mm, 3.0 μm; mobile Phase A: water (0.04% NH₃ H₂O), mobile Phase B: Acetonitrile; flow rate: 1.50 mL/min; gradient: 60% B to 95% B in 2.00 min, hold at 95% for 0.80 min, 95% B to 10% B in 0.10 min; 254 nm; RT: 1.685 min.

¹H NMR (400 MHZ, DMSO-d₆) δ 7.24 (d, J=8.0 Hz, 2H), 7.01 (s, 2H), 6.74 (t, J=8.0 Hz, 1H), 5.11 (s, 1H), 4.80 (t, J=8.0 Hz, 2H), 4.34-4.30 (m, 2H), 3.70-3.62 (m, 1H), 3.56 (s, 2H), 3.07-3.05 (m, 2H), 2.94-2.92 (m, 2H), 2.35 (s, 6H), 1.29 (s, 3H).

### Example S36. 1-(4-(3-(2,6-dichlorophenyl) azetidin-1-yl)-2,6-dimethylbenzyl)-4-methylpiperidin-4-ol (36)

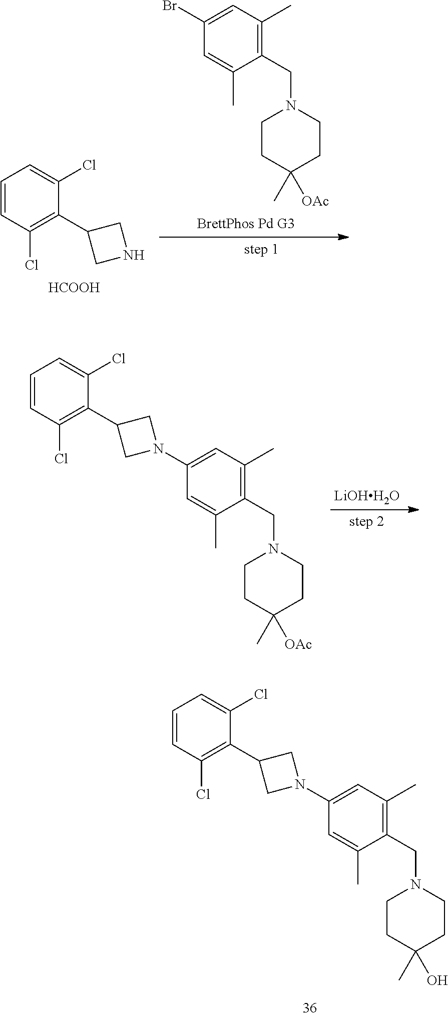 [SMILES: Clc1cccc(Cl)c1C1CNC1 ; CC(=O)OC1(C)CCN(Cc2c(C)cc(Br)cc2C)CC1 ; "HCOOH" ; CC(=O)OC1(C)CCN(Cc2c(C)cc(N3CC(c4c(Cl)cccc4Cl)C3)cc2C)CC1 ; "LiOH·H2O" ; Cc1cc(N2CC(c3c(Cl)cccc3Cl)C2)cc(C)c1CN1CCC(C)(O)CC1]

### Synthesis of [1-[[4-[3-(2,6-dichlorophenyl) azetidin-1-yl]-2,6-dimethyl-phenylmethyl]-4-methyl-4-piperidyl]acetate

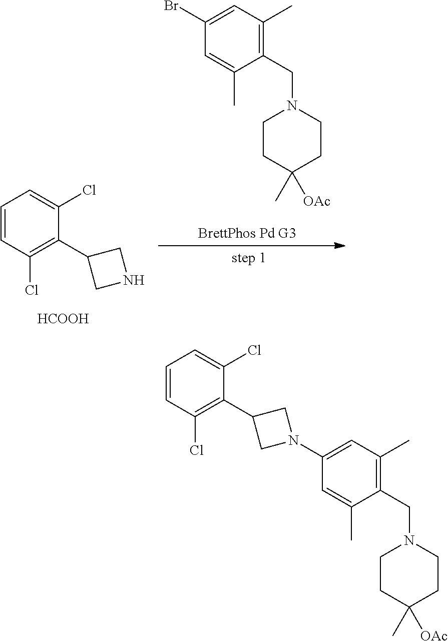 [SMILES: Clc1cccc(Cl)c1C1CNC1 ; CC(=O)OC1(C)CCN(Cc2c(C)cc(Br)cc2C)CC1 ; "HCOOH" ; CC(=O)OC1(C)CCN(Cc2c(C)cc(N3CC(c4c(Cl)cccc4Cl)C3)cc2C)CC1]

To a stirred solution of [1-[(4-bromo-2,6-dimethyl-phenyl)methyl]-4-methyl-4-piperidyl]acetate (140 mg, 0.40 mmol, 1.00 equiv.) and 3-(2,6-dichlorophenyl) azetidine (79 mg, 0.40 mmol, 1.00 equiv.) in tert-butanol (4 mL) were added BrettPhos Pd G3 (35 mg, 0.04 mmol, 0.10 equiv.) and K₂CO₃ (163 mg, 1.19 mmol, 3.00 equiv.). The mixture solution was stirred at 80° C. for 16 h. LCMS showed the reaction was completed. The reaction mixture was concentrated under reduced pressure. The residue was purified by Flash column chromatography on C18 silica (eluted with water (5 mM NH₄HCO₃)/MeCN, 1/3) to afford [1-[4-[3-(2,6-dichlorophenyl) azetidin-1-yl]-2,6-dimethyl-phenyl]methyl]-4-methyl-4-piperidyl]acetate (60 mg, 31%) as an off-white solid. LCMS (ESI, m/z): 475 [M+H]⁺.

### Synthesis of 1-[[4-[3-(2,6-dichlorophenyl) azetidin-1-yl]-2,6-dimethyl-phenylmethyl]-4-methyl-piperidin-4-ol (36)

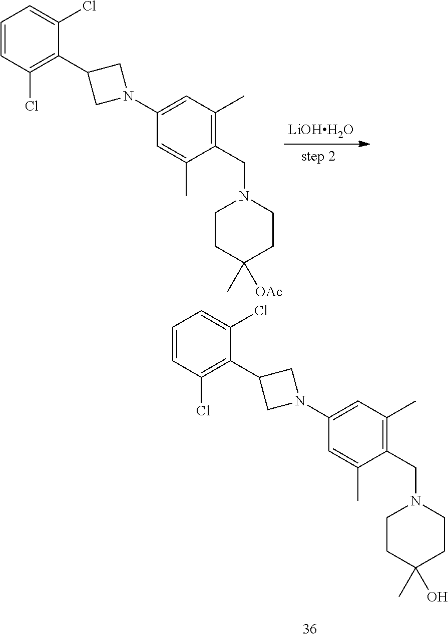

To a stirred solution of [1-[[4-[3-(2,6-dichlorophenyl) azetidin-1-yl]-2,6-dimethyl-phenyl]methyl]-4-methyl-4-piperidyl]acetate (60 mg, 0.13 mmol, 1.00 equiv.) in methanol (2 mL) were added CH₃ONa (2M in MeOH, 0.5 mL, 1.04 mmol, 8.00 equiv.). The mixture solution was stirred at 25° C. for 16 h. LCMS showed the reaction was completed. The resulting solution was diluted with water (10 mL) and extracted with ethyl acetate (3×10 mL). The organic layers were concentrated under vacuum. The residue was purified by prep-HPLC (Column: XBridge Prep OBD C18 Column, 30*150 mm, 5 μm; Mobile Phase A: water (10 mmol/L NH₄HCO₃), Mobile Phase B: ACN; Flow rate: 60 mL/min; Gradient: 72% B to 90% B in 8 min; Wave Length: 254/220 nm; RT: 7.3 min) to afford 1-[[4-[3-(2,6-dichlorophenyl) azetidin-1-yl]-2,6-dimethyl-phenyl]methyl]-4-methyl-piperidin-4-ol (6.4 mg, 11%) as an off-white solid.

¹H NMR (300 MHZ, Methanol-d₄) δ7.38 (d, J=8.1 Hz, 2H), 7.21 (d, J=8.1 Hz, 1H), 6.26 (s, 2H), 4.60-4.52 (m, 1H), 4.46 (t, J=7.5 Hz, 2H), 4.05 (t, J=7.5 Hz, 2H), 3.47 (s, 2H), 2.55-2.49 (m, 4H), 2.34 (s, 6H), 1.59-1.56 (m, 4H), 1.20 (d, 3H).

LCMS (ESI, m/z): 433 [M+H]⁺. Analytic Conditions: column: HALO C18 Column 3.0*30 mm, 2.0 μm; mobile Phase A: water/0.05% TFA, mobile Phase B: acetonitrile/0.05% TFA; flow rate: 1.2000 mL/min; gradient: 5% B to 100% B in 1.2 min, hold at 100% for 0.6 min, 100% B to 5% B in 0.03 min; 254 nm; RT: 0.902 min.

### Example S37. 1-(4-(3-(2,6-dichlorophenyl) azetidin-1-yl)-2,6-dimethylbenzyl)-3-methylazetidin-3-ol (37)

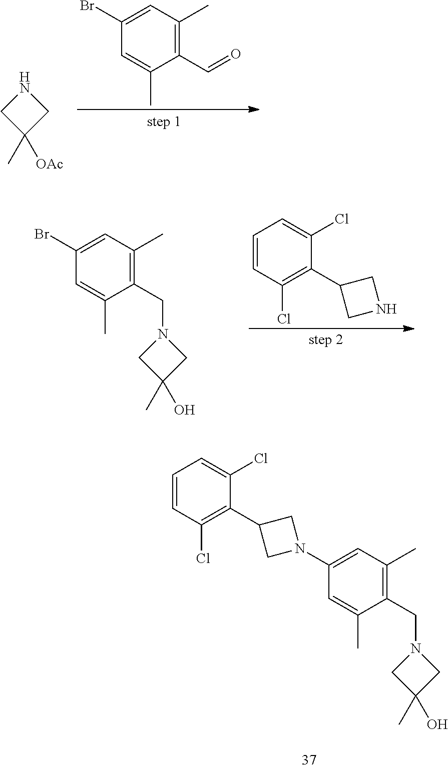

### Synthesis of 1-(4-bromo-2,6-dimethylbenzyl)-3-methylazetidin-3-ol

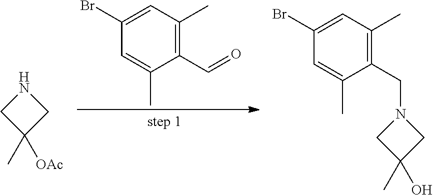

To a solution of ZnCl₂ (2 M in THF, 7.75 mL, 15.5 mmol, 2.00 equiv.) in methanol (5.0 mL) was added NaBH₃CN (1.95 g, 31.0 mmol, 4.00 equiv.). The solution was stirred for 10 min at room temperature. Then 3-methylazetidin-3-yl acetate (1.0 g, 7.75 mmol, 1.00 equiv.) and 4-bromo-2,6-dimethylbenzaldehyde (1.7 g, 7.75 mmol, 1.00 equiv.) were added to the above solution. The reaction was stirred at 80° C. for 3 h. LCMS showed the reaction was completed. The resulting solution was diluted with 10 ml of water and extracted with ethyl acetate (3*30 mL). The combined organic layers were washed with brine, and dried over anhydrous Na₂SO₄ and concentrated under reduced pressure to give 1-(4-bromo-2,6-dimethylbenzyl)-3-methylazetidin-3-ol (500 mg, 23%) as a yellow oil. LCMS (ESI, m z): 284 [M+H]⁺.

### Synthesis of 1-[[4-[3-(2,6-dichlorophenyl) azetidin-1-yl]-2,6-dimethyl-phenyl]methyl]-3-methyl-azetidin-3-ol (37)

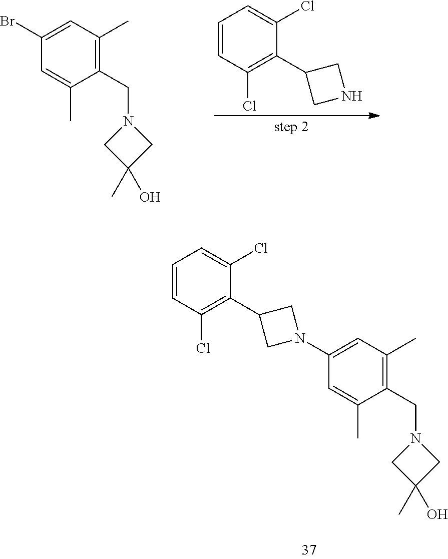 [SMILES: Cc1cc(Br)cc(C)c1CN1CC(C)(O)C1 ; Clc1cccc(Cl)c1C1CNC1 ; Cc1cc(N2CC(c3c(Cl)cccc3Cl)C2)cc(C)c1CN1CC(C)(O)C1]

To a solution of the compound 1-[(4-bromo-2,6-dimethyl-phenyl)methyl]-3-methyl-azetidin-3-ol (127 mg, 0.45 mmol, 1.00 equiv.) in tert-butanol (2.0 mL) were added 3-(2,6-dichlorophenyl) azetidine (60 mg, 0.30 mmol, 0.67 equiv.), K₂CO₃ (123 mg, 0.89 mmol, 2.00 equiv.) and BrettPhos Pd G3 (27 mg, 0.03 mmol, 0.067 equiv.) under a nitrogen atmosphere. The reaction was stirred for 12 h at 80° C. LCMS showed desired product was formed. The residue was purified by Prep_HPLC(Column: XBridge Prep OBD C18 Column, 30*150 mm, 5 μm; Mobile Phase A: Water (10 mmol/L NH+HCO₃), Mobile Phase B: ACN; Flow rate: 60 mL/min; Gradient: 49% B to 74% B in 10 min; Wave Length: 254/220 nm) to afford Purification resulted in 1-[4-[3-(2,6-dichlorophenyl) azetidin-1-yl]-2,6-dimethyl-phenyl]methyl]-3-methyl-azetidin-3-ol (16.6 mg, 13.5%) as an off-white solid.

LCMS (ESI, m/z): 405 [M+H]⁺. Analytic Conditions: Shim-pack Scepter C18, 3.0*33 mm, 3.0 μm; Mobile Phase A: Water/5 mM NH₄HCO₃. Mobile Phase B: ACN; Flow rate: 1.50 mL/min; Gradient: 50% B to 95% B in 2.0 min, hold at 95% B for 0.7 min, 95% B to 15% B in 0.15 min; 254 nm; RT: 1.164 min.

¹H NMR (400 MHz, DMSO-d₆) δ 7.45 (d, J=8.0 Hz, 2H), 7.29 (t, J=8.0 Hz, 1H), 6.15 (s, 2H), 5.07 (s, 1H), 4.51 (q, J=8.0 Hz, 1H), 4.39 (t, J=7.6 Hz, 2H), 3.95 (t, J=7.6 Hz, 2H), 3.46 (s, 2H), 3.03 (d, J=6.0 Hz, 2H), 2.88 (d, J=6.0 Hz, 2H), 2.28 (s, 6H), 1.29 (s, 3H). LC-MS Methods for the following examples

Method 1: Method info: Column: Kinetex XB-C18 (75×3.0) mm, 2.6 μm; Mobile Phase: A: 5 mm Ammonium formate pH 3.3: ACN (98:02; Mobile Phase: B: ACN: Buffer (98:02; Flow Rate: 1.0 mL/min.

Method 2: Method info: Column: XBridge C₈ (50×4.6 mm)₅ μm; Mobile phase: A: 0.1% TFA in H₂O; Mobile phase: B: 0.1% TFA in ACN; Flow Rate: 1.5 mL/min.

Method 3: Column: Aquity Uplc BEH C18 (50×3.0) mm, 1.7 μm; Mobile Phase: A: 0.1% FA in Water; Mobile Phase: B: 0.1% TFA in ACN; Flow Rate: 1.0 mL/min Example S38. 1-(4-(1-(2,6-dichlorophenyl) azetidin-3-yl)-2,6-dimethylbenzyl)-3-ethylazetidin-3-ol (38)

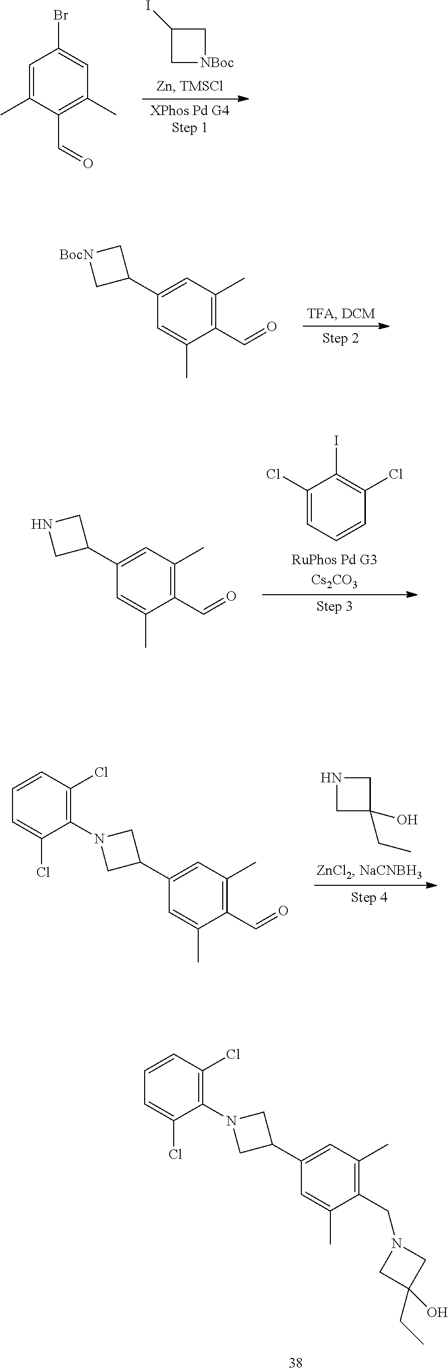

### Synthesis of tert-butyl 3-(4-formyl-3,5-dimethylphenyl) azetidine-1-carboxylate

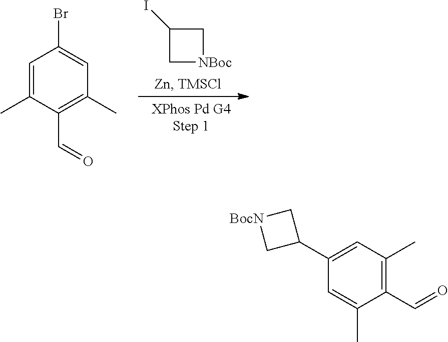

To a suspension of activated zinc (15.34 g, 235 mmol) in anhydrous DMF (200 mL) was added 1,2-dibromoethane (1.01 mL, 11.73 mmol) and heated to 75° C. After 15 mins, the reaction mixture was cooled to room temperature, chloro trimethylsilane (1.5 mL, 11.73 mmol) was added and allowed to stir at ambient temperature for additional 30 mins. Then a solution of tert-butyl 3-iodoazetidine-1-carboxylate (19.93 g, 70.4 mmol) in 50 mL of anhydrous DMF was added to the reaction mixture and stirred at room temperature for another 30 min., followed by 4-bromo-2,6-dimethylbenzaldehyde (5 g, 23.47 mmol) and XPhos Pd G4 (4.04 g, 4.69 mmol) in 50 mL of DMF was added. The reaction mixture was allowed to stir at 80° C. for 2 h. After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (250 mL), dried over anhydrous sodium sulphate and concentrated under reduced pressure. The residue thus obtained was purified by flash column chromatography on silica gel (100-200 mesh) eluting with 0-20% ethyl acetate in petroleum ether to afford tert-butyl 3-(4-formyl-3,5-dimethylphenyl) azetidine-1-carboxylate (4.3 g, 63% yield). LCMS Method 1; LCMS (ESI, m/z): 190.2 [M−100]⁺

### Synthesis of 4-(azetidin-3-yl)-2,6-dimethylbenzaldehyde

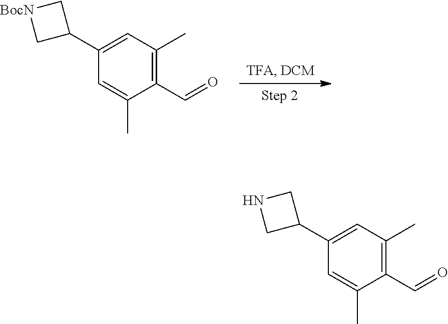 [SMILES: Cc1cc(C2CN(C(=O)OC(C)(C)C)C2)cc(C)c1C=O ; Cc1cc(C2CNC2)cc(C)c1C=O]

To a stirred solution of tert-butyl 3-(4-formyl-3,5-dimethylphenyl) azetidine-1-carboxylate (2 g, 6.91 mmol) in anhydrous dichloromethane (40 mL) was added trifluoroacetic acid (5.32 mL, 69.1 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford 4-(azetidin-3-yl)-2,6-dimethylbenzaldehyde as brown color semi-solid (1.96 g, 98% yield). LCMS Method 1; LCMS (ESI, m/z): 190.0 [M+H]⁺

### Synthesis of 4-(1-(2,6-dichlorophenyl) azetidin-3-yl)-2,6-dimethylbenzaldehyde

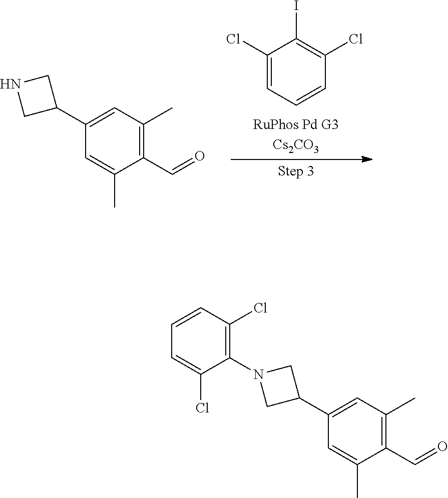 [SMILES: Cc1cc(C2CNC2)cc(C)c1C=O ; Clc1cccc(Cl)c1I ; Cc1cc(C2CN(c3c(Cl)cccc3Cl)C2)cc(C)c1C=O]

To a solution of 4-(azetidin-3-yl)-2,6-dimethylbenzaldehyde, TFA salt (1.8 g, 5.94 mmol) and 1,3-dichloro-2-iodobenzene (2.02 g, 7.42 mmol) in anhydrous 1,4 dioxane (30 mL) was added cesium carbonate (5.8 g, 17.8 mmol). The reaction mixture was degassed with nitrogen gas for 10 min and added RuPhos Pd G3 (248 mg, 0.297 mmol). The reaction mixture was allowed to stir at 80° C. for 16 h. Upon completion of the reaction, the reaction mixture was then cooled to room temperature and filtered through a celite pad and was washed with EtOAc. The filtrate was concentrated under reduced pressure and the residue thus obtained was purified by flash column chromatography on silica gel (230-400 mesh) eluting with 0-20% ethyl acetate in petroleum ether to afford 4-(1-(2,6-dichlorophenyl) azetidin-3-yl)-2,6-dimethylbenzaldehyde (600 mg, 30% yield) as a white solid. LCMS Method 1; LCMS (ESI, m/z): 334.0 [M+H]⁺.

### Synthesis of 1-(4-(1-(2,6-dichlorophenyl) azetidin-3-yl)-2,6-dimethylbenzyl)-3-ethylazetidin-3-ol (38)

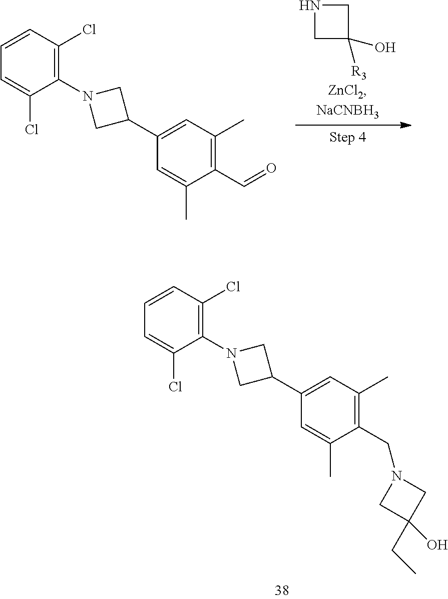

To a stirred solution of 3-ethylazetidin-3-ol, TFA salt (142 mg, 0.718 mmol) in MeOH(20 mL) was added sodium bicarbonate (240 mg, 2.8 mmol.) then stirred for 1 h at rt. The mixture was filtered through celite and concentrated to yield free amine. To the free amine 4-(1-(2,6-dichlorophenyl) azetidin-3-yl)-2,6-dimethylbenzaldehyde (200 mg, 0.598 mmol) in MeOH(8 mL) was added zinc chloride (82 mg, 0.598 mmol) and stirred for 1 h at 25° C. After 1 h, Sodium cyano borohydride (57 mg, 0.898 mmol.) was added and heated to 65° C. for 12 h. Upon completion of the reaction, the reaction mixture was diluted with dichloromethane (60 mL) and washed with sat. ammonium chloride solution and water (60 mL). The organic phase was dried over Na2SO₄, filtered and the solvents were evaporated under reduced pressure. The crude was purified by preparative HPLC(Method info: Diluent: THF: Water: ACN (50:20:30) Column: Zorbax C18 (50×21.5) mm, 5 micron Mobile phase A: 0.1% Formic acid in water Mobile phase B: Acetonitrile). Required fractions were concentrated and lyophilized to afford the title compound 1-(4-(1-(2,6-dichlorophenyl) azetidin-3-yl)-2,6-dimethylbenzyl)-3-ethylazetidin-3-ol, formic acid salt (108.5 mg, 0.226 mmol, 37.8% yield, 97% purity) as a white solid.

¹H NMR (400 MHZ, MeOD): 7.19-7.23 (m, 4H), 6.73 (t, J=8.00 Hz, 1H), 4.86-4.90 (m, 2H), 4.38-4.42 (m, 4H), 4.00-4.02 (m, 2H), 3.77-3.80 (m, 2H), 3.68-3.72 (m, 1H), 2.48 (s, 6H), 1.79-1.84 (m, 2H), 0.94-0.98 (m, 3H). LCMS Method 1; LCMS (ESI, m/z): 419.0 [M+H]⁺.

### Example S39. 1-(4-(1-(2,6-dichlorophenyl) azetidin-3-yl)-2,6-dimethylbenzyl)-3-(fluoromethyl) azetidin-3-ol (39)

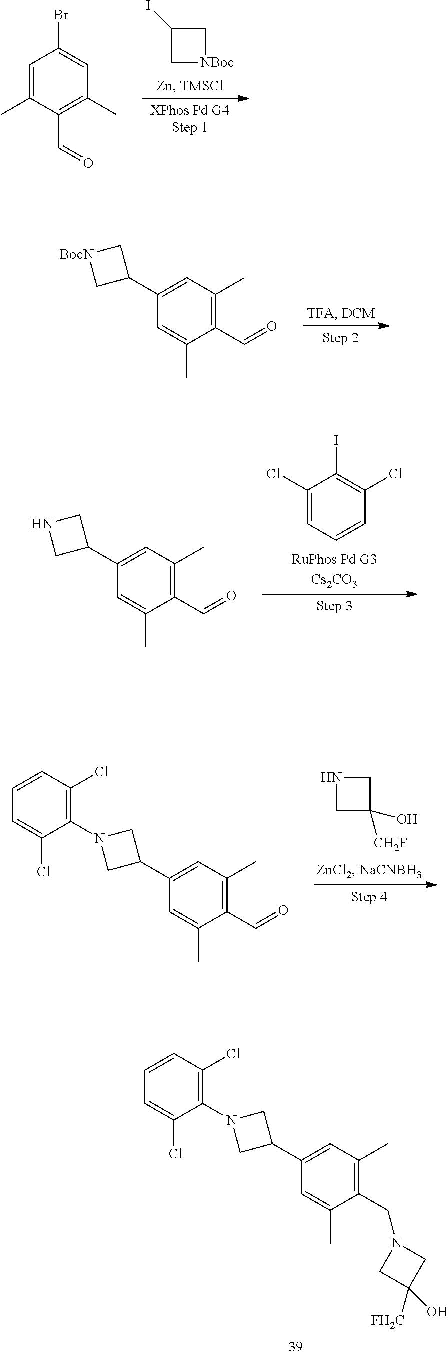

### Synthesis of 1-(4-(1-(2,6-dichlorophenyl) azetidin-3-yl)-2,6-dimethylbenzyl)-3-(fluoro-methyl) azetidin-3-ol (39)

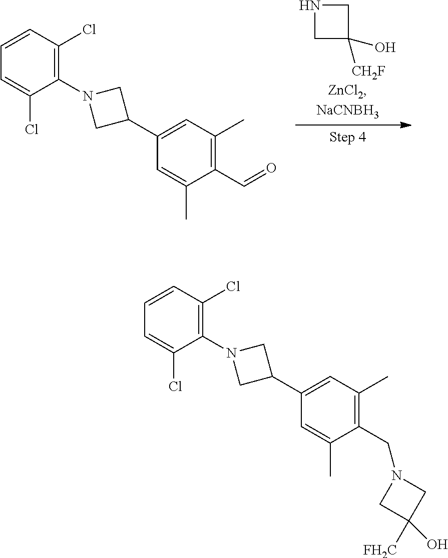

To a stirred solution of 3-(fluoromethyl) azetidin-3-ol, HCl (79 mg, 0.561 mmol) in MeOH(8 mL) was added sodium bicarbonate (94 mg, 1.122 mmol) then stirred for 1 h at rt. The mixture was filtered through celite and concentrated to yield free amine. To the free amine 4-(1-(2,6-dichlorophenyl) azetidin-3-yl)-2,6-dimethylbenzaldehyde (150 mg, 0.449 mmol)(Synthesis see example 1) in MeOH(6 mL) was added Zinc chloride (73.4 mg, 0.539 mmol) and stirred for 1 h at 25° C. After 1 h, sodium cyanoborohydride (28.2 mg, 0.449 mmol) was added and heated to 65° C. for 12 h. Upon completion of the reaction, the reaction mixture was diluted with dichloromethane (60 mL) and washed with sat. ammonium chloride solution and water (60 mL). The organic phase was dried over Na₂SO₄, filtered and the solvents were evaporated under reduced pressure. The crude was purified by preparative HPLC(Method info: Diluent: THF: Acetonitrile (30:70) Column: Xbridge C₈ (250×19) mm, 5 micron, Mobile phase A: 0.1% Formic acid in water Mobile phase B: Acetonitrile), The required fractions were concentrated and lyophilized to afford the title compound 1-(4-(1-(2,6-dichlorophenyl) azetidin-3-yl)-2,6-dimethylbenzyl)-3-(fluoromethyl) azetidin-3-ol (5 mg, 0.011 mmol, 2.55% yield, 96.8% purity) as an off white solid.

¹H NMR (400 MHZ, MeOD): δ 7.19 (d, J=8.00 Hz, 2H), 7.09 (s, 2H), 6.71 (t, J=8.40 Hz, 1H), 4.89 (m, 3H), 4.55 (s, 1H), 4.40-4.43 (m, 1H), 4.37-4.39 (m, 2H), 3.83 (s, 2H), 3.67 (m, 1H), 3.47-3.49 (m, 2H), 3.16 (s, 2H), 2.43 (s, 6H). LCMS Method 1; LCMS (ESI, m/z): 425.0 [M+H]⁺.

### Example S40. 1-(4-(1-(2,6-dichlorophenyl) azetidin-3-yl)-2,6-dimethylbenzyl)-3-(difluoromethyl) azetidin-3-ol (40)

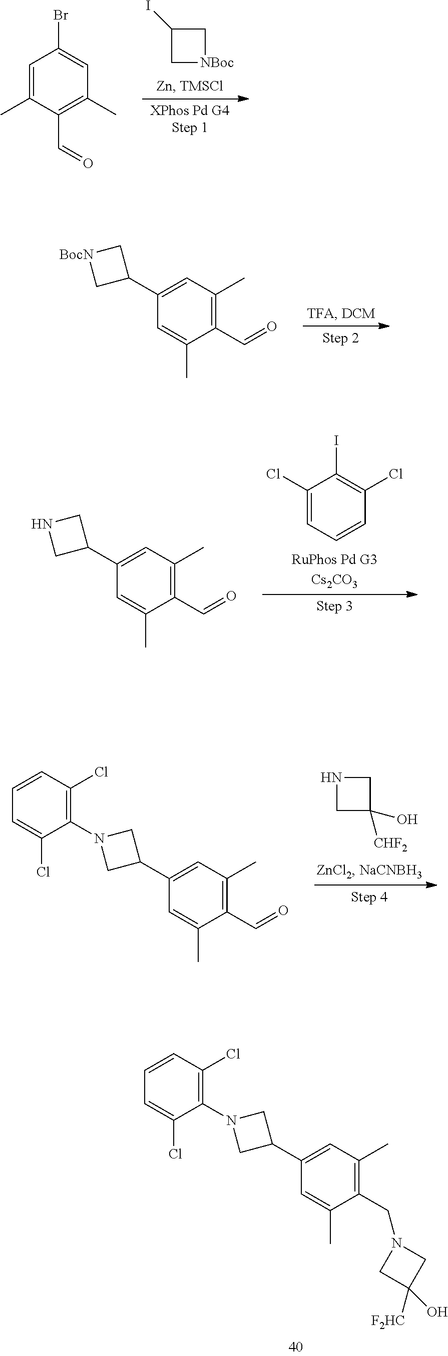

### Synthesis of 1-(4-(1-(2,6-dichlorophenyl) azetidin-3-yl)-2,6-dimethylbenzyl)-3-(difluoromethvl) azetidin-3-ol (40)

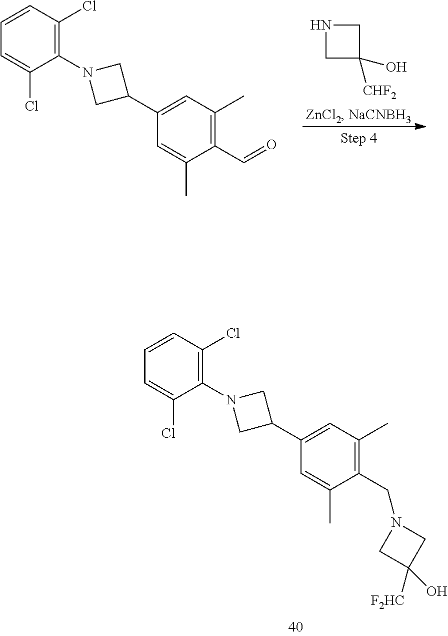

To a stirred solution of 3-(difluoromethyl) azetidin-3-ol, HCl (100 mg, 0.627 mmol) in MeOH (10 mL) was added sodium bicarbonate (106 mg, 1.257 mmol) then stirred for 1 h at rt. The mixture was filtered through celite and concentrated to yield free amine. To the free amine 4-(1-(2,6-dichlorophenyl) azetidin-3-yl)-2,6-dimethylbenzaldehyde (168 mg, 0.503 mmol)(Synthesis see example 1) in MeOH(6 mL) was added Zinc chloride (68.5 mg, 0.503 mmol) and stirred for 1 h at 25° C. After 1 h, sodium cyanoborohydride (106 mg, 1.257 mmol) was added and heated to 65° C. for 12 h. Upon completion of the reaction, the reaction mixture was diluted with dichloromethane (60 mL) and washed with sat. ammonium chloride solution and water (60 mL). The organic phase was dried over Na₂SO₄, filtered and the solvents were evaporated under reduced pressure. The crude was purified by preparative HPLC(Method info: Diluent: THF: Water: ACN (50:20:30) Column: Zorbax C18 (50×21.5) mm, 5 micron Mobile phase A: 0.1% Formic acid in water Mobile phase B: Acetonitrile). Required fractions were concentrated and lyophilized to afford the title compound 1-(4-(1-(2,6-dichlorophenyl) azetidin-3-yl)-2,6-dimethylbenzyl)-3-(difluoromethyl) azetidin-3-ol, formic acid salt (30 mg, 0.061 mmol, 12.17% yield) as a white solid.

¹H NMR (400 MHZ, MeOD): o 7.12-7.20 (m, 4H), 6.72 (t, J=19.20 Hz, 1H), 4.39 (t, J=14.00 Hz, 2H), 3.97-3.98 (m, 2H), 3.72 (t, J=25.60 Hz, 3H), 3.33-3.39 (m, 2H), 2.44 (s, 6H). Note: 2H are merged with the solvent peak. LCMS Method 1; LCMS (ESI, m/z): 443.0 [M+H]⁺.

### Example S41. 1-(4-(1-(2,6-dichlorophenyl) azetidin-3-yl)-2,6-dimethylbenzyl)-3-(trifluoromethyl) azetidin-3-ol (41)

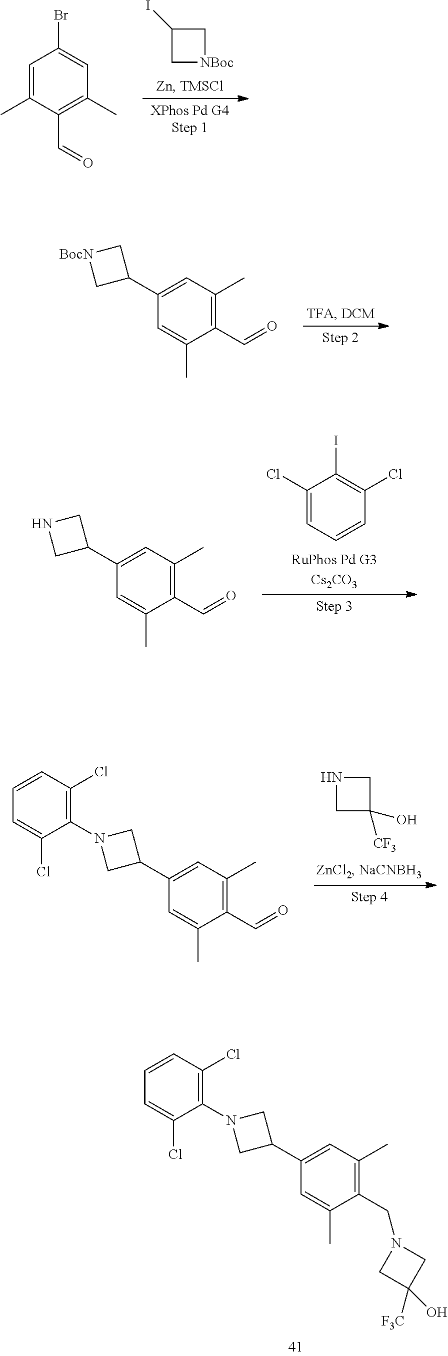

### Synthesis of 1-(4-(1-(2,6-dichlorophenyl) azetidin-3-yl)-2,6-dimethylbenzyl)-3-(trifluoromethyl) azetidin-3-ol (41)

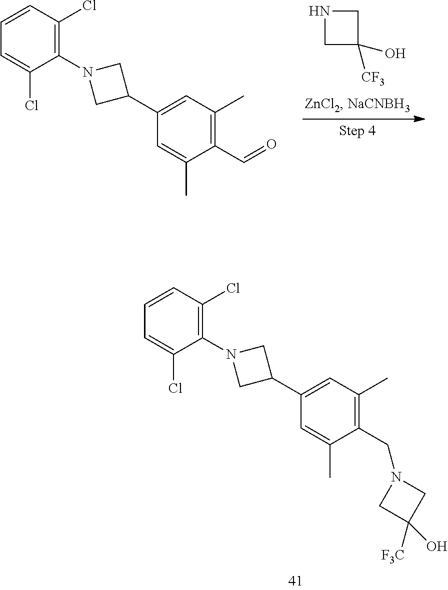 [SMILES: Cc1cc(C2CN(c3c(Cl)cccc3Cl)C2)cc(C)c1C=O ; OC1(C(F)(F)F)CNC1 ; Cc1cc(C2CN(c3c(Cl)cccc3Cl)C2)cc(C)c1CN1CC(O)(C(F)(F)F)C1]

To a stirred solution of 3-(trifluoromethyl) azetidin-3-ol (158 mg, 1.122 mmol) and 4-(1-(2,6-dichlorophenyl) azetidin-3-yl)-2,6-dimethylbenzaldehyde (250 mg, 0.748 mmol)(Synthesis see example 1) in MeOH(6 mL) was added Zinc Chloride (122 mg, 0.898 mmol) and stirred for 1 h at 25° C. After 1 h, Sodium cyanoborohydride (70.5 mg, 1.122 mmol) was added and heated to 65° C. for 12 h. The reaction mixture was diluted with dichloromethane (60 mL) and washed with sat. ammonium chloride solution and water (60 mL). The combined organic phase was dried over Na₂SO₄, filtered and the solvents were evaporated under reduced pressure. The crude was purified using flash column chromatography using 100-200 mesh silica gel and 5-25% of EtOAc/pet ether. Upon evaporation of required fractions yielded 1-(4-(1-(2,6-dichlorophenyl) azetidin-3-yl)-2,6-dimethylbenzyl)-3-(trifluoromethyl) azetidin-3-ol (55 mg, 0.119 mmol, 15.94% yield) as a off white solid.

¹H NMR (400 MHZ, DMSO-d₆): δ 7.23 (d, J=8.0 Hz, 2H), 7.02 (s, 2H), 6.85 (s, 1H), 6.74 (t, J=8.0 Hz, 1H), 4.80 (t, J=8.4 Hz, 2H), 4.32 (t, J=7.2 Hz, 2H), 3.67-3.64 (m, 3H), 3.46 (d, J=9.2 Hz, 2H), 3.19 (d, J=8.4 Hz, 2H), 2.35 (s, 6H). LCMS Method 1; LCMS (ESI, m/z): 461.0 [M+H]⁺.

### Example S42. 1-(4-(1-(2,6-difluorophenyl) azetidin-3-yl)-2,6-dimethylbenzyl)-3-methylazetidin-3-ol (42)

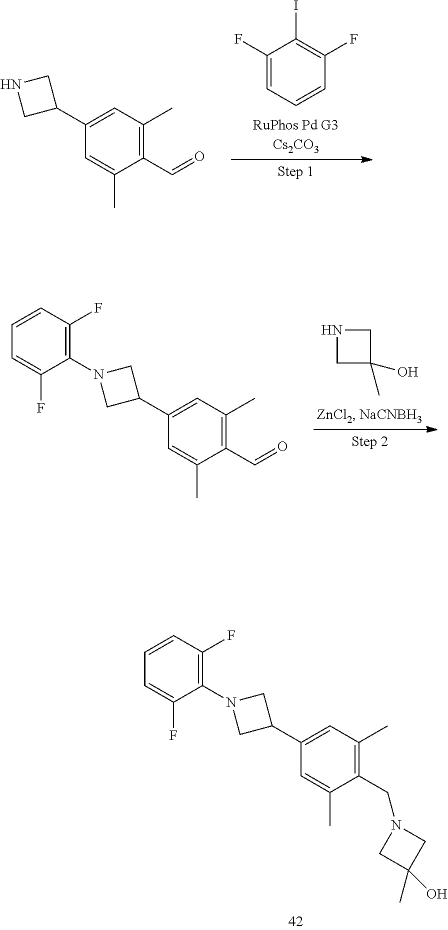

### Synthesis of 4-(1-(2,6-difluorophenyl) azetidin-3-yl)-2,6-dimethylbenzaldehyde

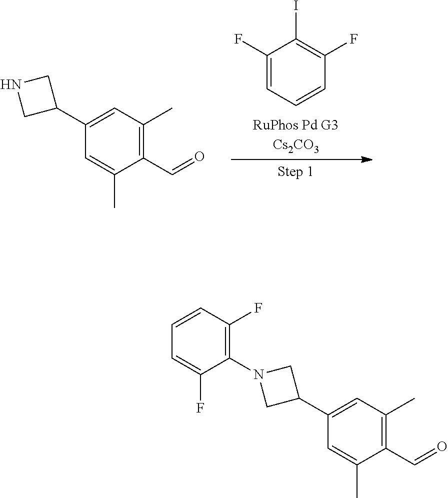 [SMILES: Cc1cc(C2CNC2)cc(C)c1C=O ; Fc1cccc(F)c1I ; Cc1cc(C2CN(c3c(F)cccc3F)C2)cc(C)c1C=O]

To a solution of 4-(azetidin-3-yl)-2,6-dimethylbenzaldehyde, TFA salt (300 mg, 0.98 mmol) and 1,3-difluoro-2-iodobenzene (356 mg, 1.48 mmol) in anhydrous 1,4 dioxane (8 mL) was added cesium carbonate (0.96 g, 2.97 mmol). The reaction mixture was then degassed with nitrogen for 10 min followed by the addition of added RuPhos Pd G3 (83 mg, 0.09 mmol) and heated 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-20% ethyl acetate in petroleum ether to afford 4-(1-(2,6-difluorophenyl) azetidin-3-yl)-2,6-dimethylbenzaldehyde (100 mg, 31% yield) as a Yellow semi-solid. LCMS (ESI, m/z): 302.0 [M+H]⁺.

### Synthesis of 1-(4-(1-(2,6-difluorophenyl) azetidin-3-yl)-2,6-dimethylbenzyl)-3-methylazetidin-3-ol (42)

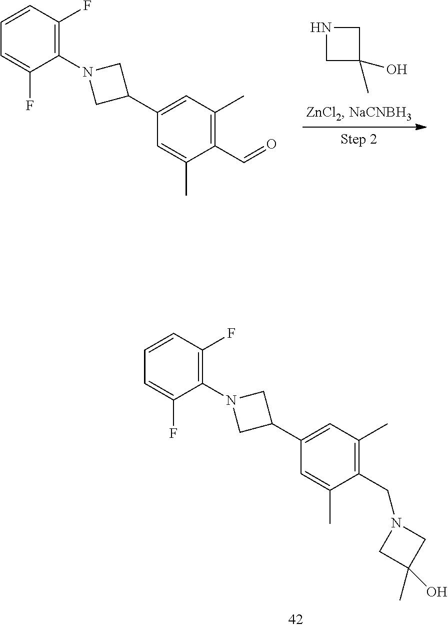

To a stirred solution of 3-methylazetidin-3-ol, TFA salt (200 mg, 0.9 mmol) in MeOH(5 mL) was added sodium bicarbonate (167 mg, 1.99 mmol.) then stirred for 1 h at rt. The mixture was filtered through celite and concentrated to yield free amine. To the free amine 4-(1-(2,6-difluorophenyl) azetidin-3-yl)-2,6-dimethylbenzaldehyde (200 mg, 0.664 mmol) in 5 ml of MeOH was added zinc chloride (90 mg, 0.664 mmol) and stirred for 1 h at 25° C. After 1 h, Sodium cyano borohydride (62 mg, 0.9 mmol.) was added and heated to 65° C. for 12 h. The reaction mixture was diluted with dichloromethane (10 mL) and washed with sat. ammonium chloride solution and water (20 mL). The combined organic phase was dried over Na₂SO₄, filtered and the solvents were evaporated under reduced pressure. The crude was purified by prep. HPLC(Diluent: THF: Acetonitrile (50:50); Column: Sunfire C18 (150×19) mm, 5 micron; Mobile phase A: 0.1% Formic acid in water; Mobile phase B: Acetonitrile 1-(4-(1-(2,6-difluorophenyl) azetidin-3-yl)-2,6-dimethylbenzyl)-3-methylazetidin-3-ol, formic acid salt (38 mg, 14% yield, 99.8% purity) as a white solid.

¹H NMR (400 MHZ, DMSO-d₆): δ 7.181 (s, 2H), 6.81-6.86 (m, 2H), 6.68-6.73 (m, 1H), 4.53-4.57 (m, 2H), 0.00 (s, 2H), 4.12-4.16 (m, 2H), 3.93-3.95 (m, 2H), 3.87-3.83 (m, 1H), 3.77-3.79 (m, 2H), 2.49 (s, 6H), 1.503 (s, 3H). LCMS method 1; LCMS (ESI, m/z): 373.2 [M+H]⁺.

### Example S43. 1-(2,6-dimethyl-4-(1-phenylazetidin-3-yl)benzyl)-3-methylazetidin-3-ol (43)

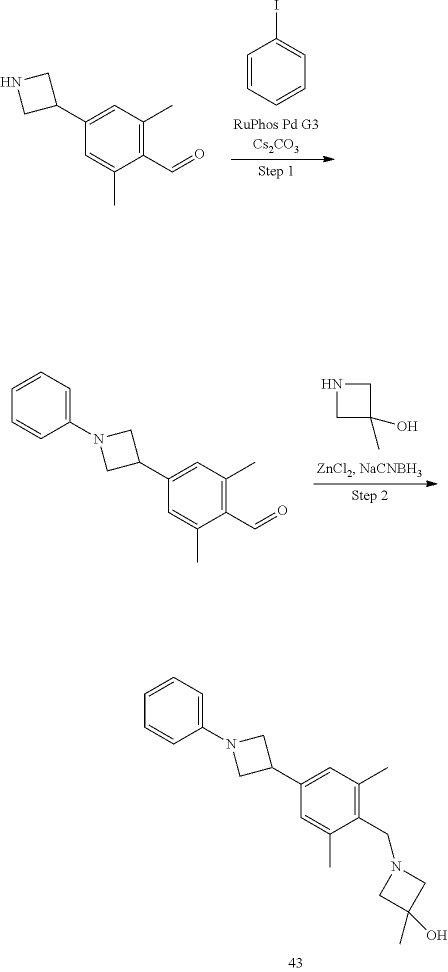

### Synthesis of 2,6-dimethyl-4-(1-phenylazetidin-3-yl)benzaldehyde

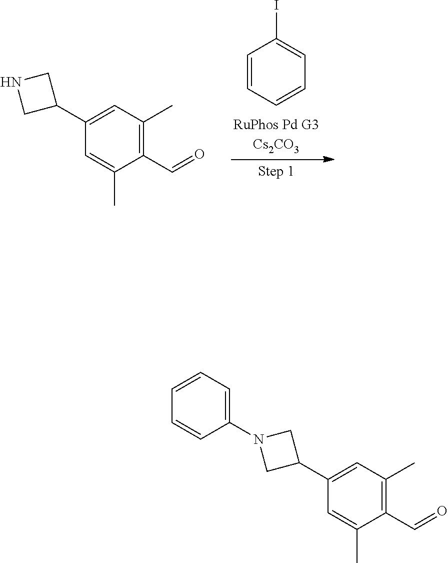 [SMILES: Cc1cc(C2CNC2)cc(C)c1C=O ; Ic1ccccc1 ; Cc1cc(C2CN(c3ccccc3)C2)cc(C)c1C=O]

To a solution of 4-(azetidin-3-yl)-2,6-dimethylbenzaldehyde, TFA salt (1 g, 3.68 mmol) and Iodobenzene (750 mg, 3.68 mmol) in anhydrous 1,4 dioxane (10 mL) was added cesium carbonate (3.5 g, 11.03 mmol). The reaction mixture was then degassed with nitrogen for 10 min followed by the addition of added RuPhos Pd G3 (307 mg, 0.368 mmol) and heated 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-20% ethyl acetate in petroleum ether to afford 2,6-dimethyl-4-(1-phenylazetidin-3-yl)benzaldehyde (302 mg, 31.1% yield) as a yellow solid. LCMS method 1; LCMS (ESI, m/z): 266.2 [M+H]⁺.

### Synthesis of 1-(2,6-dimethyl-4-(1-phenylazetidin-3-yl)benzyl)-3-methylazetidin-3-ol (43)

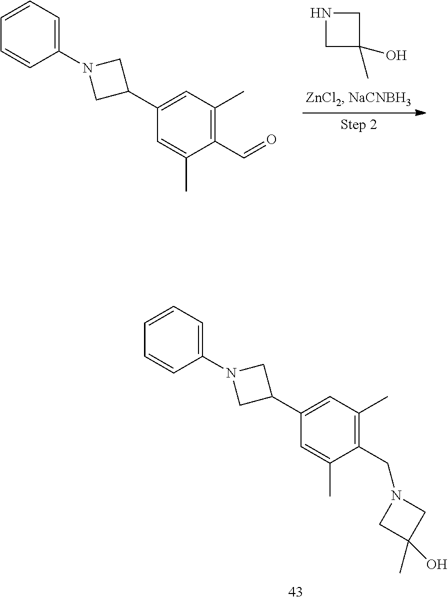

To a stirred solution of 3-methylazetidin-3-ol (157 mg, 1.80 mmol) in MeOH(10 mL) was added 2,6-dimethyl-4-(1-phenylazetidin-3-yl)benzaldehyde (400 mg, 1.507 mmol) and Zinc chloride (247 mg, 1.507 mmol) and stirred for 1 h at RT. After 1 h, Sodium cyanoborohydride (95 mg, 1.507 mmol) was added and heated to 65° C. for 12 h. The reaction mixture was diluted with dichloromethane (10 mL) and washed with sat. ammonium chloride solution and water (20 mL). The combined organic phase was dried over Na₂SO₄, filtered and the solvent was evaporated under reduced pressure. The crude was purified by prep. HPLC(Diluent: THF: Acetonitrile (30:70); Gemini NX C18 (50×21.2) mm, 10 micron, Mobile phase A: 0.1% Formic acid in water, Mobile phase B: Acetonitrile) to afford 1-(2,6-dimethyl-4-(1-phenylazetidin-3-yl)benzyl)-3-methylazetidin-3-ol, formic acid salt (51 mg, 0.131 mmol, 8.70% yield, 98.4% purity) as a white solid. ¹H-NMR (400 MHZ, MeOD): δ 7.19-7.23 (m, 2H), 7.12 (s, 2H), 6.75 (t, J=7.60 Hz, 1H), 6.54-6.56 (m, 2H), 4.24 (t, J=7.60 Hz, 2H), 4.19 (s, 2H), 3.76-3.89 (m, 5H), 3.62 (d, J=9.60 Hz, 2H), 2.44 (s, 6H), 1.48 (s, 3H). LCMS Method 2; LCMS (ESI, m/z): 337.2 [M+H]⁺

### Example S44. 1-(4-(1-(2-fluorophenyl) azetidin-3-yl)-2,6-dimethylbenzyl)-3-methylazetidin-3-ol (44)

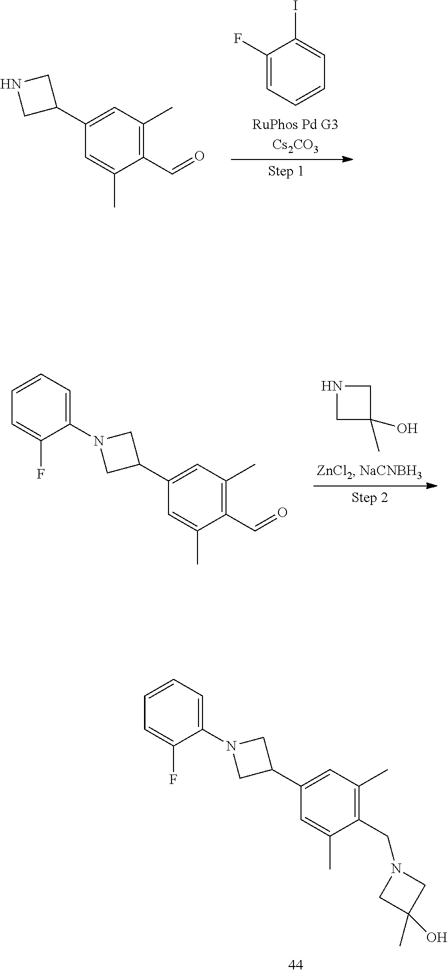

To a stirred solution of 3-methylazetidin-3-ol (157 mg, 1.80 mmol) in MeOH(10 mL) was added 2,6-dimethyl-4-(1-phenylazetidin-3-yl)benzaldehyde (400 mg, 1.507 mmol), zinc chloride (247 mg, 1.507 mmol) and the mixture was stirred at RT. After 1 h, sodium cyanoborohydride (95 mg, 1.507 mmol) was added and heated to 65° C. for 12 h. The reaction mixture was diluted with dichloromethane (10 mL) and washed with sat. ammonium chloride solution and water (20 mL). The combined organic phase was dried over Na₂SO₄, filtered and the solvent was evaporated under reduced pressure. The crude residue was purified by prep. HPLC(Diluent: THF: Acetonitrile (30:70); Gemini NX C18 (50×21.2) mm, 10 micron, Mobile phase A: 0.1% Formic acid in water, Mobile phase B: Acetonitrile) to afford 1-(2,6-dimethyl-4-(1-phenylazetidin-3-yl)benzyl)-3-methylazetidin-3-ol as formic acid salt (51 mg, 0.131 mmol, 8.70% yield, 98.4% purity) as a white solid. ¹H-NMR (400 MHZ, MeOD): δ 7.19-7.23 (m, 2H), 7.12 (s, 2H), 6.75 (t, J=7.60 Hz, 1H), 6.54-6.56 (m, 2H), 4.24 (t, J=7.60 Hz, 2H), 4.19 (s, 2H), 3.76-3.89 (m, 5H), 3.62 (d, J=9.60 Hz, 2H), 2.44 (s, 6H), 1.48 (s, 3H). LCMS Method 2; LCMS (ESI, m/z): 337.2 [M+H]⁺

### Synthesis of 4-(1-(2-fluorophenyl) azetidin-3-yl)-2,6-dimethylbenzaldehyde

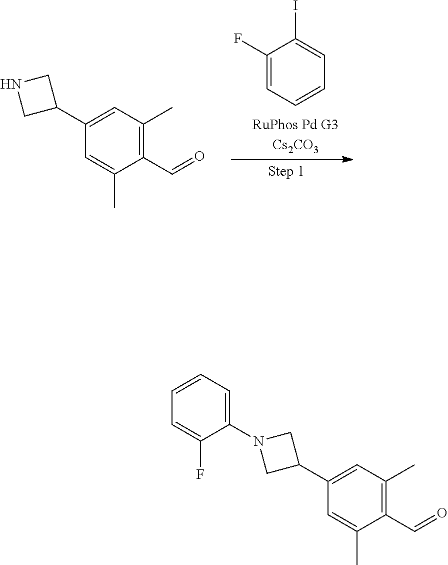 [SMILES: Cc1cc(C2CNC2)cc(C)c1C=O ; Fc1ccccc1I ; Cc1cc(C2CN(c3ccccc3F)C2)cc(C)c1C=O]

To a solution of 4-(azetidin-3-yl)-2,6-dimethylbenzaldehyde, TFA salt (300 mg,.98 mmol) and 1-fluoro-2-iodobenzene (329 mg, 1.48 mmol) in anhydrous 1,4 dioxane (8 mL) was added cesium carbonate (0.96 g, 2.97 mmol). The reaction mixture was then degassed with nitrogen for 10 min followed by the addition of added RuPhos Pd G3 (83 mg, 0.09 mmol) and heated 80° C.

After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-20% ethyl acetate in petroleum ether to afford 4-(1-(2-fluorophenyl) azetidin-3-yl)-2,6-dimethylbenzaldehyde (175 mg, 62.8% yield) as a pale yellow semi-solid. LCMS method 1, LCMS (ESI, m/z): 284.0 [M+H]⁺.

### Synthesis of 1-(4-(1-(2-fluorophenyl) azetidin-3-yl)-2,6-dimethylbenzyl)-3-methylazetidin-3-ol (44)

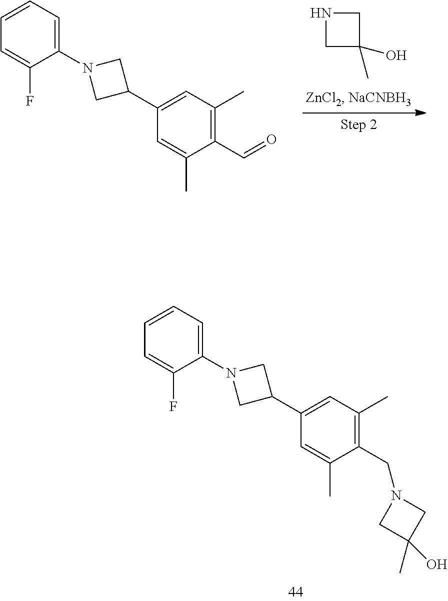

To a stirred solution of 3-methylazetidin-3-ol, TFA (213 mg, 1.059 mmol) in MeOH(5 mL) was added sodium bicarbonate (178 mg, 2.118 mmol) then stirred for 1 h at rt. The mixture was filtered through celite and concentrated to yield free amine. To the free amine 4-(1-(2-fluorophenyl) azetidin-3-yl)-2,6-dimethylbenzaldehyde (200 mg, 0.706 mmol) in 5 mL of MeOH was added zinc chloride (96 mg, 0.706 mmol) and stirred for 1 h at rt. After 1 h, sodium cyanoborohydride (66.5 mg, 1.059 mmol) was added and heated to 65° C. for 12 h. The reaction mixture was diluted with dichloromethane (10 mL) and washed with sat. ammonium chloride solution and water (20 mL). The combined organic phase was dried over Na₂SO₄, filtered and the solvents were evaporated under reduced pressure. The crude was purified by prep. HPLC (Method info: Diluent: THF: Acetonitrile (30:70); Column: Xbridge C18 (150×19) mm, 5 micron; Mobile phase A: 5 mM Ammonium formate in water; Mobile phase B: Acetonitrile) to yield 1-(4-(1-(2-fluorophenyl) azetidin-3-yl)-2,6-dimethylbenzyl)-3-methylazetidin-3-ol, formic acid salt (68 mg, 0.170 mmol, 24.03% yield, 99.9% purity) as a white solid.

¹H NMR (400 MHZ, MeOD): δ 7.19 (s, 2H), 6.95-7.06 (m, 2H), 6.74-6.79 (m, 1H), 6.63-6.65 (m, 1H), 4.43 (s, 2H), 4.33-4.37 (m, 2H), 4.02 (d, J=11.20 Hz, 2H), 3.84-3.93 (m, 5H), 2.47 (s, 6H), 1.51 (s, 3H). LCMS method 1; LCMS (ESI, m/z): 355.2 [M+H]⁺.

### Example S45. 1-(4-(1-(3-fluorophenyl) azetidin-3-yl)-2,6-dimethylbenzyl)-3-methylazetidin-3-ol (45)

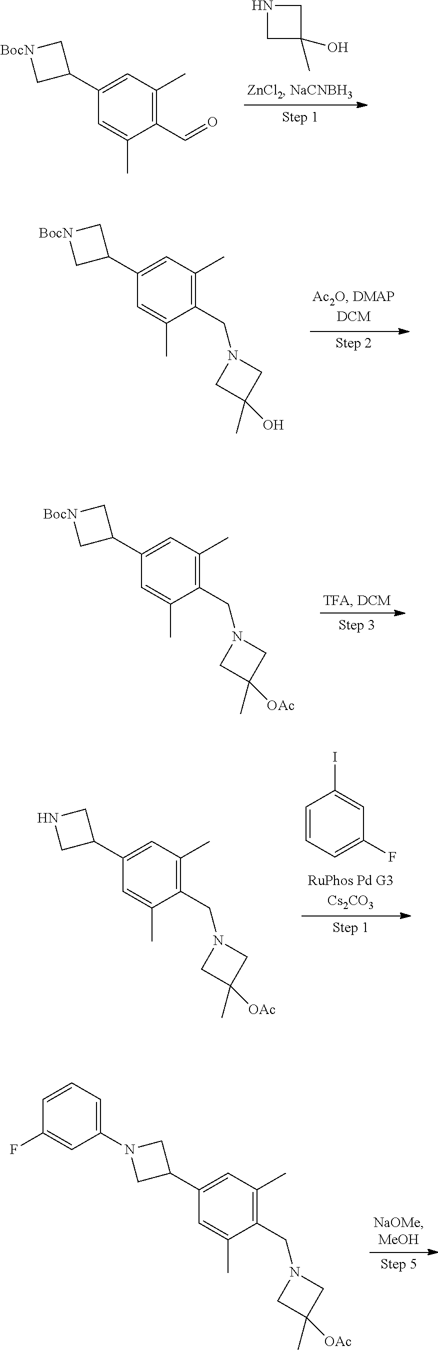

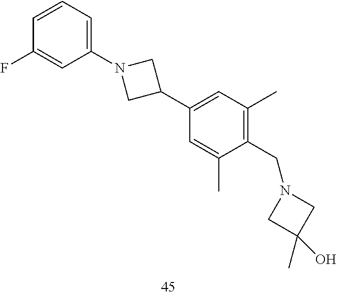 [SMILES: Cc1cc(C2CN(c3cccc(F)c3)C2)cc(C)c1CN1CC(C)(O)C1]

### Synthesis of tert-butyl 3-(4-((3-hydroxy-3-methylazetidin-1-yl)methyl)-3,5-dimethylphenyl) azetidine-1-carboxylate

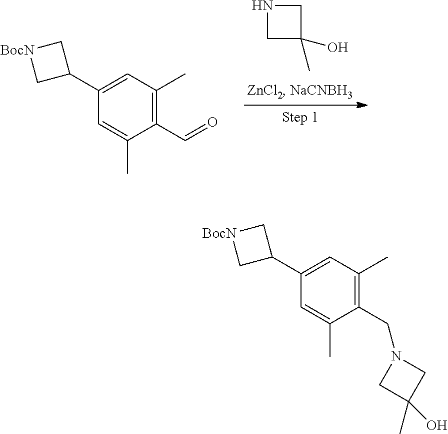

To a stirred solution of 3-methylazetidin-3-ol (2.5 g, 8.64 mmol) in MeOH(10 mL) was added tert-butyl 3-(4-formyl-3,5-dimethylphenyl) azetidine-1-carboxylate (2.5 g, 8.64 mmol) (Synthesis see example 1) and zinc chloride (1.766 g, 12.96 mmol) and stirred for 1 h at rt. After 1 h, sodium cyanoborohydride (0.814 g, 12.96 mmol) was added and heated to 65° C. for 12 h. The reaction mixture was diluted with dichloromethane (10 mL) and washed with sat. ammonium chloride solution and water (20 mL). The combined organic phase was dried over Na2SO₄, filtered and the solvents were evaporated under reduced pressure. The crude was purified by reverse phase column chromatography (Method: Diluent: THF: Acetonitrile (30:70) Column: Symmetry C8 (300× 19) mm, 7micronMobile phase A: 5 mM Ammonium formate in water Mobile phase B: Acetonitrile) to afford tert-butyl 3-(4-((3-hydroxy-3-methylazetidin-1-yl)methyl)-3,5-dimethylphenyl) azetidine-1-carboxylate (1.5 g, 3.92 mmol, 45.4% yield) as a colorless semi-solid. LCMS method 1; LCMS (ESI, m/z): 361.2 [M+H]⁺.

### Synthesis of tert-butyl 3-(4-((3-acetoxy-3-methylazetidin-1-yl)methyl)-3,5-dimethylphenyl) azetidine-1-carboxylate

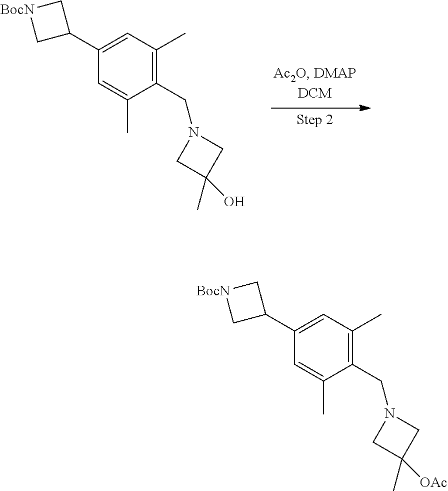 [SMILES: Cc1cc(C2CN(C(=O)OC(C)(C)C)C2)cc(C)c1CN1CC(C)(O)C1 ; CC(=O)OC1(C)CN(Cc2c(C)cc(C3CN(C(=O)OC(C)(C)C)C3)cc2C)C1]

To a stirred solution of tert-butyl 3-(4-((3-hydroxy-3-methylazetidin-1-yl)methyl)-3,5-dimethylphenyl) azetidine-1-carboxylate (1.5 g, 4.16 mmol) in DCM (20 mL) were added DMAP (0.508 g, 4.16 mmol) and Py (0.6 mL) and the reaction mixture was stirred at room temperature for 10 minutes. After that acetic anhydride (1.178 mL, 12.48 mmol) was added and the reaction mixture was stirred for 16 h at rt. The reaction mixture was diluted with saturated solution of ammonium chloride and extracted with DCM. The combined organic layer was dried over sodium sulphate, concentrated under vacuum, purified by flash column chromatography on 230-400 mesh silica gel (eluted with 20-30% ethyl acetate in pet ether) to afford tert-butyl 3-(4-((3-acetoxy-3-methylazetidin-1-yl)methyl)-3,5-dimethylphenyl) azetidine-1-carboxylate (800 mg, 1.987 mmol, 47.8% yield) as a transparent oil. LCMS method 1; LCMS (ESI, m/z): 403.2 [M+H]⁺.

### Synthesis of 1-(4-(azetidin-3-yl)-2,6-dimethylbenzyl)-3-methylazetidin-3-yl acetate

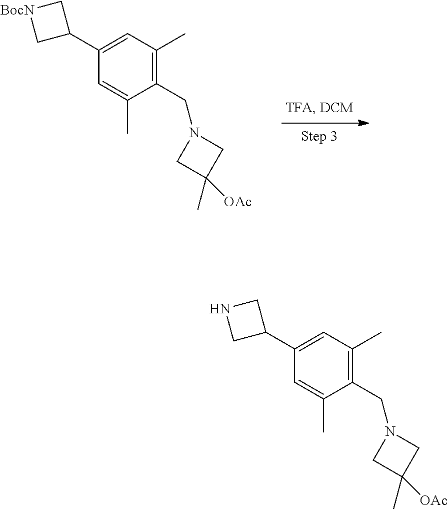 [SMILES: CC(=O)OC1(C)CN(Cc2c(C)cc(C3CN(C(=O)OC(C)(C)C)C3)cc2C)C1 ; CC(=O)OC1(C)CN(Cc2c(C)cc(C3CNC3)cc2C)C1]

To a stirred solution of tert-butyl 3-(4-((3-acetoxy-3-methylazetidin-1-yl)methyl)-3,5-dimethylphenyl) azetidine-1-carboxylate (1.5 g, 3.7 mmol) in anhydrous dichloromethane (40 mL) was added trifluoroacetic acid (2.7 mL, 69.1 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford quantitative amount of 1-(4-(azetidin-3-yl)-2,6-dimethylbenzyl)-3-methylazetidin-3-yl acetate, TFA as a quantitative amount of brown color semi-solid. LCMS method 1; LCMS (ESI, m/z): 303.0 [M+H]⁺.

### Synthesis of 1-(4-(1-(3-fluorophenyl) azetidin-3-yl)-2,6-dimethylbenzyl)-3-methylazetidin-3-yl acetate

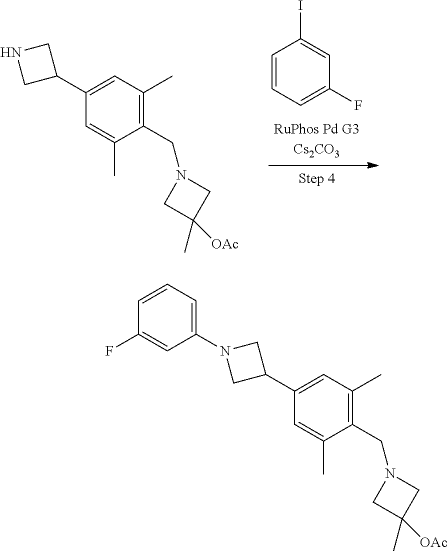

To a solution of 4 1-(4-(azetidin-3-yl)-2,6-dimethylbenzyl)-3-methylazetidin-3-yl acetate, TFA salt (300 mg,.98 mmol) and 1-fluoro-3-iodobenzene (356 mg, 1.48 mmol) in anhydrous 1,4 dioxane (8 mL) was added cesium carbonate (0.96 g, 2.97 mmol). The reaction mixture was then degassed with nitrogen for 10 min, followed by RuPhos Pd G3 (83 mg, 0.09 mmol) was added to the reaction mixture and heated to 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-20% ethyl acetate in petroleum ether to afford 1-(4-(1-(3-fluorophenyl) azetidin-3-yl)-2,6-dimethylbenzyl)-3-methylazetidin-3-yl acetate (60 mg, 16% yield) as a Yellow semi-solid. LCMS method 1; LCMS (ESI, m/z): 397.4 [M+H]⁺.

### Synthesis of 1-(4-(1-(3-fluorophenyl) azetidin-3-yl)-2,6-dimethylbenzyl)-3-methylazetidin-3-ol (45)

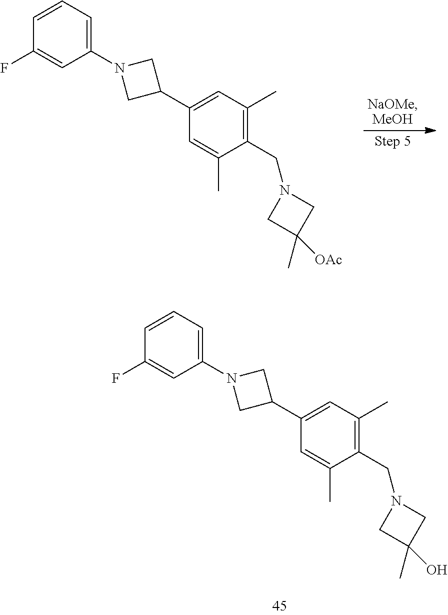

To a stirred solution of 1-(4-(1-(3-fluorophenyl) azetidin-3-yl)-2,6-dimethylbenzyl)-3-methylazetidin-3-yl acetate (60 mg, 0.151 mmol) in methanol (5 mL) was added sodium methoxide (20.44 mg, 0.378 mmol). The resulting mixture was stirred at room temperature for 1 h. The reaction mixture was diluted with saturated solution of ammonium chloride and extracted with EtOAc. The combined organic layer was dried over sodium sulphate, concentrated under vacuum. The crude was purified using Prep. HPLC(method info: Diluent: THF: Water: ACN (50:10:40); Column: Symmetry C₈ (300×19) mm, 7 micron; Mobile phase A: 0.1% Formic acid in water; Mobile phase B: Acetonitrile) to afford 1-(4-(1-(3-fluorophenyl) azetidin-3-yl)-2,6-dimethylbenzyl)-3-methylazetidin-3-ol, Formic acid salt (18 mg, 29% yield, 99.8% purity) as a white solid.

¹H NMR (400 MHZ, MeOD): 7.16-7.21 (m, 3H), 6.41-6.46 (m, 1H), 6.32-6.34 (m, 1H), 6.24-6.31 (m, 1H), 4.29-4.44 (m, 2H), 4.26-4.28 (m, 2H), 4.01-4.03 (m, 2H), 3.82-3.93 (m, 5H), 2.47 (s, 6H), 1.51 (s, 3H). LCMS method 1; LCMS (ESI, m/z): 355.2 [M+H]⁺.

### Example S46. 1-(4-(1-(3-chloro-4-cyclopropylphenyl) azetidin-3-yl)-2,6-dimethylbenzyl)-3-methylazetidin-3-ol (46)

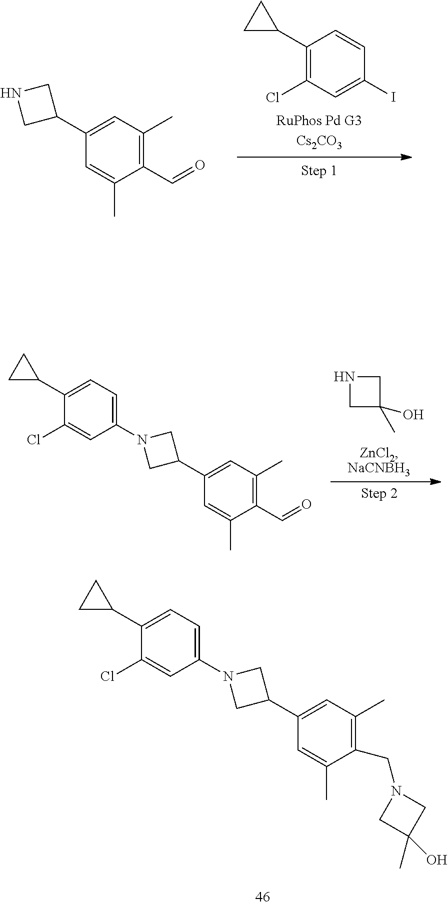

### Synthesis of 4-(1-(3-chloro-4-cyclopropylphenyl) azetidin-3-yl)-2,6-dimethylbenzaldehyde

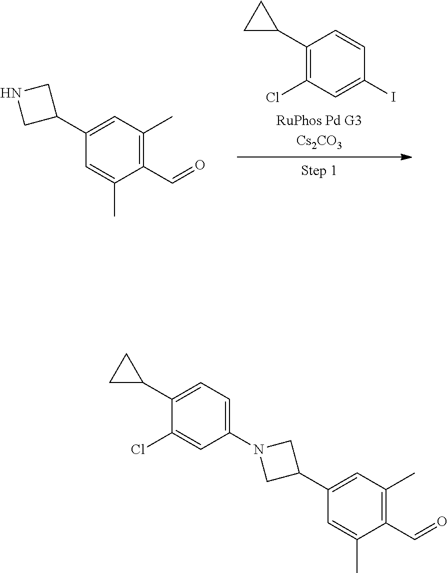 [SMILES: Cc1cc(C2CNC2)cc(C)c1C=O ; Clc1cc(I)ccc1C1CC1 ; Cc1cc(C2CN(c3ccc(C4CC4)c(Cl)c3)C2)cc(C)c1C=O]

To a solution of 4-(azetidin-3-yl)-2,6-dimethylbenzaldehyde, TFA salt (770 mg, 2.69 mmol) (Synthesis see example 1) and 2-chloro-1-cyclopropyl-4-iodobenzene (750 mg, 2.69 mmol) in anhydrous 1,4 dioxane (10 mL) was added cesium carbonate (2.6 g, 8.08 mmol). The reaction mixture was then degassed with nitrogen for 10 min followed by the addition of added RuPhos Pd G3 (225 mg, 0.269 mmol) and heated 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-20% ethyl acetate in petroleum ether to afford 4-(1-(3-chloro-4-cyclopropylphenyl) azetidin-3-yl)-2,6-dimethylbenzaldehyde (255 mg, 27.9% yield) as a yellow solid. LCMS Method 1, LCMS (ESI, m/z): 340.1 [M+H]⁺

### Synthesis of 1-(4-(1-(3-chloro-4-cyclopropylphenyl) azetidin-3-yl)-2,6-dimethylbenzyl)-3-methylazetidin-3-ol (46)

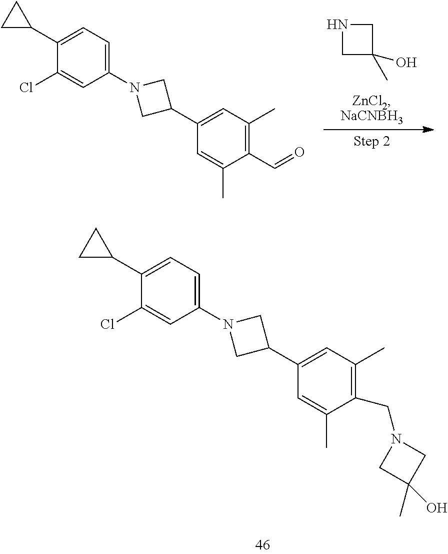

To a stirred solution of 3-methylazetidin-3-ol (123 mg, 1.34 mmol) in MeOH(10 mL) was added 4-(1-(3-chloro-4-cyclopropylphenyl) azetidin-3-yl)-2,6-dimethylbenzaldehyde (400 mg, 1.177 mmol) and Zinc chloride (192 mg, 1.412 mmol) and stirred for 1 h at rt. After 1 h, Sodium cyanoborohydride (74 mg, 1.177 mmol) was added and heated to 65° C. for 12 h. The reaction mixture was diluted with dichloromethane (10 mL) and washed with sat. ammonium chloride solution and water (20 mL). The combined organic phase was dried over Na₂SO₄, filtered and the solvents were evaporated under reduced pressure. The crude was purified by prep. HPLC (Diluent: THF: Acetonitrile (30:70); Column: Xselect C18 (150×19) mm, 5 micron; Mobile phase A: 0.1% Formic acid in water; Mobile phase B: Acetonitrile) to afford 1-(4-(1-(3-chloro-4-cyclopropylphenyl) azetidin-3-yl)-2,6-dimethylbenzyl)-3-methylazetidin-3-ol, formic acid salt (23 mg, 0.050 mmol, 4.25% yield, 99.5% purity) as a off white solid.

¹H NMR (400 MHZ, MeOD): 7.13 (s, 2H), 6.88 (d, J=8.40 Hz, 1H), 6.54 (d, J=2.40 Hz, 1H), 6.38-6.41 (m, 1H), 4.21 (s, 4H), 3.78-3.87 (m, 5H), 3.66 (d, J=9.60 Hz, 2H), 2.44 (s, 6H), 2.02-2.06 (m, 1H), 1.49 (s, 3H), 0.89-0.94 (m, 2H), 0.56-0.60 (m, 2H). LCMS method 2; LCMS (ESI, m/z): 411.1 [M+H]⁺.

### Example S47. 1-(4-(1-(4-cyclopropylphenyl) azetidin-3-yl)-2,6-dimethylbenzyl)-3-methylazetidin-3-ol (47)

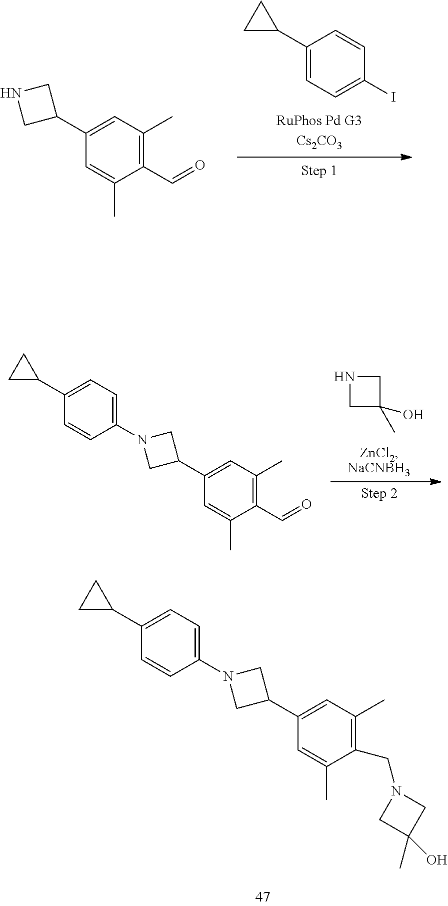

### Synthesis of 4-(1-(4-cyclopropylphenyl) azetidin-3-yl)-2,6-dimethylbenzaldehyde

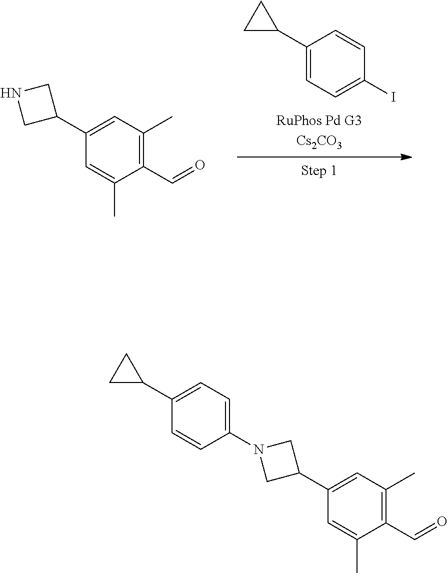 [SMILES: Cc1cc(C2CNC2)cc(C)c1C=O ; Ic1ccc(C2CC2)cc1 ; Cc1cc(C2CN(c3ccc(C4CC4)cc3)C2)cc(C)c1C=O]

To a solution of 4-(azetidin-3-yl)-2,6-dimethylbenzaldehyde, TFA salt (750 mg. 2.473 mmol) (Synthesis see example 1) and 1-bromo-4-cyclopropylbenzene (585 mg, 2.97 mmol) in anhydrous 1,4 dioxane (10 mL) was added cesium carbonate (2.4 g, 7.42 mmol). The reaction mixture was then degassed with nitrogen for 10 min followed by the addition of added RuPhos Pd G3 (207 mg, 0.247 mmol) and heated 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-20% ethyl acetate in petroleum ether to afford 4-(1-(2,6-difluorophenyl) azetidin-3-yl)-2,6-dimethylbenzaldehyde (297 mg, 39% yield) as a Off white solid; LCMS Method 1; LCMS (ESI, m/z): 306.3 [M+H]⁺.

### Synthesis of 1-(4-(1-(4-cyclopropylphenyl) azetidin-3-yl)-2,6-dimethylbenzyl)-3-methylazetidin-3-ol (47)

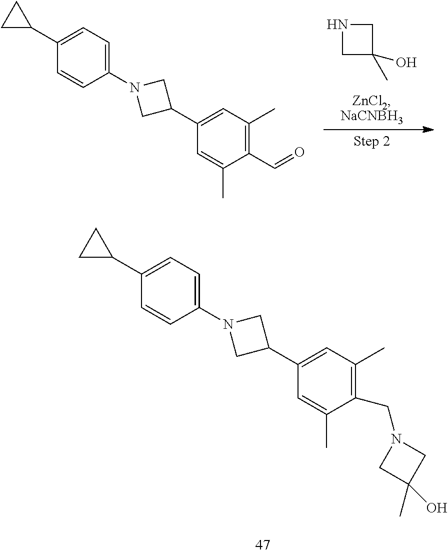

To a stirred solution of 3-methylazetidin-3-ol (171 mg, 1.965 mmol) in MeOH(10 mL) was added 4-(1-(4-cyclopropylphenyl) azetidin-3-yl)-2,6-dimethylbenzaldehyde (400 mg, 1.310 mmol) and Zinc chloride (214 mg, 1.572 mmol) and stirred for 1 h at rt. After 1 h, Sodium cyanoborohydride (123 mg, 1.965 mmol) was added and heated to 65° C. for 12 h. The reaction mixture was diluted with dichloromethane (10 mL) and washed with sat. ammonium chloride solution and water (20 mL). The combined organic phase was dried over Na₂SO₄, filtered and the solvents were evaporated under reduced pressure. The crude was purified by prep. HPLC (Diluent: THF: Acetonitrile (30:70); Column: Xselect C18 (150×19) mm, 5 micron; Mobile phase A: 0.1% Formic acid in water; Mobile phase B: Acetonitrile) to afford 1-(4-(1-(4-cyclopropylphenyl) azetidin-3-yl)-2,6-dimethylbenzyl)-3-methylazetidin-3-ol, formic acid salt (210 mg, 0.493 mmol, 37.6% yield, 99.1% purity) as a light brown solid.

¹H NMR (400 MHZ, DMSO-d₆): δ 7.04 (s, 2H), 6.92 (d, J=8.8 Hz, 2H)₂H), 6.41 (d, J=8.4 Hz, 2H), 4.15 (t, J=7.2 Hz, 2H), 3.83 (m, 1H), 3.69 (m, 3H), 2.36 (s, 6H), 1.82-1.78 (m, 1H), 1.34 (s, 3H), 0.86-0.81 (m, 2H), 0.54-0.51 (m, 2H). Few protons are merged with solvent signals. LCMS Mehtod 1, LCMS (ESI, m/z): 377.2 [M+H]⁺.

### Example S48. 1-(4-(1-(2,6-dichlorophenyl) azetidin-3-yl)-2-fluoro-6-methylbenzyl)-3-methylazetidin-3-ol (48)

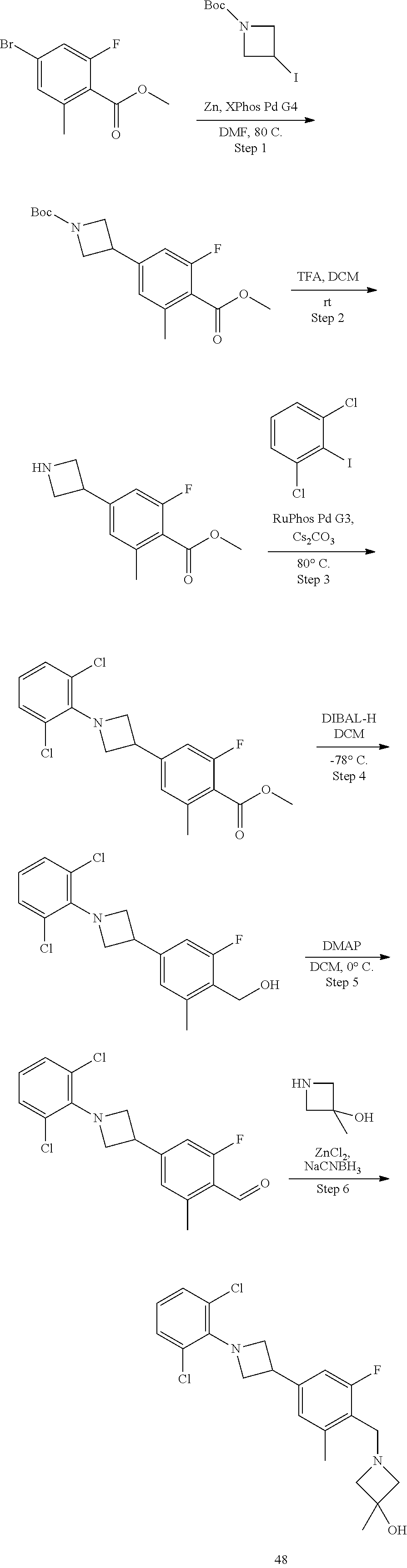

### Synthesis of tert-butyl 3-(3-fluoro-4-(methoxycarbonyl)-5-methylphenyl) azetidine-1-carboxylate

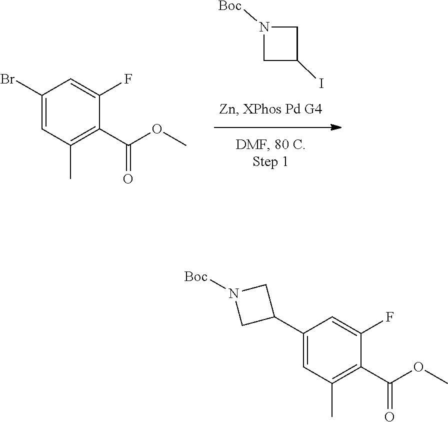 [SMILES: COC(=O)c1c(C)cc(Br)cc1F ; CC(C)(C)OC(=O)N1CC(I)C1 ; COC(=O)c1c(C)cc(C2CN(C(=O)OC(C)(C)C)C2)cc1F]

To a suspension of activated zinc (5.3 g, 81 mmol) in anhydrous DMF (40 mL) was added 1,2-dibromoethane (0.34 mL, 4.05 mmol) and heated to 75° C. After 15 mins, the reaction mixture was cooled to room temperature, chloro trimethylsilane (0.285 mL, 2.229 mmol) was added and allowed to stir at ambient temperature for additional 30 mins. Then a solution of tert-butyl 3-iodoazetidine-1-carboxylate (6.8 g, 24.29 mmol) in 20 mL of anhydrous DMF was added to the reaction mixture and stirred at room temperature for another 30 min., followed by methyl 4-bromo-2-fluoro-6-methylbenzoate (2 g, 8.10 mmol) and XPhos Pd G4 (1 g, 1.214 mmol) in 20 mL of DMF was added. The reaction mixture was allowed to stir at 80° C. for 2 h. After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (50 mL), dried over anhydrous sodium sulphate and concentrated under reduced pressure. The residue thus obtained was purified by flash column chromatography on silica gel (100-200 mesh) eluting with 0-50% ethyl acetate in petroleum ether to afford tert-butyl 3-(3-fluoro-4-(methoxycarbonyl)-5-methylphenyl) azetidine-1-carboxylate (2.2 g, 87% purity, 73% yield); LCMS method 1, LCMS (ESI, m/z): 224.2 [M−100]⁺ .

### Synthesis of methyl 4-(azetidin-3-yl)-2-fluoro-6-methylbenzoate; TFA salt

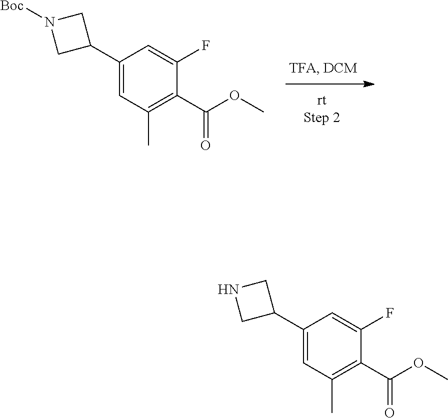 [SMILES: COC(=O)c1c(C)cc(C2CN(C(=O)OC(C)(C)C)C2)cc1F ; COC(=O)c1c(C)cc(C2CNC2)cc1F]

To a stirred solution of tert-butyl 3-(3-fluoro-4-(methoxycarbonyl)-5-methylphenyl) azetidine-1-carboxylate (2.2 g, 6.80 mmol) in anhydrous dichloromethane (30 mL) was added trifluoroacetic acid (5.24 mL, 68 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford methyl 4-(azetidin-3-yl)-2-fluoro-6-methylbenzoate; TFA salt as brown color semi-solid (2.2 g, 96% yield); LCMS method 1, LCMS (ESI, m/z): 224.2 [M+H]⁺.

### Synthesis of methyl 4-(1-(2,6-dichlorophenyl) azetidin-3-yl)-2-fluoro-6-methylbenzoate

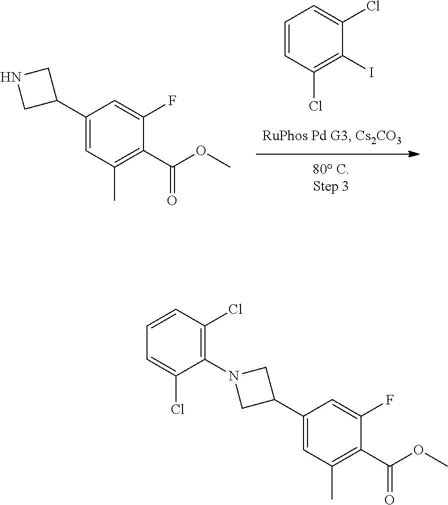 [SMILES: COC(=O)c1c(C)cc(C2CNC2)cc1F ; Clc1cccc(Cl)c1I ; COC(=O)c1c(C)cc(C2CN(c3c(Cl)cccc3Cl)C2)cc1F]

To a solution of 1 methyl 4-(azetidin-3-yl)-2-fluoro-6-methylbenzoate, TFA salt (1.2 g, 3.55 mmol) and 1,3-chloro-2-iodobenzene (1.2 g, 4.44 mmol) in anhydrous 1,4 dioxane (20 mL) was added cesium carbonate (3.4 g, 10.68 mmol). The reaction mixture was then degassed with nitrogen for 10 min followed by added RuPhos Pd G3 (149 mg, 0.17 mmol) and heated to 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford methyl 4-(1-(2,6-dichlorophenyl) azetidin-3-yl)-2-fluoro-6-methylbenzoate (550 mg, 42% yield) as a Orange brown solid; LCMS method 1, LCMS (ESI, m/z): 369.8 [M+H]⁺.

### Synthesis of (4-(1-(2,6-dichlorophenyl) azetidin-3-yl)-2-fluoro-6-methylphenyl) methanol

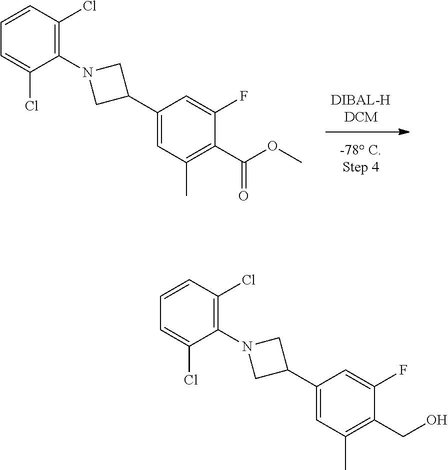 [SMILES: COC(=O)c1c(C)cc(C2CN(c3c(Cl)cccc3Cl)C2)cc1F ; Cc1cc(C2CN(c3c(Cl)cccc3Cl)C2)cc(F)c1CO]

To a stirred solution of methyl 4-(1-(2,6-dichlorophenyl) azetidin-3-yl)-2-fluoro-6-methylbenzoate (550 mg, 1.494 mmol) in DCM (20 mL) was added DIBAL-H in THF (2.99 mL, 2.99 mmol) at −78° C. and stirred for 4 h at −78° C. The reaction mixture was quenched with saturated ammonium chloride solution (10 ml) and extracted with DCM (60 mL) and washed with brine. The combined organic layer was dried over Na₂SO₄, filtered and the solvents were evaporated under reduced pressure. The crude was purified by silicagel coumn chromatography by using ethyl acetate/pet ether 0-30% to afford the title compound (4-(1-(2,6-dichlorophenyl) azetidin-3-yl)-2-fluoro-6-methylphenyl) methanol (530 mg, 1.496 mmol, 100% yield) as a yellow liquid; LCMS method 1, LCMS (ESI, m/z): 341.8 [M+H]⁺.

### Synthesis of 4-(1-(2,6-dichlorophenyl) azetidin-3-yl)-2-fluoro-6-methylbenzaldehyde

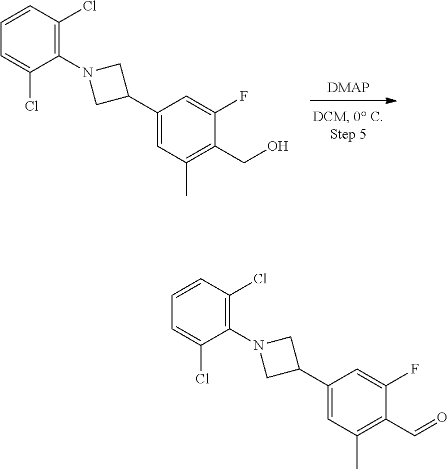 [SMILES: Cc1cc(C2CN(c3c(Cl)cccc3Cl)C2)cc(F)c1CO ; Cc1cc(C2CN(c3c(Cl)cccc3Cl)C2)cc(F)c1C=O]

To a stirred solution of (4-(1-(2,6-dichlorophenyl) azetidin-3-yl)-2-fluoro-6-methyl-phenyl) methanol (530 mg, 1.558 mmol) in DCM (10 mL) was added dess-martinperiodinane (793 mg, 1.869 mmol) at 0° C. under nitrogen atmosphere, after 1 h, TLC analysis indicated complete conversion of the starting material. The reaction mixture was filtered through a celite pad washed with EtOAc. The filtrate was concentrated under reduced pressure and the residue thus obtained was purified by flash column chromatography on silica gel (230-400 mesh) eluting with 0-20% ethyl acetate in petroleum ether to afford 4-(1-(2,6-dichlorophenyl) azetidin-3-yl)-2-fluoro-6-methylbenzaldehyde (371 mg, 70%) as a yellow semi-solid; LCMS method 1, LCMS (ESI, m/z): 338.0 [M+H]⁺.

### Synthesis of 1-(4-(1-(2,6-dichlorophenyl) azetidin-3-yl)-2-fluoro-6-methylbenzyl)-3-methylazetidin-3-ol (48)

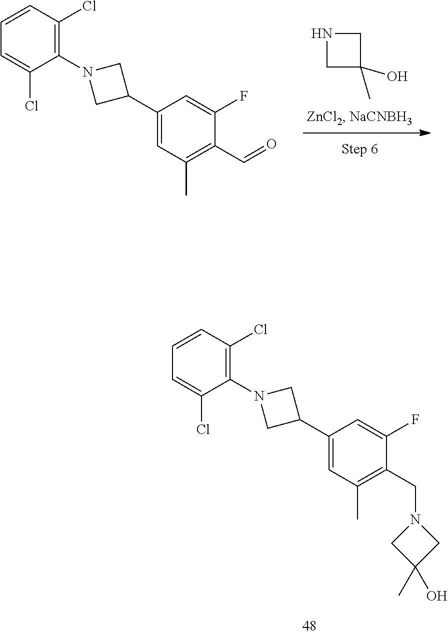

To a stirred solution of 3-methylazetidin-3-ol, TFA salt (122 mg, 0.66 mmol) in MeOH(5 mL) was added sodium bicarbonate (75 mg, 0.88 mmol.) then stirred for 1 h at rt. The mixture was filtered through celite and concentrated to yield free amine. To the free amine 4-(1-(2,6-dichlorophenyl) azetidin-3-yl)-2-fluoro-6-methylbenzaldehyde (150 mg, 0.44 mmol) in 5 mL of MeOH was added zinc chloride (60 mg, 0.44 mmol) and stirred for 1 h at 25° C. After 1 h, Sodium cyano borohydride (42 mg, 0.66 mmol.) was added and heated to 65° C. for 12 h. The reaction mixture was diluted with dichloromethane (10 mL) and washed with sat. ammonium chloride solution and water (20 mL). The combined organic phase was dried over Na₂SO₄, filtered and the solvents were evaporated under reduced pressure, the crude was purified by prep. HPLC(Diluent: THF: Water: ACN (50:10:40; Column: Luna C18 (250x21.2) mm, 10 micron Mobile phase A: 0.1% Formic acid in water, Mobile phase B: Acetonitrile). The required fractions were lyophilized to afford 1-(4-(1-(2,6-dichlorophenyl) azetidin-3-yl)-2-fluoro-6-methylbenzyl)-3-methylazetidin-3-ol (73 mg, 35.5%, 98.1% Purity) as a off white semi-solid.

¹H NMR (400 MHZ, MeOD): δ 7.17-7.23 (m, 4H), 6.74 (t, J=8.00 Hz, 1H), 4.86-4.90 (m, 2H), 4.38-4.42 (m, 2H), 4.29 (s, 2H), 3.90 (d, J=10.00 Hz, 2H), 3.72-3.79 (m, 3H), 2.48 (s, 3H), 1.51 (s, 3H); LCMS method 1, LCMS (ESI, m/z): 409.0 [M]⁺.

### Example S49. 1-(2-chloro-4-(1-(2,6-dichlorophenyl) azetidin-3-yl)-6-methylbenzyl)-3-methylazetidin-3-ol (49)

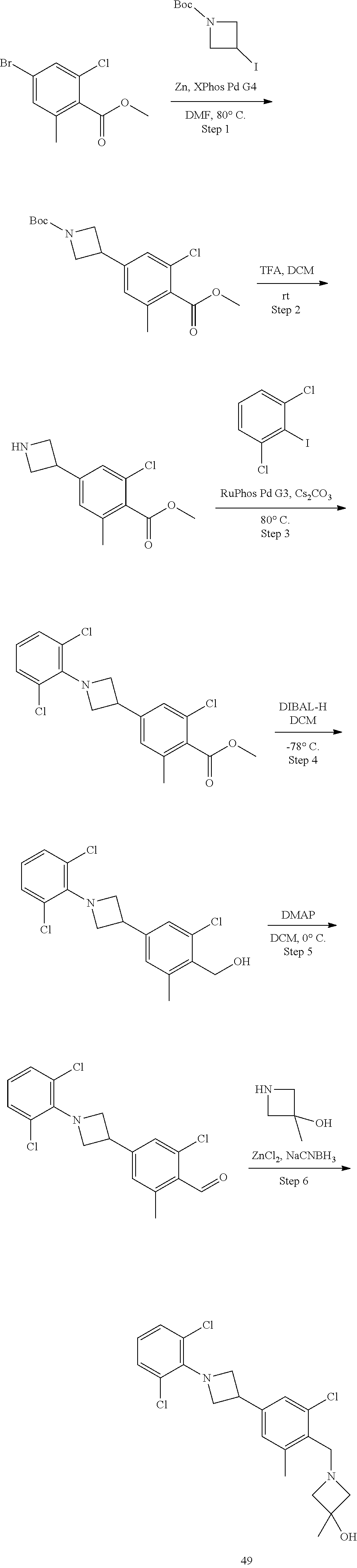

### Synthesis of tert-butyl 3-(3-chloro-4-(methoxycarbonyl)-5-methylphenyl) azetidine-1-carboxylate

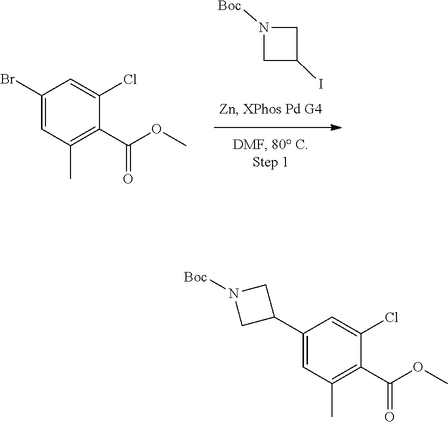 [SMILES: COC(=O)c1c(C)cc(Br)cc1Cl ; CC(C)(C)OC(=O)N1CC(I)C1 ; COC(=O)c1c(C)cc(C2CN(C(=O)OC(C)(C)C)C2)cc1Cl]

To a suspension of activated zinc (4.96 g, 76 mmol) in anhydrous DMF (20 mL) was added 1,2-dibromoethane (0.065 mL, 0.759 mmol) and heated to 75° C. After 15 mins, the reaction mixture was cooled to room temperature, chloro trimethylsilane (0.097 mL, 0.759 mmol) was added and allowed to stir at ambient temperature for additional 30 mins. Then a solution of tert-butyl 3-iodoazetidine-1-carboxylate (8.59 g, 30.4 mmol) in 10 mL of anhydrous DMF was added to the reaction mixture and stirred at room temperature for another 30 min., followed by methyl 4-bromo-2-chloro-6-methylbenzoate (2.0 g, 7.59 mmol) and XPhos Pd G4 (0.653 g, 0.759 mmol) in 10 mL of DMF was added. The reaction mixture was allowed to stir at 80° C. for 2 h. After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (50 mL), dried over anhydrous sodium sulphate and concentrated under reduced pressure. The residue thus obtained was purified by flash column chromatography on silica gel (100-200 mesh) eluting with 0-50% ethyl acetate in petroleum ether to afford tert-butyl 3-(3-chloro-4-(methoxycarbonyl)-5-methylphenyl) azetidine-1-carboxylate (1.58 g, 61.3% yield) as a colorless semi-solid; LCMS method 1, LCMS (ESI, m/z): 240.0 [M−100]*.

### Synthesis of methyl 4-(azetidin-3-yl)-2-chloro-6-methylbenzoate

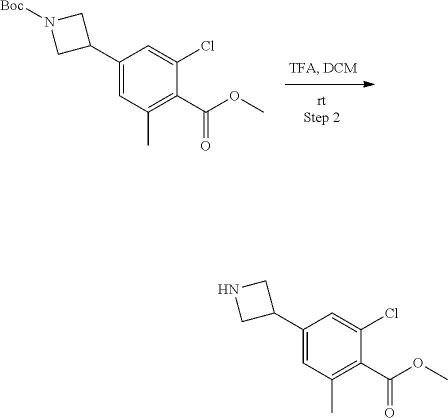 [SMILES: COC(=O)c1c(C)cc(C2CN(C(=O)OC(C)(C)C)C2)cc1Cl ; COC(=O)c1c(C)cc(C2CNC2)cc1Cl]

To a stirred solution of tert-butyl 3-(3-chloro-4-(methoxycarbonyl)-5-methylphenyl) azetidine-1-carboxylate (1.75 g, 5.15 mmol) in anhydrous dichloromethane (30 mL) was added trifluoroacetic acid (1.190 mL, 15.45 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford methyl 4-(azetidin-3-yl)-2-chloro-6-methylbenzoate, TFA (1.74 g, 96% yield) as a yellow semi-solid; LCMS method 3, LCMS (ESI, m/z): 240.0 [M+H]⁺.

### Synthesis of methyl 2-chloro-4-(1-(2,6-dichlorophenyl) azetidin-3-yl)-6-methylbenzoate

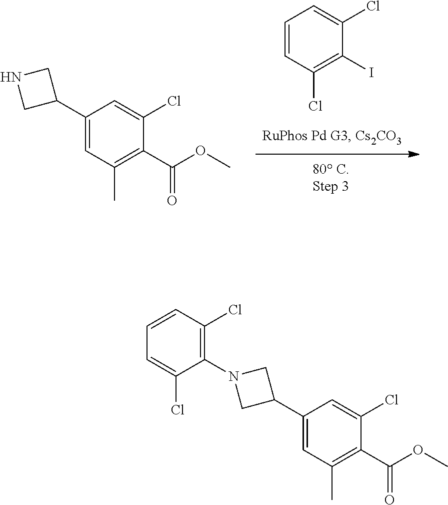 [SMILES: COC(=O)c1c(C)cc(C2CNC2)cc1Cl ; Clc1cccc(Cl)c1I ; COC(=O)c1c(C)cc(C2CN(c3c(Cl)cccc3Cl)C2)cc1Cl]

To a solution of methyl 4-(azetidin-3-yl)-2-chloro-6-methylbenzoate, TFA (1.296 g, 3.66 mmol) and 1,3-dichloro-2-iodobenzene (1 g, 3.66 mmol) in anhydrous 1,4 dioxane (10 mL) was added cesium carbonate (3.58 g, 10.99 mmol). The reaction mixture was then degassed with nitrogen for 10 min followed by added RuPhos Pd G3 (0.306 g, 0.366 mmol) and heated to 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford methyl 2-chloro-4-(1-(2,6-dichlorophenyl) azetidin-3-yl)-6-methylbenzoate (574 mg, 41.2% yield) as a yellow solid; LCMS method 2, LCMS (ESI, m/z): 385.8 [M+H]⁺.

### Synthesis of (2-chloro-4-(1-(2,6-dichlorophenyl) azetidin-3-yl)-6-methylphenyl) methanol

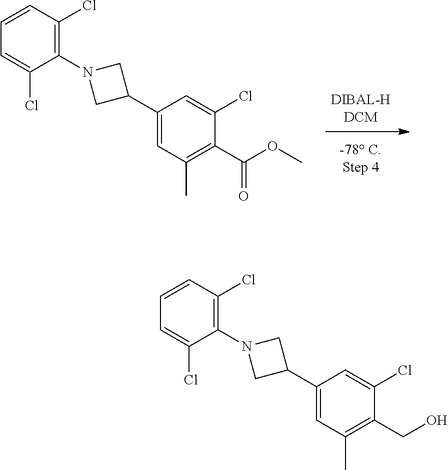 [SMILES: COC(=O)c1c(C)cc(C2CN(c3c(Cl)cccc3Cl)C2)cc1Cl ; Cc1cc(C2CN(c3c(Cl)cccc3Cl)C2)cc(Cl)c1CO]

To a stirred solution of methyl 2-chloro-4-(1-(2,6-dichlorophenyl) azetidin-3-yl)-6-methylbenzoate (700 mg, 1.820 mmol) in DCM (10 mL) was added DIBAL-H(3.03 mL, 3.64 mmol) at −78° C. and stirred for 4 h at −78° C. The reaction mixture was quenched with saturated ammonium chloride solution (10 ml) and extracted with DCM (30 mL) and washed with brine. The combined organic layer was dried over Na₂SO₄, filtered and the solvents were evaporated under reduced pressure. The crude was purified by silicagel coumn chromatography by using ethyl acetate/pet ether 0-50% to afford the title compound (2-chloro-4-(1-(2,6-dichlorophenyl) azetidin-3-yl)-6-methylphenyl) methanol (565 mg, 87% yield) as a white semi-solid; LCMS method 3, LCMS (ESI, m/z): 357.8 [M+H]⁺.

### Synthesis of 2-chloro-4-(1-(2,6-dichlorophenyl) azetidin-3-yl)-6-methylbenzaldehyde

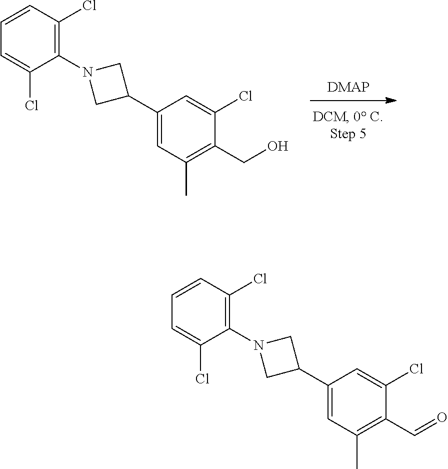 [SMILES: Cc1cc(C2CN(c3c(Cl)cccc3Cl)C2)cc(Cl)c1CO ; Cc1cc(C2CN(c3c(Cl)cccc3Cl)C2)cc(Cl)c1C=O]

To a stirred solution of (2-chloro-4-(1-(2,6-dichlorophenyl) azetidin-3-yl)-6-methylphenyl) methanol (580 mg, 1.626 mmol) in DCM (10 mL) was added dess-martinperiodinane (828 mg, 1.951 mmol) at 0° C. under nitrogen atmosphere, after 1 h, TLC analysis indicated complete conversion of the starting material. The reaction mixture was filtered through a celite pad washed with EtOAc. The filtrate was concentrated under reduced pressure and the residue thus obtained was purified by flash column chromatography on silica gel (230-400 mesh) eluting with 0-20% ethyl acetate in petroleum ether to afford 2-chloro-4-(1-(2,6-dichlorophenyl) azetidin-3-yl)-6-methylbenzaldehyde (467 mg, 81%) as a yellow solid; LCMS method 3, LCMS (ESI, m/z): 356.0 [M+H]⁺.

### Synthesis of 1-(2-chloro-4-(1-(2,6-dichlorophenyl) azetidin-3-yl)-6-methylbenzyl)-3-methylazetidin-3-ol; Formic acid salt (49)

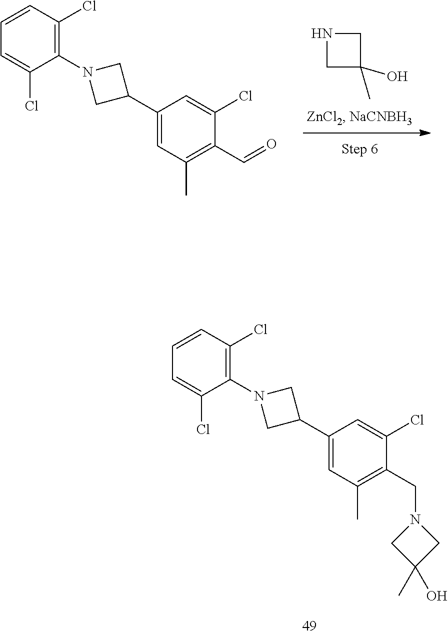

To a stirred solution of 3-methylazetidin-3-ol, TFA (102 mg, 0.508 mmol) in MeOH(5 mL) was added sodium bicarbonate (71.1 mg, 0.846 mmol) then stirred for 1 h at rt. The mixture was filtered through celite and concentrated to yield free amine. To the free amine and 2-chloro-4-(1-(2,6-dichlorophenyl) azetidin-3-yl)-6-methylbenzaldehyde (150 mg, 0.423 mmol) in 5 mL of MeOH was added zinc chloride (86 mg, 0.634 mmol) and stirred for 1 h at 25° C. After 1 h, sodium cyanoborohydride (26.6 mg, 0.423 mmol) was added and heated to 65° C. for 12 h. The reaction mixture was diluted with dichloromethane (10 mL) and washed with sat. ammonium chloride solution and water (20 mL). The combined organic phase was dried over Na₂SO₄, filtered and the solvents were evaporated under reduced pressure, the crude was purified by prep. HPLC(Diluent: THF: Water: ACN (50:10:40; Column: Luna C18 (250x21.2) mm, 10 micron Mobile phase A: 0.1% Formic acid in water, Mobile phase B: Acetonitrile). The required fractions were lyophilized to afford 1-(2-chloro-4-(1-(2,6-dichlorophenyl) azetidin-3-yl)-6-methylbenzyl)-3-methylazetidin-3-ol, formic acid salt (45 mg, 0.094 mmol, 22.21% yield, 98.5% purity) as an off white semi-solid.

¹H NMR (400 MHZ, MeOD): δ 7.43 (s, 1H), 7.34 (s, 1H), 7.20 (d, J=8.00 Hz, 2H), 6.74 (t, J=8.00 Hz, 1H), 4.88-4.90 (m, 2H), 4.38-4.41 (m, 2H), 4.29 (s, 2H), 3.67-3.78 (m, 5H), 2.50 (s, 3H), 1.49 (s, 3H); LCMS method 1, LCMS (ESI, m/z): 427.0 [M+H]⁺.

### Example S50. 1-(2-cyclopropyl-4-(1-(2,6-dichlorophenyl) azetidin-3-yl)benzyl)-3-methylazetidin-3-ol (50)

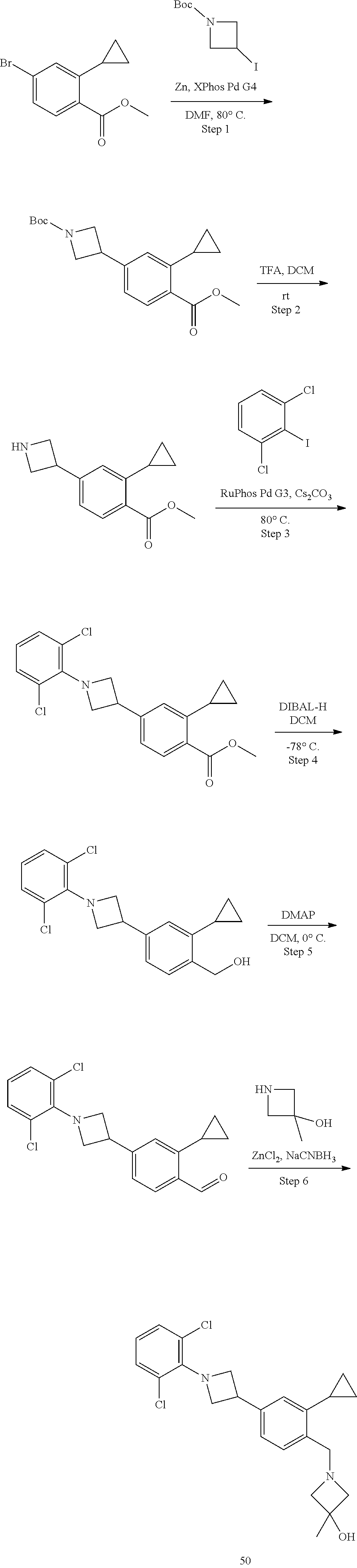

### Synthesis of tert-butyl 3-(3-cyclopropyl-4-(methoxycarbonyl)phenyl) azetidine-1-carboxylate

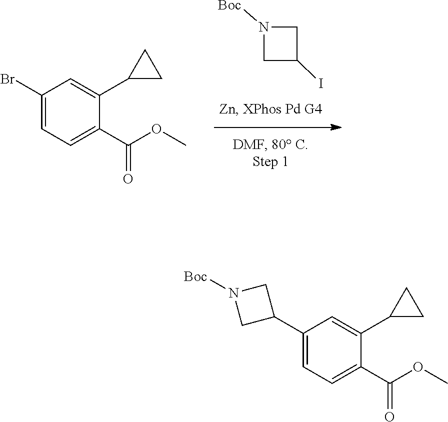 [SMILES: COC(=O)c1ccc(Br)cc1C1CC1 ; CC(C)(C)OC(=O)N1CC(I)C1 ; COC(=O)c1ccc(C2CN(C(=O)OC(C)(C)C)C2)cc1C1CC1]

To a suspension of activated zinc (6.41 g, 98 mmol) in anhydrous DMF (30 mL) was added 1,2-dibromoethane (0.422 ml, 4.90 mmol) and heated to 75° C. After 15 mins, the reaction mixture was cooled to room temperature, chloro trimethylsilane (0.285 ml, 2.229 mmol) was added and allowed to stir at ambient temperature for additional 30 mins. Then a solution of tert-butyl 3-iodoazetidine-1-carboxylate (8.32 g, 29.4 mmol) in 10 mL of anhydrous DMF was added to the reaction mixture and stirred at room temperature for another 30 min., followed by methyl 4-bromo-2-cyclopropylbenzoate (2.5 g, 9.80 mmol) and XPhos Pd G4 (1.265 g, 1.470 mmol) in 10 mL of DMF was added. The reaction mixture was allowed to stir at 80° C. for 2 h. After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (50 mL), dried over anhydrous sodium sulphate and concentrated under reduced pressure. The residue thus obtained was purified by flash column chromatography on silica gel (100-200 mesh) eluting with 0-50% ethyl acetate in petroleum ether to afford tert-butyl 3-(3-cyclopropyl-4-(methoxycarbonyl)phenyl) azetidine-1-carboxylate (1.72 g, 53.2% yield) as a yellow semi-solid; LCMS method 1, LCMS (ESI, m/z): 232.0 [M−100]⁺ .

### Synthesis of methyl 4-(azetidin-3-yl)-2-cyclopropylbenzoate

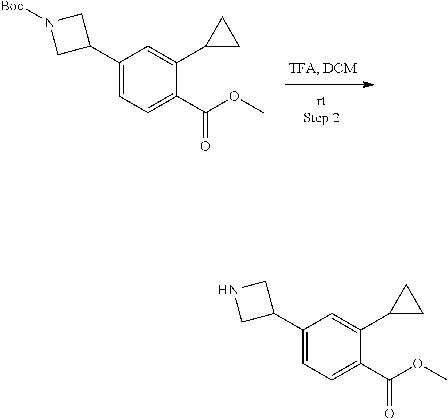 [SMILES: COC(=O)c1ccc(C2CN(C(=O)OC(C)(C)C)C2)cc1C1CC1 ; COC(=O)c1ccc(C2CNC2)cc1C1CC1]

To a stirred solution of tert-butyl 3-(3-cyclopropyl-4-(methoxycarbonyl)phenyl) azetidine-1-carboxylate (1.9 g, 5.73 mmol) in anhydrous dichloromethane (30 mL) was added trifluoroacetic acid (4.0 mL, 51.9 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford methyl 4-(azetidin-3-yl)-2-cyclopropylbenzoate, TFA (0.97 g, 53.9% yield) as a brown color semi-solid; LCMS method 1, LCMS (ESI, m/z): 232.2 [M+H]⁺

### Synthesis of methyl 2-cyclopropyl-4-(1-(2,6-dichlorophenyl) azetidin-3-yl)benzoate

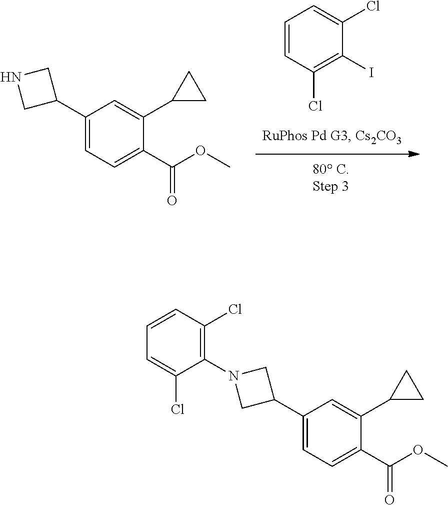 [SMILES: COC(=O)c1ccc(C2CNC2)cc1C1CC1 ; Clc1cccc(Cl)c1I ; COC(=O)c1ccc(C2CN(c3c(Cl)cccc3Cl)C2)cc1C1CC1]

To a solution of methyl 4-(azetidin-3-yl)-2-cyclopropylbenzoate, TFA (1.2 g, 3.48 mmol) and 1,3-dichloro-2-iodobenzene (1.138 g, 4.17 mmol) in anhydrous 1,4 dioxane (20 mL) was added cesium carbonate (10.43 mmol). The reaction mixture was then degassed with nitrogen for 10 min followed by added Ruphos pd G3 (0.291 g, 0.348 mmol) and heated to 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford methyl 2-cyclopropyl-4-(1-(2,6-dichlorophenyl) azetidin-3-yl)benzoate (759 mg, 58.1% yield) as a yellow solid; LCMS method 1, LCMS (ESI, m/z): 376.2 [M+H]⁺.

### Synthesis of (2-cyclopropyl-4-(1-(2,6-dichlorophenyl) azetidin-3-yl)phenyl) methanol

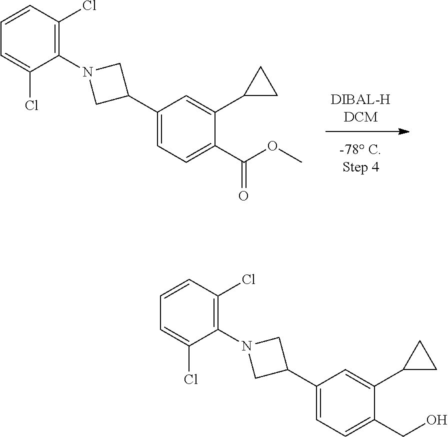 [SMILES: COC(=O)c1ccc(C2CN(c3c(Cl)cccc3Cl)C2)cc1C1CC1 ; OCc1ccc(C2CN(c3c(Cl)cccc3Cl)C2)cc1C1CC1]

To a stirred solution of methyl 2-cyclopropyl-4-(1-(2,6-dichlorophenyl) azetidin-3-yl)benzoate (700 mg, 1.860 mmol) in DCM (20 mL) was added DIBAL-H in THF (3.72 mL, 3.72 mmol) at −78° C. and stirred for 4 h at −78° C. The reaction mixture was quenched with saturated ammonium chloride solution (10 ml) and extracted with DCM (30 mL) and washed with brine. The combined organic layer was dried over Na₂SO₄, filtered and the solvents were evaporated under reduced pressure. The crude was purified by silicagel coumn chromatography by using ethyl acetate/pet ether 0-30% to afford the title compound (2-cyclopropyl-4-(1-(2,6-dichlorophenyl) azetidin-3-yl)phenyl) methanol (462 mg, 71.4% yield) as a yellow liquid; LCMS method 1, LCMS (ESI, m/z): 350.2 [M+2H]⁺ .

### Synthesis of 2-cyclopropyl-4-(1-(2,6-dichlorophenyl) azetidin-3-yl)benzaldehyde

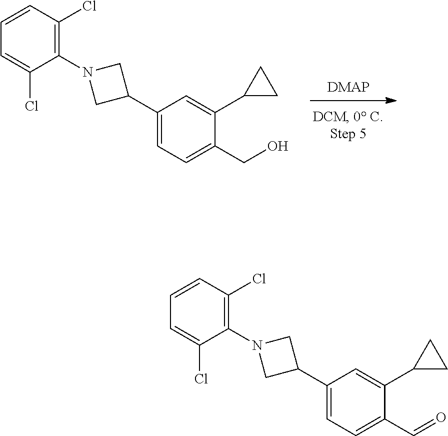 [SMILES: OCc1ccc(C2CN(c3c(Cl)cccc3Cl)C2)cc1C1CC1 ; O=Cc1ccc(C2CN(c3c(Cl)cccc3Cl)C2)cc1C1CC1]

To a stirred solution of (2-cyclopropyl-4-(1-(2,6-dichlorophenyl) azetidin-3-yl)phenyl) methanol (420 mg, 1.206 mmol) in DCM (10 mL) was added dess-martinperiodinane (614 mg, 1.447 mmol) at 0° C. under nitrogen atmosphere, after 1 h, TLC analysis indicated complete conversion of the starting material. The reaction mixture was filtered through a celite pad washed with EtOAc. The filtrate was concentrated under reduced pressure and the residue thus obtained was purified by flash column chromatography on silica gel (230-400 mesh) eluting with 0-20% ethyl acetate in petroleum ether to afford 2-cyclopropyl-4-(1-(2,6-dichlorophenyl) azetidin-3-yl)benzaldehyde (326 mg, 78% yield) as a yellow semi-solid; LCMS method 2; LCMS (ESI, m/z): 348.1 [M+2H]⁺ .

### Synthesis of 1-(2-cyclopropyl-4-(1-(2,6-dichlorophenyl) azetidin-3-yl)benzyl)-3-methylazetidin-3-ol (50)

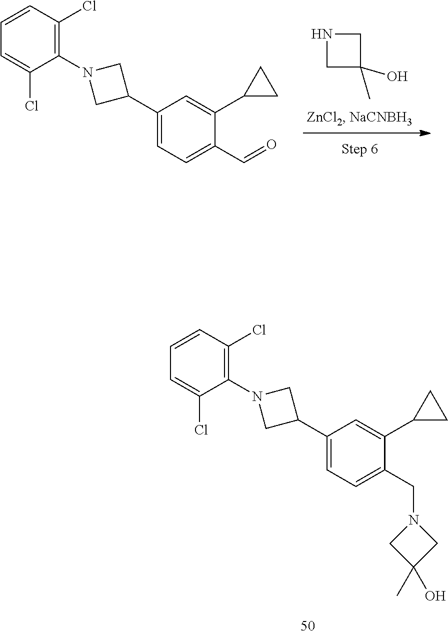

To a stirred solution of 3-methylazetidin-3-ol, TFA salt (87 mg, 0.433 mmol) in MeOH(5 mL) was added sodium bicarbonate (75 mg, 0.88 mmol.) then stirred for 1 h at rt. The mixture was filtered through celite and concentrated to yield free amine. To the free amine and 2-cyclopropyl-4-(1-(2,6-dichlorophenyl) azetidin-3-yl)benzaldehyde (150 mg, 0.433 mmol) in 5 mL of MeOH was added zinc chloride (59.0 mg, 0.433 mmol) and stirred for 1 h at 25° C. After 1 h, Sodium cyano borohydride (40.8 mg, 0.65 mmol.) was added and heated to 65° C. for 12 h. The reaction mixture was diluted with dichloromethane (10 mL) and washed with sat. ammonium chloride solution and water (20 mL). The combined organic phase was dried over Na₂SO₄, filtered and the solvents were evaporated under reduced pressure, the crude was purified by prep. HPLC(Diluent: THF: Water: ACN (50:10:40; Column: Luna C18 (250×21.2) mm, 10 micron Mobile phase A: 0.1% Formic acid in water, Mobile phase B: Acetonitrile). The required fractions were lyophilized to afford 1-(2-cyclopropyl-4-(1-(2,6-dichlorophenyl) azetidin-3-yl)benzyl)-3-methylazetidin-3-ol, formic acid salt (39.0 mg, 0.082 mmol, 18.89% yield, 97.2% purity) as a off white semi-solid.

¹H NMR (400 MHZ, MeOD): 7.32-7.37 (m, 1H), 7.15-7.21 (m, 3H), 6.74 (t, J=8.00 Hz, 1H), 4.34-4.39 (m, 4H), 4.86 (s, 3H), 3.84-3.86 (m, 2H), 3.66-3.75 (m, 3H), 2.06-2.12 (m, 1H), 1.53 (s, 3H), 1.03-1.08 (m, 2H), 0.73-0.76 (m, 2H); LCMS method 1, LCMS (ESI, m/z): 417.2 [M+H]⁺.

### Example S51. 1-(4-(1-(2,6-dichlorophenyl) azetidin-3-yl)-2-ethyl-6-methylbenzyl)-3-methylazetidin-3-ol (51)

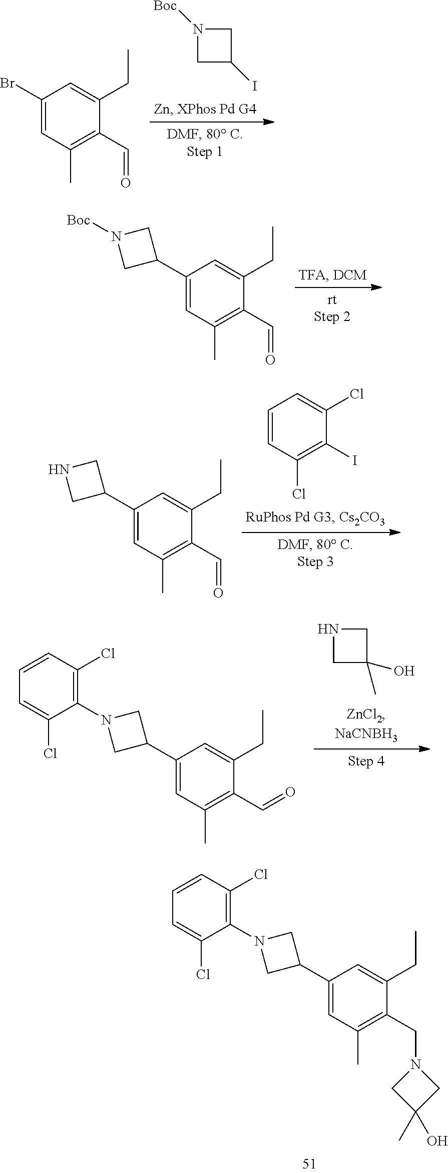

### Synthesis of tert-butyl 3-(3-ethyl-4-formyl-5-methylphenyl) azetidine-1-carboxylate

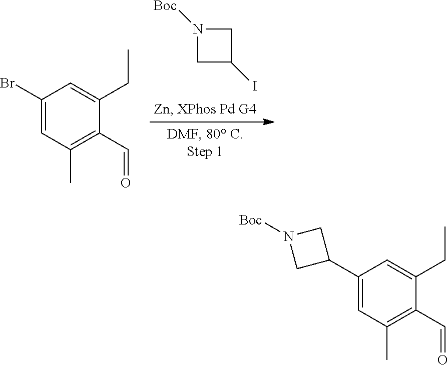 [SMILES: CCc1cc(Br)cc(C)c1C=O ; CC(C)(C)OC(=O)N1CC(I)C1 ; CCc1cc(C2CN(C(=O)OC(C)(C)C)C2)cc(C)c1C=O]

To a suspension of activated zinc (13.82 g, 211 mmol) in anhydrous DMF (40 mL) was added 1,2-dibromoethane (0.121 mL, 1.409 mmol) and heated to 75° C. After 15 mins, the reaction mixture was cooled to room temperature, trimethylchlorosilane (0.180 mL, 1.409 mmol) was added and allowed to stir at ambient temperature for additional 30 mins. Then a solution of tert-butyl 3-iodoazetidine-1-carboxylate (9.97 g, 35.2 mmol) in 10 mL of anhydrous DMF was added to the reaction mixture and stirred at room temperature for another 30 min., followed by 4-bromo-2-ethyl-6-methylbenzaldehyde (3.2 g, 14.09 mmol) and XPhos Pd G4 (1.2 g, 1.409 mmol) in 20 mL of DMF was added. The reaction mixture was allowed to stir at 80° C. for 2 h. After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (50 mL), dried over anhydrous sodium sulphate and concentrated under reduced pressure. The residue thus obtained was purified by flash column chromatography on silica gel (100-200 mesh) eluting with 0-50% ethyl acetate in petroleum ether to afford tert-butyl 3-(3-ethyl-4-formyl-5-methylphenyl) azetidine-1-carboxylate (3.0 g, 9.89 mmol, 70.2% yield) as a white solid; LCMS method 1; LCMS (ESI, m/z): 204.2 [M−100]⁺ .

### Synthesis of 4-(azetidin-3-yl)-2-ethyl-6-methylbenzaldehyde

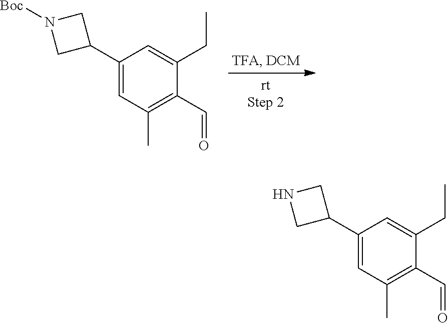 [SMILES: CCc1cc(C2CN(C(=O)OC(C)(C)C)C2)cc(C)c1C=O ; CCc1cc(C2CNC2)cc(C)c1C=O]

To a stirred solution of tert-butyl 3-(3-ethyl-4-formyl-5-methylphenyl) azetidine-1-carboxylate (1.0 g, 3.30 mmol) in anhydrous dichloromethane (20 mL) was added trifluoroacetic acid (1.524 mL, 19.78 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford 4-(azetidin-3-yl)-2-ethyl-6-methylbenzaldehyde, TFA (1.0 g, 89% yield) as a brown color semi-solid; LCMS method 1; LCMS (ESI, m/z): 204.2 [M+H]⁺.

### Synthesis of 4-(1-(2,6-dichlorophenyl) azetidin-3-yl)-2-ethyl-6-methylbenzaldehyde

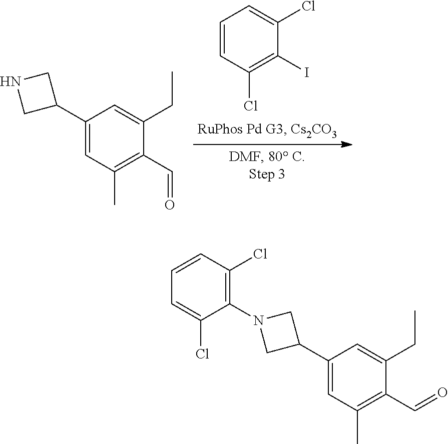 [SMILES: CCc1cc(C2CNC2)cc(C)c1C=O ; Clc1cccc(Cl)c1I ; CCc1cc(C2CN(c3c(Cl)cccc3Cl)C2)cc(C)c1C=O]

To a solution of 4-(azetidin-3-yl)-2-ethyl-6-methylbenzaldehyde, TFA (1.0 g, 3.15 mmol) and 1,3-dichloro-2-iodobenzene (1.032 g, 3.78 mmol) in anhydrous 1,4 dioxane (20 mL) was added Cs₂CO₃ (3.59 g, 11.03 mmol). The reaction mixture was then degassed with nitrogen for 10 min followed by added RuPhos Pd G3 (0.264 g, 0.315 mmol) and heated to 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 4-(1-(2,6-dichlorophenyl) azetidin-3-yl)-2-ethyl-6-methylbenzaldehyde (1 g, 2.464 mmol, 78% yield) as a green color solid; LCMS method 1; LCMS (ESI, m/z): 348.0 [M+H]⁺.

### Synthesis of 1-(4-(1-(2,6-dichlorophenyl) azetidin-3-yl)-2-ethyl-6-methylbenzyl)-3-methylazetidin-3-ol (51)

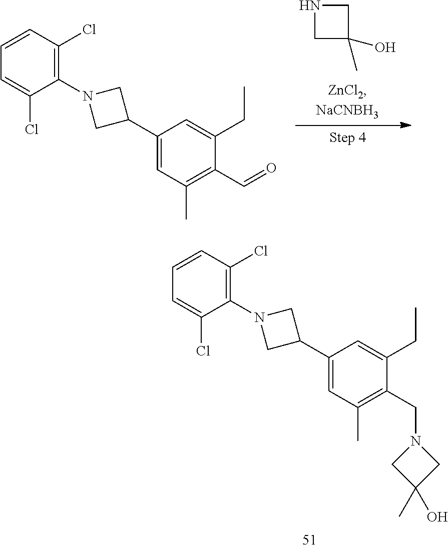

To a stirred solution of 4-(1-(2,6-dichlorophenyl) azetidin-3-yl)-2-ethyl-6-methylbenzaldehyde (200 mg, 0.574 mmol) and 3-methylazetidin-3-ol (60.0 mg, 0.689 mmol) in 10 mL of MeOH was added zinc chloride (117 mg, 0.861 mmol) and stirred for 1 h at 25° C. After 1 h, sodium cyanoborohydride (54.1 mg, 0.861 mmol) was added and heated to 65° C. for 12 h. The reaction mixture was diluted with dichloromethane (10 mL) and washed with sat. ammonium chloride solution and water (20 mL). The combined organic phase was dried over Na₂SO₄, filtered and the solvents were evaporated under reduced pressure, the crude was purified by prep. HPLC (Diluent: THF: Water: ACN (50:10:40; Column: Luna C18 (250×21.2) mm, 10 micron Mobile phase A: 0.1% Formic acid in water, Mobile phase B: Acetonitrile). The required fractions were lyophilized to afford 1-(4-(1-(2,6-dichlorophenyl) azetidin-3-yl)-2-ethyl-6-methylbenzyl)-3-methylazetidin-3-ol, formic acid salt (13 mg, 0.028 mmol, 4.86% yield, 99.84% purity) as a white semi-solid.

¹H NMR (400 MHZ, MeOD): δ 7.22-7.19 (m, 4H), 8 6.75-6.71 (t, J=8 Hz, 1H), 8 4.41-4.38 (t, J=6.4 Hz, 2H), 8 4.32 (s, 2H), 8 3.88-3.86 (m, 2H), 8 3.75-3.69 (m, 3H), 8 2.84-2.78 (q, J=7.6 Hz, 2H), 8 2.47 (s, 3H), 8 1.49 (s, 3H), 8 1.26-1.22 (t, J=7.6 Hz, 3H)(two protons are merged with solvent signal); LCMS method 1, LCMS (ESI, m/z): 419.2 [M+H]⁺.

### Example S52. 1-(4-(1-(2,6-dichlorophenyl) azetidin-3-yl)-2,6-diethylbenzyl)-3-methylazetidin-3-ol (52)

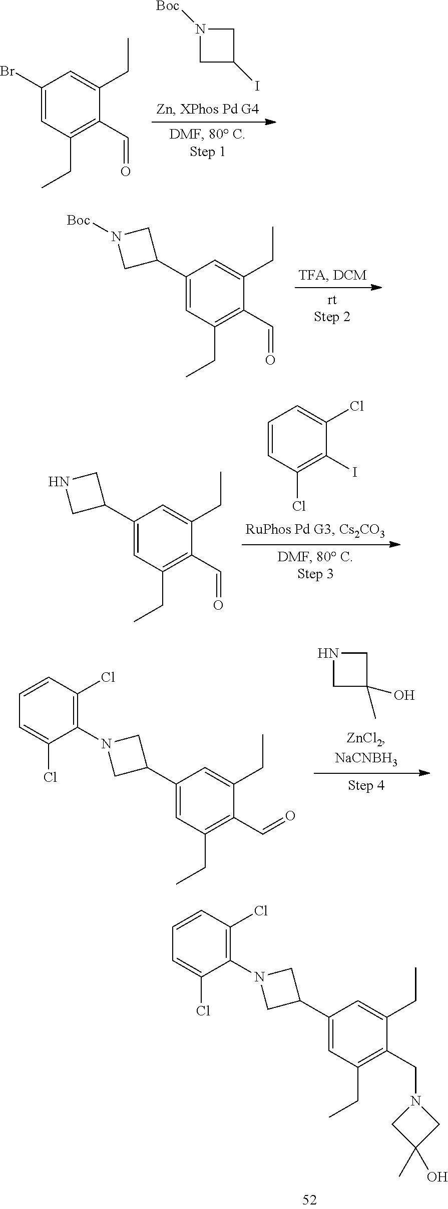

### Synthesis of tert-butyl 3-(3,5-diethyl-4-formylphenyl) azetidine-1-carboxylate

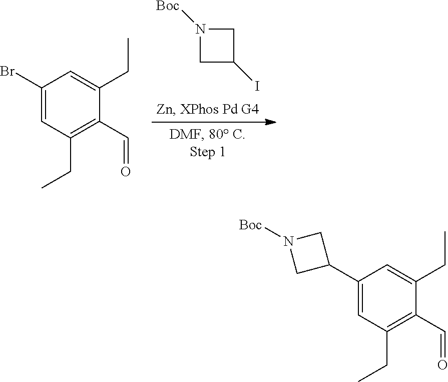 [SMILES: CCc1cc(Br)cc(CC)c1C=O ; CC(C)(C)OC(=O)N1CC(I)C1 ; CCc1cc(C2CN(C(=O)OC(C)(C)C)C2)cc(CC)c1C=O]

To a suspension of activated zinc (2.71 g, 41.5 mmol) in anhydrous DMF (30 mL) was added 1,2-dibromoethane (4.78 μL, 0.055 mmol) and heated to 75° C. After 15 mins, the reaction mixture was cooled to room temperature, chloro trimethylsilane (265 μL, 2.074 mmol) was added and allowed to stir at ambient temperature for additional 30 mins. Then a solution of tert-butyl 3-iodoazetidine-1-carboxylate (3.52 g, 12.44 mmol) in 10 mL of anhydrous DMF was added to the reaction mixture and stirred at room temperature for another 30 min, followed by 4-bromo-2,6-diethylbenzaldehyde (1.0 g, 4.15 mmol) and XPhos Pd G4 (0.535 g, 0.622 mmol) in 20 mL of DMF was added. The reaction mixture was allowed to stir at 80° C. for 2 h. After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (50 mL), dried over anhydrous sodium sulphate and concentrated under reduced pressure. The residue thus obtained was purified by flash column chromatography on silica gel (100-200 mesh) eluting with 0-50% ethyl acetate in petroleum ether to afford tert-butyl 3-(3,5-diethyl-4-formylphenyl) azetidine-1-carboxylate (910 mg, 69.3% yield) as a yellow liquid; LCMS method 1, LCMS (ESI, m/z): 218.1 [M−100]⁺ .

### Synthesis of 4-(azetidin-3-yl)-2,6-diethylbenzaldehyde

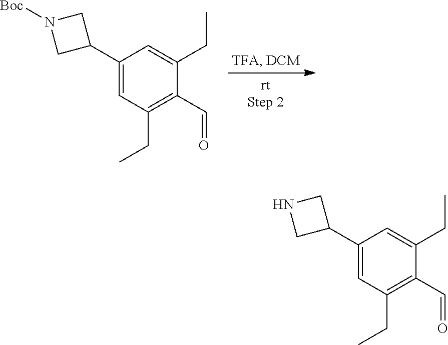 [SMILES: CCc1cc(C2CN(C(=O)OC(C)(C)C)C2)cc(CC)c1C=O ; CCc1cc(C2CNC2)cc(CC)c1C=O]

To a stirred solution of tert-butyl 3-(3,5-diethyl-4-formylphenyl) azetidine-1-carboxylate (950 mg, 2.99 mmol) in anhydrous dichloromethane (20 mL) was added trifluoroacetic acid (2.5 mL, 32.4 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford 4-(azetidin-3-yl)-2,6-diethylbenzaldehyde, TFA (800 mg, 2.390 mmol, 80% yield) as a yellow liquid; LCMS method 1; LCMS (ESI, m/z): 218.2 [M+H]⁺.

### Synthesis of 4-(1-(2,6-dichlorophenyl) azetidin-3-yl)-2,6-diethylbenzaldehyde

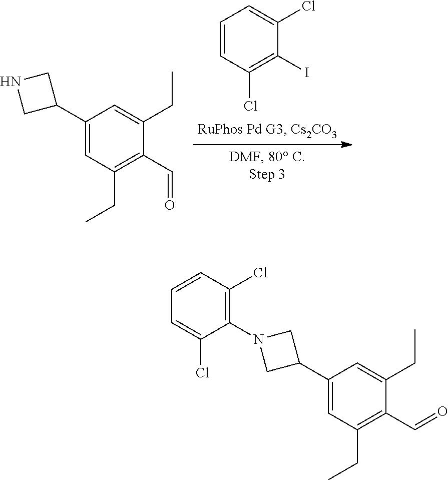 [SMILES: CCc1cc(C2CNC2)cc(CC)c1C=O ; Clc1cccc(Cl)c1I ; CCc1cc(C2CN(c3c(Cl)cccc3Cl)C2)cc(CC)c1C=O]

To a solution of 4-(azetidin-3-yl)-2,6-diethylbenzaldehyde, TFA salt (1.1 g, 3.68 mmol) and 1,3-dichloro-2-iodobenzene (1206 mg, 4.42 mmol) in anhydrous 1,4 dioxane (20 mL) was added cesium carbonate (3.7 g, 11.04 mmol). The reaction mixture was then degassed with nitrogen for 10 min followed by added RuPhos Pd G3 (308 mg, 0.368 mmol) and heated to 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 4-(1-(2,6-dichlorophenyl) azetidin-3-yl)-2,6-diethylbenzaldehyde (365 mg, 27.3% yield) as a pale yellow solid; LCMS method 1, LCMS (ESI, m/z): 362.0 [M+H]⁺.

### Synthesis of 1-(4-(1-(2,6-dichlorophenyl) azetidin-3-yl)-2,6-diethylbenzyl)-3-methylazetidin-3-ol (52)

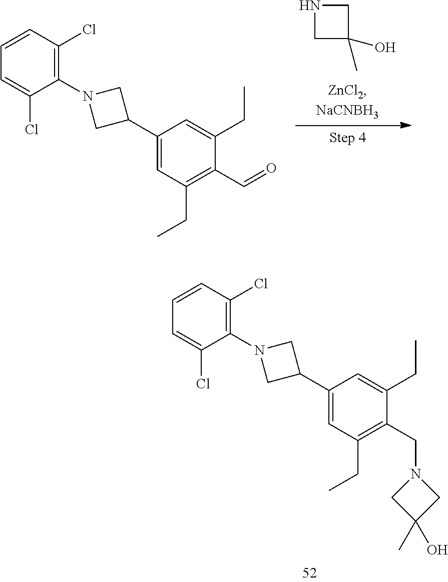

To a stirred solution of 3-methylazetidin-3-ol, TFA salt (250 mg, 1.242 mmol) in MeOH(5 mL) was added sodium bicarbonate (208 mg, 2.484 mmol) then stirred for 1 h at rt. The mixture was filtered through celite and concentrated to yield free amine. To the free amine and 4-(1-(2,6-dichlorophenyl) azetidin-3-yl)-2,6-diethylbenzaldehyde (450 mg, 1.242 mmol) in 5 mL of MeOH was added zinc chloride (169 mg, 1.242 mmol) and stirred for 1 h at 25° C. After 1 h, Sodium cyano borohydride (117 mg, 1.863 mmol) was added and heated to 65° C. for 12 h. The reaction mixture was diluted with dichloromethane (10 mL) and washed with sat. ammonium chloride solution and water (20 mL). The combined organic phase was dried over Na₂SO₄, filtered and the solvents were evaporated under reduced pressure, the crude was purified by prep. HPLC(Diluent: THF: Water: ACN (50:10:40; Column: Luna C18 (250×21.2) mm, 10 micron Mobile phase A: 0.1% Formic acid in water, Mobile phase B: Acetonitrile). The required fractions were lyophilized to afford 1-(4-(1-(2,6-dichlorophenyl) azetidin-3-yl)-2,6-diethylbenzyl)-3-methylazetidin-3-ol, formic acid salt (201.2 mg, 0.415 mmol, 33.4% yield, 98.9% purity) as a white semi-solid.

¹H NMR (400 MHZ,MeOD): 7.14-7.20 (m, 2H), 6.72-6.74 (m, 2H), 6.70 (m, 1H), 4.38-4.40 (m, 2H), 3.73-3.90 (m, 2H), 3.69-3.72 (m, 1H), 3.40 (m, 2H), 3.28-3.33 (m, 2H), 2.78-2.84 (m, 4H), 1.45 (s, 3H), 1.23 (t, J=7.60 Hz, 6H)(two protons are merged with solvent signal); LCMS method 2; LCMS (ESI, m/z): 433.2 [M+H]⁺.

### Example S53. 1-(4-(1-(2,6-dichlorophenyl) azetidin-3-yl)-2,6-diisopropylbenzyl)-3-methylazetidin-3-ol (53)

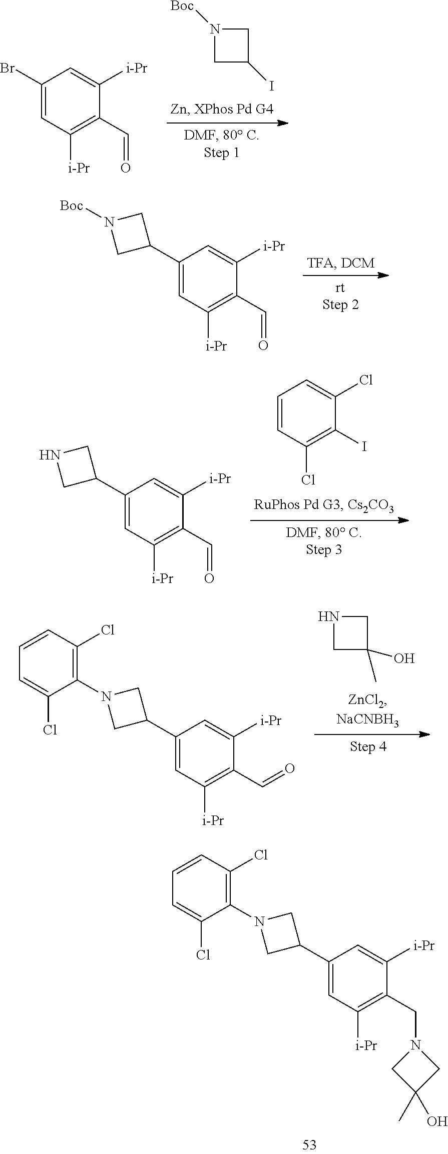

### Synthesis of tert-butyl 3-(4-formyl-3,5-diisopropylphenyl) azetidine-1-carboxylate

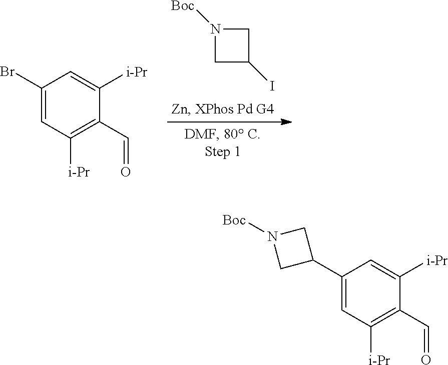 [SMILES: CC(C)c1cc(Br)cc(C(C)C)c1C=O ; CC(C)(C)OC(=O)N1CC(I)C1 ; CC(C)c1cc(C2CN(C(=O)OC(C)(C)C)C2)cc(C(C)C)c1C=O]

To a suspension of activated zinc (2.91 g, 44.6 mmol) in anhydrous DMF (30 mL) was added 1,2-dibromoethane (0.192 ml, 2.229 mmol) and heated to 75° C. After 15 mins, the reaction mixture was cooled to room temperature, chloro trimethylsilane (0.285 ml, 2.229 mmol) was added and allowed to stir at ambient temperature for additional 30 mins. Then a solution of tert-butyl 3-iodoazetidine-1-carboxylate (3.79 g, 13.37 mmol) in 10 mL of anhydrous DMF was added to the reaction mixture and stirred at room temperature for another 30 min., followed by 4-bromo-2,6-diisopropylbenzaldehyde (1.2 g, 4.46 mmol) and XPhos Pd G4 (0.575 g, 0.669 mmol) in 20 mL of DMF was added. The reaction mixture was allowed to stir at 80° C. for 2 h. After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (50 mL), dried over anhydrous sodium sulphate and concentrated under reduced pressure. The residue thus obtained was purified by flash column chromatography on silica gel (100-200 mesh) eluting with 0-50% ethyl acetate in petroleum ether to afford tert-butyl 3-(4-formyl-3,5-diisopropylphenyl) azetidine-1-carboxylate (841 mg, 54.5% yield); LCMS method 1, LCMS (ESI, m/z): 246.2 [M−100]⁺ .

### Synthesis of 4-(azetidin-3-yl)-2,6-diisopropylbenzaldehyde

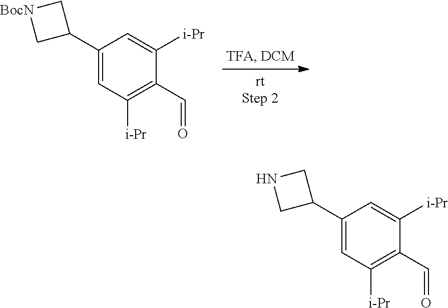 [SMILES: CC(C)c1cc(C2CN(C(=O)OC(C)(C)C)C2)cc(C(C)C)c1C=O ; CC(C)c1cc(C2CNC2)cc(C(C)C)c1C=O]

To a stirred solution of tert-butyl 3-(4-formyl-3,5-diisopropylphenyl) azetidine-1-carboxylate (1.15 g, 3.33 mmol) in anhydrous dichloromethane (30 mL) was added trifluoroacetic acid (2.0 mL, 26.0 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford 4-(azetidin-3-y1)-2,6-diisopropylbenzaldehyde, TFA (1.04 g, 87% yield) as a brown semi-solid; LCMS method 2; LCMS (ESI, m/z): 246.1 [M+H]⁺.

### Synthesis of 4-(1-(2,6-dichlorophenyl) azetidin-3-yl)-2,6-diisopropylbenzaldehyde

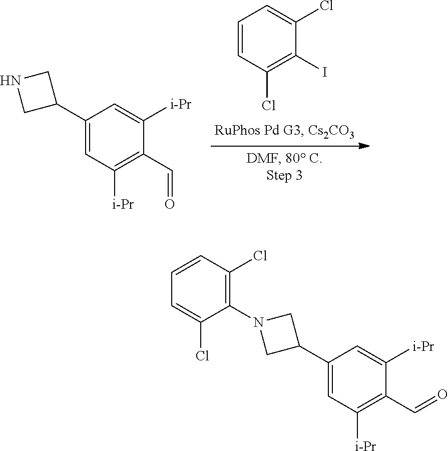 [SMILES: CC(C)c1cc(C2CNC2)cc(C(C)C)c1C=O ; Clc1cccc(Cl)c1I ; CC(C)c1cc(C2CN(c3c(Cl)cccc3Cl)C2)cc(C(C)C)c1C=O]

To a solution of 4-(azetidin-3-yl)-2,6-diisopropylbenzaldehyde, TFA salt (836 mg, 2.445 mmol) and 1,3-dichloro-2-iodobenzene (801 mg, 2.93 mmol) in anhydrous 1,4 dioxane (20 mL) was added cesium carbonate (7.34 mmol). The reaction mixture was then degassed with nitrogen for 10 min followed by added RuPhos Pd G3 (205 mg, 0.245 mmol) and heated to 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 4-(1-(2,6-dichlorophenyl) azetidin-3-yl)-2,6-diisopropylbenzaldehyde (207 mg, 21.69% yield) as a pale yellow semi-solid; LCMS method 1, LCMS (ESI, m/z): 390.2 [M+H]⁺.

### Synthesis of 1-(4-(1-(2,6-dichlorophenyl) azetidin-3-yl)-2,6-diisopropylbenzyl)-3-methylazetidin-3-ol (53)

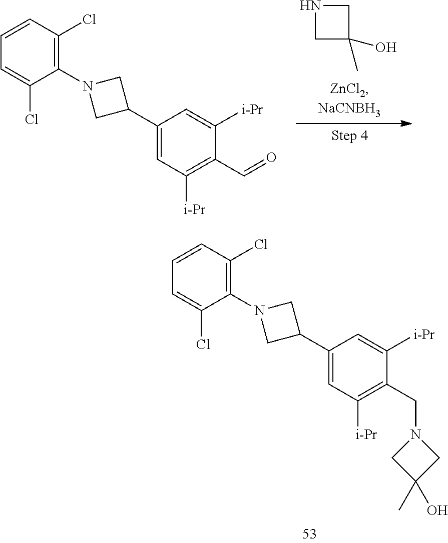

To a stirred solution of 3-methylazetidin-3-ol, TFA salt (232 mg, 1.153 mmol) in MeOH(5 mL) was added sodium bicarbonate (193 mg, 2.3 mmol) then stirred for 1 h at rt. The mixture was filtered through celite and concentrated to yield free amine. To the free amine and 4-(1-(2,6-dichlorophenyl) azetidin-3-yl)-2,6-diisopropylbenzaldehyde (450 mg, 1.153 mmol) in 5 mL of MeOH was added zinc chloride (157 mg, 1.153 mmol) and stirred for 1 h at 25° C. After 1 h, Sodium cyano borohydride (109 mg, 1.729 mmol) was added and heated to 65° C. for 12 h. The reaction mixture was diluted with dichloromethane (10 mL) and washed with sat. ammonium chloride solution and water (20 mL). The combined organic phase was dried over Na₂SO₄, filtered and the solvents were evaporated under reduced pressure, the crude was purified by prep. HPLC(Diluent: THF: Water: ACN (50:10:40; Column: Luna C18 (250×21.2) mm, 10 micron Mobile phase A: 0.1% Formic acid in water, Mobile phase B: Acetonitrile). The required fractions were lyophilized to afford 1-(4-(1-(2,6-dichlorophenyl) azetidin-3-yl)-2,6-diisopropylbenzyl)-3-methylazetidin-3-ol, formic acid salt (194.9 mg, 0.373 mmol, 32.4% yield, 97.2% yield) as a white semi-solid.

¹H NMR (400 MHZ, MeOD): 7.34 (s, 2H), 7.20-7.22 (m, 2H), 6.73-6.77 (m, 1H), 4.93 (s, 2H), 4.36-4.40 (m, 2H), 4.21 (s, 2H), 3.68-3.77 (m, 3H), 3.54-3.56 (m, 2H), 3.38 (m, 2H), 1.48 (s, 3H), 1.28-1.33 (m, 12H); LCMS method 1, LCMS (ESI, m/z): 461.2 [M+H]⁺.

### Example S54. 1-(2-cyclopropyl-4-(1-(2,6-dichlorophenyl) azetidin-3-yl)-6-methylbenzyl)-3-methylazetidin-3-ol (54)

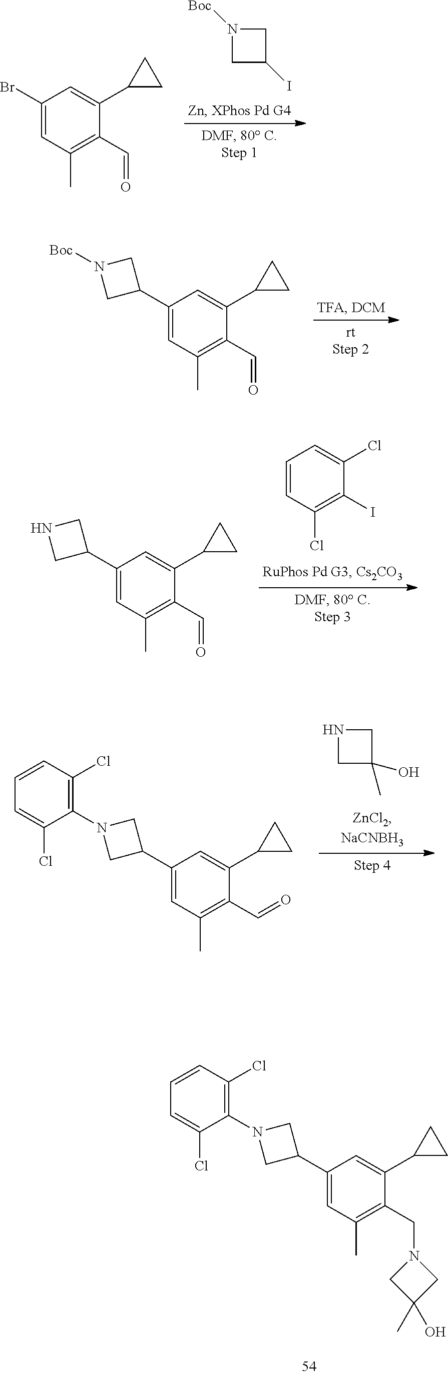

### Synthesis of tert-butyl 3-(3-cyclopropyl-4-formyl-5-methylphenyl) azetidine-1-carboxylate

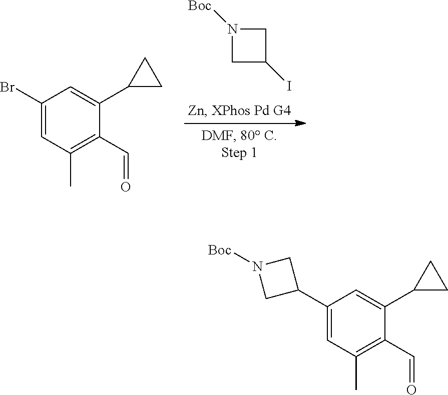 [SMILES: Cc1cc(Br)cc(C2CC2)c1C=O ; CC(C)(C)OC(=O)N1CC(I)C1 ; Cc1cc(C2CN(C(=O)OC(C)(C)C)C2)cc(C2CC2)c1C=O]

To a suspension of activated Zinc dust (4.37 g, 66.9 mmol) in anhydrous DMF (20 mL) was added 1,2-Dibromoethane (0.126 g, 0.669 mmol) and heated to 75° C. After 15 mins, the reaction mixture was cooled to room temperature, Trimethylsilyl chloride (0.073 g, 0.669 mmol) was added and allowed to stir at ambient temperature for additional 30 mins. Then a solution of tert-butyl 3-iodoazetidine-1-carboxylate (5.68 g, 20.07 mmol) in 5 mL of anhydrous DMF was added to the reaction mixture and stirred at room temperature for another 30 min., followed by 4-bromo-2-cyclopropyl-6-methylbenzaldehyde (1.6 g, 6.69 mmol) and XPhos Pd G4 (0.576 g, 0.669 mmol) in 10 mL of DMF was added. The reaction mixture was allowed to stir at 80° C. for 2 h. After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (50 mL), dried over anhydrous sodium sulphate and concentrated under reduced pressure. The residue thus obtained was purified by flash column chromatography on silica gel (100-200 mesh) eluting with 0-50% ethyl acetate in petroleum ether to afford tert-butyl 3-(3-cyclopropyl-4-formyl-5-methylphenyl) azetidine-1-carboxylate (0.858 g, 40.7% yield); LCMS method 1; LCMS (ESI, m/z): 216.1 [M−100]*.

### Synthesis of 4-(azetidin-3-yl)-2-cyclopropyl-6-methylbenzaldehyde

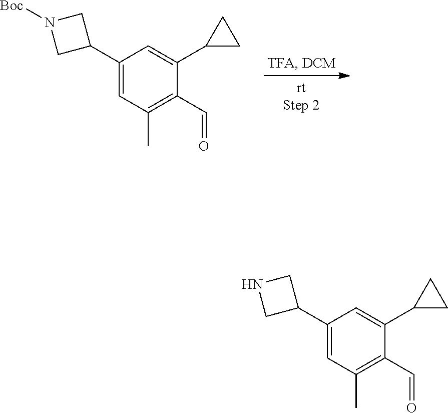 [SMILES: Cc1cc(C2CN(C(=O)OC(C)(C)C)C2)cc(C2CC2)c1C=O ; Cc1cc(C2CNC2)cc(C2CC2)c1C=O]

To a stirred solution of tert-butyl 3-(3-cyclopropyl-4-formyl-5-methylphenyl) azetidine-1-carboxylate (1 g, 3.17 mmol) in anhydrous dichloromethane (30 mL) was added trifluoroacetic acid (0.733 mL, 9.51 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford 4-(azetidin-3-yl)-2-cyclopropyl-6-methylbenzaldehyde, TFA (0.82, 79% yield) as a yellow solid; LCMS method 1, LCMS (ESI, m/z): 216.0 [M+H]⁺.

### Synthesis of 2-cyclopropyl-4-(1-(2,6-dichlorophenyl) azetidin-3-yl)-6-methylbenzaldehyde

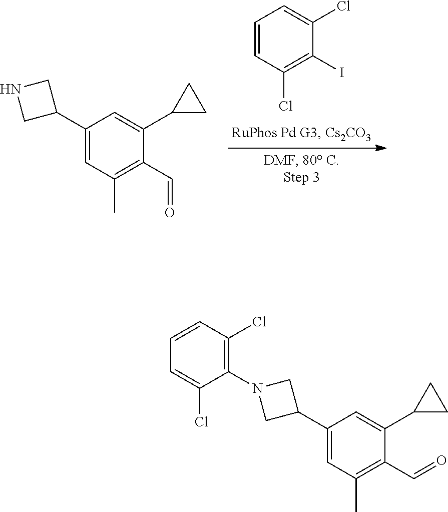 [SMILES: Cc1cc(C2CNC2)cc(C2CC2)c1C=O ; Clc1cccc(Cl)c1I ; Cc1cc(C2CN(c3c(Cl)cccc3Cl)C2)cc(C2CC2)c1C=O]

To a solution of 4-(azetidin-3-yl)-2-cyclopropyl-6-methylbenzaldehyde, TFA (724 mg, 2.199 mmol) and 1,3-dichloro-2-iodobenzene (600 mg, 2.199 mmol) in anhydrous 1,4 dioxane (20 mL) was added Cesium carbonate (2149 mg, 6.60 mmol). The reaction mixture was then degassed with nitrogen for 10 min followed by added RuPhos Pd G3 (92 mg, 0.110 mmol) and heated to 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 2-cyclopropyl-4-(1-(2,6-dichlorophenyl) azetidin-3-yl)-6-methylbenzaldehyde (400 mg, 0.910 mmol, 41.4% yield) as a yellow solid; LCMS method 1, LCMS (ESI, m/z): 360.0 [M+H]⁺.

### Synthesis of 1-(2-cyclopropyl-4-(1-(2,6-dichlorophenyl) azetidin-3-yl)-6-methylbenzyl)-3-methylazetidin-3-ol (54)

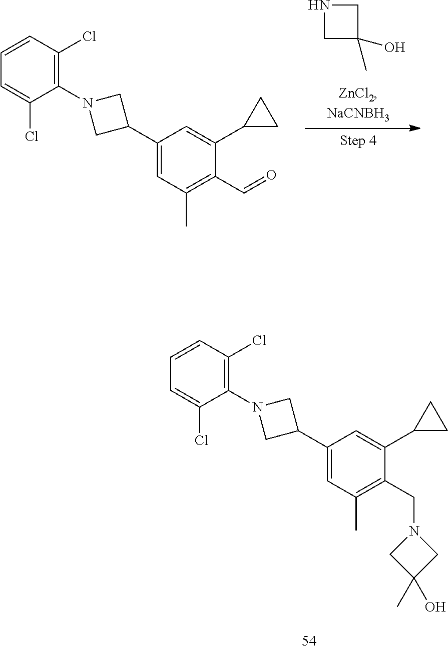

To a stirred solution of 3-methylazetidin-3-ol, TFA (84 mg, 0.416 mmol) in MeOH(5 mL) was added sodium bicarbonate (70.0 mg, 0.833 mmol) then stirred for 1 h at rt. The mixture was filtered through celite and concentrated to yield free amine. To the free amine and 2-cyclopropyl-4-(1-(2,6-dichlorophenyl) azetidin-3-yl)-6-methylbenzaldehyde (150 mg, 0.416 mmol) in 5 mL of MeOH was added zinc chloride (68.1 mg, 0.500 mmol) and stirred for 1 h at 25° C. After 1 h, Sodium cyano borohydride (52.4 mg, 0.832 mmol) was added and heated to 65° C. for 12 h. The reaction mixture was diluted with dichloromethane (10 mL) and washed with sat. ammonium chloride solution and water (20 mL). The combined organic phase was dried over Na₂SO₄, filtered and the solvents were evaporated under reduced pressure, the crude was purified by prep. HPLC(Diluent: THF: Water: ACN (50:10:40); Column-1: Xbridge C₈ (250×19) mm, 5 micron; Mobile phase A: 0.1% Formic acid in water; Mobile phase B: Acetonitrile). The required fractions were lyophilized to afford 1-(2-cyclopropyl-4-(1-(2,6-dichlorophenyl) azetidin-3-yl)-6-methylbenzyl)-3-methylazetidin-3-ol, formic acid salt (30 mg, 0.062 mmol, 14.94% yield, 99% purity) as an off white solid.

¹H NMR (400 MHZ, MeOD): δ 7.21 (t, J=8.00 Hz, 3H), 7.01 (s, 1H), 6.74 (t, J=7.60 Hz, 1H), 4.51 (s, 2H), 4.35-4.38 (m, 2H), 3.91-3.93 (m, 2H), 3.81 (m, 2H), 3.65-3.81 (m, 1H), 2.47 (s, 3H), 2.11-2.17 (m, 1H), 1.51 (s, 3H), 1.06-1.11 (m, 2H), 0.74-0.77 (m, 2H)(2H are merged with solvent signal); LCMS method 1, LCMS (ESI, m/z): 433.2 [M+2H]*.

### Example S55. 1-((4-(1-(2,6-dichlorophenyl) azetidin-3-yl) naphthalen-1-yl)methyl)-3-methylazetidin-3-ol (55)

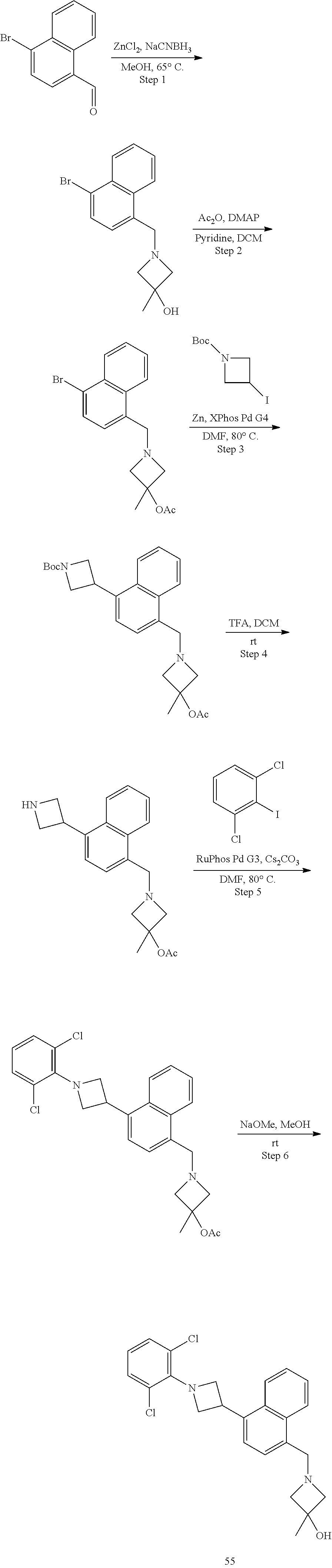

### Synthesis of 1-((4-bromonaphthalen-1-yl)methyl)-3-methylazetidin-3-ol

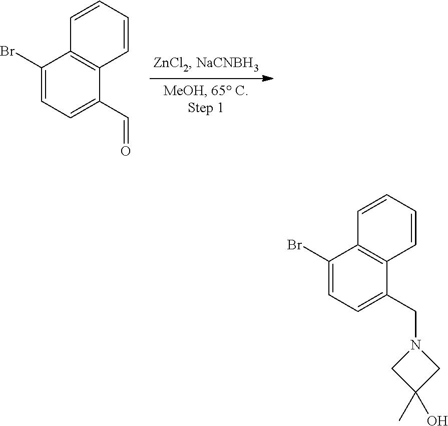 [SMILES: O=Cc1ccc(Br)c2ccccc12 ; CC1(O)CN(Cc2ccc(Br)c3ccccc23)C1]

To a stirred solution of 3-methylazetidin-3-ol, TFA salt (2.57 g, 12.76 mmol) in MeOH(5 mL) was added sodium bicarbonate (2.1 g, 25.2 mmol.) then stirred for 1 h at rt. The mixture was filtered through celite and concentrated to yield free amine. To the free amine and 4-bromo-1-naphthaldehyde (3.0 g, 12.76 mmol) in 5 mL of MeOH was added zinc chloride (1.739 g, 12.76 mmol) and stirred for 1 h at 25° C. After 1 h, Sodium cyano borohydride (1.2 g, 12.76 mmol.) was added and heated to 65° C. for 12 h. The reaction mixture was diluted with dichloromethane (10 mL) and washed with sat. ammonium chloride solution and water (20 mL). The combined organic phase was dried over Na₂SO₄, filtered and the solvents were evaporated under reduced pressure, the crude was purified by flash column chromatography on 230-400 mesh silica gel (eluted with 0-50% ethyl acetate in pet ether) to afford 1-((4-bromonaphthalen-1-yl)methyl)-3-methylazetidin-3-ol (0.79 g, 20.27% yield) as a yellow semi-solid, LCMS method 1, LCMS (ESI, m/z): 307.8 [M+2H]⁺

### Synthesis of 1-((4-bromonaphthalen-1-yl)methyl)-3-methylazetidin-3-yl acetate

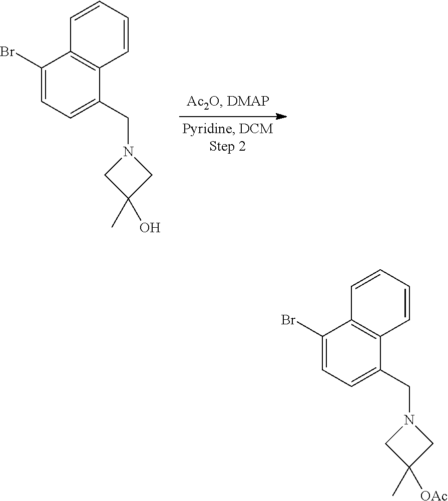 [SMILES: CC1(O)CN(Cc2ccc(Br)c3ccccc23)C1 ; CC(=O)OC1(C)CN(Cc2ccc(Br)c3ccccc23)C1]

To a stirred solution of 1-((4-bromonaphthalen-1-yl)methyl)-3-methylazetidin-3-ol (1.2 g, 3.92 mmol) in DCM (20 mL) were added DMAP (0.479 g, 3.92 mmol) and Pyridine (0.317 mL, 3.92 mmol) and the reaction mixture was stirred at room temperature for 10 minutes. After that acetic anhydride (1.109 mL, 11.76 mmol) was added and the reaction mixture was stirred for 16 h at rt. The reaction mixture was diluted with saturated solution of ammonium chloride and extracted with DCM. The combined organic layer was dried over sodium sulphate, concentrated under vacuum, purified by flash column chromatography on 230-400 mesh silica gel (eluted with 0-30% ethyl acetate in pet ether) to afford 1-((4-bromonaphthalen-1-yl)methyl)-3-methylazetidin-3-yl acetate (0.6 g, 89% purity, 39% yield) as a transparent semi-solid, LCMS method 1, LCMS (ESI, m/z): 349.0 [M+2H]⁺ .

### Synthesis of tert-butyl 3-(4-((3-acetoxy-3-methylazetidin-1-yl)methyl) naphthalen-1-yl) azetidine-1-carboxylate

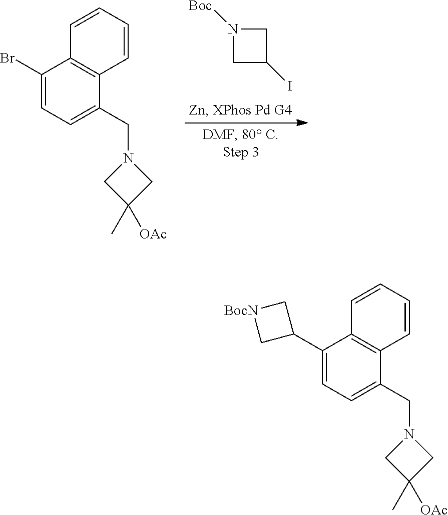 [SMILES: CC(=O)OC1(C)CN(Cc2ccc(Br)c3ccccc23)C1 ; CC(C)(C)OC(=O)N1CC(I)C1 ; CC(=O)OC1(C)CN(Cc2ccc(C3CN(C(=O)OC(C)(C)C)C3)c3ccccc23)C1]

To a suspension of activated zinc (1126 mg, 17.23 mmol) in anhydrous DMF (10 mL) was added 1,2-dibromoethane (0.074 ml, 0.861 mmol) and heated to 75° C. After 15 mins, the reaction mixture was cooled to room temperature, trimethylchlorosilane (0.285 mL, 2.229 mmol) was added and allowed to stir at ambient temperature for additional 30 mins. Then a solution of tert-butyl 3-iodoazetidine-1-carboxylate (1463 mg, 5.17 mmol) in 5 mL of anhydrous DMF was added to the reaction mixture and stirred at room temperature for another 30 min., followed by 1-((4-bromonaphthalen-1-yl)methyl)-3-methylazetidin-3-yl acetate (600 mg, 1.723 mmol) and XPhos Pd G4 (222 mg, 0.258 mmol) in 5 mL of DMF was added. The reaction mixture was allowed to stir at 80° C. for 2 h. After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (50 mL), dried over anhydrous sodium sulphate and concentrated under reduced pressure. The residue thus obtained was purified by flash column chromatography on silica gel (100-200 mesh) eluting with 0-50% ethyl acetate in petroleum ether to afford tert-butyl 3-(4-((3-acetoxy-3-methylazetidin-1-yl)methyl) naphthalen-1-yl) azetidine-1-carboxylate (248 mg, 34.0% yield) as a yellow semi-solid; LCMS method 1, LCMS (ESI, m/z): 425.5 [M+H]⁺.

### Synthesis of 1-((4-(azetidin-3-yl) naphthalen-1-yl)methyl)-3-methylazetidin-3-yl acetate

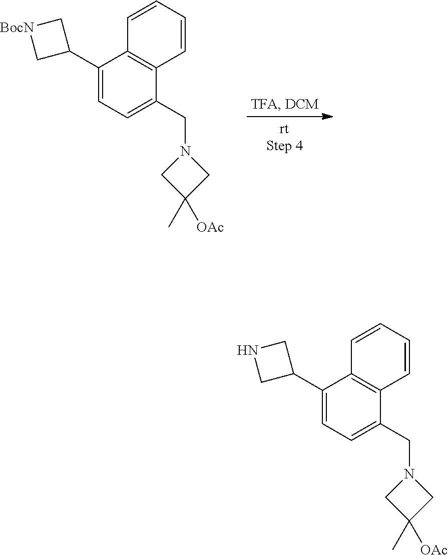 [SMILES: CC(=O)OC1(C)CN(Cc2ccc(C3CN(C(=O)OC(C)(C)C)C3)c3ccccc23)C1 ; CC(=O)OC1(C)CN(Cc2ccc(C3CNC3)c3ccccc23)C1]

To a stirred solution of tert-butyl 3-(4-((3-acetoxy-3-methylazetidin-1-yl)methyl) naphthalen-1-yl) azetidine-1-carboxylate (270 mg, 0.636 mmol) in anhydrous dichloromethane (10 mL) was added trifluoroacetic acid (2.0 mL, 26.0 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to 1-((4-(azetidin-3-yl) naphthalen-1-yl)methyl)-3-methylazetidin-3-yl acetate, TFA (279 mg, 100% yield) as a brown color semi-solid; LCMS method 1, LCMS (ESI, m/z): 325.2 [M+H]⁺.

### Synthesis of 1-((4-(1-(2,6-dichlorophenyl) azetidin-3-yl) naphthalen-1-yl)methyl)-3-methylazetidin-3-yl acetate

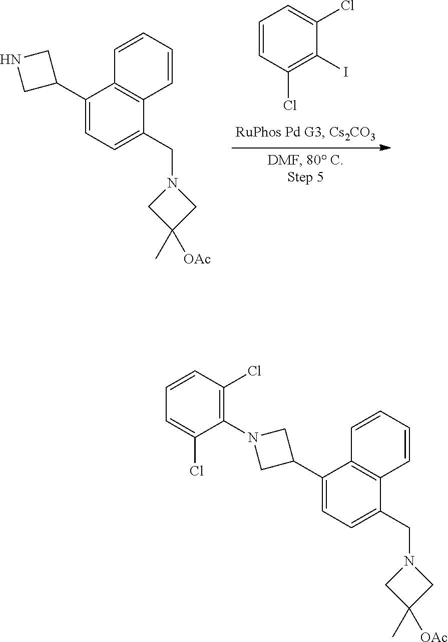 [SMILES: CC(=O)OC1(C)CN(Cc2ccc(C3CNC3)c3ccccc23)C1 ; Clc1cccc(Cl)c1I ; CC(=O)OC1(C)CN(Cc2ccc(C3CN(c4c(Cl)cccc4Cl)C3)c3ccccc23)C1]

To a solution of 1-((4-(azetidin-3-yl) naphthalen-1-yl)methyl)-3-methylazetidin-3-yl acetate, TFA (300 mg, 0.684 mmol) and 1,3-dichloro-2-iodobenzene (224 mg, 0.821 mmol) in anhydrous 1,4 dioxane (5 mL) was added Cs₂CO₃ (3.59 g, 11.03 mmol). The reaction mixture was then degassed with nitrogen for 10 min followed by added Ruphos pd G3 (57.2 mg, 0.068 mmol) and heated to 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 1-((4-(1-(2,6-dichlorophenyl) azetidin-3-yl) naphthalen-1-yl)methyl)-3-methylazetidin-3-yl acetate (100 mg, 30.8% yield) as a pale yellow solid; LCMS method 1, LCMS (ESI, m/z): 469.0 [M+H]⁺

### Synthesis of 1-((4-(1-(2,6-dichlorophenyl) azetidin-3-yl) naphthalen-1-yl)methyl)-3-methylazetidin-3-ol (55)

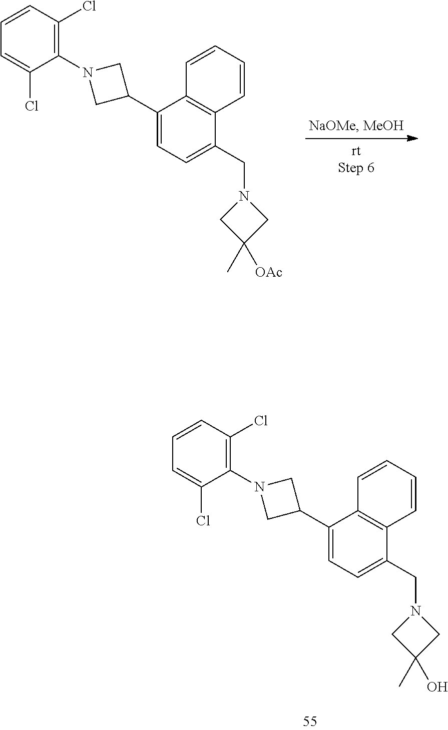 [SMILES: CC(=O)OC1(C)CN(Cc2ccc(C3CN(c4c(Cl)cccc4Cl)C3)c3ccccc23)C1 ; CC1(O)CN(Cc2ccc(C3CN(c4c(Cl)cccc4Cl)C3)c3ccccc23)C1]

To a stirred solution of 1-((4-(1-(2,6-dichlorophenyl) azetidin-3-yl) naphthalen-1-yl)methyl)-3-methylazetidin-3-yl acetate (100 mg, 0.213 mmol) in methanol (5 mL) was added sodium methoxide (28.8 mg, 0.533 mmol). The resulting mixture was stirred at room temperature for 1 h. The reaction mixture was quenched with saturated solution of ammonium chloride and extracted with EtOAc. The combined organic layer was dried over sodium sulphate, concentrated under vacuum. The crude was purified using Prep. HPLC(Diluent: THF: Water: ACN (50:20:30); Column: Luna C18 (250×21.2) mm, 10 micron; Mobile phase A: 0.1% Formic acid in water; Mobile phase B: Acetonitrile) to afford 1-((4-(1-(2,6-dichlorophenyl) azetidin-3-yl) naphthalen-1-yl)methyl)-3-methylazetidin-3-ol, formic acid salt (34 mg, 34% yield, 99.6% purity).

¹H NMR (400 MHZ, MeOD): 8.22-8.25 (m, 1H), 8.00-8.03 (m, 1H), 7.58-7.69 (m, 4H), 7.20 (d, J=8.00 Hz, 2H), 6.73 (t, J=8.00 Hz, 1H), 5.11-5.14 (m, 2H), 4.49-4.59 (m, 5H), 3.80 (d, J=9.60 Hz, 2H), 3.66 (d, J=−9.60 Hz, 2H), 1.51 (s, 3H); LCMS method 1, LCMS (ESI, m/z): 427.0 [M+H]⁺.

### Example S56. 1-(4-(1-(2,6-dichlorophenyl) azetidin-3-yl)-2,5-dimethylbenzyl)-3-methylazetidin-3-ol (56)

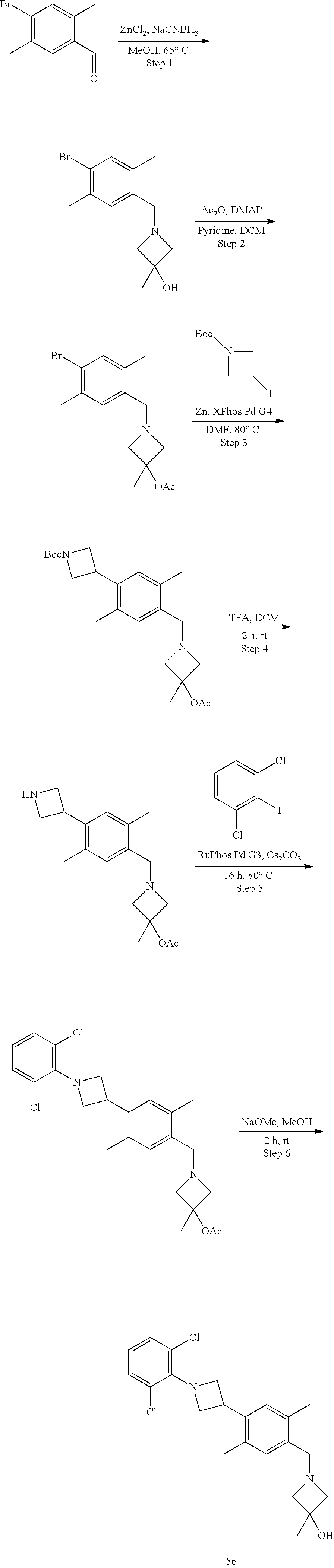

### Synthesis of 1-(4-bromo-2,5-dimethylbenzyl)-3-methylazetidin-3-ol

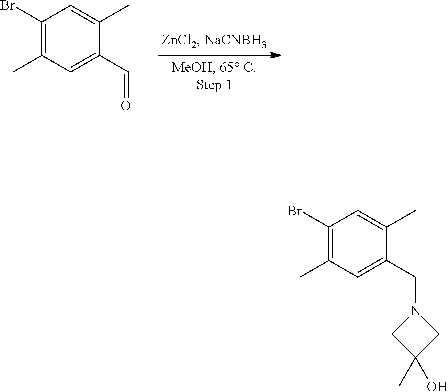 [SMILES: Cc1cc(C=O)c(C)cc1Br ; Cc1cc(CN2CC(C)(O)C2)c(C)cc1Br]

To a stirred solution of 3-methylazetidin-3-ol, TFA salt (2.124 g, 10.5 mmol) in MeOH(20 mL) was added sodium bicarbonate (1.47 g, 17.60 mmol.) then stirred for 1 h at rt. The mixture was filtered through celite and concentrated to yield free amine. To the free 4-bromo-2,5-dimethylbenzaldehyde (1.5 g, 7.04 mmol) in MeOH(20 mL) and zinc chloride (0.95 mg, 7.04 mmol) was added and stirred for 1 h at 25° C. After 1 h, Sodium cyano borohydride (664 mg, 10.56 mmol.) was added and heated to 65° C. for 12 h. The reaction mixture was diluted with dichloromethane (60 mL) and washed sat. ammonium chloride solution and water (60 mL). The combined organic phase was dried over Na₂SO₄, filtered and the solvents were evaporated under reduced pressure, the crude was purified by flash column chromatography on silica gel (230-400 mesh) eluting with 50-100% ethyl acetate in petroleum ether to afford 1-(4-bromo-2,5-dimethylbenzyl)-3-methylazetidin-3-ol (1.9 g, 95%) as a light yellow semi-solid. LCMS (ESI, m/z): 283.9 [M+H]⁺.

### Synthesis of 1-(4-bromo-2,5-dimethylbenzyl)-3-methylazetidin-3-yl acetate

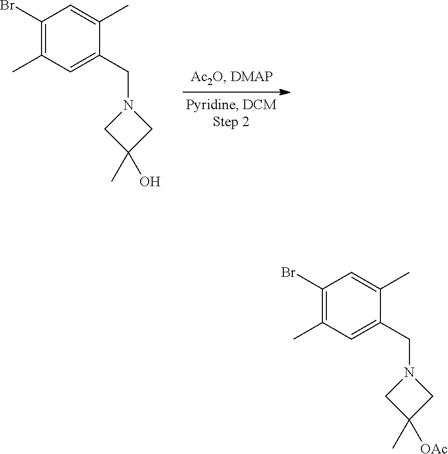 [SMILES: Cc1cc(CN2CC(C)(O)C2)c(C)cc1Br ; CC(=O)OC1(C)CN(Cc2cc(C)c(Br)cc2C)C1]

To a stirred solution of 1-(4-bromo-2,5-dimethylbenzyl)-3-methylazetidin-3-ol (1.8 g, 6.33 mmol) in DCM (20 mL) were added DMAP (0.77 g, 6.33 mmol) and Pyridine (0.5 mL, 6.33 mmol) and the reaction mixture was stirred at room temperature for 10 minutes. After that acetic anhydride (1.79 mL, 19 mmol) was added and the reaction mixture was stirred for 16 h at rt. The reaction mixture was diluted with saturated solution of ammonium chloride and extracted with DCM. The combined organic layer was dried over sodium sulphate, concentrated under vacuum, purified by flash column chromatography on 230-400 mesh silica gel (eluted with 20-30% ethyl acetate in pet ether) to afford 1-(4-bromo-2,5-dimethylbenzyl)-3-methylazetidin-3-yl acetate (1.1 g, 54% yield) as a transparent semi-solid. LCMS (ESI, m/z): 326.0 [M+H]⁺.

### Synthesis of tert-butyl 3-(4-((3-acetoxy-3-methylazetidin-1-yl)methyl)-2,5-dimethylphenyl) azetidine-1-carboxylate

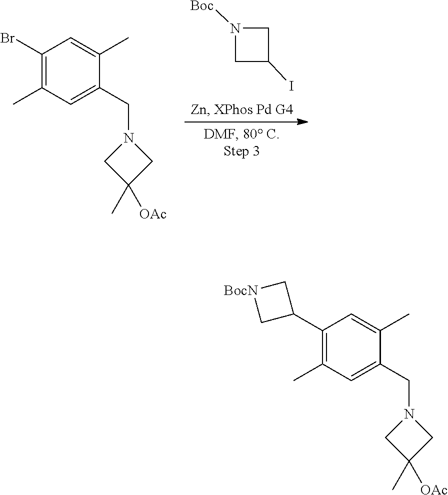 [SMILES: CC(=O)OC1(C)CN(Cc2cc(C)c(Br)cc2C)C1 ; CC(C)(C)OC(=O)N1CC(I)C1 ; CC(=O)OC1(C)CN(Cc2cc(C)c(C3CN(C(=O)OC(C)(C)C)C3)cc2C)C1]

To a suspension of activated zinc (2 g, 30.7 mmol) in anhydrous DMF (30 mL) was added 1,2-dibromoethane (0.132 mL, 1.53 mmol) and heated to 75° C. After 15 mins, the reaction mixture was cooled to room temperature, chloro trimethylsilane (0.19 mL, 1.53 mmol) was added and allowed to stir at ambient temperature for additional 30 mins. Then a solution of tert-butyl 3-iodoazetidine-1-carboxylate (2.60 g, 9.20 mmol) in 10 mL of anhydrous DMF was added to the reaction mixture and stirred at room temperature for another 30 min., followed by 1-(4-bromo-2,5-dimethylbenzyl)-3-methylazetidin-3-yl acetate (1 g, 3.07 mmol) and XPhos Pd G4 (0.528 g, 0.613 mmol) in 10 mL of DMF was added. The reaction mixture was allowed to stir at 80° C. for 2 h. After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (50 mL), dried over anhydrous sodium sulphate and concentrated under reduced pressure. The residue thus obtained was purified by flash column chromatography on silica gel (100-200 mesh) eluting with 0-50% ethyl acetate in petroleum ether to afford tert-butyl 3-(4-((3-acetoxy-3-methylazetidin-1-yl)methyl)-2,5-dimethylphenyl) azetidine-1-carboxylate (900 mg, 73% yield) as yellow semi-solid. LCMS method 1, LCMS (ESI, m/z): 403.4 [M+H]⁺

### Synthesis of 1-(4-(azetidin-3-yl)-2,5-dimethylbenzyl)-3-methylazetidin-3-yl acetate

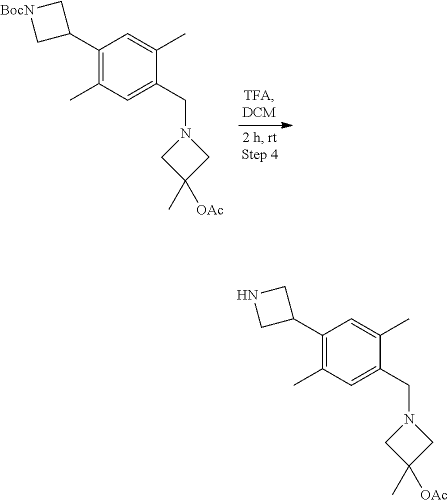 [SMILES: CC(=O)OC1(C)CN(Cc2cc(C)c(C3CN(C(=O)OC(C)(C)C)C3)cc2C)C1 ; CC(=O)OC1(C)CN(Cc2cc(C)c(C3CNC3)cc2C)C1]

To a stirred solution of tert-butyl 3-(4-((3-acetoxy-3-methylazetidin-1-yl)methyl)-2,5-dimethylphenyl) azetidine-1-carboxylate (0.9 g, 2.23 mmol) in anhydrous dichloromethane (10 mL) was added trifluoroacetic acid (1.72 mL, 22.36 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford 1-(4-(azetidin-3-yl)-2,5-dimethylbenzyl)-3-methylazetidin-3-yl acetate, TFA salt as brown color semi-solid (0.91 g, 98% yield). LCMS method 1, LCMS (ESI, m/z): 303.2 [M+H]⁺.

### Synthesis of 1-(4-(1-(2,6-dichlorophenyl) azetidin-3-yl)-2,5-dimethylbenzyl)-3-methylazetidin-3-yl acetate

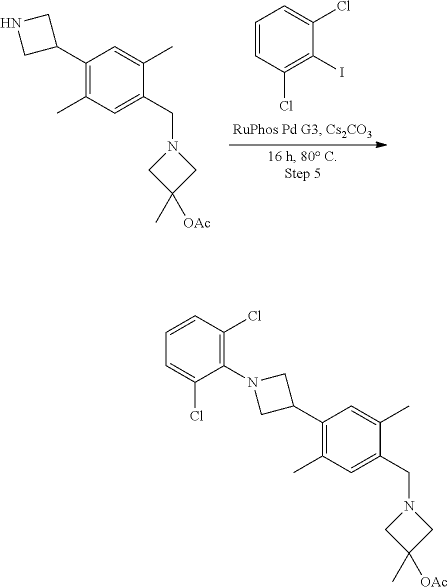 [SMILES: CC(=O)OC1(C)CN(Cc2cc(C)c(C3CNC3)cc2C)C1 ; Clc1cccc(Cl)c1I ; CC(=O)OC1(C)CN(Cc2cc(C)c(C3CN(c4c(Cl)cccc4Cl)C3)cc2C)C1]

To a solution of 1-(4-(azetidin-3-yl)-2,5-dimethylbenzyl)-3-methylazetidin-3-yl acetate, TFA salt (500 mg, 1.201 mmol) and 1,3-difluoro-2-iodobenzene (410 mg, 1.25 mmol) in anhydrous 1,4 dioxane (8 mL) was added cesium carbonate (1.17 g, 3.60 mmol). The reaction mixture was then degassed with nitrogen for 10 min followed by the addition of added RuPhos Pd G3 (50.2 mg, 0.06 mmol) and heated to 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 1-(4-(1-(2,6-dichlorophenyl) azetidin-3-yl)-2,5-dimethylbenzyl)-3-methylazetidin-3-yl acetate (327 mg, 61% yield) as a Orange brown solid. LCMS (ESI, m/z): 447.2 [M+H]⁺.

### Synthesis of 1-(4-(1-(2,6-dichlorophenyl) azetidin-3-yl)-2,5-dimethylbenzyl)-3-methylazetidin-3-ol (56)

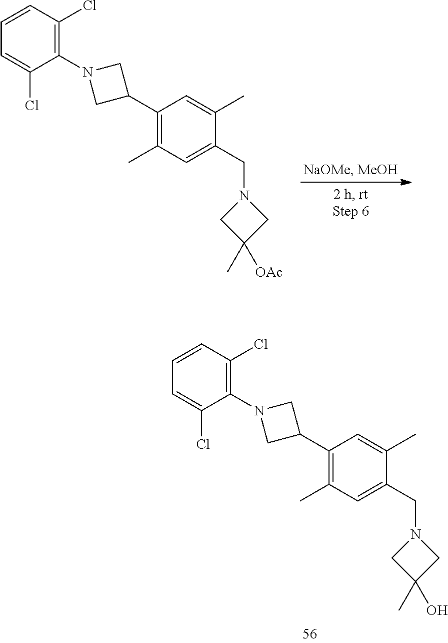 [SMILES: CC(=O)OC1(C)CN(Cc2cc(C)c(C3CN(c4c(Cl)cccc4Cl)C3)cc2C)C1 ; Cc1cc(C2CN(c3c(Cl)cccc3Cl)C2)c(C)cc1CN1CC(C)(O)C1]

To a stirred solution of 1-(4-(1-(3-fluorophenyl) azetidin-3-yl)-2,6-dimethylbenzyl)-3-methylazetidin-3-yl acetate (324 mg, 0.724 mmol) in methanol (10 mL) was added sodium methoxide (98 mg, 1.810 mmol). The resulting mixture was stirred at room temperature for 1 h. The reaction mixture was diluted with saturated solution of ammonium chloride and extracted with EtOAc. The combined organic layer was dried over sodium sulphate, concentrated under vacuum. The crude was purified using Prep. HPLC(Diluent: THF: Water: ACN (50:10:40); Column: Kintex EVO C18 (250×21.2) mm, 5 micron; Mobile phase A: 0.1% Formic acid in water; Mobile phase B: Acetonitrile) to afford 1-(4-(1-(2,6-dichlorophenyl) azetidin-3-yl)-2,5-dimethylbenzyl)-3-methylazetidin-3-ol, formic acid salt (41 mg, 0.090 mmol, 12.48% yield, 99.9% purity) as an off white solid.

¹H NMR (400 MHZ, MeOD): δ 7.33 (s, 1H), 7.19 (d, J=8.00 Hz, 2H), 7.12 (s, 1H), 6.72 (t, J=8.00 Hz, 1H), 4.90-4.94 (m, 2H), 4.36-4.40 (m, 2H), 4.03 (s, 2H), 3.94-4.00 (m, 1H), 3.72-3.74 (m, 2H), 3.53-3.56 (m, 2H), 2.380 (s, 3H), 2.26 (s, 3H), 1.51 (s, 3H). LCMS method 1; LCMS (ESI, m/z): 405.2 [M+H]⁺.

### Example S57. 1-(4-(1-(2,6-dichlorophenyl) azetidin-3-yl)-3,5-dimethylbenzyl)-3-methylazetidin-3-ol (57)

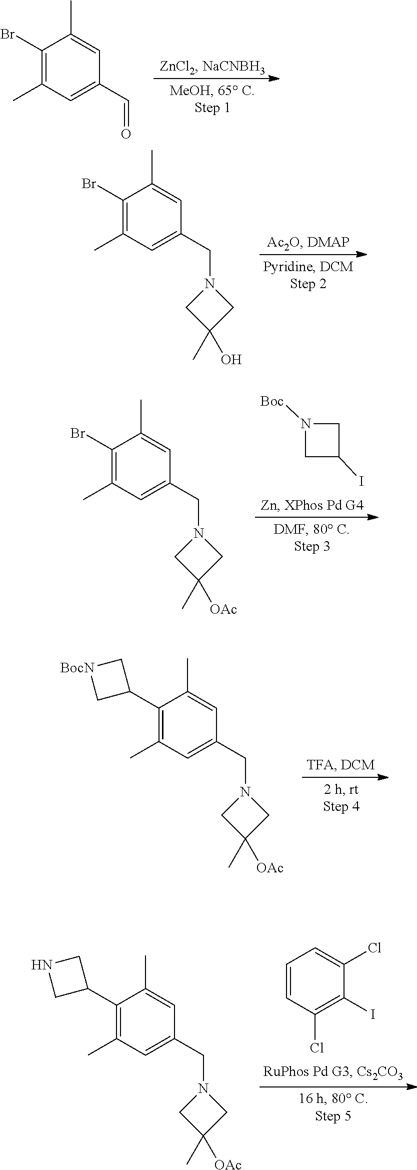

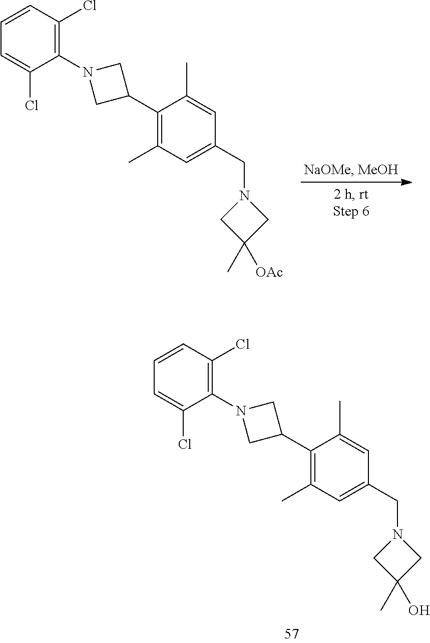 [SMILES: CC(=O)OC1(C)CN(Cc2cc(C)c(C3CN(c4c(Cl)cccc4Cl)C3)c(C)c2)C1 ; Cc1cc(CN2CC(C)(O)C2)cc(C)c1C1CN(c2c(Cl)cccc2Cl)C1]

### Synthesis of 1-(4-bromo-3,5-dimethylbenzyl)-3-methylazetidin-3-ol

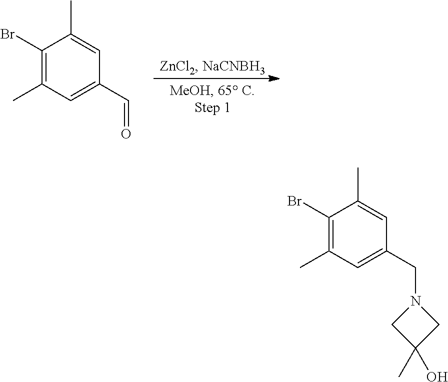 [SMILES: Cc1cc(C=O)cc(C)c1Br ; Cc1cc(CN2CC(C)(O)C2)cc(C)c1Br]

To a stirred solution of 3-methylazetidin-3-ol, TFA (2.124 g, 10.56 mmol) in MeOH(10 mL) was added sodium bicarbonate (1.478 g, 17.60 mmol) then stirred for 1 h at rt. The mixture was filtered through celite and concentrated to yield free amine. To the free amine and 4-bromo-3,5-dimethylbenzaldehyde (1.5 g, 7.04 mmol) in MeOH(20 mL zinc chloride (0.664 g, 10.56 mmol) was added and stirred for 1 h at 25° C. After 1 h, Sodium cyano borohydride (664 mg, 10.56 mmol) was added and heated to 65° C. for 12 h. The reaction mixture was diluted with dichloromethane (60 mL) and washed sat. ammonium chloride solution and water (60 mL). The combined organic phase was dried over Na₂SO₄, filtered and the solvents were evaporated under reduced pressure, the crude was purified by flash column chromatography on silica gel (230-400 mesh) eluting with 50-100% ethyl acetate in petroleum ether to afford 1-(4-bromo-3,5-dimethylbenzyl)-3-methylazetidin-3-ol (1.35 g, 67.5% yield) as a yellow semi-solid; LCMS method 1; LCMS (ESI, m/z): 284.2 [M+H]⁺.

### Synthesis of 1-(4-bromo-3,5-dimethylbenzyl)-3-methylazetidin-3-yl acetate

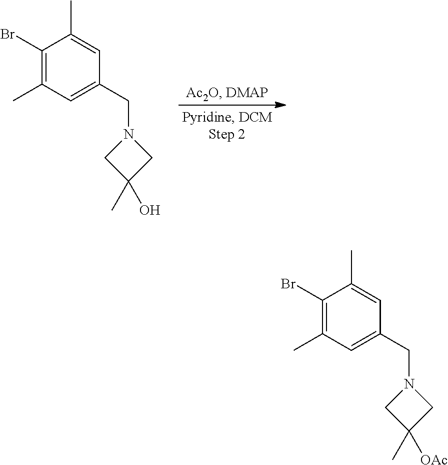 [SMILES: Cc1cc(CN2CC(C)(O)C2)cc(C)c1Br ; CC(=O)OC1(C)CN(Cc2cc(C)c(Br)c(C)c2)C1]

To a stirred solution of 1-(4-bromo-3,5-dimethylbenzyl)-3-methylazetidin-3-ol (1.0 g, 3.52 mmol) in DCM (20 mL) were added DMAP (0.430 g, 3.52 mmol) and pyridine (0.285 mL, 3.52 mmol) and the reaction mixture was stirred at room temperature for 10 minutes. After that acetic anhydride (0.996 mL, 10.56 mmol) was added and the reaction mixture was stirred for 16 h at rt. The reaction mixture was diluted with saturated solution of ammonium chloride and extracted with DCM. The combined organic layer was dried over sodium sulphate, concentrated under vacuum, purified by flash column chromatography on 230-400 mesh silica gel (eluted with 20-30% ethyl acetate in pet ether) to afford 1-(4-bromo-3,5-dimethylbenzyl)-3-methylazetidin-3-yl acetate (840 mg, 69.7% yield) as a Colorless semi-solid; LCMS method 1, LCMS (ESI, m/z): 326.0 [M]⁺.

### Synthesis of tert-butyl 3-(4-((3-acetoxy-3-methylazetidin-1-yl)methyl)-2,6-dimethylphenyl) azetidine-1-carboxylate

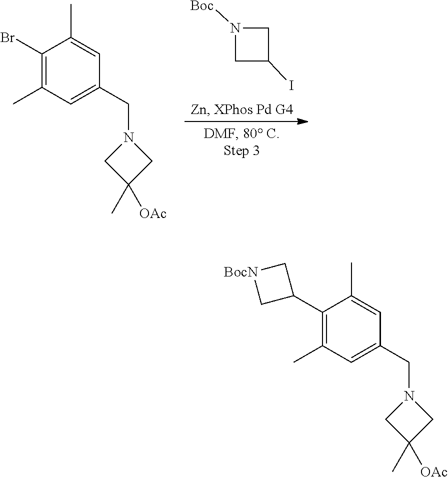 [SMILES: CC(=O)OC1(C)CN(Cc2cc(C)c(Br)c(C)c2)C1 ; CC(C)(C)OC(=O)N1CC(I)C1 ; CC(=O)OC1(C)CN(Cc2cc(C)c(C3CN(C(=O)OC(C)(C)C)C3)c(C)c2)C1]

To a suspension of activated zinc (1503 mg, 22.99 mmol) in anhydrous DMF (30 mL) was added 1,2-dibromoethane (0.099 mL, 1.149 mmol) and heated to 75° C. After 15 mins, the reaction mixture was cooled to room temperature, trimethylchlorosilane (0.147 mL, 1.149 mmol) was added and allowed to stir at ambient temperature for addition 30 mins. Then a solution of tert-butyl 3-iodoazetidine-1-carboxylate (1953 mg, 6.90 mmol) in 10 mL of anhydrous DMF was added to the reaction mixture and stirred at room temperature for another 30 min., followed by 1-(4-bromo-3,5-dimethylbenzyl)-3-methylazetidin-3-yl acetate (750 mg, 2.299 mmol) and Xphos Pd G4 (396 mg, 0.460 mmol) in 10 mL of DMF was added. The reaction mixture was allowed to stir at 80° C. for 2 h. After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (50 mL), dried over anhydrous sodium sulphate and concentrated under reduced pressure. The residue thus obtained was purified by flash column chromatography on silica gel (100-200 mesh) eluting with 0-50% ethyl acetate in petroleum ether to afford tert-butyl 3-(4-((3-acetoxy-3-methylazetidin-1-yl)methyl)-2,6-dimethylphenyl)-azetidine-1-carboxylate (400 mg, 0 982 mmol, 42.7% yield) as a Yellow semi-solid; LCMS method 1, LCMS (ESI, m/z): 403.2 [M+H]⁺.

### Synthesis of 1-(4-(azetidin-3-yl)-3,5-dimethylbenzyl)-3-methylazetidin-3-yl acetate

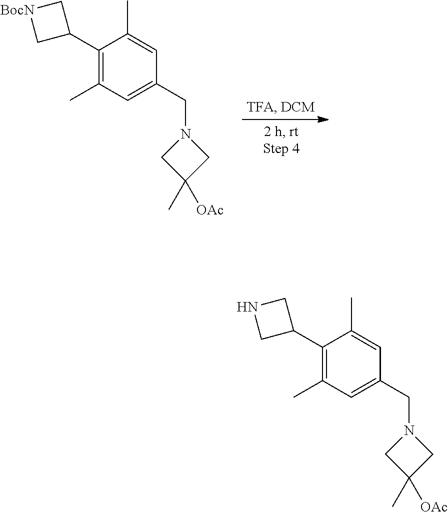 [SMILES: CC(=O)OC1(C)CN(Cc2cc(C)c(C3CN(C(=O)OC(C)(C)C)C3)c(C)c2)C1 ; CC(=O)OC1(C)CN(Cc2cc(C)c(C3CNC3)c(C)c2)C1]

To a stirred solution of tert-butyl 3-(4-((3-acetoxy-3-methylazetidin-1-yl)methyl)-2,6-dimethylphenyl) azetidine-1-carboxylate (400 mg, 0.994 mmol) in anhydrous dichloromethane (5 mL) was added TFA (0.766 mL, 9.94 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford 1-(4-(azetidin-3-yl)-3,5-dimethylbenzyl)-3-methylazetidin-3-yl acetate, TFA (310 mg, 0.744 mmol, 74.9% yield) as a Yellow semi-solid; LCMS method 1, LCMS (ESI, m/z): 303.0 [M+H]⁺.

### Synthesis of 1-(4-(1-(2,6-dichlorophenyl) azetidin-3-yl)-3,5-dimethylbenzyl)-3-methylazetidin-3-yl acetate

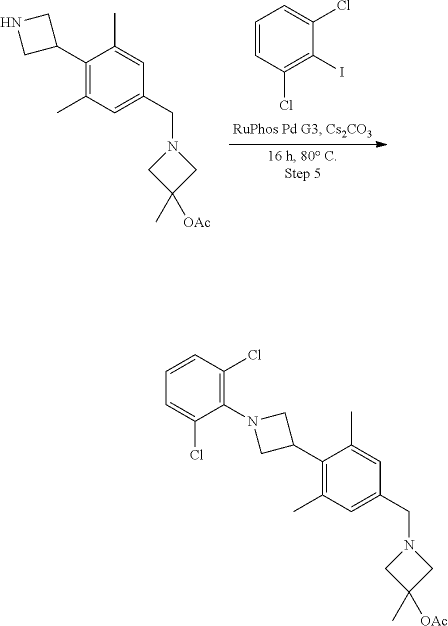 [SMILES: CC(=O)OC1(C)CN(Cc2cc(C)c(C3CNC3)c(C)c2)C1 ; Clc1cccc(Cl)c1I ; CC(=O)OC1(C)CN(Cc2cc(C)c(C3CN(c4c(Cl)cccc4Cl)C3)c(C)c2)C1]

To a solution of 1-(4-(azetidin-3-yl)-3,5-dimethylbenzyl)-3-methylazetidin-3-yl acetate, TFA (300 mg, 0.720 mmol) and 1,3-dichloro-2-iodobenzene (295 mg, 1.081 mmol) in anhydrous 1,4 dioxane (8 mL) was added cesium carbonate (704 mg, 2.161 mmol). The reaction mixture was then degassed with nitrogen for 10 min followed by the addition of added Ruphos Pd G3 (60.2 mg, 0.072 mmol) and heated to 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 1-(4-(1-(2,6-dichlorophenyl) azetidin-3-yl)-3,5-dimethylbenzyl)-3-methylazetidin-3-yl acetate (38 mg, 11% yield) as a Yellow semi-solid; LCMS method 1, LCMS (ESI, m/z): 447.1 [M+H]⁺.

### Synthesis of 1-(4-(1-(2,6-dichlorophenyl) azetidin-3-yl)-3,5-dimethylbenzyl)-3-methylazetidin-3-ol (57)

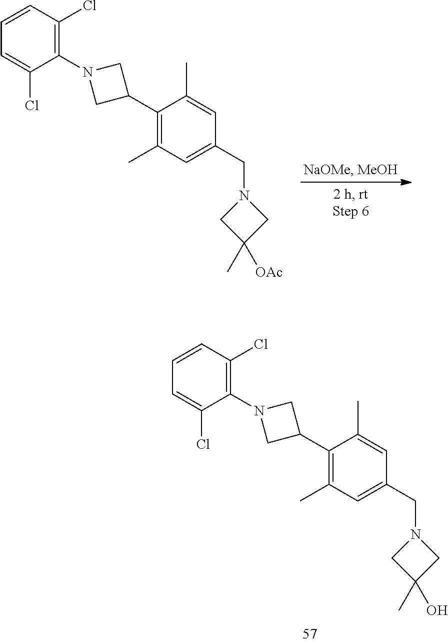 [SMILES: CC(=O)OC1(C)CN(Cc2cc(C)c(C3CN(c4c(Cl)cccc4Cl)C3)c(C)c2)C1 ; Cc1cc(CN2CC(C)(O)C2)cc(C)c1C1CN(c2c(Cl)cccc2Cl)C1]

To a stirred solution of 1-(4-(1-(2,6-dichlorophenyl) azetidin-3-yl)-3,5-dimethylbenzyl)-3-methylazetidin-3-yl acetate (35 mg, 0.078 mmol) in methanol (10 mL) was added sodium methoxide (8.5 mg, 1.156 mmol). The resulting mixture was stirred at room temperature for 1 h. The reaction mixture was diluted with saturated solution of ammonium chloride and extracted with EtOAc. The combined organic layer was dried over sodium sulphate, concentrated under vacuum. The crude was purified using Prep. HPLC(Diluent: THF: Water: ACN (50:20:30); Column: Zorbax C18 (50×21.5) mm, 5 micron; Mobile phase A: 0.1% Formic acid in water; Mobile phase B: Acetonitrile) to afford 1-(4-(1-(2,6-dichlorophenyl) azetidin-3-yl)-3,5-dimethylbenzyl)-3-methylazetidin-3-ol, formic acid salt (5.34 mg, 0.012 mmol, 14.80% yield, 97.9% purity) as an Off white solid.

¹H NMR (400 MHZ, MeOD): δ 7.18 (d, J=7.60 Hz, 2H), 6.99 (s, 2H), 6.70 (t, J=16.00 Hz, 1H), 5.06 (m, 2H), 4.63-4.44 (m, 2H), 4.25 (d, J=8.40 Hz, 1H), 3.80 (s, 2H), 3.55 (d, J=9.60 Hz, 2H), 3.39-3.32 (m, 2H), 2.36 (s, 6H), 1.54 (s, 3H); LCMS method 1, LCMS (ESI, m/z): 405.2 [M+H]⁺.

### Example S58. 1-((6-(1-(2,6-dichlorophenyl) azetidin-3-yl) pyridin-3-yl)methyl)-3-methylazetidin-3-ol (58)

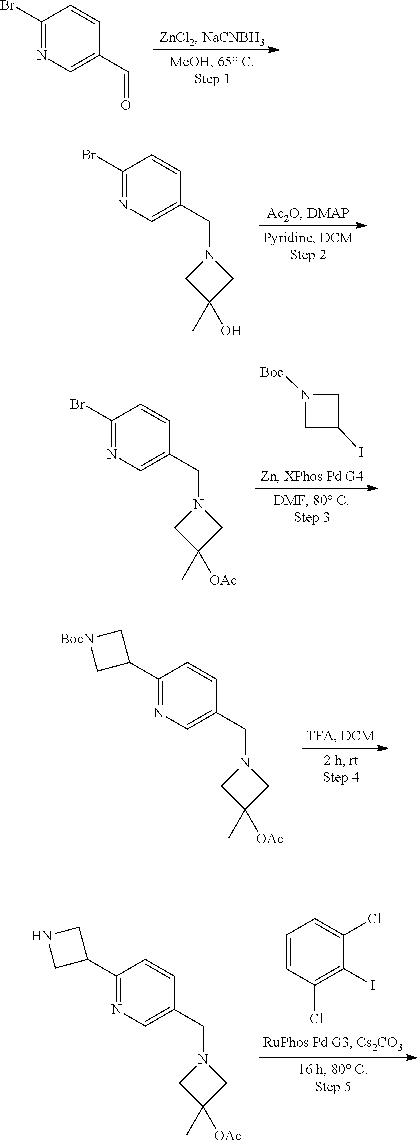

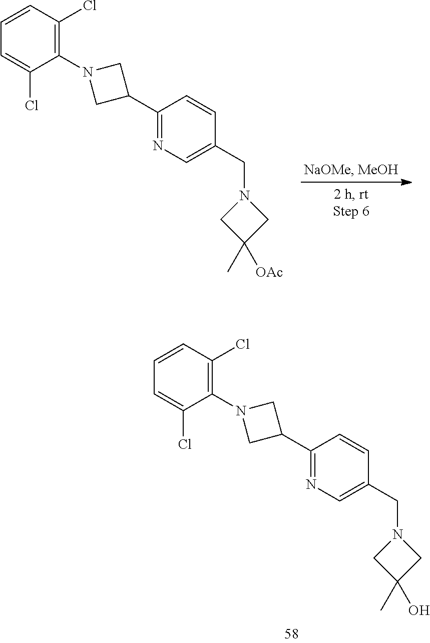 [SMILES: CC(=O)OC1(C)CN(Cc2ccc(C3CN(c4c(Cl)cccc4Cl)C3)nc2)C1 ; CC1(O)CN(Cc2ccc(C3CN(c4c(Cl)cccc4Cl)C3)nc2)C1]

### Synthesis of 1-((6-bromopyridin-3-yl)methyl)-3-methylazetidin-3-ol

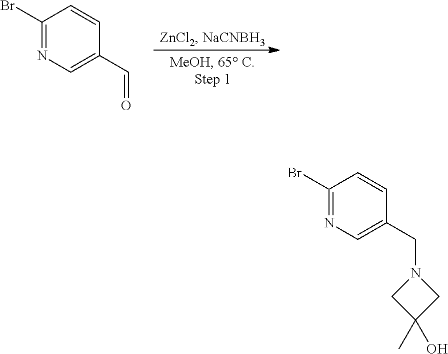

To a stirred solution of 3-methylazetidin-3-ol, TFA (3.24 g, 16.13 mmol) in MeOH(20 mL) was added sodium bicarbonate (2.82 g, 33.6 mmol) then stirred for 1 h at rt. The mixture was filtered through celite and concentrated to yield free amine. To the free amine and 6-bromonicotinaldehyde (2.5 g, 13.44 mmol) in MeOH(20 mL), zinc chloride (1.832 g, 13.44 mmol) was added and stirred for 1 h at 25° C. After 1 h, sodium cyanoborohydride (1.267 g, 20.16 mmol) was added and heated to 65° C. for 12 h. The reaction mixture was diluted with dichloromethane (60 mL) and washed sat. ammonium chloride solution and water (60 mL). The combined organic phase was dried over Na₂SO₄, filtered and the solvents were evaporated under reduced pressure, the crude was purified by flash column chromatography on silica gel (230-400 mesh) eluting with 50-100% ethyl acetate in petroleum ether to afford 1-((6-bromopyridin-3-yl)methyl)-3-methylazetidin-3-ol (1.5 g, 5.83 mmol, 43.4% yield) as a light yellow semi-solid; LCMS method 1, LCMS (ESI, m/z): 258.0 [M+3H]⁺.

### Synthesis of 1-((6-bromopyridin-3-yl)methyl)-3-methylazetidin-3-yl acetate

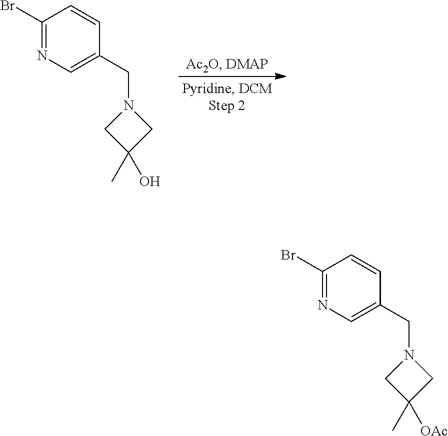 [SMILES: CC1(O)CN(Cc2ccc(Br)nc2)C1 ; CC(=O)OC1(C)CN(Cc2ccc(Br)nc2)C1]

To a stirred solution of 1-((6-bromopyridin-3-yl)methyl)-3-methylazetidin-3-ol (1.5 g, 5.83 mmol) in DCM (20 mL) were added DMAP (0.713 g, 5.83 mmol) pyridine (0.472 mL, 5.83 mmol) and the reaction mixture was stirred at room temperature for 10 minutes. Afterwards, acetic anhydride (1.651 mL, 17.50 mmol) was added and the reaction mixture was stirred for 16 h at rt. The reaction mixture was diluted with saturated solution of ammonium chloride and extracted with DCM. The combined organic layer was dried over sodium sulphate, concentrated under vacuum, purified by flash column chromatography on 230-400 mesh silica gel (eluted with 20-30% ethyl acetate in pet ether) to afford 1-((6-bromopyridin-3-yl)methyl)-3-methylazetidin-3-yl acetate (1.0 g, 57.3% yield) as a Colorless oil; LCMS method 1, LCMS (ESI, m/z): 299.3 [M+H]⁺.

### Synthesis of tert-butyl 3-(5-((3-acetoxy-3-methylazetidin-1-yl)methyl) pyridin-2-yl) azetidine-1-carboxylate

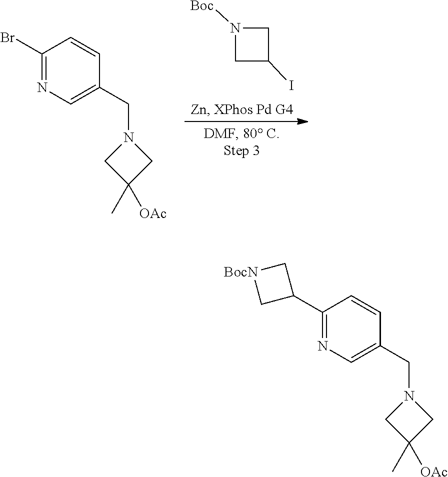 [SMILES: CC(=O)OC1(C)CN(Cc2ccc(Br)nc2)C1 ; CC(C)(C)OC(=O)N1CC(I)C1 ; CC(=O)OC1(C)CN(Cc2ccc(C3CN(C(=O)OC(C)(C)C)C3)nc2)C1]

To a suspension of activated zinc (2.185 g, 33.4 mmol) in anhydrous DMF (30 mL) was added 1,2-dibromoethane (0.144 mL, 1.671 mmol) and heated to 75° C. After 15 mins, the reaction mixture was cooled to room temperature, trimethylchlorosilane (0.214 mL, 1.671 mmol) was added and allowed to stir at ambient temperature for additional 30 mins. Then a solution of tert-butyl 3-iodoazetidine-1-carboxylate (2.84 g, 10.03 mmol) in 10 mL of anhydrous DMF was added to the reaction mixture and stirred at room temperature for another 30 min., followed by 1-((6-bromopyridin-3-yl)methyl)-3-methylazetidin-3-yl acetate (1 g, 3.34 mmol) and XPhos Pd G4 (0.575 g, 0.669 mmol) in 10 mL of DMF was added. The reaction mixture was allowed to stir at 80° C. for 2 h. After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (50 mL), dried over anhydrous sodium sulphate and concentrated under reduced pressure. The residue thus obtained was purified by flash column chromatography on silica gel (100-200 mesh) eluting with 0-50% ethyl acetate in petroleum ether to afford tert-butyl 3-(5-((3-acetoxy-3-methylazetidin-1-yl)methyl) pyridin-2-yl) azetidine-1-carboxylate (360 mg, 28.7% yield) as a Light brown semi-solid, LCMS method 3, LCMS (ESI, m/z): 376.2 [M+H]⁺.

### Synthesis of 1-((6-(azetidin-3-yl) pyridin-3-yl)methyl)-3-methylazetidin-3-yl acetate

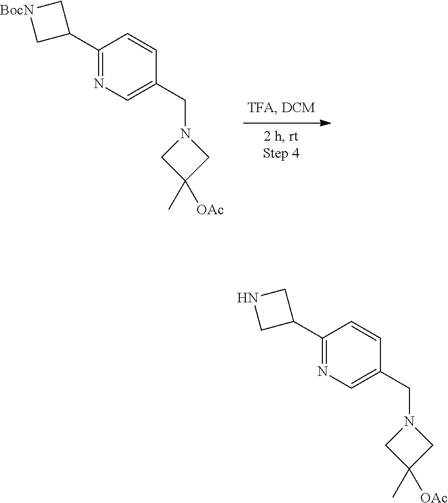 [SMILES: CC(=O)OC1(C)CN(Cc2ccc(C3CN(C(=O)OC(C)(C)C)C3)nc2)C1 ; CC(=O)OC1(C)CN(Cc2ccc(C3CNC3)nc2)C1]

To a stirred solution of tert-butyl 3-(5-((3-acetoxy-3-methylazetidin-1-yl)methyl) pyridin-2-yl) azetidine-1-carboxylate (350 mg, 0.932 mmol) in anhydrous dichloromethane (10 mL) was added trifluoroacetic acid (0.72 mL, 9.32 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford tert-butyl 3-(5-((3-acetoxy-3-methylazetidin-1-yl)methyl) pyridin-2-yl) azetidine-1-carboxylate (350 mg, 0.932 mmol) as a Light green semi-solid; LCMS method 1, LCMS (ESI, m/z): 276.2 [M+H]⁺.

### Synthesis of 1-((6-(1-(2,6-dichlorophenyl) azetidin-3-yl) pyridin-3-yl)methyl)-3-methyl-azetidin-3-yl acetate

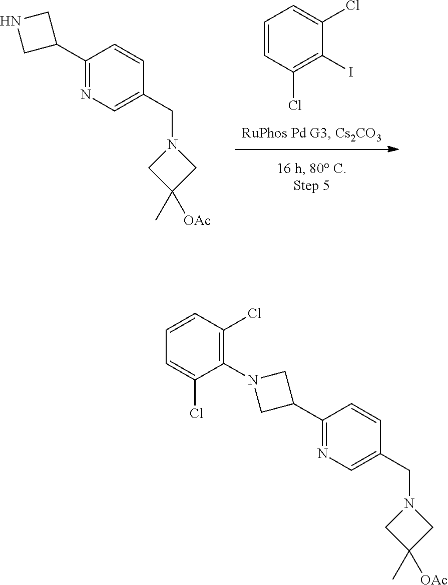 [SMILES: CC(=O)OC1(C)CN(Cc2ccc(C3CNC3)nc2)C1 ; Clc1cccc(Cl)c1I ; CC(=O)OC1(C)CN(Cc2ccc(C3CN(c4c(Cl)cccc4Cl)C3)nc2)C1]

To a solution of 1-((6-(azetidin-3-yl) pyridin-3-yl)methyl)-3-methylazetidin-3-yl acetate, TFA (350 mg, 0.899 mmol) and 1,3-dichloro-2-iodobenzene (307 mg, 1.124 mmol) in anhydrous 1,4 dioxane (8 mL) was added cesium carbonate (879 mg, 2.70 mmol). The reaction mixture was then degassed with nitrogen for 10 min followed by the addition of added RuPhos Pd G3 (75 mg, 0.090 mmol) and heated to 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 1-((6-(1-(2,6-dichlorophenyl) azetidin-3-yl) pyridin-3-yl)methyl)-3-methylazetidin-3-yl acetate (184 mg, 0.438 mmol, 48.7% yield) as a Colorless semi-solid; LCMS method 1, LCMS (ESI, m/z): 420.0 [M+H]⁺.

### Synthesis of 1-((6-(1-(2,6-dichlorophenyl) azetidin-3-yl) pyridin-3-yl)methyl)-3-methylazetidin-3-ol (58)

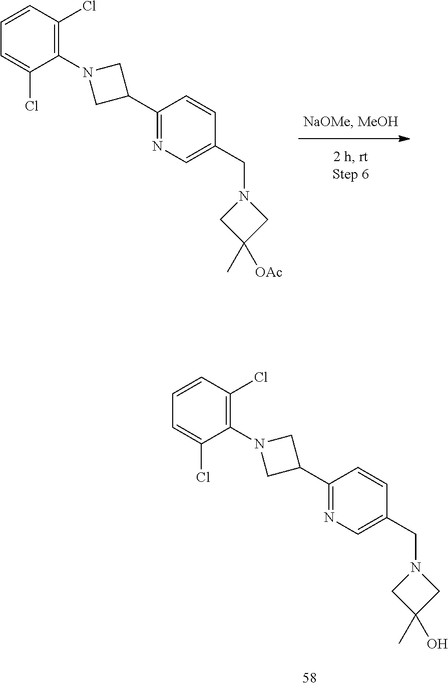 [SMILES: CC(=O)OC1(C)CN(Cc2ccc(C3CN(c4c(Cl)cccc4Cl)C3)nc2)C1 ; CC1(O)CN(Cc2ccc(C3CN(c4c(Cl)cccc4Cl)C3)nc2)C1]

To a stirred solution of 1-((6-(1-(2,6-dichlorophenyl) azetidin-3-yl) pyridin-3-yl)methyl)-3-methylazetidin-3-yl acetate (180 mg, 0.428 mmol) in methanol (10 mL) was added sodium methoxide (57.8 mg, 1.071 mmol). The resulting mixture was stirred at room temperature for 1 h. The reaction mixture was diluted with saturated solution of ammonium chloride and extracted with EtOAc. The combined organic layer was dried over sodium sulphate, concentrated under vacuum. The crude was purified using Prep. HPLC(Diluent: THF: Water: ACN (50:10:40); Column: Kintex EVO C18 (250×21.2) mm, 5 micron; Mobile phase A: 0.1% Formic acid in water; Mobile phase B: Acetonitrile) to afford 1-((6-(1-(2,6-dichlorophenyl) azetidin-3-y1)pyridin-3-yl)methyl)-3-methylazetidin-3-ol, formic acid salt (19 mg, 0.044 mmol, 10.30% yield, 98.5% purity) as a Colorless semi-solid.

¹H NMR (400 MHZ, DMSO-d6): δ 8.58 (d, J=2.00 Hz, 1H), 7.86 (dd, J=2.40, 8.20 Hz, 1H), 7.55 (d, J=8.00 Hz, 1H), 7.19 (t, J=8.00 Hz, 2H), 6.71 (t, J=8.00 Hz, 1H), 4.91-4.93 (m, 2H), 4.58-4.62 (m, 2H), 4.15 (s, 2H), 3.94-3.98 (m, 1H), 3.80 (d, J=10.00 Hz, 2H), 1.51 (s, 3H). LCMS method 1; LCMS (ESI, m/z): 380.2 [M+3H]⁺.

### Example S59. 1-((5-(1-(2,6-dichlorophenyl) azetidin-3-yl) pyridin-2-yl)methyl)-3-methylazetidin-3-ol (59)

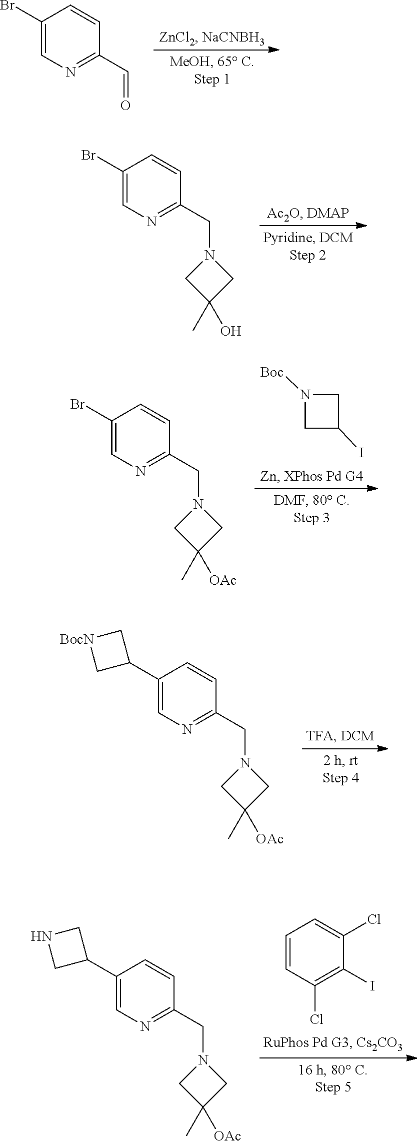

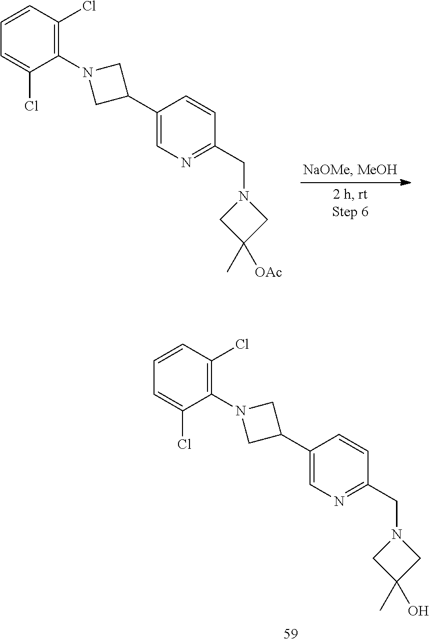 [SMILES: CC(=O)OC1(C)CN(Cc2ccc(C3CN(c4c(Cl)cccc4Cl)C3)cn2)C1 ; CC1(O)CN(Cc2ccc(C3CN(c4c(Cl)cccc4Cl)C3)cn2)C1]

### Synthesis of 1-((5-bromopyridin-2-yl)methyl)-3-methylazetidin-3-ol

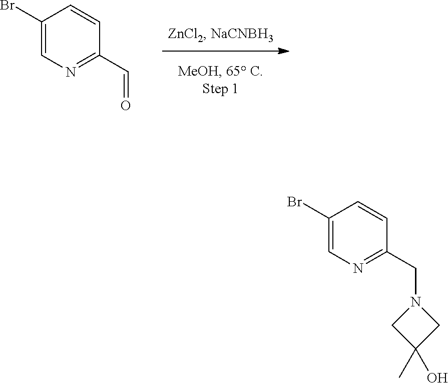

To a stirred solution of 3-methylazetidin-3-ol, TFA (3.89 g, 19.35 mmol) in MeOH(20 mL) was added sodium bicarbonate (3.39 g, 40.3 mmol) then stirred for 1 h at rt. The mixture was filtered through celite and concentrated to yield free amine. To the free amine and 5-bromopicolinaldehyde (3 g, 16.13 mmol) in MeOH(20 mL), zinc chloride (2.198 g, 16.13 mmol) was added and stirred for 1 h at 25° C. After 1 h, sodium cyanoborohydride (1.520 g, 24.19 mmol) was added and heated to 65° C. for 12 h. The reaction mixture was diluted with dichloromethane (60 mL) and washed sat. ammonium chloride solution and water (60 mL). The combined organic phase was dried over Na₂SO₄, filtered and the solvents were evaporated under reduced pressure, the crude was purified by flash column chromatography on silica gel (230-400 mesh) eluting with 50-100% ethyl acetate in petroleum ether to afford 1-((5-bromopyridin-2-yl)methyl)-3-methylazetidin-3-ol (2.7 g, 10.50 mmol, 65.1% yield) as a Light yellow semi-solid, Yield 65%; LCMS method 1, LCMS (ESI, m/z): 259.1 [M+3H]⁺.

### Synthesis of 1-((5-bromopyridin-2-yl)methyl)-3-methylazetidin-3-yl acetate

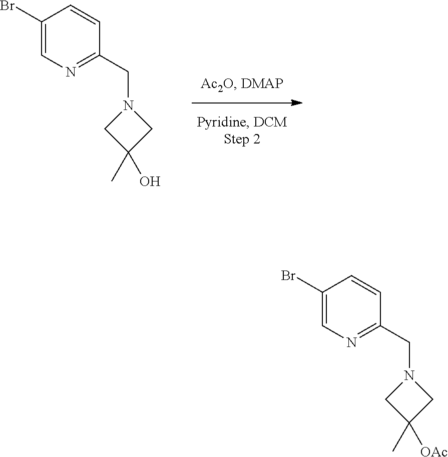 [SMILES: CC1(O)CN(Cc2ccc(Br)cn2)C1 ; CC(=O)OC1(C)CN(Cc2ccc(Br)cn2)C1]

To a stirred solution of 1-((5-bromopyridin-2-yl)methyl)-3-methylazetidin-3-ol (2.7 g, 10.50 mmol) in DCM (30 mL) were added DMAP (1.283 g, 10.50 mmol) and pyridine (0.849 mL, 10.50 mmol) and the reaction mixture was stirred at room temperature for 10 minutes. After that acetic anhydride (2.97 mL, 31.5 mmol) was added and the reaction mixture was stirred for 16 h at rt. The reaction mixture was diluted with saturated solution of ammonium chloride and extracted with DCM. The combined organic layer was dried over sodium sulphate, concentrated under vacuum, purified by flash column chromatography on 230-400 mesh silica gel (eluted with 20-30% ethyl acetate in pet ether) to afford 1-((5-bromopyridin-2-yl)methyl)-3-methylazetidin-3-yl acetate (1.7 g, 54.1% yield) as a Colorless oil, Yield 54.1%; LCMS method 1, LCMS (ESI, m/z): 299.1 [M+H]⁺.

### Synthesis of tert-butyl 3-(6-((3-acetoxy-3-methylazetidin-1-yl)methyl) pyridin-3-yl) azetidine-1-carboxylate

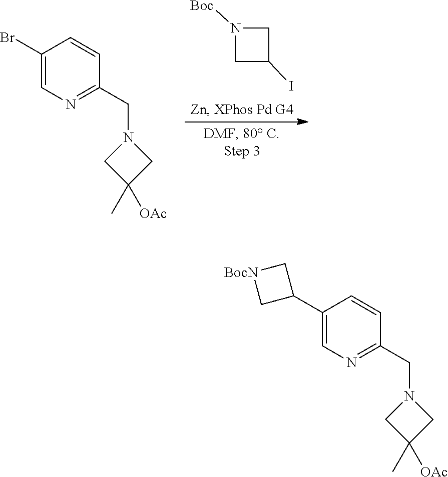 [SMILES: CC(=O)OC1(C)CN(Cc2ccc(Br)cn2)C1 ; CC(C)(C)OC(=O)N1CC(I)C1 ; CC(=O)OC1(C)CN(Cc2ccc(C3CN(C(=O)OC(C)(C)C)C3)cn2)C1]

To a suspension of activated zinc (2.185 g, 33.4 mmol) in anhydrous DMF (30 mL) was added 1,2-dibromoethane (0.144 mL, 1.671 mmol) and heated to 75° C. After 15 mins, the reaction mixture was cooled to room temperature, trimethylchlorosilane (0.214 mL, 1.671 mmol) was added and allowed to stir at ambient temperature for additional 30 mins. Then a solution of tert-butyl 3-iodoazetidine-1-carboxylate (2.84 g, 10.03 mmol) in 10 mL of anhydrous DMF was added to the reaction mixture and stirred at room temperature for another 30 min., followed by 1-((5-bromopyridin-2-yl)methyl)-3-methylazetidin-3-yl acetate (1 g, 3.34 mmol) and XPhos Pd G4 (0.575 g, 0.669 mmol) in 10 mL of DMF was added. The reaction mixture was allowed to stir at 80° C. for 2 h. After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (50 mL), dried over anhydrous sodium sulphate and concentrated under reduced pressure. The residue thus obtained was purified by flash column chromatography on silica gel (100-200 mesh) eluting with 0-50% ethyl acetate in petroleum ether to afford tert-butyl 3-(6-((3-acetoxy-3-methylazetidin-1-yl)methyl) pyridin-3-yl) azetidine-1-carboxylate (0.9 g, 71.7% yield) as Light brown semi-solid; LCMS method 1. LCMS (ESI, m/z): 376.2 [M+H]⁺.

### Synthesis of 1-((5-(azetidin-3-yl) pyridin-2-yl)methyl)-3-methylazetidin-3-yl acetate

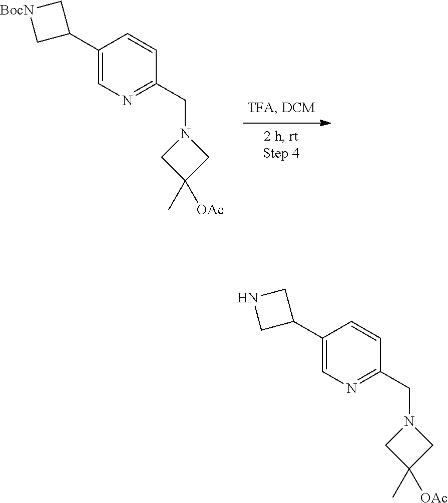 [SMILES: CC(=O)OC1(C)CN(Cc2ccc(C3CN(C(=O)OC(C)(C)C)C3)cn2)C1 ; CC(=O)OC1(C)CN(Cc2ccc(C3CNC3)cn2)C1]

To a stirred solution of tert-butyl 3-(6-((3-acetoxy-3-methylazetidin-1-yl)methyl) pyridin-3-yl) azetidine-1-carboxylate (900 mg, 2.397 mmol) in anhydrous dichloromethane (10 mL) was added trifluoroacetic acid (0.92 mL, 11.99 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford 1-((5-(azetidin-3-yl) pyridin-2-yl)methyl)-3-methylazetidin-3-yl acetate, TFA (400 mg, 42.9% yield) as Yellow liquid; LCMS method 1, LCMS (ESI, m/z): 276.2 [M+H]⁺.

### Synthesis of 1-((5-(1-(2,6-dichlorophenyl) azetidin-3-yl) pyridin-2-yl)methyl)-3-methylazetidin-3-yl acetate

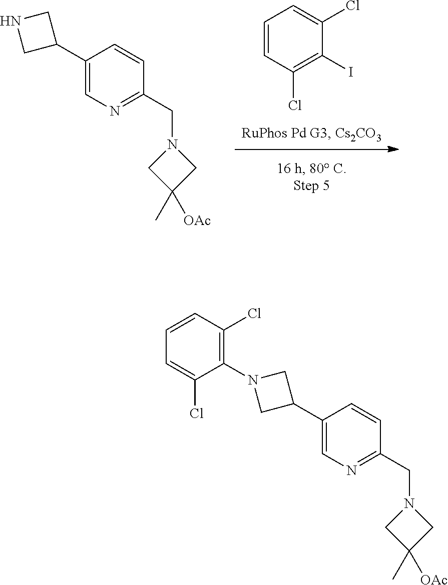 [SMILES: CC(=O)OC1(C)CN(Cc2ccc(C3CNC3)cn2)C1 ; Clc1cccc(Cl)c1I ; CC(=O)OC1(C)CN(Cc2ccc(C3CN(c4c(Cl)cccc4Cl)C3)cn2)C1]

To a solution of 1-((5-(azetidin-3-yl) pyridin-2-yl)methyl)-3-methylazetidin-3-yl acetate, TFA (713 mg, 1.832 mmol) and 1,3-dichloro-2-iodobenzene (500 mg, 1.832 mmol) in anhydrous 1,4 dioxane (10 mL) was added cesium carbonate (1194 mg, 3.66 mmol). The reaction mixture was then degassed with nitrogen for 10 min followed by the addition of added RuPhos Pd G3 (153 mg, 0.183 mmol) and heated to 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 1-((5-(1-(2,6-dichlorophenyl) azetidin-3-yl) pyridin-2-yl)methyl)-3-methylazetidin-3-yl acetate (211 mg, 27.5% yield) as a yellow solid, Yield 27.5%; LCMS method 1, LCMS (ESI, m/z): 422.1 [M+3H]*.

### Synthesis of 1-((5-(1-(2,6-dichlorophenyl) azetidin-3-yl) pyridin-2-yl)methyl)-3-methylazetidin-3-ol, formic acid salt (59)

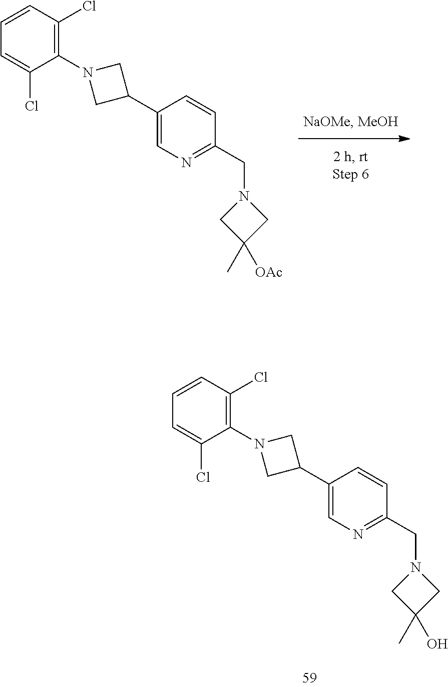 [SMILES: CC(=O)OC1(C)CN(Cc2ccc(C3CN(c4c(Cl)cccc4Cl)C3)cn2)C1 ; CC1(O)CN(Cc2ccc(C3CN(c4c(Cl)cccc4Cl)C3)cn2)C1]

To a stirred solution of 1-((5-(1-(2,6-dichlorophenyl) azetidin-3-yl) pyridin-2-yl)methyl)-3-methylazetidin-3-yl acetate (200 mg, 0.476 mmol) in methanol (5 mL) was added sodium methoxide (51.4 mg, 0.952 mmol). The resulting mixture was stirred at room temperature for 1 h. The reaction mixture was diluted with saturated solution of ammonium chloride and extracted with EtOAc. The combined organic layer was dried over sodium sulphate, concentrated under vacuum. The crude was purified using Prep. HPLC(Diluent: THF: Water: ACN (50:10:40); Column-1: Luna C18 (250×21.2) mm, 10 micron; Mobile phase A: 0.1% TFA in water; Mobile phase B: Acetonitrile) to afford 1-((5-(1-(2,6-dichlorophenyl) azetidin-3-yl) pyridin-2-y1)methyl)-3-methylazetidin-3-ol, formic acid salt (36 mg, 0.083 mmol, 17.51% yield, 98.2% purity) as a yellow semi-solid.

¹H NMR (400 MHZ, MeOD): δ 8.66 (d, J=2.00 Hz, 1H), 8.46 (s, 1H), 7.48 (d, J=8.00 Hz, 1H), 7.21 (d, J=8.00 Hz, 2H), 6.76 (t, J=8.00 Hz, 1H), 4.89-4.95 (m, 2H), 4.42-4.46 (m, 2H), 4.39 (s, 2H), 4.00 (d, J=10.40 Hz, 1H), 3.80-3.85 (m, 3H), 1.55 (s, 3H)(Two protons are merged with solvent signal). LCMS method 1; LCMS (ESI, m/z): 378.0 [M+H]⁺.

### Example S60. 1-(4-(1-(2,6-dichlorophenyl) azetidin-3-yl)-3-fluorobenzyl)-3-methylazetidin-3-ol (60)

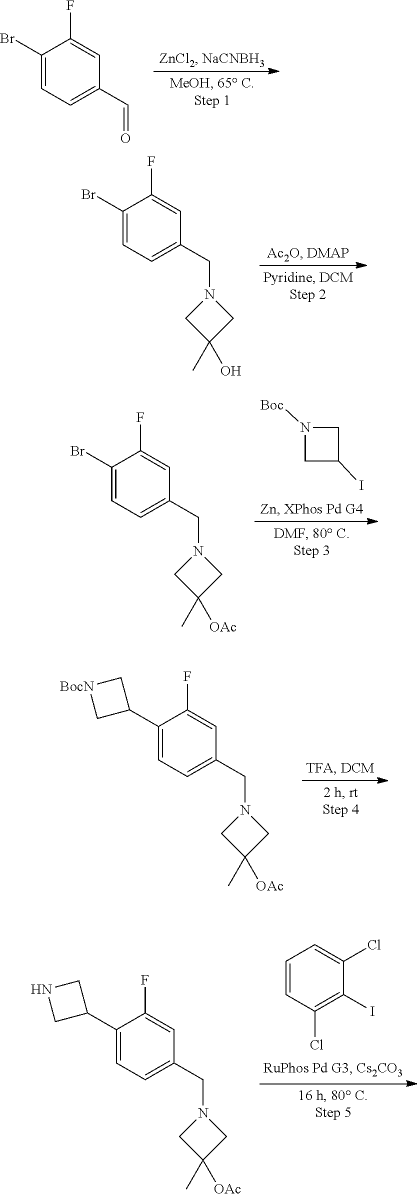

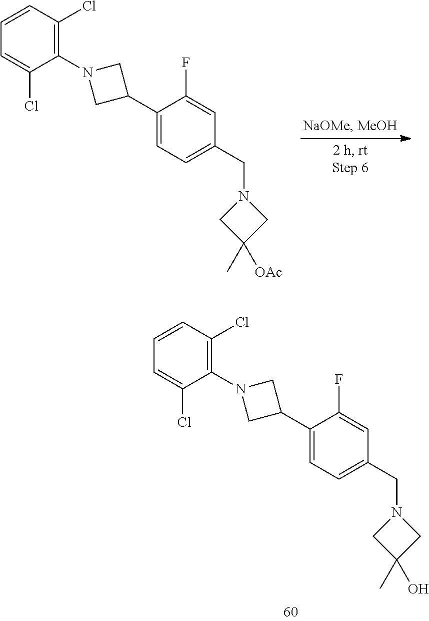 [SMILES: CC(=O)OC1(C)CN(Cc2ccc(C3CN(c4c(Cl)cccc4Cl)C3)c(F)c2)C1 ; CC1(O)CN(Cc2ccc(C3CN(c4c(Cl)cccc4Cl)C3)c(F)c2)C1]

### Synthesis of 1-(4-bromo-3-fluorobenzyl)-3-methylazetidin-3-ol

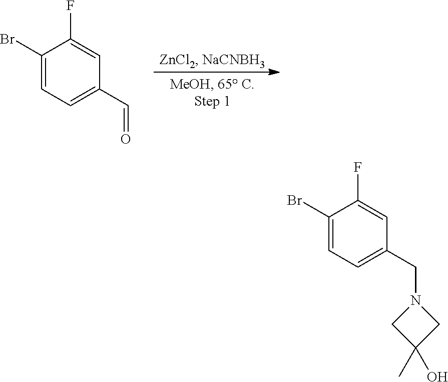

To a stirred solution of 3-methylazetidin-3-ol, TFA (1.486 g, 7.39 mmol) in MeOH(20 mL) was added sodium bicarbonate (1.035 g, 12.31 mmol) then stirred for 1 h at rt. The mixture was filtered through celite and concentrated to yield free amine. To a stirred solution of free amine and 4-bromo-3-fluorobenzaldehyde (1 g, 4.93 mmol) in MeOH(20 mL), zinc chloride (0.671 g, 4.93 mmol) was added and stirred for 1 h at 25° C. After 1 h, Sodium cyano borohydride (464 mg, 7.39 mmol.) was added and heated to 65° C. for 12 h. The reaction mixture was diluted with dichloromethane (60 mL) and washed sat. ammonium chloride solution and water (60 mL). The combined organic phase was dried over Na₂SO₄, filtered and the solvents were evaporated under reduced pressure, the crude was purified by flash column chromatography on silica gel (230-400 mesh) eluting with 50-100% ethyl acetate in petroleum ether to afford 1-(4-bromo-3-fluorobenzyl)-3-methylazetidin-3-ol (1.1 g, 82% yield) as Yellow semi-solid; LCMS method 1, LCMS (ESI, m/z): 275.8 [M+H]⁺.

### Synthesis of 1-(4-bromo-3-fluorobenzyl)-3-methylazetidin-3-yl acetate

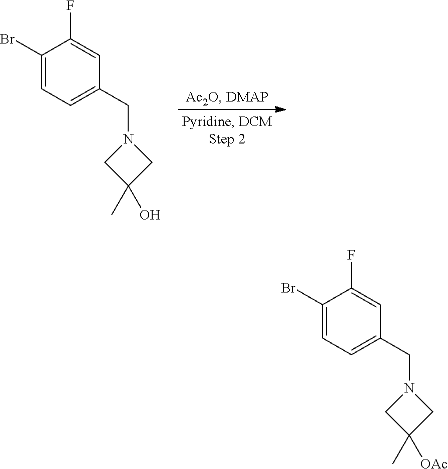 [SMILES: CC1(O)CN(Cc2ccc(Br)c(F)c2)C1 ; CC(=O)OC1(C)CN(Cc2ccc(Br)c(F)c2)C1]

To a stirred solution of 1-(4-bromo-3-fluorobenzyl)-3-methylazetidin-3-ol (1.5 g, 5.47 mmol) in DCM (20 mL) were added DMAP (0.668 g, 5.47 mmol) and pyridine (0.443 mL, 5.47 mmol) and the reaction mixture was stirred at room temperature for 10 minutes. After that acetic anhydride (1.549 mL, 16.42 mmol) was added and the reaction mixture was stirred for 16 h at rt. The reaction mixture was diluted with saturated solution of ammonium chloride and extracted with DCM. The combined organic layer was dried over sodium sulphate, concentrated under vacuum, purified by flash column chromatography on 230-400 mesh silica gel (eluted with 20-30% ethyl acetate in pet ether) to afford 1-(4-bromo-3-fluorobenzyl)-3-methylazetidin-3-yl acetate (1.43 g, 83% yield) as a Colorless oil, Yield 83%; LCMS method 1, LCMS (ESI, m/z): 316.0 [M+H]⁺.

### Synthesis of tert-butyl 3-(4-((3-acetoxy-3-methylazetidin-1-yl)methyl)-2-fluorophenyl)-azetidine-1-carboxylate

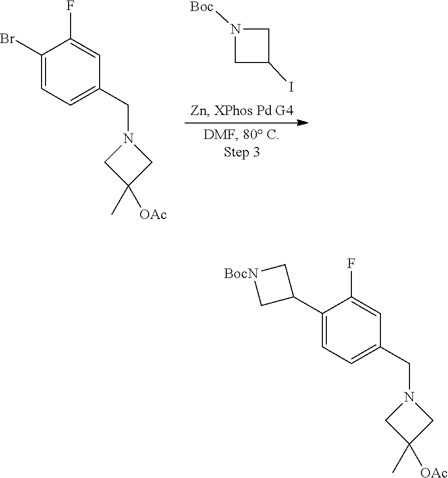 [SMILES: CC(=O)OC1(C)CN(Cc2ccc(Br)c(F)c2)C1 ; CC(C)(C)OC(=O)N1CC(I)C1 ; CC(=O)OC1(C)CN(Cc2ccc(C3CN(C(=O)OC(C)(C)C)C3)c(F)c2)C1]

To a suspension of activated zinc (2.481 g, 38.0 mmol) in anhydrous DMF (30 mL) was added 1,2-dibromoethane (0.164 mL, 1.898 mmol) and heated to 75° C. After 15 mins, the reaction mixture was cooled to room temperature, trimethylchlorosilane (0.243 mL, 1.898 mmol) was added and allowed to stir at ambient temperature for additional 30 mins. Then a solution of tert-butyl 3-iodoazetidine-1-carboxylate (3.22 g, 11.39 mmol) in 10 mL of anhydrous DMF was added to the reaction mixture and stirred at room temperature for another 30 min., followed by 1-(4-bromo-3-fluorobenzyl)-3-methylazetidin-3-yl acetate (1.2 g, 3.80 mmol) and Xphos Pd G4 (0.653 g, 0.759 mmol) in 10 mL of DMF was added. The reaction mixture was allowed to stir at 80° C. for 2 h. After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (50 mL), dried over anhydrous sodium sulphate and concentrated under reduced pressure. The residue thus obtained was purified by flash column chromatography on silica gel (100-200 mesh) eluting with 0-50% ethyl acetate in petroleum ether to afford tert-butyl 3-(4-((3-acetoxy-3-methylazetidin-1-yl)methyl)-2-fluorophenyl) azetidine-1-carboxylate (0.95 g, 64.4% yield) as Yellow semi-solid, Yield 64.4%, LCMS method 1, LCMS (ESI, m/z): 393.2 [M+H]⁺.

### Synthesis of 1-(4-(azetidin-3-yl)-3-fluorobenzyl)-3-methylazetidin-3-yl acetate

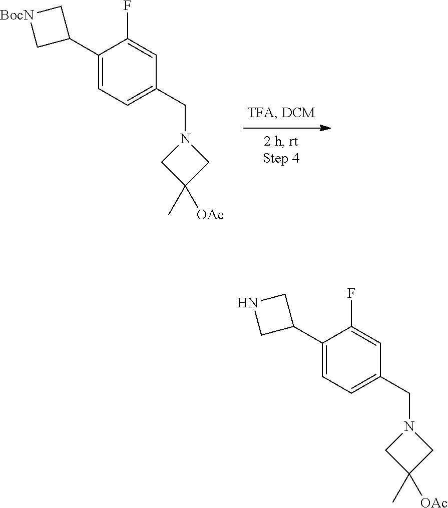 [SMILES: CC(=O)OC1(C)CN(Cc2ccc(C3CN(C(=O)OC(C)(C)C)C3)c(F)c2)C1 ; CC(=O)OC1(C)CN(Cc2ccc(C3CNC3)c(F)c2)C1]

To a stirred solution of tert-butyl tert-butyl 3-(4-((3-acetoxy-3-methylazetidin-1-yl)methyl)-2-fluorophenyl) azetidine-1-carboxylate (1.0 g, 2.55 mmol) in anhydrous dichloromethane (10 mL) was added trifluoroacetic acid (1.96 mL, 25.5 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford 1-(4-(azetidin-3-yl)-3-fluorobenzyl)-3-methylazetidin-3-yl acetate, TFA (900 mg, 2.215 mmol, 87% yield) as aYellow semi-solid, Yield 87%; LCMS method 1, LCMS (ESI, m/z): 293.1 [M+H]⁺.

### Synthesis of 1-(4-(1-(2,6-dichlorophenyl) azetidin-3-yl)-3-fluorobenzyl)-3-methylazetidin-3-yl acetate

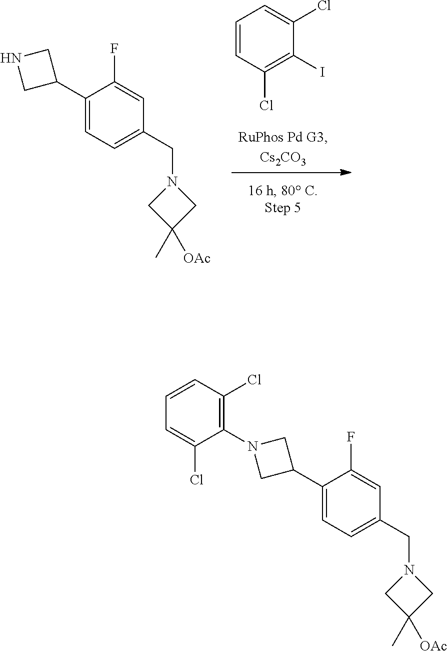 [SMILES: CC(=O)OC1(C)CN(Cc2ccc(C3CNC3)c(F)c2)C1 ; Clc1cccc(Cl)c1I ; CC(=O)OC1(C)CN(Cc2ccc(C3CN(c4c(Cl)cccc4Cl)C3)c(F)c2)C1]

To a solution of 1-(4-(azetidin-3-yl)-3-fluorobenzyl)-3-methylazetidin-3-yl acetate, TFA (500 mg, 1.230 mmol) and 1,3-difluoro-2-iodobenzene (504 mg, 1.84 mmol) in anhydrous 1,4 dioxane (8 mL) was added cesium carbonate (1.2 g, 3.69 mmol). The reaction mixture was then degassed with nitrogen for 10 min followed by the addition of added RuPhos Pd G3 (103 mg, 0.123 mmol) and heated to 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 1-(4-(1-(2,6-dichlorophenyl) azetidin-3-yl)-3-fluorobenzyl)-3-methylazetidin-3-yl acetate (230 mg, 0.526 mmol, 42.7% yield) as Yellow semi-solid, Yield 42.7%; LCMS method 1, LCMS (ESI, m/z): 439.2 [M+2H]⁺ .

### Synthesis of 1-(4-(1-(2,6-dichlorophenyl) azetidin-3-yl)-3-fluorobenzyl)-3-methylazetidin-3-ol (60)

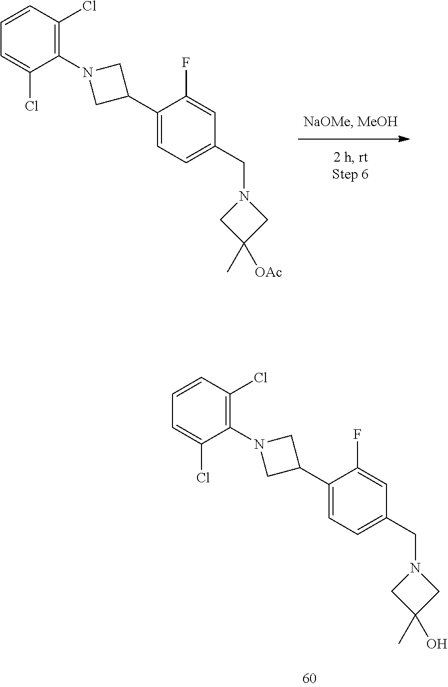 [SMILES: CC(=O)OC1(C)CN(Cc2ccc(C3CN(c4c(Cl)cccc4Cl)C3)c(F)c2)C1 ; CC1(O)CN(Cc2ccc(C3CN(c4c(Cl)cccc4Cl)C3)c(F)c2)C1]

To a stirred solution of 1-(4-(1-(2,6-dichlorophenyl) azetidin-3-yl)-3-fluorobenzyl)-3-methylazetidin-3-yl acetate (230 mg, 0.526 mmol) in methanol (8 mL) was added sodium methoxide (56.8 mg, 1.0 mmol). The resulting mixture was stirred at room temperature for 1 h. The reaction mixture was diluted with saturated solution of ammonium chloride and extracted with EtOAc. The combined organic layer was dried over sodium sulphate, concentrated under vacuum. The crude was purified using Prep. HPLC(Diluent: THF: Water: ACN (50:20:30); Column: Zorbax C18 (50×21.5) mm, 5 micron; Mobile phase A: 0.1% Formic acid in water; Mobile phase B: Acetonitrile) to afford 1-(4-(1-(2,6-dichlorophenyl) azetidin-3-yl)-3-fluorobenzyl)-3-methylazetidin-3-ol, formic acid salt (43 mg, 0.097 mmol, 18.40% yield, 99.3% purity) as a White solid, Yield 18.4% (99.3% purity).

¹H NMR (400 MHZ, MeOD): δ 7.60 (t, J=15.60 Hz, 1H), 7.24-7.26 (m, 1H), 7.15-7.20 (m, 3H), 6.70-6.74 (m, 1H), 4.90-4.94 (m, 2H), 4.43-4.47 (m, 2H), 3.98-4.06 (m, 3H), 3.69-3.72 (m, 2H), 3.52-3.55 (m, 2H), 1.50 (s, 3H). LCMS method 1, LCMS (ESI, m/z): 395.0 [M+H]⁺.

### Example S61. 1-(4-(1-(2,6-dichlorophenyl) azetidin-3-yl)-2-fluorobenzyl)-3-methylazetidin-3-ol (61)

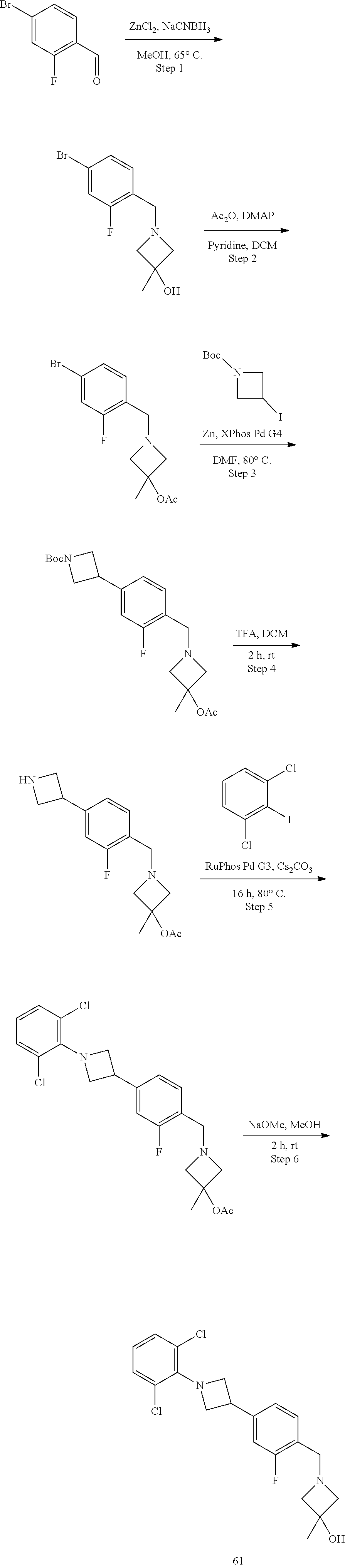

### Synthesis of 1-(4-bromo-2-fluorobenzyl)-3-methylazetidin-3-ol

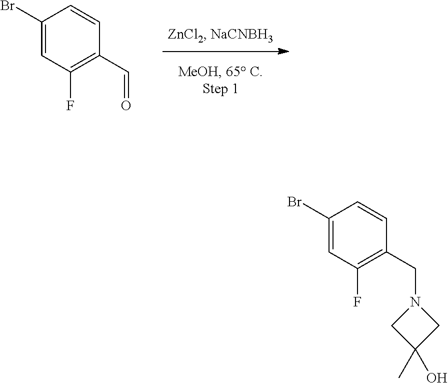

To a stirred solution of 3-methylazetidin-3-ol, TFA (3.10 g, 15.39 mmol) in MeOH(20 mL) was added sodium bicarbonate (3.10 g, 36.9 mmol) then stirred for 1 h at rt. The mixture was filtered through celite and concentrated to yield free amine. To the free amine 4-bromo-2-fluorobenzaldehyde (2.5 g, 12.31 mmol) in MeOH(20 mL) and zinc chloride (1.678 g, 12.31 mmol) was added and stirred for 1 h at 25° C. After 1 h, sodium cyanoborohydride (1.161 g, 18.47 mmol) was added and heated to 65° C. for 12 h. The reaction mixture was diluted with dichloromethane (60 mL) and washed sat. ammonium chloride solution and water (60 mL). The combined organic phase was dried over Na₂SO₄, filtered and the solvents were evaporated under reduced pressure, the crude was purified by flash column chromatography on silica gel (230-400 mesh) eluting with 50-100% ethyl acetate in petroleum ether to afford 1-(4-bromo-2-fluorobenzyl)-3-methylazetidin-3-ol (3.3 g, 97% yield) as yellow liquid; LCMS method 1, LCMS (ESI, m/z): 274.0 [M+H]⁺.

### Synthesis of 1-(4-bromo-2-fluorobenzyl)-3-methylazetidin-3-yl acetate

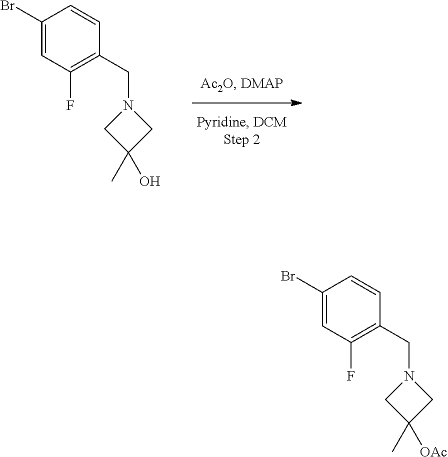 [SMILES: CC1(O)CN(Cc2ccc(Br)cc2F)C1 ; CC(=O)OC1(C)CN(Cc2ccc(Br)cc2F)C1]

To a stirred solution of 1-(4-bromo-2-fluorobenzyl)-3-methylazetidin-3-ol (3.2 g, 11.67 mmol) in DCM (40 mL) were added DMAP (1.426 g, 11.67 mmol) and pyridine (0.944 mL, 11.67 mmol) and the reaction mixture was stirred at room temperature for 10 minutes. After that acetic anhydride (4.41 mL, 46.7 mmol) was added and the reaction mixture was stirred for 16 h at rt. The reaction mixture was diluted with saturated solution of ammonium chloride and extracted with DCM. The combined organic layer was dried over sodium sulphate, concentrated under vacuum, purified by flash column chromatography on 230-400 mesh silica gel (eluted with 20-50% ethyl acetate in pet ether) to afford 1-(4-bromo-2-fluorobenzyl)-3-methylazetidin-3-yl acetate (2.58 g, 70.0% yield) as a Pale yellow semi-solid, Yield 70%, LCMS method 3, LCMS (ESI, m/z): 318.0 [M+2H]⁺ .

### Synthesis of tert-butyl 3-(4-((3-acetoxy-3-methylazetidin-1-yl)methyl)-3-fluorophenyl) azetidine-1-carboxylate

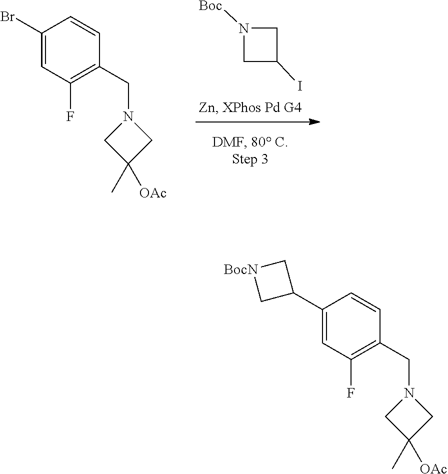 [SMILES: CC(=O)OC1(C)CN(Cc2ccc(Br)cc2F)C1 ; CC(C)(C)OC(=O)N1CC(I)C1 ; CC(=O)OC1(C)CN(Cc2ccc(C3CN(C(=O)OC(C)(C)C)C3)cc2F)C1]

To a suspension of activated zinc (4.14 g, 63.3 mmol) in anhydrous DMF (30 mL) was added 1,2-dibromoethane (0.273 mL, 3.16 mmol) and heated to 75° C. After 15 mins, the reaction mixture was cooled to room temperature, trimethylchlorosilane (0.404 mL, 3.16 mmol) was added and allowed to stir at ambient temperature for additional 30 mins. Then a solution of tert-butyl 3-iodoazetidine-1-carboxylate (5.37 g, 18.98 mmol) in 10 mL of anhydrous DMF was added to the reaction mixture and stirred at room temperature for another 30 min., followed by 1-(4-bromo-2-fluorobenzyl)-3-methylazetidin-3-yl acetate (2 g, 6.33 mmol) and Xphos Pd G4 (0.544 g, 0.633 mmol) in 10 mL of DMF was added. The reaction mixture was allowed to stir at 80° C. for 2 h. After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (50 mL), dried over anhydrous sodium sulphate and concentrated under reduced pressure. The residue thus obtained was purified by flash column chromatography on silica gel (100-200 mesh) eluting with 0-50% ethyl acetate in petroleum ether to afford tert-butyl 3-(4-((3-acetoxy-3-methylazetidin-1-yl)methyl)-3-fluorophenyl) azetidine-1-carboxylate (556 mg, 22.91% yield) as a Yellow semi-solid, Yield 23%; LCMS method 1, LCMS (ESI, m/z): 393.2 [M+H]⁺.

### Synthesis of 1-(4-(azetidin-3-yl)-2-fluorobenzyl)-3-methylazetidin-3-yl acetate

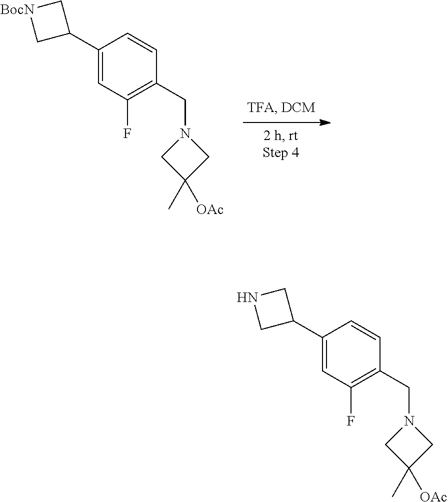 [SMILES: CC(=O)OC1(C)CN(Cc2ccc(C3CN(C(=O)OC(C)(C)C)C3)cc2F)C1 ; CC(=O)OC1(C)CN(Cc2ccc(C3CNC3)cc2F)C1]

To a stirred solution of tert-butyl 3-(4-((3-acetoxy-3-methylazetidin-1-yl)methyl)-3-fluorophenyl) azetidine-1-carboxylate (900 mg, 2.293 mmol) in anhydrous dichloromethane (10 mL) was added trifluoroacetic acid (1.76 mL, 22.93 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford 1-(4-(azetidin-3-yl)-2-fluorobenzyl)-3-methylazetidin-3-yl acetate, TFA (700 mg, 1.723 mmol, 75% yield) as a Yellow semi-solid; LCMS method 1, LCMS (ESI, m/z): 293.2 [M+H]⁺.

### Synthesis of 1-(4-(1-(2,6-dichlorophenyl) azetidin-3-yl)-2-fluorobenzyl)-3-methylazetidin-3-yl acetate

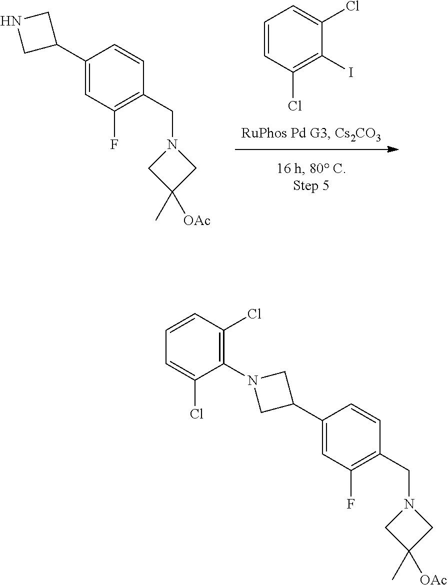 [SMILES: CC(=O)OC1(C)CN(Cc2ccc(C3CNC3)cc2F)C1 ; Clc1cccc(Cl)c1I ; CC(=O)OC1(C)CN(Cc2ccc(C3CN(c4c(Cl)cccc4Cl)C3)cc2F)C1]

To a solution of 1-(4-(azetidin-3-yl)-2-fluorobenzyl)-3-methylazetidin-3-yl acetate, TFA (400 mg, 0.984 mmol) and 1,3-difluoro-2-iodobenzene (336 mg, 1.230 mmol) in anhydrous 1,4 dioxane (8 mL) was added cesium carbonate (0.96 g, 2.95 mmol). The reaction mixture was then degassed with nitrogen for 10 min followed by the addition of added RuPhos Pd G3 (82 mg, 0.098 mmol) and heated to 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 1-(4-(1-(2,6-dichlorophenyl) azetidin-3-yl)-2-fluorobenzyl)-3-methylazetidin-3-yl acetate (174 mg, 40.6% yield) as a Light yellow semi-solid; LCMS method 3, LCMS (ESI, m/z): 438.0 [M+H]⁺

### Synthesis of 1-(4-(1-(2,6-dichlorophenyl) azetidin-3-yl)-2-fluorobenzyl)-3-methylazetidin-3-ol, formic acid salt (61)

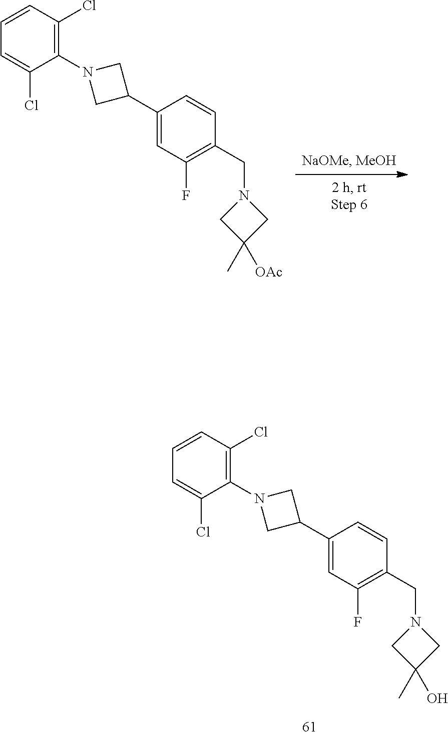 [SMILES: CC(=O)OC1(C)CN(Cc2ccc(C3CN(c4c(Cl)cccc4Cl)C3)cc2F)C1 ; CC1(O)CN(Cc2ccc(C3CN(c4c(Cl)cccc4Cl)C3)cc2F)C1]

To a stirred solution of 1-(4-(1-(2,6-dichlorophenyl) azetidin-3-yl)-2-fluorobenzyl)-3-methylazetidin-3-yl acetate (95 mg, 0.217 mmol) in methanol (3 mL) was added sodium methoxide (23.47 mg, 0.434 mmol). The resulting mixture was stirred at room temperature for 1 h. The reaction mixture was diluted with saturated solution of ammonium chloride and extracted with EtOAc. The combined organic layer was dried over sodium sulphate, concentrated under vacuum. The crude was purified using Prep. HPLC(Diluent: THF: Water: ACN (50:10:40); Column-1: Zorbax C18 (50×21.5) mm, 5 micron; Mobile phase A: 0.1% Formic acid in water; Mobile phase B: Acetonitrile) to afford 1-(4-(1-(2,6-dichlorophenyl) azetidin-3-yl)-2-fluorobenzyl)-3-methylazetidin-3-ol, formic acid salt (31 mg, 0.069 mmol, 31.9% yield, 98.61% purity) as a white solid.

¹H NMR (400 MHZ, MeOD): δ 7.81 (s, 1H), 7.57 (t, J=−148.00 Hz, 1H), 7.33 (t, J=10.40 Hz, 2H), 7.18 (d, J=8.00 Hz, 2H), 6.72 (t, J=7.60 Hz, 1H), 4.83-4.92 (m, 2H), 4.42 (t, J=6.00 Hz, 2H), 4.18 (s, 2H), 3.83 (d, J=9.60 Hz, 2H), 3.75-3.78 (m, 1H), 1.51 (s, 3H)(2H are merged with solvent signal). LCMS method 1, LCMS (ESI, m/z): 395.0 [M+H]⁺.

### Example S62. 1-(4-(3-(2,6-dichlorophenyl) azetidin-1-yl)-2,5-dimethylbenzyl)-3-methylazetidin-3-ol (62)

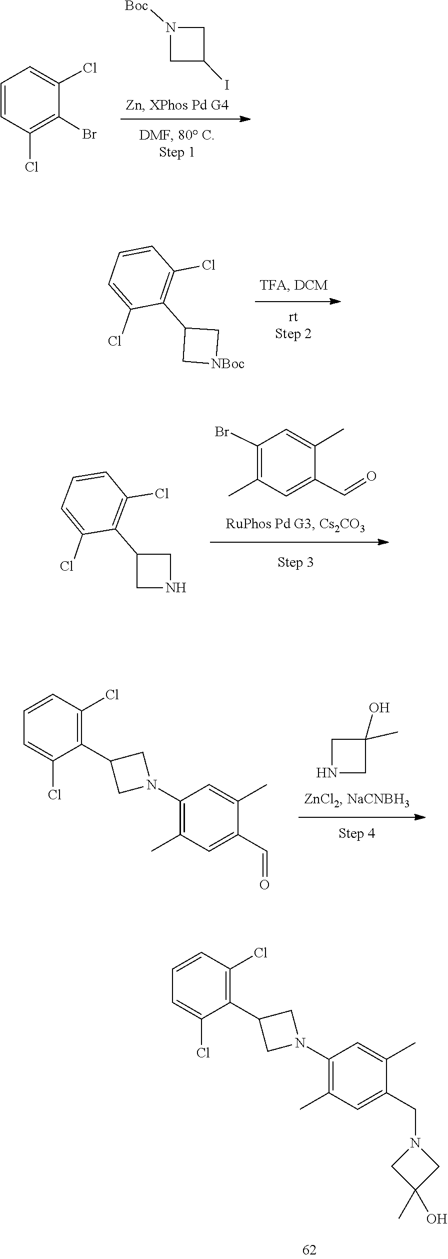

### Synthesis of tert-butyl 3-(2,6-dichlorophenyl) azetidine-1-carboxylate

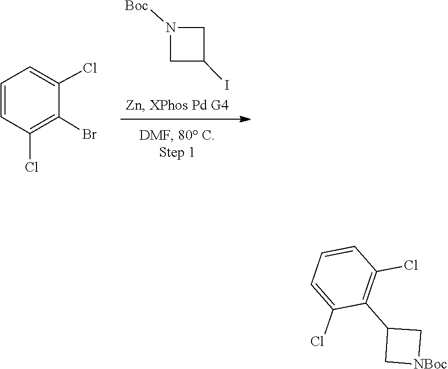 [SMILES: Clc1cccc(Cl)c1Br ; CC(C)(C)OC(=O)N1CC(I)C1 ; CC(C)(C)OC(=O)N1CC(c2c(Cl)cccc2Cl)C1]

To a suspension of activated Zinc Dust (5.79 g, 89 mmol) in anhydrous DMF (20 mL) was added 1,2-Dibromoethane (0.383 mL, 4.43 mmol) and heated to 75° C. After 15 mins, the reaction mixture was cooled to room temperature, Trimethylsilyl chloride (0.562 mL, 4.43 mmol) was added and allowed to stir at ambient temperature for addition 30 mins. Then a solution of tert-butyl 3-iodoazetidine-1-carboxylate (2.507 g, 8.85 mmol) in 5 mL of anhydrous DMF was added to the reaction mixture and stirred at room temperature for another 30 min., followed by 2-bromo-1,3-dichlorobenzene (2.0 g, 8.85 mmol) and XPhos Pd G4 (1.143 g, 1.328 mmol) in 5 mL of DMF was added. The reaction mixture was allowed to stir at 80° C. for 2 h.

After completion of the reaction, the reaction mixture was cooled to ambient temperature and quenched with sat. ammonium chloride solution. The crude was filtered through a pad of celite and washed with ethyl acetate. The filtrate was then transferred to a separating funnel and washed with cold water (50 mL), dried over anhydrous sodium sulphate and concentrated under reduced pressure. The residue thus obtained was purified by flash column chromatography on silica gel (100-200 mesh) eluting with 0-50% ethyl acetate in petroleum ether to afford tert-butyl 3-(2,6-dichlorophenyl) azetidine-1-carboxylate (1.3 g, 4.30 mmol, 48.6% yield) as a colorless liquid; LCMS method 2, LCMS (ESI, m/z): 202.1 [M−100]*.

### Synthesis of 3-(2,6-dichlorophenyl) azetidine

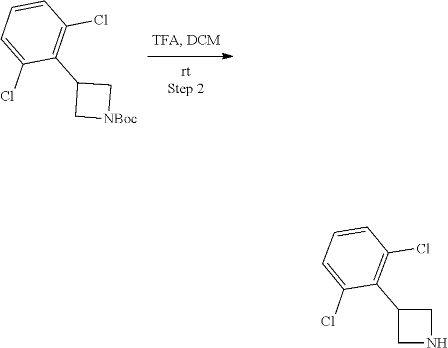 [SMILES: CC(C)(C)OC(=O)N1CC(c2c(Cl)cccc2Cl)C1 ; Clc1cccc(Cl)c1C1CNC1]

To a stirred solution of tert-butyl 3-(2,6-dichlorophenyl) azetidine-1-carboxylate (1.3 g, 4.30 mmol) in anhydrous dichloromethane (15 mL) was added trifluoroacetic acid (1.647 mL, 21.51 mmol) at 0° C. Then reaction mixture was stirred at ambient temperature and the progress of the reaction monitored by TLC analysis. After 1 h, TLC analysis indicated complete conversion of the starting material. The reaction mixture was concentrated under reduced pressure and the residue thus obtained was triturated with diethyl ether to afford 3-(2,6-dichlorophenyl) azetidine, TFA (0.86 g, 63.4% yield) as light blue semi-solid. LCMS method 1, LCMS (ESI, m/z): 202.1 [M+H]⁺.

### Synthesis of 4-(3-(2,6-dichlorophenyl) azetidin-1-yl)-2,5-dimethylbenzaldehyde

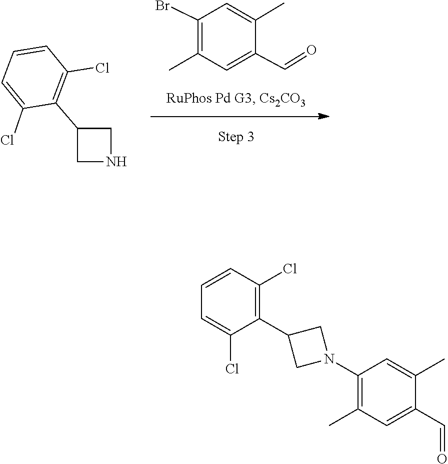 [SMILES: Clc1cccc(Cl)c1C1CNC1 ; Cc1cc(C=O)c(C)cc1Br ; Cc1cc(N2CC(c3c(Cl)cccc3Cl)C2)c(C)cc1C=O]

To a solution of 3-(2,6-dichlorophenyl) azetidine, TFA salt (150 mg, 0.475 mmol) and 4-bromo-2,5-dimethylbenzaldehyde (152 mg, 0.712 mmol) in anhydrous 1,4 dioxane (5 mL) was added cesium carbonate (464 mg, 1.424 mmol). The reaction mixture was then degassed with nitrogen for 10 min followed by the addition of added RuPhos Pd G3 (39.7 mg, 0.047 mmol) and heated 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 4-(1-(2,6-dichlorophenyl) azetidin-3-yl)-2,5-dimethylbenzaldehyde (60 mg, 38% yield) as a Orange brown solid. LCMS (ESI, m/z): 334 [M]⁺.

### Synthesis of 1-(4-(3-(2,6-dichlorophenyl) azetidin-1-yl)-2,5-dimethylbenzyl)-3-methylazetidin-3-ol (62)

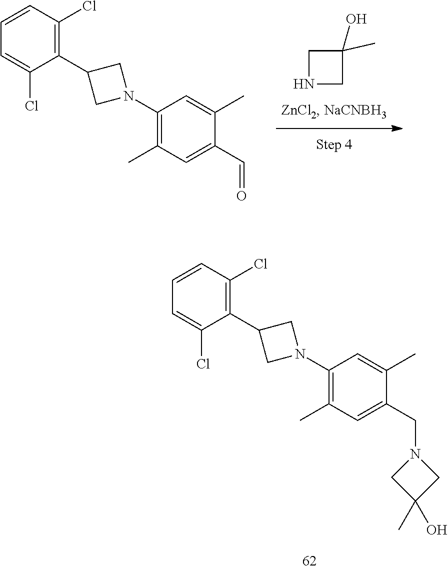

To a stirred solution of 3-methylazetidin-3-ol, TFA salt (74.5 mg, 0.606 mmol) in MeOH(5 mL) was added sodium bicarbonate (127 mg, 1.51 mmol.) then stirred for 1 h at rt. The mixture was filtered through celite and concentrated to yield free amine. To the free amine and 4-(3-(2,6-dichlorophenyl) azetidin-1-yl)-2,5-dimethylbenzaldehyde (135 mg, 0.404 mmol), zinc chloride (83 mg, 0.606 mmol) was added and stirred for 1 h at 25° C. After 1 h, Sodium cyano borohydride (38.1 mg, 0.606 mmol.) was added and heated to 65° C. for 12 h. The reaction mixture was diluted with dichloromethane (10 mL) and washed sat. ammonium chloride solution and water (20 mL). The combined organic phase was dried over Na₂SO₄, filtered and the solvents were evaporated under reduced pressure. The crude was purified by prep. HPLC method (Diluent: THF: Water: ACN (50:20:30), Column: Xbridge C₈ (250×19) mm, 5 micron, Mobile phase A: 10 mM Ammonium bicarbonate, Mobile phase B: Acetonitrile), required fractions were concentrated and lyophilized to afford the title compound 1-(4-(1-(2,6-dichlorophenyl) azetidin-3-yl)-2,5-dimethylbenzyl)-3-methylazetidin-3-ol (21 mg, 13% yield) as a white solid.

¹H NMR (400 MHZ, DMSO-d₆): δ 7.45 (d, J=7.60 Hz, 2H), 7.29 (t, J=8.00 Hz, 1H), 6.83 (s, 1H), 6.32 (s, 1H), 5.09 (s, 1H), 4.47-4.39 (m, 3H), 3.99-3.96 (m, 2H), 3.42 (s, 2H), 3.11 (d, J=6.00 Hz, 2H), 2.85 (d, J=5.20 Hz, 2H), 2.18 (s, 3H), 2.15 (s, 3H), 1.33 (s, 3H). LCMS (ESI, m/z): 405.2 [M+H]⁺.

### Example S63. 1-(4-(3-(2,6-dichlorophenyl) azetidin-1-yl)-3,5-dimethylbenzyl)-3-methylazetidin-3-ol (63)

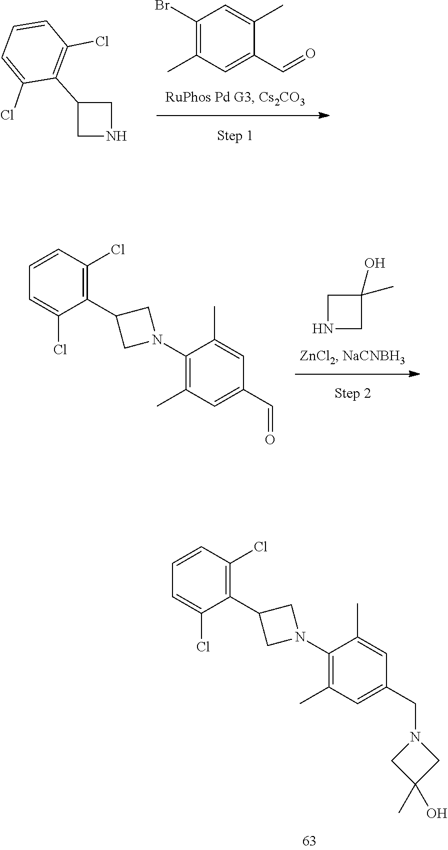

### Synthesis of 4-(3-(2,6-dichlorophenyl) azetidin-1-yl)-3,5-dimethylbenzaldehyde

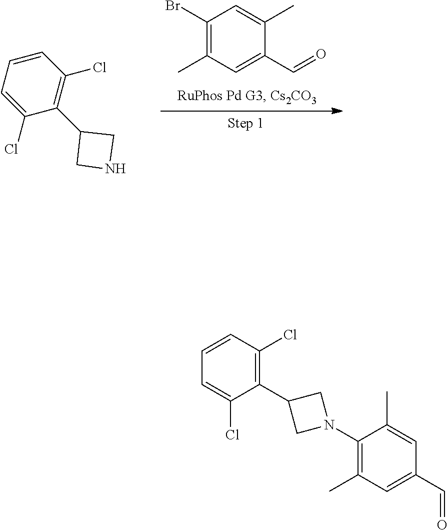 [SMILES: Clc1cccc(Cl)c1C1CNC1 ; Cc1cc(C=O)c(C)cc1Br ; Cc1cc(C=O)cc(C)c1N1CC(c2c(Cl)cccc2Cl)C1]

To a solution of 3-(2,6-dichlorophenyl) azetidine, TFA salt (221 mg, 0.742 mmol) and 4-bromo-3,5-dimethylbenzaldehyde (237 mg, 1.113 mmol) in anhydrous 1,4 dioxane (5 mL) was added Cesium carbonaterbonate (726 mg, 2.227 mmol). The reaction mixture was then degassed with nitrogen for 10 min followed by the addition of added RuPhos-Pd-G3 (62.1 mg, 0.074 mmol) and heated 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 4-(3-(2,6-dichlorophenyl) azetidin-1-yl)-3,5-dimethylbenzaldehyde (52 mg, 20.97% yield) as a white solid; LCMS method 1, LCMS (ESI, m/z): 334.2 [M]⁺.

### Synthesis of 1-(4-(3-(2,6-dichlorophenyl) azetidin-1-yl)-3,5-dimethylbenzyl)-3-methyl-azetidin-3-ol (63)

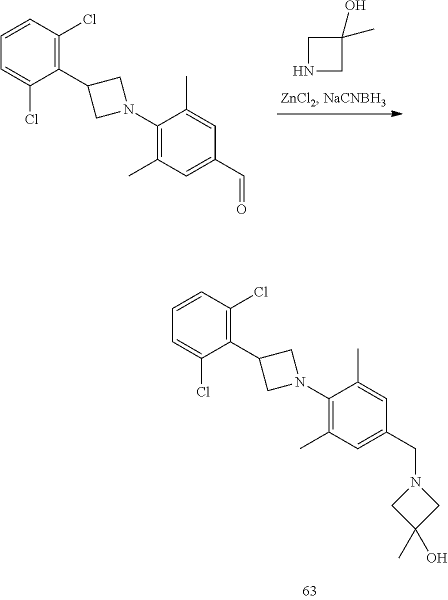

To a stirred solution of 4-(3-(2,6-dichlorophenyl) azetidin-1-yl)-3,5-dimethylbenzaldehyde (120 mg, 0.359 mmol) in MeOH(8 mL) was added 3-methylazetidin-3-ol (46.9 mg, 0.539 mmol) and Zinc chloride (73.4 mg, 0.539 mmol). The reaction mixture was stirred for 1 h at 25° C. After 1 h, sodium cyanoborohydride (33.8 mg, 0.539 mmol) was added and heated to 65° C. for 12 h. The reaction mixture was diluted with dichloromethane (10 mL) and washed sat. ammonium chloride solution and water (20 mL). The combined organic phase was dried over Na₂SO₄, filtered and the solvents were evaporated under reduced pressure. The crude was purified by prep. HPLC method (Diluent: THF: Acetonitrile (30:70); Column: Xbridge C₈ (150×19) mm, 5 micron; Mobile phase A: 0.1% Formic acid in water; Mobile phase B: Acetonitrile), required fractions were concentrated and lyophilized to afford the title compound 1-(4-(3-(2,6-dichlorophenyl) azetidin-1-yl)-3,5-dimethylbenzyl)-3-methylazetidin-3-ol, formic acid salt (40 mg, 0.088 mmol, 24.59% yield, 99.6% purity) as a white solid.

¹H NMR (400 MHZ, DMSO-d₆): δ 7.44 (d, J=8.00 Hz, 2H), 7.28 (t, J=7.60 Hz, 1H), 6.71 (s, 2H), 5.13 (s, 1H), 4.66 (m, 2H), 4.28 (m, 3H), 3.41 (s, 2H), 3.16 (d, J=8.00 Hz, 2H), 2.89 (d, J=7.60 Hz, 2H), 2.26 (s, 6H), 1.32 (s, 3H); LCMS method 1, LCMS (ESI, m/z): 405.2 [M+H]⁺.

### Example S64. 1-((6-(3-(2,6-dichlorophenyl) azetidin-1-yl) pyridin-3-yl)methyl)-3-methylazetidin-3-ol (64)

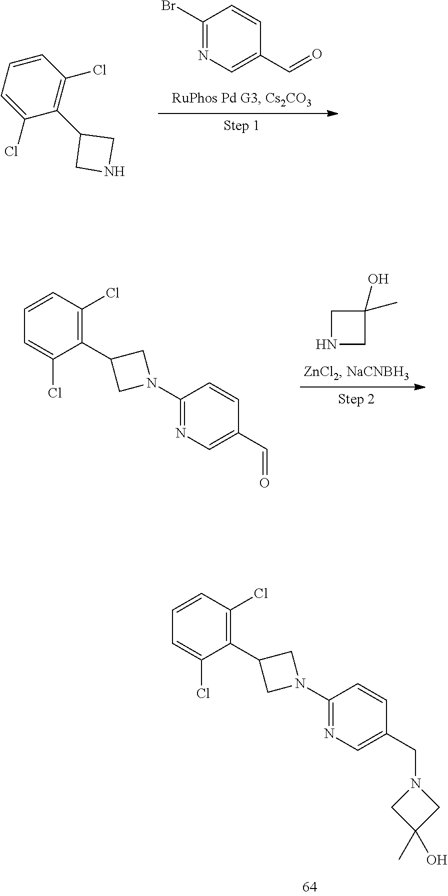

### Synthesis of 6-(3-(2,6-dichlorophenyl) azetidin-1-yl) nicotinaldehyde

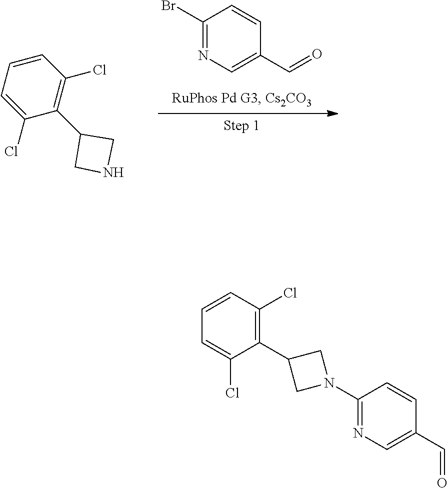 [SMILES: Clc1cccc(Cl)c1C1CNC1 ; O=Cc1ccc(Br)nc1 ; O=Cc1ccc(N2CC(c3c(Cl)cccc3Cl)C2)nc1]

To a solution of 3-(2,6-dichlorophenyl) azetidine, TFA salt (240 mg, 0.806 mmol) and 6-bromonicotinaldehyde (150 mg, 0.806 mmol) in anhydrous 1,4 dioxane (5 mL) was added Cesium carbonate (788 mg, 2.419 mmol). The reaction mixture was then degassed with nitrogen for 10 min followed by the addition of added RuPhosPdG3 (67.4 mg, 0.081 mmol) and heated 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 6-(3-(2,6-dichlorophenyl) azetidin-1-yl) nicotinaldehyde (78 mg, 31.5% yield) as a yellow solid; LCMS method 1; LCMS (ESI, m/z): 306.9 [M]⁺.

### Synthesis of 1-((6-(3-(2,6-dichlorophenyl) azetidin-1-yl) pyridin-3-yl)methyl)-3-methyl-azetidin-3-ol (64)

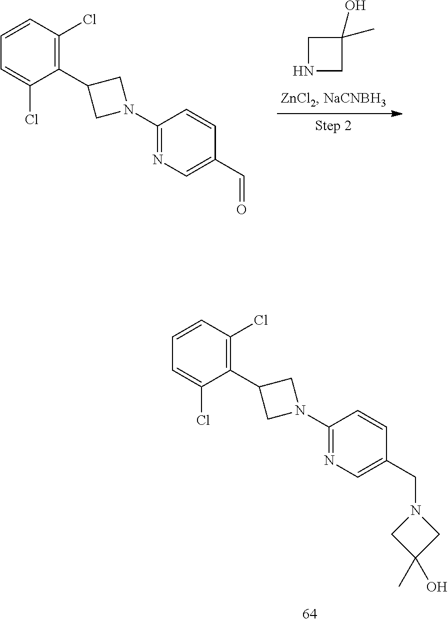

To a stirred solution of 3-methylazetidin-3-ol, TFA (98 mg, 0.488 mmol) in MeOH(5 mL) was added sodium bicarbonate (103 mg, 1.221 mmol) then stirred for 1 h at rt. The mixture was filtered through celite and concentrated to yield free amine. To the free amine and 6-(3-(2,6-dichlorophenyl) azetidin-1-yl) nicotinaldehyde (150 mg, 0.488 mmol) in MeOH(3 mL) was added Zinc chloride (80 mg, 0.586 mmol) and stirred for 1 h at 25° C. After 1 h, sodium cyanoborohydride (30.7 mg, 0.488 mmol) was added and heated to 65° C. for 12 h. The reaction mixture was diluted with dichloromethane (10 mL) and washed sat. ammonium chloride solution and water (20 mL). The combined organic phase was dried over Na₂SO₄, filtered and the solvents were evaporated under reduced pressure. The crude was purified by prep. HPLC method (Diluent: THF: Water: ACN (50:20:30), Column: Xbridge C₈ (250×19) mm, 5 micron, Mobile phase A: 10 mM Ammonium bicarbonate, Mobile phase B: Acetonitrile), required fractions were concentrated and lyophilized to afford the title compound 1-((6-(3-(2,6-dichlorophenyl) azetidin-1-yl) pyridin-3-yl)methyl)-3-methylazetidin-3-ol, formic acid salt (12 mg, 0.027 mmol, 5.53% yield, 95.5% purity) as a pale yellow solid.

¹H NMR (400 MHZ, DMSO-d₆): δ 8.10 (d, J=2.00 Hz, 1H), 7.96 (d, J=1.60 Hz, 1H), 7.43-7.46 (m, 3H), 7.30 (t, J=8.40 Hz, 1H), 6.43 (d, J=8.40 Hz, 1H), 5.18 (s, 1H), 4.63 (m, 1H), 4.41 (t, J=8.40 Hz, 2H), 4.25 (t, J=8.00 Hz, 2H), 3.13-3.15 (m, 2H), 2.91 (d, J=7.60 Hz, 2H), 1.33 (s, 3H); LCMS method 1, LCMS (ESI, m/z): 380.0 [M+H]⁺.

### Example S65. 1-(4-(3-(2,6-dichlorophenyl) azetidin-1-yl)-2-fluorobenzyl)-3-methylazetidin-3-ol (65)

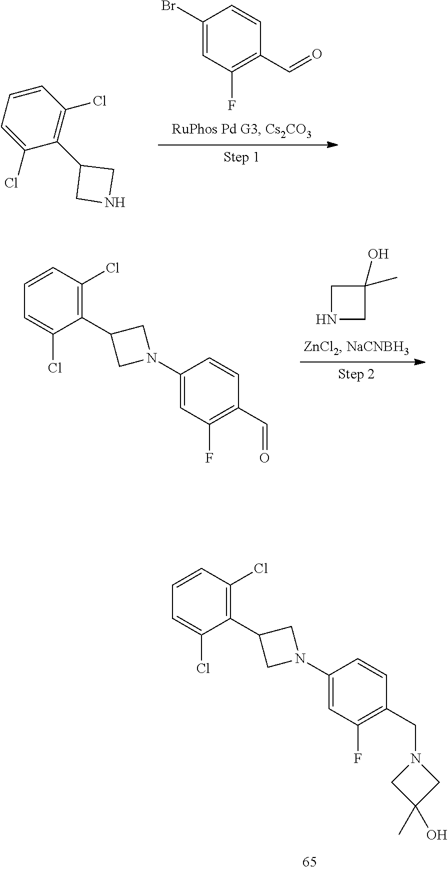

### Synthesis of 4-(3-(2,6-dichlorophenyl) azetidin-1-yl)-2-fluorobenzaldehyde

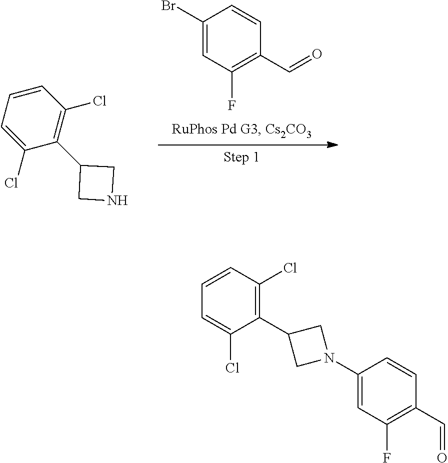 [SMILES: Clc1cccc(Cl)c1C1CNC1 ; O=Cc1ccc(Br)cc1F ; O=Cc1ccc(N2CC(c3c(Cl)cccc3Cl)C2)cc1F]

To a solution of 3-(2,6-dichlorophenyl) azetidine (150 mg, 0.742 mmol) and 4-bromo-2-fluorobenzaldehyde (226 mg, 1.113 mmol) in anhydrous 1,4 dioxane (5 mL) was added Cesium carbonaterbonate (726 mg, 2.227 mmol). The reaction mixture was then degassed with nitrogen for 10 min followed by the addition of added RuPhos-Pd-G3 (62.1 mg, 0.074 mmol) and heated 80° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 4-(3-(2,6-dichlorophenyl) azetidin-1-yl)-2-fluorobenzaldehyde (55.23 mg, 22.92% yield) as a white solid; LCMS method 1, LCMS (ESI, m/z): 324.0 [M]⁺.

### Synthesis of 1-(4-(3-(2,6-dichlorophenyl) azetidin-1-yl)-2-fluorobenzyl)-3-methylazetidin-3-ol (65)

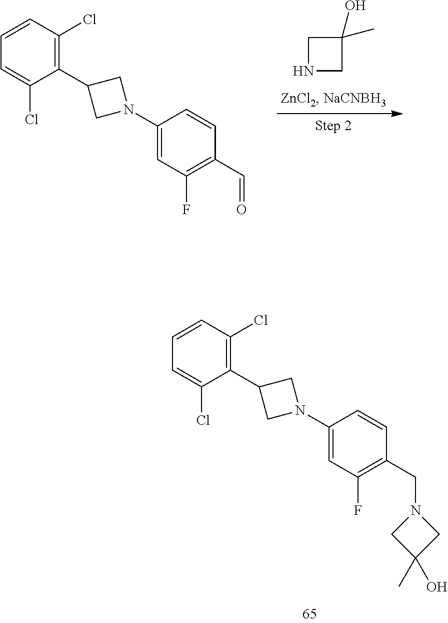

To a stirred solution of 4-(3-(2,6-dichlorophenyl) azetidin-1-yl)-2-fluorobenzaldehyde (110 mg, 0.339 mmol) in MeOH(8 mL) was added 3-methylazetidin-3-ol (44.3 mg, 0.509 mmol) and Zinc chloride (69.4 mg, 0.509 mmol). The reaction mixture was stirred for 1 h at 25° C. After 1 h, sodium cyanoborohydride (32.0 mg, 0.509 mmol) was added and heated to 65° C. for 12 h. The reaction mixture was diluted with dichloromethane (10 mL) and washed sat. ammonium chloride solution and water (20 mL). The combined organic phase was dried over Na₂SO₄, filtered and the solvents were evaporated under reduced pressure. The crude was purified by prep. HPLC method (Diluent: THF: Acetonitrile (30:70); Column: Xbridge C₈ (150×19) mm, 5 micron; Mobile phase A: 0.1% Formic acid in water; Mobile phase B: Acetonitrile), required fractions were concentrated and lyophilized to afford the title compound 1-(4-(3-(2,6-dichlorophenyl) azetidin-1-yl)-2-fluorobenzyl)-3-methylazetidin-3-ol, formic acid salt (61 mg, 0.138 mmol, 40.7% yield, 99.9% purity) as a white solid.

¹H NMR (400 MHZ, DMSO-d₆): δ 7.47 (d, J=8.00 Hz, 2H), 7.31 (t, J=8.00 Hz, 1H), 7.15 (t, J=8.40 Hz, 1H), 6.31 (t, J=10.00 Hz, 2H), 5.27 (s, 1H), 4.63-4.58 (m, 1H), 4.41 (t, J=8.00 Hz, 2H), 4.08 (t, J=8.00 Hz, 2H), 3.61 (s, 2H), 3.06 (s, 2H), 1.33 (s, 3H)(two protons are merged with solvent signal). LCMS method 1, LCMS (ESI, m/z): 395.0 [M]⁺.

### Example S66. 1-((5-(3-(2,6-dichlorophenyl) azetidin-1-yl) pyridin-2-yl)methyl)-3-methylazetidin-3-ol (66)

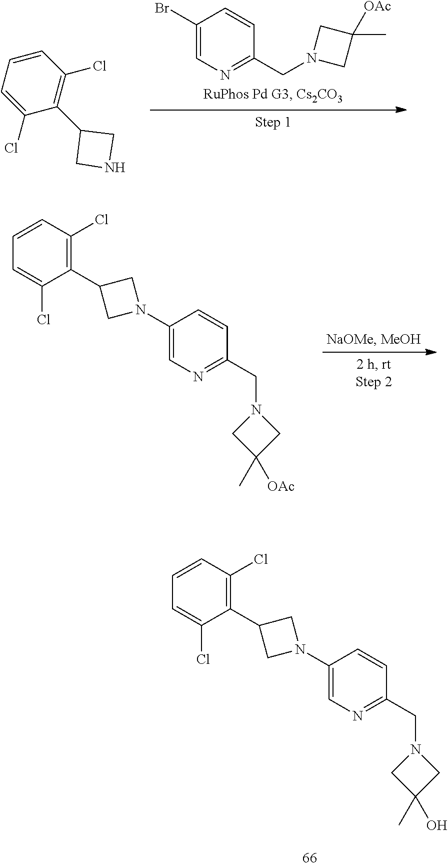 [SMILES: Clc1cccc(Cl)c1C1CNC1 ; CC(=O)OC1(C)CN(Cc2ccc(Br)cn2)C1 ; CC(=O)OC1(C)CN(Cc2ccc(N3CC(c4c(Cl)cccc4Cl)C3)cn2)C1 ; CC1(O)CN(Cc2ccc(N3CC(c4c(Cl)cccc4Cl)C3)cn2)C1]

### Synthesis of 1-((5-(3-(2,6-dichlorophenyl) azetidin-1-yl) pyridin-2-yl)methyl)-3-methylazetidin-3-yl acetate

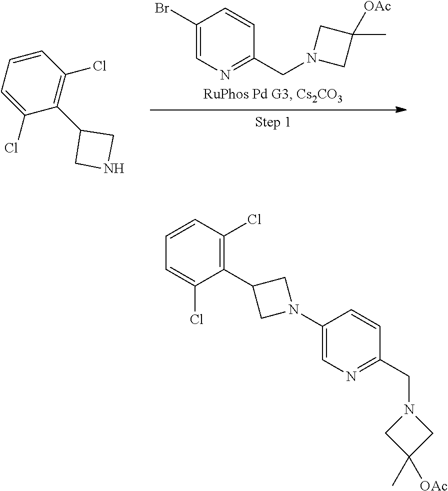 [SMILES: Clc1cccc(Cl)c1C1CNC1 ; CC(=O)OC1(C)CN(Cc2ccc(Br)cn2)C1 ; CC(=O)OC1(C)CN(Cc2ccc(N3CC(c4c(Cl)cccc4Cl)C3)cn2)C1]

To a solution of 3-(2,6-dichlorophenyl) azetidine, TFA (211 mg, 0.669 mmol) and 1-((5-bromopyridin-2-yl)methyl)-3-methylazetidin-3-yl acetate (200 mg, 0.669 mmol) in anhydrous 1,4 dioxane (5 mL) was added cesium carbonate (653 mg, 2.006 mmol). The reaction mixture was then degassed with nitrogen for 10 min followed by the addition of added XPhos Pd G4 (57.5 mg, 0.067 mmol) and heated 100° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 1-((5-(3-(2,6-dichlorophenyl) azetidin-1-yl) pyridin-2-yl)methyl)-3-methylazetidin-3-yl acetate (94 mg, 38% yield) as a brown solid. LCMS method 1; LCMS (ESI, m/z): 422.0 [M+H]⁺.

### Synthesis of Synthesis of 1-((5-(3-(2,6-dichlorophenyl) azetidin-1-yl) pyridin-2-yl)methyl)-3-methylazetidin-3-ol (66)

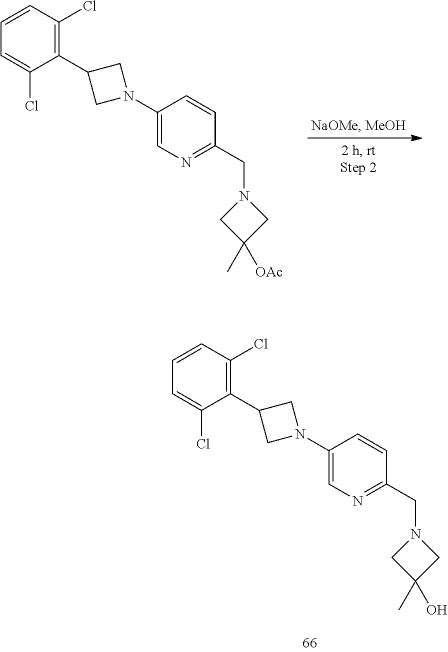 [SMILES: CC(=O)OC1(C)CN(Cc2ccc(N3CC(c4c(Cl)cccc4Cl)C3)cn2)C1 ; CC1(O)CN(Cc2ccc(N3CC(c4c(Cl)cccc4Cl)C3)cn2)C1]

To a stirred solution of 1-((5-(3-(2,6-dichlorophenyl) azetidin-1-yl) pyridin-2-yl)methyl)-3-methylazetidin-3-yl acetate (450 mg, 1.071 mmol) in methanol (5 mL) was added sodium methoxide (87 mg, 1.606 mmol). The resulting mixture was stirred at room temperature for 1 h. The reaction mixture was quenched with saturated solution of ammonium chloride and extracted with EtOAc. The combined organic layer was dried over sodium sulphate, concentrated under vacuum. The crude was purified using Prep. HPLC(Diluent: THF: Water: ACN (50:20:30); Column: Luna C18 (250×21.2) mm, 10 micron; Mobile phase A: 0.1% Formic acid in water; Mobile phase B: Acetonitrile) to afford 1-((5-(3-(2,6-dichlorophenyl) azetidin-1-yl) pyridin-2-yl)methyl)-3-methylazetidin-3-ol, formic acid salt (12 mg, 2.5% yield).

¹H NMR (400 MHZ, MeOD): ò 7.88 (d, J=2.80 Hz, 1H), 7.40 (d, J=8.00 Hz, 2H), 7.22-7.30 (m, 2H), 6.99 (dd, J=2.80, 8.40 Hz, 1H), 4.78-4.82 (m, 2H), 4.55 (t, J=8.40 Hz, 2H), 4.29 (t, J=8.00 Hz, 2H), 4.13 (s, 2H), 3.84 (d, J=10.40 Hz, 2H), 3.70 (d, J=10.00 Hz, 2H), 1.50 (s, 1H)(2H are merged with solvent signal); LCMS method 1, LCMS (ESI, m/z): 380.0 [M+H]⁺.

### Example S67. 1-(4-(3-(2,6-dichlorophenyl) azetidin-1-yl)-3-fluorobenzyl)-3-methylazetidin-3-ol (67)

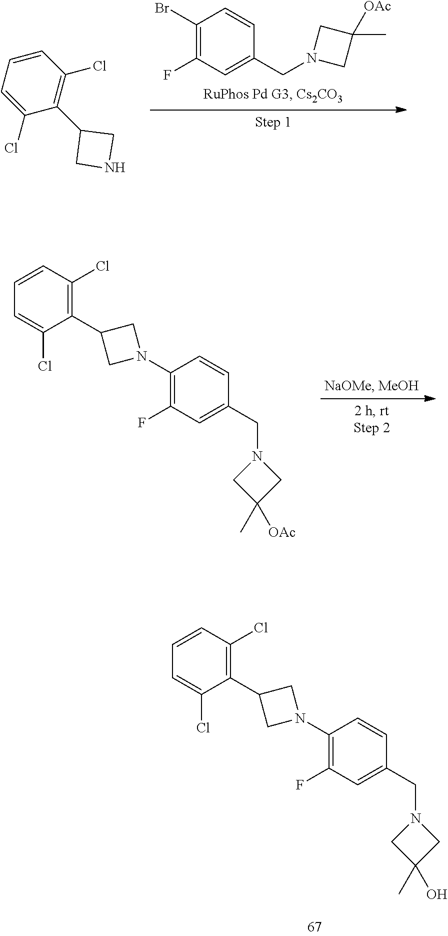 [SMILES: Clc1cccc(Cl)c1C1CNC1 ; CC(=O)OC1(C)CN(Cc2ccc(Br)c(F)c2)C1 ; CC(=O)OC1(C)CN(Cc2ccc(N3CC(c4c(Cl)cccc4Cl)C3)c(F)c2)C1 ; CC1(O)CN(Cc2ccc(N3CC(c4c(Cl)cccc4Cl)C3)c(F)c2)C1]

### Synthesis of 1-(4-(3-(2,6-dichlorophenyl) azetidin-1-yl)-3-fluorobenzyl)-3-methylazetidin-3-yl acetate

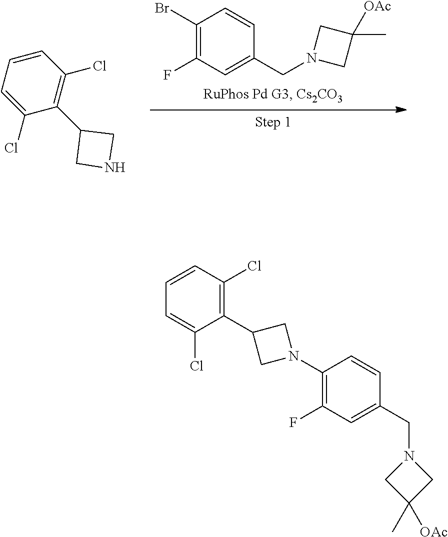 [SMILES: Clc1cccc(Cl)c1C1CNC1 ; CC(=O)OC1(C)CN(Cc2ccc(Br)c(F)c2)C1 ; CC(=O)OC1(C)CN(Cc2ccc(N3CC(c4c(Cl)cccc4Cl)C3)c(F)c2)C1]

To a solution of 3-(2,6-dichlorophenyl) azetidine, TFA (180 mg, 0.569 mmol) and 1-(4-bromo-3-fluorobenzyl)-3-methylazetidin-3-yl acetate (200 mg, 0.633 mmol) in anhydrous 1,4 dioxane (5 mL) was added cesium carbonate (618 mg, 1.898 mmol). The reaction mixture was then degassed with nitrogen for 10 min followed by the addition of added XPhos Pd G4 (54.4 mg, 0.063 mmol) and heated 100° C. After 16 h, TLC analysis indicated complete conversion of the starting material. The reaction mixture was then cooled to room temperature and filtered through a celite pad washed with EtOAc. The filtrate was concentrated under reduced pressure and the residue thus obtained was purified by flash column chromatography on silica gel (230-400 mesh) eluting with 0-50% ethyl acetate in petroleum ether to afford 1-(4-(3-(2,6-dichlorophenyl) azetidin-1-yl)-3-fluorobenzyl)-3-methylazetidin-3-yl acetate (74 mg, 19.5% yield) as a Orange brown solid; LCMS method 3, LCMS (ESI, m/z): 437.0 [M]⁺.

### Synthesis of 1-(4-(3-(2,6-dichlorophenyl) azetidin-1-yl)-3-fluorobenzyl)-3-methylazetidin-3-ol (67)

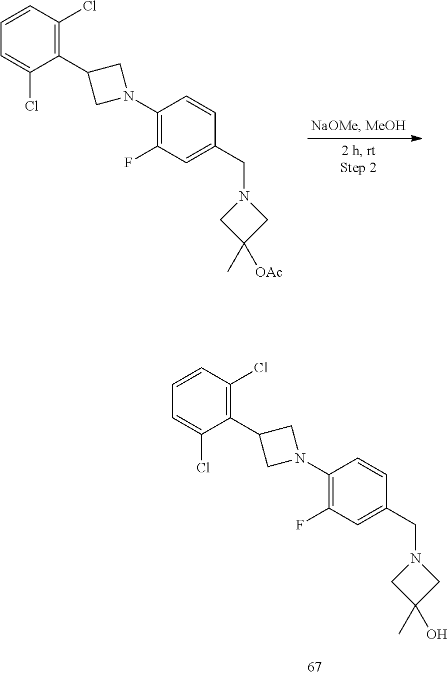 [SMILES: CC(=O)OC1(C)CN(Cc2ccc(N3CC(c4c(Cl)cccc4Cl)C3)c(F)c2)C1 ; CC1(O)CN(Cc2ccc(N3CC(c4c(Cl)cccc4Cl)C3)c(F)c2)C1]

To a stirred solution of 1-(4-(3-(2,6-dichlorophenyl) azetidin-1-yl)-3-fluorobenzyl)-3-methylazetidin-3-yl acetate (200 mg, 0.457 mmol) in methanol (5 mL) was added sodium methoxide (24.71 mg, 0.457 mmol). The resulting mixture was stirred at room temperature for 1 h. The reaction mixture was quenched with saturated solution of ammonium chloride and extracted with EtOAc. The combined organic layer was dried over sodium sulphate, concentrated under vacuum. The crude was purified using Prep. HPLC(Diluent: THF: Acetonitrile (30:70); Column-1: Sunfire C18 (150×19) mm, 5 micron; Mobile phase A: 0.1% Formic acid in water; Mobile phase B: Acetonitrile) to afford 1-(4-(3-(2,6-dichlorophenyl) azetidin-1-yl)-3-fluorobenzyl)-3-methylazetidin-3-ol, formic acid salt (17 mg, 0.038 mmol, 8.41% yield) as a pale yellow semi-solid.

¹H NMR (400 MHZ, MeOD): δ 7.39 (d, J=8.00 Hz, 2H), 7.23 (t, J=8.00 Hz, 1H), 7.08-7.09 (m, 2H), 6.68 (d, J=8.00 Hz, 1H), 4.57-4.64 (m, 3H), 4.27-4.31 (m, 2H), 4.12 (s, 2H), 3.89 (d, J=10.40 Hz, 2H), 3.76 (d, J=10.40 Hz, 2H), 1.51 (s, 3H). LCMS method 1; LCMS (ESI, m/z): 396.0 [M+H]⁺.

### Biological Examples

### Example B1. Cell membrane preparations

CHO cells expressing recombinant S1P5 receptors were cultured in 500 cm² culture trays and, once confluent, rinsed and detached with cell-lifting buffer (10 mM HEPES, 154 mM NaCl, 6.85 mM EDTA, pH 7.4). Cells were then pelleted by centrifugation, resuspended, and homogenized in membrane preparation buffer (10 mM HEPES and 10 mM EDTA, pH 7.4) using a Polytron PT 1200E homogenizer (Kinematica, Luzern, Switzerland). Cellular proteins were pelleted by centrifugation at 48,000×g at 4° C. for 30 minutes. The resulting supernatant was discarded, and the pellet was re-suspended again in membrane preparation buffer, homogenized for a second time, and then centrifuged again as described above. The final cellular protein pellet was suspended in ice cold resuspension buffer (10 mM HEPES and 0.1 mM EDTA, pH 7.4), divided into aliquots, and stored at −80° C. until use.

### Example B2. GTPγS binding assay

Functional binding assays for [³⁵S]-GTPγS were performed in 96-well non-binding surface plates with a final volume of 200 μL. The test compounds were serially diluted in DMSO and added to assay plates using a Tecan D300E digital printer with a total volume of 0.4 μL. The control sphingosine-1-phosphate (SIP) was prepared separately by preparing a 400 UM stock solution from a 100 nmol pellet of S1P in 10 mM Na₂CO₃ with 2% β-cyclodextrin. The serial dilution of S1P was done using complete assay buffer (20 mM HEPES, 10 mM MgCl₂, 100 mM NaCl, 1 mM EDTA, 0.1% fatty acid free bovine serum albumin (BSA), and 30 μg/mL saponin, pH 7.4) and transferred to wells already containing 0.4 μL DMSO. All the wells were then loaded to a total volume of 40 μL of complete assay buffer, except the non-specific binding (NSB) wells. For NSB wells, 40 μL/well of 50 μM GTPγS (Sigma Aldrich, cat #G8634, St. Louis, MO) was added to wells containing 0.4 μL of DMSO. The assay was started by the addition of 120 μL/well of CHO-SIP receptor membrane solution containing 40 μg/mL of membrane protein, 16.67 μM guanosine diphosphate (GDP; Sigma Aldrich, cat #G7127, St. Louis, MO), and 2.5 mg/mL of WGA PVT SPA beads in complete buffer. Assay plates were then sealed and incubated at room temperature with gentle agitation for 30 minutes. Next, 40 μL/well of 1 nM of [³⁵S]-GTPγS (PerkinElmer, cat #NEG030X²⁵⁰UC, Waltham, MA) in basic assay buffer (20 mM HEPES, 10 mM MgCl₂, 100 mM NaCl, and 1 mM EDTA, pH7.4) was added to the assay plates to yield a final concentration of 200 μM and the plates were further incubated for 40 minutes at room temperature with gentle agitation. The assay was terminated by centrifugation of the plates at 1000 rpm for 3 minutes using an Eppendorf 5810R centrifuge (Eppendorf, Hamburg, Germany) and G protein bound radioactivity was quantitated using a MicroBeta2 microplate scintillation counter (PerkinElmer, Waltham, MA). As G protein bound radioactivity directly correlates to receptor activation and coupling to the G protein, this assay is a measure of S1P5 agonism. Results are shown in Table 2.

TABLE 2
S1P5 GTPγS Binding of Exemplary Compounds.

| Compound No. | Activity nM |
|---|---|
| 1a | +++ |
| 1b | +++ |
| 2a | ++ |
| 2b | +++ |
| 3a | ++++ |
| 3b | +++ |
| 4a | +++ |
| 4b | ++ |
| 5a | +++ |
| 5b | +++ |
| 6 | + |
| 7 | ++ |
| 8 | + |
| 9 | ++ |
| 10a | ++++ |
| 10b | ++++ |
| 11 | + |
| 12 | ++ |
| 13a | +++ |
| 13b | +++ |
| 14a | ++++ |
| 14b | ++++ |
| 15a | ++++ |
| 15b | ++++ |
| 16a | ++++ |
| 16b | ++++ |
| 17a | +++ |
| 17b | +++ |
| 18a | ++++ |
| 18b | ++++ |
| 19a | ++++ |
| 19b | ++++ |
| 20a | +++ |
| 20b | ++ |
| 21a | +++ |
| 21b | ++++ |
| 22a | +++ |
| 22b | +++ |
| 23a | +++ |
| 23b | +++ |
| 24a | ++++ |
| 24b | ++++ |
| 25a | ++++ |
| 25b | ++++ |
| 26a | ++++ |
| 26b | ++++ |
| 27a | ++++ |
| 27b | +++ |
| 28 | ++ |
| 29 | ++ |
| 30 | + |
| 31 | +++ |
| 32 | + |
| 33 | ++ |
| 34 | ++ |
| 35 | +++ |
| 36 | ++ |
| 37 | ++++ |
| 38 | ++ |
| 39 | ++ |
| 40 | ++ |
| 41 | ++ |
| 42 | +++ |
| 43 | + |
| 44 | + |
| 45 | + |
| 46 | ++ |
| 47 | ND |
| 48 | +++ |
| 49 | +++ |
| 50 | +++ |
| 51 | +++ |
| 52 | ++ |
| 53 | + |
| 54 | +++ |
| 55 | +++ |
| 56 | +++ |
| 57 | +++ |
| 58 | + |
| 59 | ++ |
| 60 | ++ |
| 61 | ++ |
| 62 | ++++ |
| 63 | ++++ |
| 64 | ++ |
| 65 | +++ |
| 66 | ++ |
| 67 | +++ |

ND = not determined
++++ indicates binding between greater than 1 nM and ≤ 10 nM
+++ indicates binding between greater than 10 nM and ≤ 100 nM
++ indicates binding between greater than 100 nM and ≤ 1,000 nM
+ indicates binding between greater than 1,000 nM and ≤ 10,000 nM

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A compound of Formula (I): 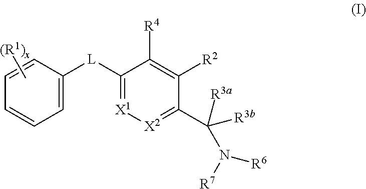
or a pharmaceutically acceptable salt thereof, wherein:
L is —C≡C—, —HC═CH—, —CH₂CH₂—, —CH₂O—, 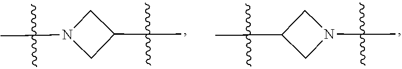 [SMILES: *C1CN(*)C1 ; *C1CN(*)C1]
or a bond;
each R¹ is independently halo, —CN, C₁-C₆ alkyl, C₁-C₆ haloalkyl, C₁-C₆ alkoxy, or C₃-C₆ cycloalkyl;
x is 0-5;
R² is H, halo, C₁-C₆ alkyl, C₃-C₆ cycloalkyl, or C₁-C₆ haloalkyl;
R^{3a} and R^{3b} are each H;
or R² and R^{3a} are taken together with the carbon atoms to which they are attached to form a fused cyclopentyl;
or R² and R⁴ are taken together with the carbon atoms to which they are attached to form a fused phenyl;
R⁴ is H, halo, —CN, C₁-C₆ alkyl, C₁-C₆ haloalkyl, C₁-C₆ alkoxy, or C₃-C₆ cycloalkyl;
X¹ and X² are independently N or CR⁵;
each R⁵ is independently H, halo, —CN, C₁-C₆ alkyl, C₁-C₆ haloalkyl, C₁-C₆ alkoxy, or C₃-C₆ cycloalkyl;
R⁶ is H;
R⁷ is C₁-C₆ alkyl-OH;
or R⁶ and R⁷ are taken together with the nitrogen atom to which they are attached to form a 4- to 6-membered heterocyclyl substituted with n R⁸ groups;
n is 1-5; and
each R⁸ is independently halo, —CN, C₁-C₆ alkyl, C₁-C₆ haloalkyl, C₁-C₆ alkoxy, or —OH, provided that at least one R⁸ is —OH.

2. The compound of claim 1, or a pharmaceutically acceptable salt thereof, wherein:
L is —C≡C—, —CH₂CH₂—, —CH₂O—, or a bond.

3. The compound of claim 1, or a pharmaceutically acceptable salt thereof, wherein:
L is

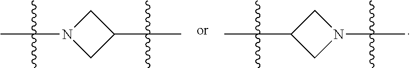 [SMILES: *C1CN(*)C1 ; *C1CN(*)C1]

4. The compound of any one of claims 1-3, or a pharmaceutically acceptable salt thereof, wherein:
each R¹ is independently halo, —CN, C₁-C₃ alkyl, C₁-C₃ haloalkyl, C₁-C₃ alkoxy, or C₃-C₆ cycloalkyl.

5. The compound of any one of claims 1-4, or a pharmaceutically acceptable salt thereof, wherein:

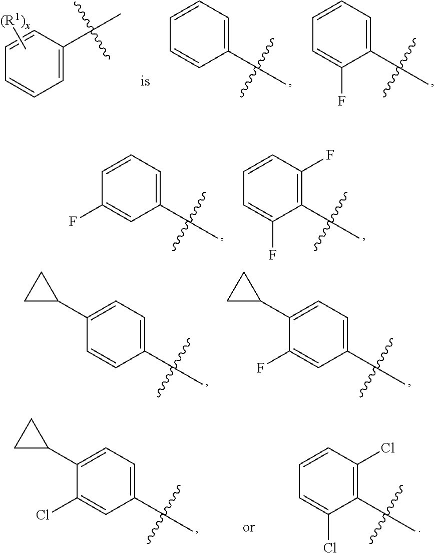

6. The compound of any one of claims 1-5, or a pharmaceutically acceptable salt thereof, wherein:
R² is H, halo, C₁-C₃ alkyl, C₃-C₆ cycloalkyl, or C₁-C₃ haloalkyl; and
R^{3a} and R^{3b} are each H.

7. The compound of any one of claims 1-5, or a pharmaceutically acceptable salt thereof, wherein:
R² and R^{3a} are taken together with the carbon atoms to which they are attached to form a fused cyclopentyl; and
R^{3b} is H.

8. The compound of any one of claims 1-5, or a pharmaceutically acceptable salt thereof, wherein:
R² and R⁴ are taken together with the carbon atoms to which they are attached to form a fused phenyl.

9. The compound of any one of claims 1-7, or a pharmaceutically acceptable salt thereof, wherein:
R⁴ is H, halo, —CN, C₁-C₃ alkyl, C₁-C₃ haloalkyl, C₁-C₃ alkoxy, or C₃-C₆ cycloalkyl.

10. The compound of any one of claims 1-9, or a pharmaceutically acceptable salt thereof, wherein:
each R⁵ is independently H, halo, —CN, C₁-C₃ alkyl, C₁-C₃ haloalkyl, C₁-C₃ alkoxy, or C₃-C₆ cycloalkyl.

11. The compound of any one of claims 1-10, or a pharmaceutically acceptable salt thereof, wherein:

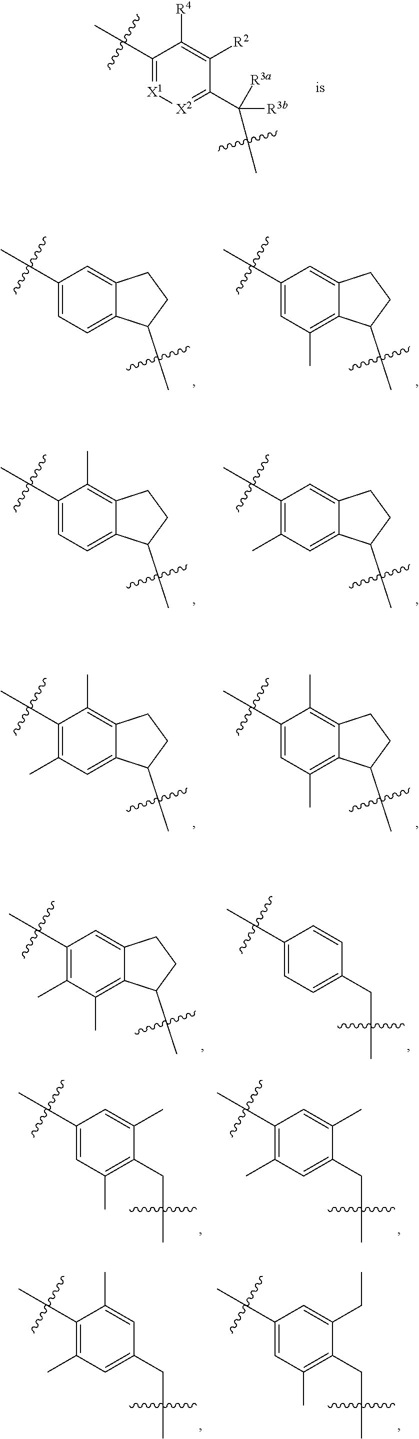

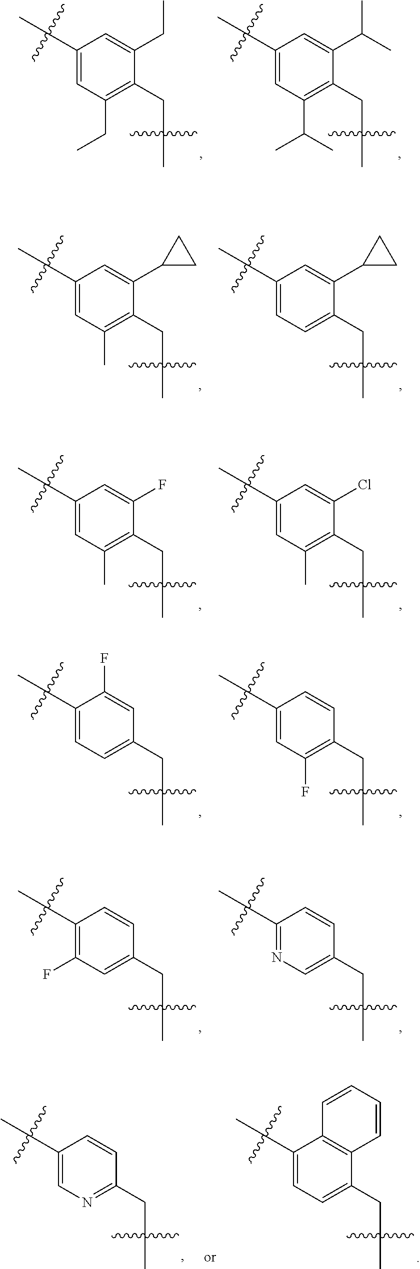

12. The compound of any one of claims 1-11, or a pharmaceutically acceptable salt thereof, wherein:
R⁶ is H; and
R⁷ is C₁-C₆ alkyl-OH.

13. The compound of any one of claims 1-11, or a pharmaceutically acceptable salt thereof, wherein:
R⁶ and R⁷ are taken together with the nitrogen atom to which they are attached to form 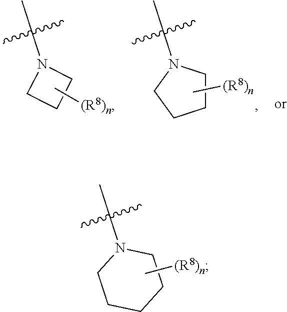
and
each R⁸ is independently halo, —CN, C₁-C₃ alkyl, C₁-C₃ haloalkyl, C₁-C₃ alkoxy, or —OH.

14. The compound of any one of claims 1-11 and 13, or a pharmaceutically acceptable salt thereof, wherein:

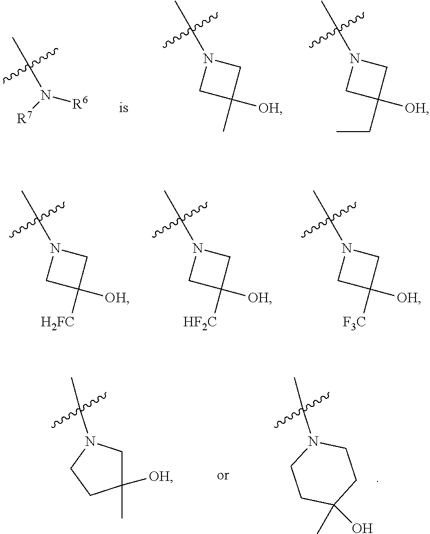

15. The compound of any one of claims 1-5, 7, and 9-14, or a pharmaceutically acceptable salt thereof, wherein the compound is of Formula (II):

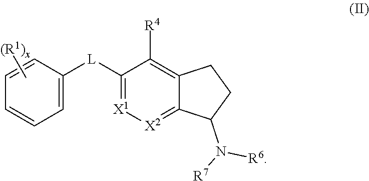

16. The compound of any one of claims 1-6 and 8-14, or a pharmaceutically acceptable salt thereof, wherein the compound is of Formula (III):

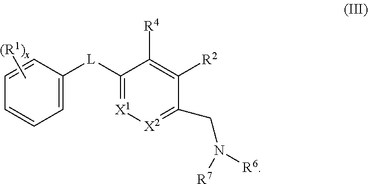

17. A compound selected from the compounds of Table 1 and pharmaceutically acceptable salts thereof.

18. A pharmaceutical composition comprising the compound of any one of claims 1-17, or a pharmaceutically acceptable salt thereof, and a pharmaceutically acceptable excipient.

19. A method of modulating sphingosine 1-phosphate receptor 5 (S1P5) comprising contacting SIPS with an effective amount of the compound of any one of claims 1-17, or a pharmaceutically acceptable salt thereof, or the pharmaceutical composition of claim 18.

20. A method of treating a neurological disease in a subject in need thereof, comprising administering to the subject an effective amount of the compound of any one of claims 1-17, or a pharmaceutically acceptable salt thereof, or the pharmaceutical composition of claim 18, optionally wherein the neurological disease is Alzheimer's disease, multiple sclerosis, migraine, and amyotrophic lateral sclerosis.
